(19) United States
(12) Patent Application Publication
(10) Pub. No.: US 2022/0409704 A1
(43) Pub. Date: Dec. 29, 2022

(54) Methods for Treating Cancer That Has A Phosphatidylinositol 3,4,5-Trisphosphate Rac Exchange Factor 2 (PREX2) Protein Expressed Thereon

(71) Applicant: Kaohsiung Medical University, Kaohsiung City (TW)

(72) Inventor: Yi-Ming Chen, Kaohsiung City (TW)

(73) Assignee: Kaohsiung Medical University, Kaohsiung City (TW)

(21) Appl. No.: 17/929,407

(22) Filed: Sep. 2, 2022

Related U.S. Application Data
(63) Continuation-in-part of application No. 16/488,439, filed on Aug. 23, 2019.
(60) Provisional application No. 62/462,964, filed on Feb. 24, 2017.

(51) Int. Cl. A61K 38/45; A61P 35/00; C12N 15/113; A61K 48/00

(52) U.S. Cl. CPC A61K 38/45; A61P 35/00; C12N 15/1135; A61K 48/005

(57) ABSTRACT

Disclosed herein is a method of treating a cancer in a subject having or suspected of having the cancer that has a mutated PREX2 expressed thereon. According to the embodiment of the present disclosure, the mutation is G258V, S1113R, E1346D or K400fs. The method includes the step of administering an effective amount of a composition to the subject, wherein the composition includes a therapeutic molecule for producing a polypeptide that exhibits a binding affinity to the mutated PREX2 protein expressed in the cancer in the subject.

### CROSS-REFERENCE TO RELATED APPLICATION AND CLAIM OF PRIORITY

This application is a Continuation-In-Part of co-pending U.S. patent application Ser. No. 16/488,439, which was filed on Aug. 23, 2019, for which priority is claimed under 35 U.S.C. § 120, and which is a 35 U.S.C. § 371 national stage application of PCT/CN2018/076711, which was filed on Feb. 13, 2018, at the National Intellectual Property Administration of the People's Republic of China (PRC) as the Receiving Office of the World Intellectual Property Office (WIPO), and claimed the benefit of U.S. Patent Application No. 62/462,964, which was filed on Feb. 24, 2017, at the U.S. Patent and Trademark Office (USPTO), all of which are incorporated herein by reference as if fully set forth.

The contents of the electronic sequence listing filed herewith, having a file size of 299,128 bytes and created on Sep. 2, 2022, is incorporated herein by reference as if fully set forth.

### FIELD OF THE INVENTION

The present disclosure in general relates to the field of cancer treatment and medical applications. More particularly, the present disclosure relates to a method for treating a cancer in a subject having or suspected of having the cancer that has a phosphatidylinositol 3,4,5-trisphosphate Rac exchange factor 2 (PREX2) protein expressed thereon.

### BACKGROUND OF THE INVENTION

Cancer is a complex disease, in which the cells in a specific tissue are no longer fully responsive to the signals within the tissue that regulate cellular differentiation, survival, proliferation and death. As a result, these cells accumulate within the tissue, and then cause local damage and inflammation. More than 200 different types of cancer have been identified in humans.

Hepatocellular carcinoma (HCC) is one of the most prevalent cancers in the world. In adult men, it is the fifth most frequently diagnosed cancer worldwide, and is the second leading cause of cancer-related death. As for adult women, it is the seventh most commonly diagnosed cancer and the sixth leading cause of cancer death. Different factors have been reported to be associated with the development of HCC, including alcohol abuse, viral infection (e.g., hepatitis B virus (HBV) or hepatitis C virus (HCV) infection), liver cirrhosis, obesity, type II diabetes and food contamination (e.g., aflatoxin and arsenic).

Nowadays, transplantation remains the best option for patients with HCC. However, there is a limited supply of good-quality deceased donor organs. Several alternative treatments have thus been developed to bridge patients before receiving a transplant or to delay recurrence, such as resection, radiofrequency ablation (RFA), chemotherapy, transcatheter arterial chemoembolization and radiation therapy. In general, early diagnosis of HCC is crucial for optimizing treatment strategy. Compared with advanced HCC cases lacking the possibility of effective treatment, the detection of HCC at an early stage allows the use of potentially curative treatment which can improve the survival chances of patients. It has been reported that the 5-year survival rate of patients diagnosed as having advanced HCC is 0% to 10%. In contrast, when HCC is detected at an early stage, the 5-year survival rate is more than 50%. Unfortunately, most HCC patients are diagnosed at a late stage due to lack of symptoms or signs of early stage of HCC.

In view of the foregoing, there exists in the related art a need for a method for early detection of HCC so that a proper and prompt treatment may be provided to the patient. In addition, there is also a need for a method that can effectively treat the subject having or suspected of having HCC.

### SUMMARY OF THE INVENTION

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

The present invention discloses a method of treating a cancer in a subject having or suspected of having the cancer that has a phosphatidylinositol 3,4,5-trisphosphate Rac exchange factor 2 (PREX2) protein expressed thereon, wherein the expressed PREX2 protein is a mutated PREX2 protein including at least one mutation selected from the group consisting of G258V, S1113R, E1346D and K400fs when compared to SEQ ID NO: 7, the method including the step of administering an effective amount of a composition to the subject having or suspected of having the cancer that has the PREX2 protein expressed thereon, wherein the composition includes a therapeutic molecule for producing a polypeptide that exhibits a binding affinity to the mutated PREX2 protein expressed in the cancer in the subject.

Many of the attendant features and advantages of the present disclosure will becomes better understood with reference to the following detailed description considered in connection with the accompanying drawings.

### BRIEF DESCRIPTION OF THE DRAWINGS

The patent or application file contains at least one drawing executed in color. Copies of this patent or patent application publication with color drawings will be provided by the Office upon request and payment of the necessary fee.

The present description will be better understood from the following detailed description read in light of the accompanying drawings.

FIG. 1A depicts that Huh7-GNMT stable cells were harvested for co-immunoprecipitation (co-IP) assay followed by immunoblot (IB) analysis.

FIG. 1B depicts that mouse liver lysates were used for reciprocal co-IP assay followed by IB analysis.

FIG. 1C depicts that HepG2-GNMT stable cells were treated with or without proteasome inhibitor MG132 and harvested for IB analysis.

FIG. 1D depicts that Huh7 cells were transfected with indicated plasmids and 35S-Methionine/Cysteine incorporation measurements were used to determine PREX2 half-life.

FIG. 1E depicts the in vivo ubiquitination assay in Huh7 cells being transfected with Myc-PREX2, HA-Ub (ubiquitin), along with Flag-GNMT.

FIG. 1F depicts that Huh7 cells were infected with Lentiviruses expression short hairpin RNA (shRNA) targeting GNMT or LacZ (shLacZ) and harvested for IB analysis.

FIG. 1G depicts that the IB and quantification analysis of PREX2 and pAKT (Ser473) protein expression in the livers of 14- to 15-month-old female wild type mice (5 mice) and GNMT^{-/-} mice (18 mice). The quantified results are presented as means±standard error of the mean (SEM), *p<0.05.

FIG. 2A depicts the in vivo ubiquitination assay in HEK293T cells being transfected with Myc-PREX2, His-Ub, along with various E3 ligases.

FIG. 2B depicts that Huh7 cells with control or HectH9 knockdown were harvested for IB analysis.

FIG. 2C depicts that Huh7 cells with control or HectH9 knockdown were treated with cycloheximide (CHX) for the indicated hour and harvested for IB analysis.

FIG. 2D depicts that the control- and HectH9-knockdown Huh7 cells were treated with the proteasome inhibitor MG132 and harvested for the in vivo ubiquitination assay.

FIG. 3A depicts the IB analysis of lysed Huh7 cells with control or HectH9 knockdown.

FIG. 3B depicts that cell proliferation was measured in Huh7 cells with control or HectH9 knockdown.

FIG. 3C depicts the HCC tumor development in non-obese diabetes/severe combined immunodeficiency (NOD/SCID) mice bearing Huh7 cells with control- or HectH9-knockdown (n=5/group).

FIG. 3D depicts the histological and quantification analysis of Ki-67 protein expression in xenograft tumors. Scale bar represents 300 μm, *p<0.05 and **p<0.01.

FIG. 4A depicts the IB analysis of PREX2 expression in 51 pairs of HCC tumor and tumor-adjacent (TA) tissues. **p<0.01.

FIG. 4B depicts the real-time PCR analysis of PREX2 mRNA levels and quantification analysis of PREX2 expression in 51 pairs of HCC tumor and TA tissues.

FIG. 4C depicts the Pearson correlation analysis of PREX2 protein expression with GNMT.

FIG. 4D depicts the Kaplan-Meier plot analysis of 51 cases of HCC patients with low or high protein expression of PREX2.

FIG. 5 depicts the non-silent mutations within PREX2 in HCC tumors.

FIG. 6 depicts the sequencing depth of PREX2 genome coverage. Histograms of reads over the entirety of PREX2 in chromosome 8 (chr8) from human HCC and matched germline DNA and ˜288 kbp reading region (chr8:68864603-69143897) is covered.

### DETAILED DESCRIPTION OF THE PREFERRED EMBODIMENTS

The detailed description provided below in connection with the appended drawings is intended as a description of the present examples and is not intended to represent the only forms in which the present example may be constructed or utilized.

The description sets forth the functions of the example and the sequence of steps for constructing and operating the example. However, the same or equivalent functions and sequences may be accomplished by different examples.

In the present invention, unless otherwise required by context, the term “comprise”, “include” and “contain” will be comprehended to indicate to include steps or elements or the combination of the steps or the elements, but it does not exclude other steps or elements or the combination of the steps or the elements. Thus, the use of the term “comprise” and so on means that the listed elements are necessary or compulsory, but other elements are optional and may be absent. The term “consist of . . . ” refers to include and limit to the elements behind the term “consist of . . . ”. Thus, the term “consist of . . . ” means that the listed elements are necessary or compulsory, and no other elements exist. The term “basically consist of . . . ” refers to include any elements listed behind this term, and is limited to not interfere or promote the indicated activities or functions of other elements in the listed elements of the present invention, but other elements are optional depending on whether other elements influence the activities or functions of the listed elements.

The present invention describes the functions of the examples, and illustrates that the examples are used to combine or operate the sequences of the steps of the examples. However, the same or equivalent functions or sequences still can be accomplished by other additional examples.

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “effective amount” as referred to herein designate the quantity of a component which is sufficient to yield a desired response. For therapeutic purposes, the effective amount is also one in which any toxic or detrimental effects of the component are outweighed by the therapeutically beneficial effects. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, in grams, milligrams or micrograms or as milligrams per kilogram of body weight (mg/Kg). Alternatively, the effective amount can be expressed in the concentration of the active component (e.g., the therapeutic molecule of the present disclosure), such as molar concentration, mass concentration, volume concentration, molality, mole fraction, mass fraction and mixing ratio. Specifically, the term “therapeutically effective amount” used in connection with the therapeutic molecule described herein refers to the quantity of the therapeutic molecule, which is sufficient to alleviate or ameliorate the symptoms associated with the cancers in the subject. Persons having ordinary skills could calculate the human equivalent dose (HED) for the medicament (such as the present therapeutic molecule) based on the doses determined from animal models. For example, one may follow the guidance for industry published by U.S. Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The term “subject” refers to a mammal including the human species that is treatable with methods of the present invention. The term “subject” is intended to refer to both the male and female gender unless one gender is specifically indicated.

The glycine-N-methyltransferase (GNMT)-binding protein, phosphatidylinositol 3,4,5-trisphosphate Rac exchange factor 2 (PREX2) (SEQ ID NO: 7), is a phosphatase and tensin homologue deleted on chromosome 10 (PTEN)-binding protein to inhibit the activity of PTEN. However, the present invention is based, at least in part, on the result that some point mutations in PREX2 gene (SEQ ID NO: 8) are associated with the development, metastasis and/or recurrence of cancers.

According to some embodiments of the present disclosure, these point mutations include three non-silent mutations and one frameshift truncation mutation; the PREX2 polypeptides encoded thereby thus respectively comprise the mutation of G258V, S1113R, E1346D and K400fs (a truncated form of PREX2 polypeptide) when compared to SEQ ID NO: 7. The expression profile of PREX2 gene provides a potential means to efficiently detect cancer cells or make a prognosis of a subject if he/she has or is at the risk of developing cancer.

Accordingly, the first aspect of the present disclosure is directed to a method for making a diagnosis or a risk evaluation of a cancer in a subject. The method includes the steps of: (a) obtaining a biological sample from the subject; (b) extracting DNA from the biological sample; and (c) detecting the presence or absence of a mutation in the PREX2 gene.

According to certain embodiments of the present disclosure, the biological sample is obtained from the subject having or at risk of developing a cancer. The biological sample may be a biopsy sample, a whole blood sample, a plasma sample, a serum sample, a urine sample or a mucus sample. In one preferred embodiment, the biological sample is a whole blood sample including circulating cancer cells therein. Then, the DNA can be extracted from the biological sample by use of a commercial kit (e.g., Quagen® DNA extraction kit) or by any method familiar to the skilled artisan, for example, the treatment of lysis buffer or sonication. The extracted DNA then serves as a template to analyze the gene profile by an assay, such as direct sequencing, single-strand conformation polymorphism (SSCP), denaturing gradient gel electrophoresis (DGG) or temperature gradient gel electrophoresis (TGGE). According to one working example, the extracted DNA is analyzed by HaloPlex® target enrichment sequencing in the purpose of determining whether or not a mutation is present in the biological sample.

According to the embodiment of the present disclosure, the mutation is G773T, A3337C, A4038T or 1200 delG when compared to SEQ ID NO: 8; and the presence of the mutation indicates the subject has or is at risk of developing a cancer, wherein G773T means that guanine (G) at position 773 of the PREX2 gene is replaced by thymine (T), A3337C means that adenine (A) at position 3337 of the PREX2 gene is replaced by cytosine (C), A4038T means that adenine (A) at position 4038 of the PREX2 gene is replaced by thymine (T), and 1200 delG means that guanine (G) at position 1200 of the PREX2 gene is deleted.

According to some embodiments of the present disclosure, the cancer cell may simultaneously include more than one mutation in PREX2 gene; that is, the cancer cell may simultaneously include any two, three or four mutations of G773T, A3337C, A4038T or 1200 delG in PREX2 gene.

According to certain embodiments of the present disclosure, the mutations of G773T, A3337C, A4038T or 1200 delG in PREX2 gene (SEQ ID NO: 8) respectively result in G258V, 51113R, E1346D and K400fs mutations in PREX2 polypeptide (SEQ ID NO: 7). Thus, instead of detecting the mutation of PREX2 gene, the cancer cell may be identified or predicted via analyzing the PREX2 protein sequence. Thus, the method for making a diagnosis or a risk evaluation of a cancer in a subject via detecting the mutated polypeptide includes the steps of: (a) obtaining a biological sample from the subject; (b) isolating the PREX2 protein from the biological sample; and (c) detecting the presence or absence of a mutation in the PREX2 polypeptide, wherein the mutation is G258V, S1113R, E1346D or K400fs.

According to the embodiment of the present disclosure, the mutation occurs between the nucleotide 773 and the nucleotide 4038 of the PREX2 gene.

According to the embodiment of the present disclosure, the mutation is selected from the group consisting of G773T, A3337C, A4038T, 1200 delG and the combination thereof.

According to the embodiment of the present disclosure, when mutation is detected in the PREX2 polypeptide, the subject has or is at risk of developing a cancer.

The biological sample may be a biopsy sample, a whole blood sample, a plasma sample, a serum sample, a urine sample or a mucus sample. According to one working example, the biological sample is a whole blood sample including circulating cancer cells therein. Then, total protein is isolated from the biological sample. Exemplary methods suitable for isolating protein from the biological sample include, but are not limited to, repeated freezing and thawing, sonication, homogenization (e.g., the use of French press or beads) and the treatment of detergent (e.g., According to the embodiment of the present disclosure, sodium dodecyl sulfate (SDS), Triton X-100 or NP-40) with or without enzyme (e.g., lysozyme) contained therein. The isolated protein may be subject to a sequencing assay, such as mass spectrometry, thioacylation reaction or Edman degradation reaction, so as to determine the presence or absence of the mutation described above.

As mentioned above, the cancer cell may simultaneously include more than one mutation in PREX2 gene, which then encodes a PREX2 polypeptide including more than one mutation. In one embodiment, the cancer cell having a mutated PREX2 polypeptide expressed thereon that includes any two of G258V, S1113R, E1346D and K400fs. In another embodiment, the cancer cell having a mutated PREX2 polypeptide expressed thereon that includes any three of G258V, S1113R, E1346D and K400fs. In still another embodiment, the mutated PREX2 polypeptide expressed on the surface of cancer cell simultaneously includes G258V, S1113R, E1346D and K400fs.

As would be appreciated, the presence of a mutation (either in PREX2 gene or PREX2 polypeptide) may be applicable to make a prognosis as to whether or not a cancer spreads or relapses in a subject. In clinical practice, metastatic and recurrent cancer cells usually develop drug resistance and increased invasive property as compared to the original cancer (also known as primary cancer). It is reported that more than half of cancer patients die from metastatic or recurrent cancers that develop months, years or even decades (depending on the cancer types) after primary tumor removal. Early identification of cancer metastasis or relapse allows the appropriate treatments to be promptly administered to the subject so as to improve his/her therapeutic outcome and lifespan.

According to some embodiments of the present disclosure, the presence of the mutation in PREX2 gene (i.e., G773T, A3337C, A4038T or 1200 delG) or PREX2 polypeptide (i.e., G258V, S1113R, E1346D or K400fs) indicates the subject is at risk of developing metastatic and/or recurrent cancers.

Basically, the subject is a mammal; preferably a human. According to some embodiments of the present disclosure, the subject is an Asian. In one specific example, the subject is a Chinese.

The cancer cell diagnosed or predicted by any of the present methods may be isolated from gastric cancer, lung cancer, bladder cancer, breast cancer, pancreatic cancer, renal cancer, colorectal cancer, cervical cancer, ovarian cancer, brain tumor, prostate cancer, hepatocellular carcinoma (HCC), melanoma, esophageal carcinoma, multiple myeloma, or head and neck squamous cell carcinoma. According to one specific example, the cancer is HCC.

Another aspect of the present disclosure is directed to a method for treating the subject identified by the present method as having a primary cancer, a metastatic cancer and/or a recurrent cancer. The method includes: administering to the subject an effective amount of a therapeutic molecule.

According to one embodiment of the present disclosure, the therapeutic molecule is a molecule for producing glycine-N-methyltransferase (GNMT) including the amino acid sequence of SEQ ID NO: 1. Preferably, the therapeutic molecule is a polynucleotide encoding GNMT that including the amino acid sequence of SEQ ID NO: 1. According to another embodiment of the present disclosure, the therapeutic molecule is a molecule for producing ubiquitin ligase (homologous to E6AP carboxyl terminus homologous protein 9, HectH9) including the amino acid sequence of SEQ ID NO: 2. Preferably, the therapeutic molecule is a polynucleotide encoding HectH9 including the amino acid sequence of SEQ ID NO: 2. According to still another embodiment of the present disclosure, the therapeutic molecule includes a first polynucleotide encoding the amino acid sequence of SEQ ID NO: 1 and a second polynucleotide encoding the amino acid sequence of SEQ ID NO: 2.

According to still another embodiment of the present disclosure, the therapeutic molecule is a small interfering RNA (siRNA) that downregulates the expression of PREX2 mRNA. Alternatively, the therapeutic molecule can be an anti-cancer drug selected from the group consisting of anti-estrogens (e.g. tamoxifen, raloxifene, and megestrol), luteinising-hormone releasing hormone (LHRH) agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (Abraxane), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel (e.g. 2′-paclitaxel methyl 2-glucopyranosyl succinate), docetaxel, taxol, epipodophyllins (e.g. etoposide, etoposide phosphate, teniposide, topotecan, 9-aminocamptothecin, camptoirinotecan, irinotecan, crisnatol, and mytomycin C), anti-metabolites, dihydrofolate reductase (DHFR) inhibitors (e.g. methotrexate, dichloromethotrexate, trimetrexate, edatrexate), inosine monophosphate (IMP) dehydrogenase inhibitors (e.g. mycophenolic acid, tiazofurin, ribavirin, and 5-ethynyl-1-beta-D-ribofuranosylimidazole-4-carboxamide (EICAR)), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mammalian target of rapamycin (mTOR) inhibitors (e.g., rap amycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

As would be appreciated, the subject having the mutation (either in PREX2 gene or in PREX2 polypeptide) can be alternatively treated by a conventional treatment (such as resection, radiofrequency ablation (RFA), chemotherapy, transcatheter arterial chemoembolization and radiation therapy), an anti-angiogenic therapy or an immunotherapy.

Another aspect of the present disclosure pertains to a method of treating a subject having or suspected of having a cancer. According to some embodiments of the present disclosure, the cancer has PREX2 overexpressed thereon/therein. According to other embodiments of the present disclosure, the cancer has mutated PREX2 expressed thereon/therein, in which the mutated PREX2 includes at least one mutation selected from the group consisting of G258V, S1113R, E1346D and K400fs. The present method includes administering to the subject an effective amount of a therapeutic molecule.

Preferably, the therapeutic molecule is an inhibitor of PREX2. According to one embodiment of the present disclosure, the inhibitor of PREX2 is a siRNA that downregulates the expression of PREX2 mRNA. According to another embodiment of the present disclosure, the inhibitor of PREX2 is a molecule for producing a polypeptide including the sequence of SEQ ID NO: 1 or 2 that inhibits the proliferation and/or induces the death (i.e., necrosis or apoptosis) of cancer cells via enhancing the degradation of PREX2. According to still another embodiment of the present disclosure, two different inhibitors of PREX2 (such as a first molecule for producing a first polypeptide including the sequence of SEQ ID NO: 1 and a second molecule for producing a second polypeptide including the sequence of SEQ ID NO: 2) can be used together to inhibit the proliferation and/or induce the death of cancer cells. According to one working embodiment of the present disclosure, the polypeptide of SEQ ID NO: 1 or 2 increases the sensitivity of cancer cells to an anti-cancer drug, for example, sorafenib.

In the embodiments of the present invention, to verify the feasibility of the above-mentioned method, the polypeptide of SEQ ID NO: 1 or 2 was produced by mammalian system. Specifically, the polynucleotide encoding the polypeptide of SEQ ID NO: 1 or 2 may be introduced into a mammalian cell (e.g., 293T cell) via calcium phosphate co-precipitation, electroporation, microinjection, nucleofection, cell squeezing (gently squeezing the cell membrane), sonoporation (inducing pore formation in cell membrane by high-intensity ultrasound), optical transfection (generating a tiny hole in cell membrane by highly focused laser), impalefection (inserting into a cell DNA bound to the surface of a nanofiber), gene gun (“shooting” into the cell nucleus DNA coupled to a nanoparticle of an inert solid), magnetofection (using magnetic force to deliver DNA into target cells), viral transduction (using viruses as a carrier to deliver DNA into target cells), or transfection via a dendrimer, a liposome, or a cationic polymer. The cell introduced with the polynucleotide is then incubated under suitable condition (depending on the cell types; for example, 37° C. with 5% CO₂ for 293T cell) so as to produce the present polypeptide. Alternatively, the cell introduced with the polynucleotide may be delivered to the subject through surgical techniques (i.e. cell implantation).

Preferably, the present therapeutic molecule is conjugated with a targeting molecule, which exhibits binding affinity to the polypeptide expressed on cancer cells (i.e., PREX2 or mutated PREX2 polypeptide including G258V, 51113R, E1346D and/or K400fs). Thus, once administered into the subject, the therapeutic molecule can be directed to the cancer cells via the interaction between the targeting molecule and the polypeptide. Depending on the desired purpose, the targeting molecule can be an antibody or an aptamer.

Exemplary cancers treatable by the method in accordance with any aspect and embodiment of the present disclosure include, but are not limited to, gastric cancer, lung cancer, bladder cancer, breast cancer, pancreatic cancer, renal cancer, colorectal cancer, cervical cancer, ovarian cancer, brain tumor, prostate cancer, hepatocellular carcinoma (HCC), melanoma, esophageal carcinoma, multiple myeloma, and head and neck squamous cell carcinoma. According to one example, the cancer is HCC.

According to one specific embodiment, the cancer is a drug-resistant cancer.

Basically, the subject treatable by the method in accordance with any aspect and embodiment of the present disclosure is a mammal, for example, a human, a mouse, a rat, a hamster, a guinea pig, a rabbit, a dog, a cat, a cow, a goat, a sheep, a monkey, and a horse. Preferably, the subject is a human. According to one working example, the subject is an Asian.

The present method can be applied to the subject, alone or in combination with additional therapies that have some beneficial effects on the treatment of cancer. Depending on the therapeutic purpose, the present method can be applied to the subject before, during, or after the administration of the additional therapies.

The therapeutic molecule in accordance with any of the aspects and embodiments of the present disclosure may be administered to the subject via a route selected from the group consisting of oral, enteral, nasal, topical, transmucosal, and parenteral administration, in which the parenteral administration is any of subcutaneous, intratumoral, intradermal, intramuscular, intraarticular, intravenous, intraspinal, or intraperitoneal injection.

The following examples are provided to elucidate certain aspects of the present invention and to aid those skilled in the art in practicing this invention. These examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

Materials and Methods

PREX2 Expression

The tumorous (T) tissue, tumor-adjacent (TA) tissue and peripheral blood mononuclear cells were isolated from HCC patients followed by reverse transcription and real-time polymerase chain reaction (PCR) and immunoblotting analysis. The primers used for the real-time PCR were PREX2-F: 5′-GAGATTGCCG CACCAGAGA-3′ (SEQ ID NO: 3) and PREX2-R: 5′-TCAAGGACAT GGTGCATAAA TCC-3′ (SEQ ID NO: 4) for PREX2; and TBP-F: 5′-CAGAAGTTGG GTTTTCCAGT CAA-3′ (SEQ ID NO: 5) and TBP-R: 5′-ACATCACAGC TCCCCACCAT-3′ (SEQ ID NO: 6) for TATA-box binding protein (TBP). Predicted cycle threshold (CT) values were exported into EXCEL worksheets for analysis. Comparative CT methods were used to determine fold difference in gene expression relative to TBP. The antibodies for immunoblotting were anti-PREX2 antibody (Sigma) and anti-β-actin (Sigma).

Detection of Gene Mutation

The DNA extracted from the tumor, TA tissue or peripheral blood mononuclear cells of 30 HCC patients was analyzed by HaloPlex target enrichment sequencing.

HaloPlex Target Enrichment Sequencing

All DNA samples were evaluated for several quality criteria prior to Illumina sequencing. DNA concentration was measured using Epoch system (BioTek) and DNA amount more than 500 ng were taken into consideration. In addition, structural integrity of DNA was checked by gel electrophoresis, and degraded samples were removed from consideration. The HaloPlex target enrichment system (Agilent Technologies, Santa Clara, Calif., USA) was used to capture and determined the PREX2 gnomic DNA sequence by the specifically designed probes as recommended by manufacturer's instruction. The genomic DNA (200-250 ng) from HCC specimens were fragmented by restriction enzyme digestion and circularized by hybridization to probes whose ends are complementary to the target fragments. The probe contains a method-specific sequencing motif that is incorporated during the circularization. The circular molecules were then closed by ligation and target DNA were captured using streptavidin beads. Amplified circular DNA targets were enriched and subjected to sequencing.

Sequence Data Processing and Identification of Somatic Mutation

Adapter sequence trimming, quality filtering, coverage determination and the initial assembly of sequence contigs were performed using GEMINI mainframe program and CLC Genomics Workbench (www.cicbio.com/products/clc-genomics-workbench/). Candidate single nucleotide polymorphism (SNP) and insertion-deletion (indel) mutations were identified by comparison of read realignment with Single Nucleotide Polymorphism database (dbSNP). To identify somatic substitutions and indels, the realignment from HCC tumor and matched germline DNA with reference genome (Ref_NCBI_GRCh37_hg19) were compared by using GEMINI, and known SNPs present in the dbSNP database were filtered out.

### Example 1: Regulation of PREX2 Expression

1.1 Interaction Between GNMT and PREX2

In this embodiment, a polynucleotide encoding GNMT was introduced into HEK293T, Huh7 or HepG2 cells via a plasmid to overexpress GNMT in these cells, such that the interaction between GNMT and PREX2 as well as the resulting effects can be investigated. To test whether GNMT interacts with PREX2, immunoprecipitation (IP) assays were performed using cell lysates from HEK293T cells co-transfected with PREX2 and GNMT expressing plasmids. The data of reciprocal co-IP assays confirmed that GNMT co-immunoprecipitated with PREX2. Moreover, the recombinant GNMT overexpressed in Huh7 cells was purified followed by the analysis of co-IP and immunoblot assays.

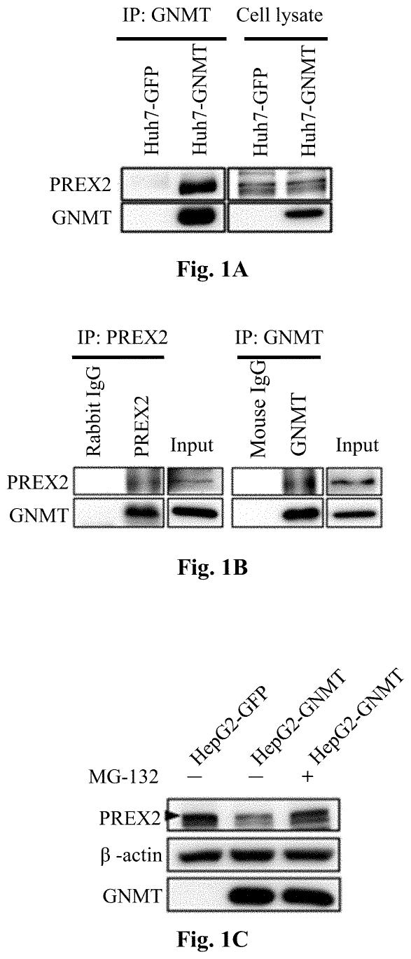

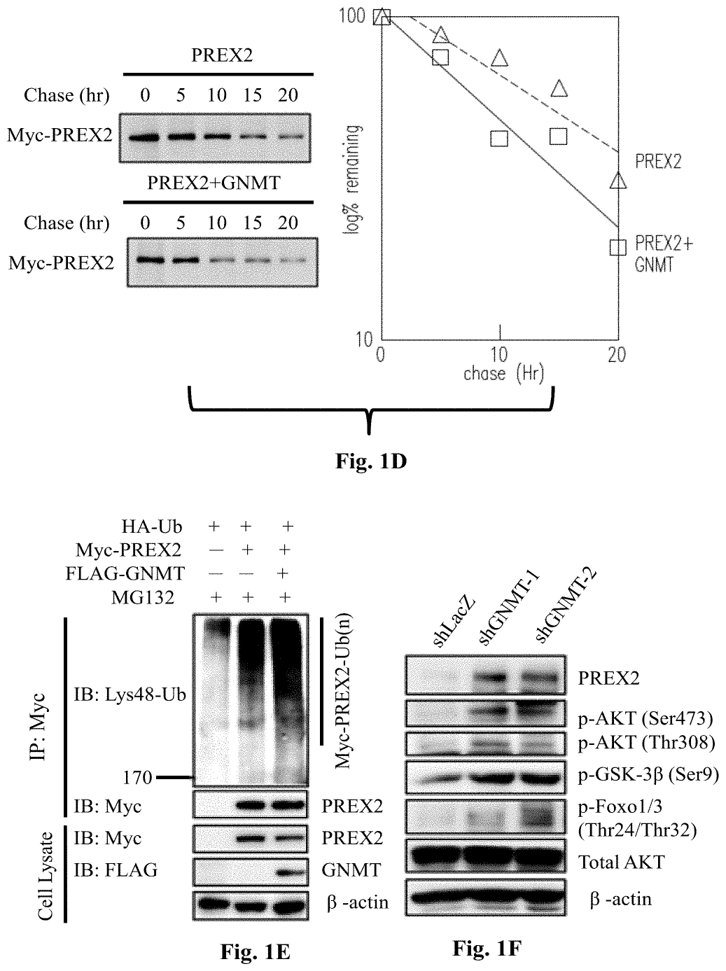

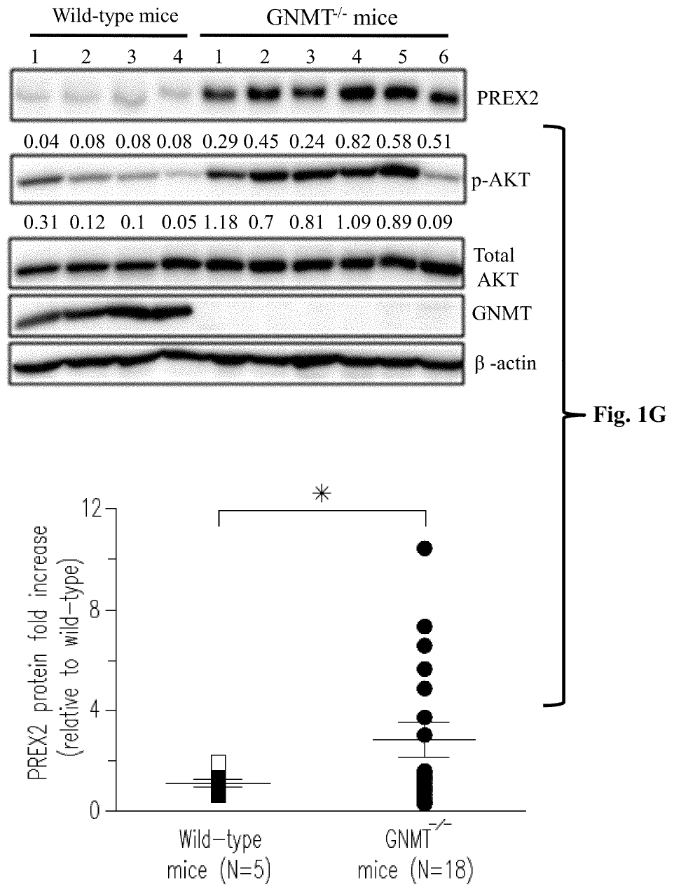

Please refer to FIG. 1A to FIG. 1G, which illustrates that GNMT interacts with PREX2 and negatively regulates PREX2-mediated AKT signaling. As the data in FIG. 1A illustrated, GNMT interacted with endogenous PREX2. To demonstrate that GNMT interacted with PREX2 under physiological condition, reciprocal co-IP assays were performed using mouse liver lysates, and the results confirmed that endogenous GNMT co-immunoprecipitated with endogenous PREX2 specifically (referring to FIG. 1B). To map the binding domain, different Myc-tagged PREX2 truncated mutants were co-expressed with Flag-tagged GNMT in HEK293T cells. It is found that the paired PDZ domain mediated its interaction with GNMT. Furthermore, in vitro pull-down experiment using purified GST-GNMT and His-Myc-tagged PDZ domains of PREX2 demonstrated that GNMT bind to PREX2 directly.

To determine the effects of PREX2 interacting with GNMT, the expression of PREX2 in HCC cells overexpressed GNMT was monitored. It was found that endogenous expression PREX2 was significantly reduced in HepG2 cells overexpressed GNMT, and this effect was reversed by treatment with proteasome inhibitor-MG132 (referring to FIG. 1C). Moreover, when a mutant GNMT-N140S which contains <0.5% its enzymatic activity was expressed in HepG2 cells, similar phenomenon was observed. The data suggested that the reduction of endogenous PREX2 caused by GNMT was independent of its methyltransferase activity. Furthermore, pulse-chase experiments revealed that expression of GNMT significantly shortened the half-life of PREX2 from 15 hours to 9.4 hours (referring to FIG. 1D). A similar result was observed when the cells were treated with cycloheximide (CHX). Since the K48-linked poly-ubiquitin chain on the target protein is the major signal for proteasomal degradation, the ubiquitination assay was performed so as to investigate whether GNMT promoted K48-linked ubiquitination formation on PREX2 protein. The data of FIG. 1E illustrated that GNMT overexpression promoted K48-linked ubiquitination of PREX2 in the presence of MG132. Since PREX2 is a component of the PI3K-AKT pathway, the immunoblot assay was performed to determine the effects of this interaction on PI3K-AKT cascade. Knockdown of GNMT in Huh7 cells resulted in a significant increase of the PREX2 protein while it did not affect the PREX2 mRNA levels (referring to FIG. 1F). However, subsequent increases of the phosphorylation of AKT at Thr308 and Ser473 were found (referring to FIG. 1F). This increased AKT phosphorylation was found to be correlated with the phosphorylation level of glycogen synthase kinase 38 (referring to GSK38, a known AKT substrate, in FIG. 1F). Further, the increase of AKT phosphorylation was PREX2-dependent as inhibition of both GNMT and PREX2 expression reversed the AKT activation. To further investigate whether GNMT deficiency correlated with PREX2 expression in vivo, PREX2 abundance in the liver of wild-type and GNMT^{-/-} mice was measured by immunoblot and quantification analysis. Compared to the wild-type mice, GNMT^{-/-} mice had significantly higher PREX2 protein levels and such correlation was associated with AKT activation (referring to FIG. 1G). Taken together, these results suggest that GNMT negatively regulates PREX2 function through the ubiquitin-proteasome pathway. These data suggest that the overexpressed GNMT can interact with endogenous PREX2, downregulate the function of PREX2 in cancer cells, and inhibit tumor growth.

1.2 Interaction Between HectH9 and PREX2

In the ubiquitination pathway, E3 ligases serve as the specific substrate-recognition element of the system. To identify which E3 ligase is responsible for ubiquitin-dependent PREX2 degradation, a panel of E3 ligases for PREX2 ubiquitination was screened in the presence of MG132.

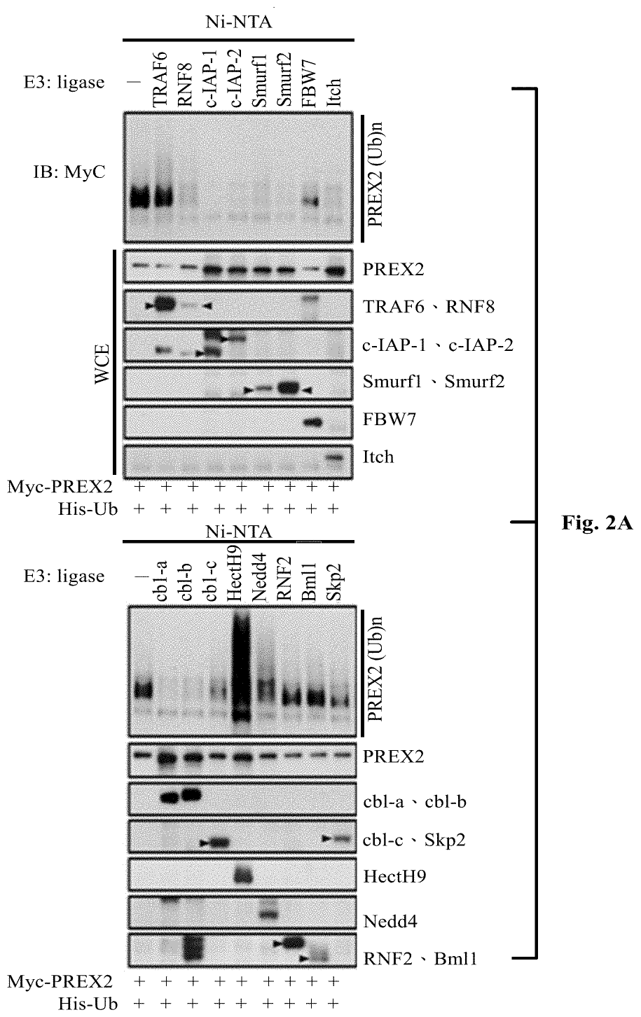

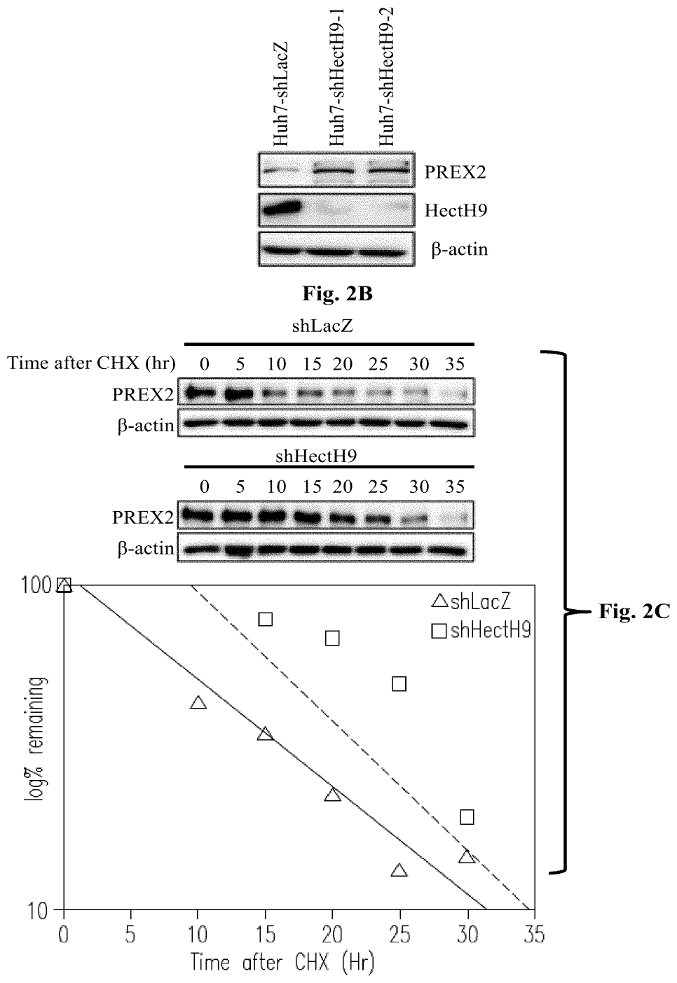

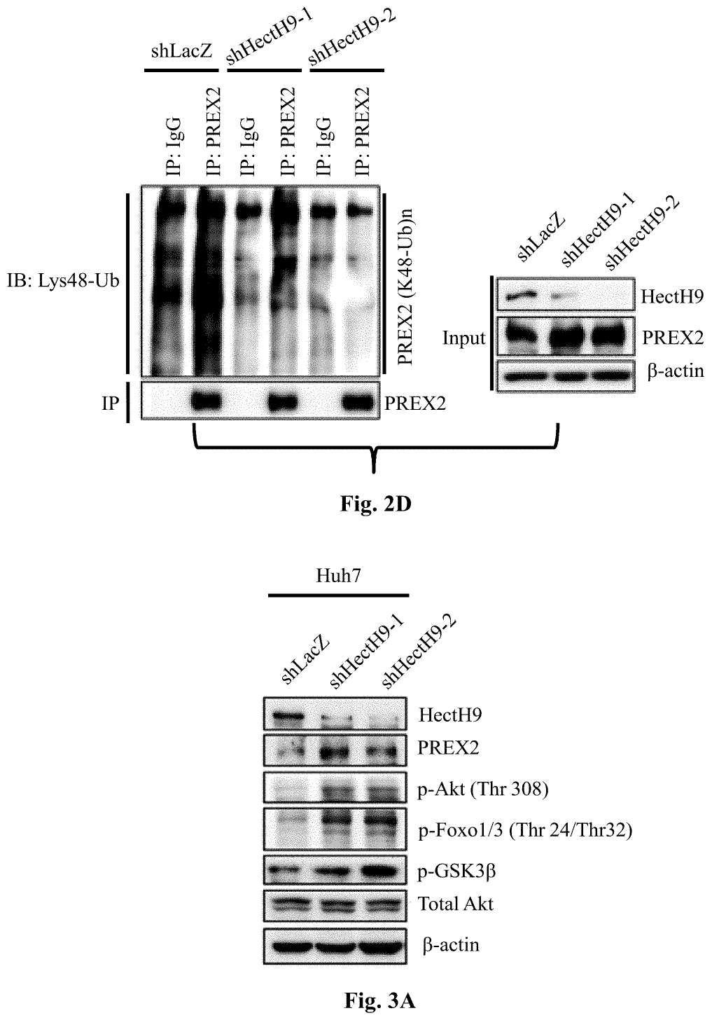

Please refer to FIGS. 2A to 2D, which illustrate that HectH9 associates with PREX2, and leads to its ubiquitination and degradation. In the experiment as shown in FIG. 2A, polynucleotides encoding various E3 ligases were each introduced into HEK293T cells via a plasmid to overexpress E3 ligases in these cells. The results indicated that among those E3 ligases, overexpression of homologous to E6AP carboxyl terminus homologous protein 9 (HectH9, also known as HuWe1, Mule or ARF-BP1) strongly enhanced PREX2 ubiquitination in vivo (referring to FIG. 2A). HectH9 belongs to the Hect-domain family of ubiquitin ligases, which are characterized by a conserved carboxy-terminal catalytic domain. Various substrates of HectH9 have been reported to be involved in apoptosis (Md-1) and transcriptional regulation (p53, c-Myc and N-Myc). Co-IP assay demonstrated that HectH9 interacted with the paired PDZ and Inspx4 domains of PREX2. To determine whether HectH9 affected the steady-state levels of PREX2, endogenous PREX2 expression in HCC cells with HectH9 knockdown was detected by immunoblot analysis. Compared to the control, Huh7 cells infected with lentiviruses expressing short hairpin RNAs (shRNAs) targeting HectH9 had increased levels of PREX2 proteins (referring to FIG. 2B). Similar effect was also observed in HepG2 cells. Cycloheximide treatment revealed that the increase in PREX2 abundance following HectH9 depletion was greatly due to the increase in the half-life of PREX2 protein (referring to FIG. 2C). Notably, the level of endogenous K-48 linked ubiquitination of PREX2 was markedly diminished upon HectH9 depletion (referring to FIG. 2D).

1.3 HectH9 Inhibits Tumor Growth

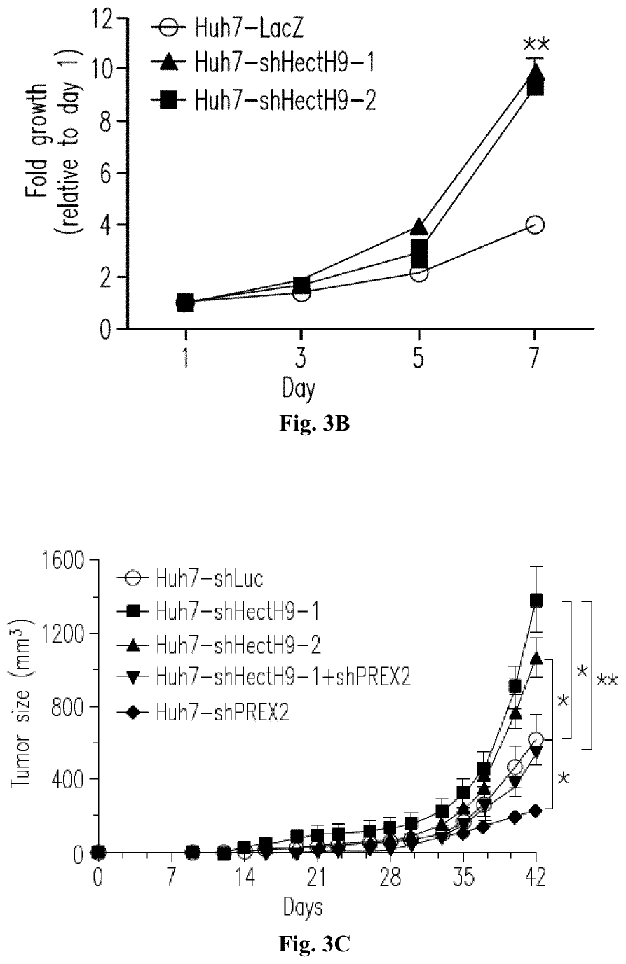

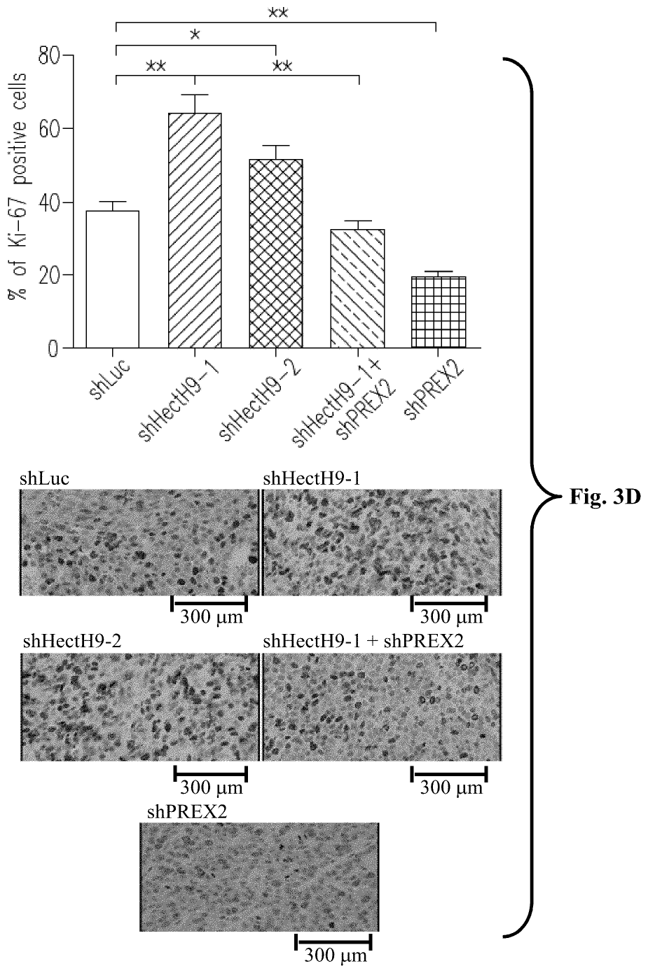

To determine the biological significance of PREX2 degradation by HectH9 in HCC cells, HectH9 was knocked down in a pair of PTEN-wild type cell lines. Huh7 and HepG2 followed by measuring the activity of the AKT pathway. Please refer to FIGS. 3A to 3D, which illustrate that HectH9 regulates PREX2-mediated AKT signaling, cell proliferation and HCC tumor growth. HectH9 suppression in Huh7 cells increased the phosphorylation of AKT and AKT substrates, including GSK3@, Foxo1 and Foxo3a (referring to FIG. 3A), and enhanced cell proliferation (referring to FIG. 3B). Likewise, elevated cell proliferation was observed in HepG2 cells. Moreover, the increased AKT phosphorylation and cell proliferation were dependent on PREX2 function, as suppressing both HectH9 and PREX2 expression reversed AKT activation and cell proliferation.

To further investigate whether HectH9 regulated liver cancer development in vivo, the impact of HectH9 expression on tumor growth in a xenograft model was monitored. The knockdown of HectH9 through RNAi interference largely increased the growth of xenograft tumors (referring to FIG. 3C). Moreover, suppression of both HectH9 and PREX2 expression reversed tumor growth, indicating that HectH9-mediated HCC tumor growth was dependent on PREX2 function (referring to FIG. 3C). The data of immunohistochemical staining indicated that HectH9 suppression led to upregulation of Ki-67 expression, whereas such effect could be rescued by further knockdown of PREX2 expression (FIG. 3D). Thus, the regulation of HectH9 in HCC cells was dependent on PREX2 function. These data suggest that the overexpressed HectH9 can interact with endogenous PREX2, lead to its degradation, and inhibit tumor growth.

Based on the experimental data above, it can be concluded that a molecule for producing a polypeptide that exhibits a binding affinity to the PREX2 protein, such as the molecule resulting in overexpression of GNMT or HectH9, can be employed as the therapeutic molecule in the present invention for treating the cancer expressing PREX2 protein.

1.4 Interaction Among GNMT, PREX2 and HectH9

In this embodiment, Huh7 cells were co-transfected with three plasmids for overexpressing GNMT, PREX2 and HectH9 proteins and harvested for co-IP and immunoblotting assays. Sequential co-IP was performed so as to evaluate the interactive relationship among GNMT, PREX2 and HectH9. The results illustrated that GNMT, paired PDZ domains of PREX2 and HectH9 were all present in the second co-IP, indicating that they form a complex. In addition, co-IP experiments revealed that HectH9 interacted more efficiently with PREX2 when GNMT was co-expressed. To determine whether GNMT-mediated regulation of PREX2 is associated with HectH9, HectH9 was knocked down in GNMT overexpressed HCC cells. Strikingly, depletion of HectH9 in Huh7 cells reversed GNMT-mediated down-regulation of PREX2 expression. Likewise, similar effect on PREX2 expression was observed in HepG2 cells. Moreover, depletion of HectH9 in Huh7 cells resulted in suppression of GNMT-promoted K48-linked ubiquitination of PREX2. Overexpression of GNMT significantly decreased Huh7 proliferation, whereas this effect was reversed after depletion of HectH9. Thus, the regulation of PREX2 by GNMT was associated with HectH9. Based on the experimental data above, it can be concluded that the therapeutic molecule in the present invention may include more than one molecule for producing polypeptides that exhibit binding affinity to the PREX2 protein. Preferably, the therapeutic molecule in the present invention may include a first polynucleotide encoding GNMT and a second polynucleotide encoding HectH9.

### Example 2: PREX2 Expression in HCC Patients

2.1 PREX2 Overexpression in HCC Patients

GNMT expression was down-regulated in both human HCC cell lines and tumor tissues. To investigate the expression profiles of PREX2 in clinical specimen, PREX2 expression in tumorous (T) and tumor-adjacent (TA) tissues isolated from HCC patients was examined.

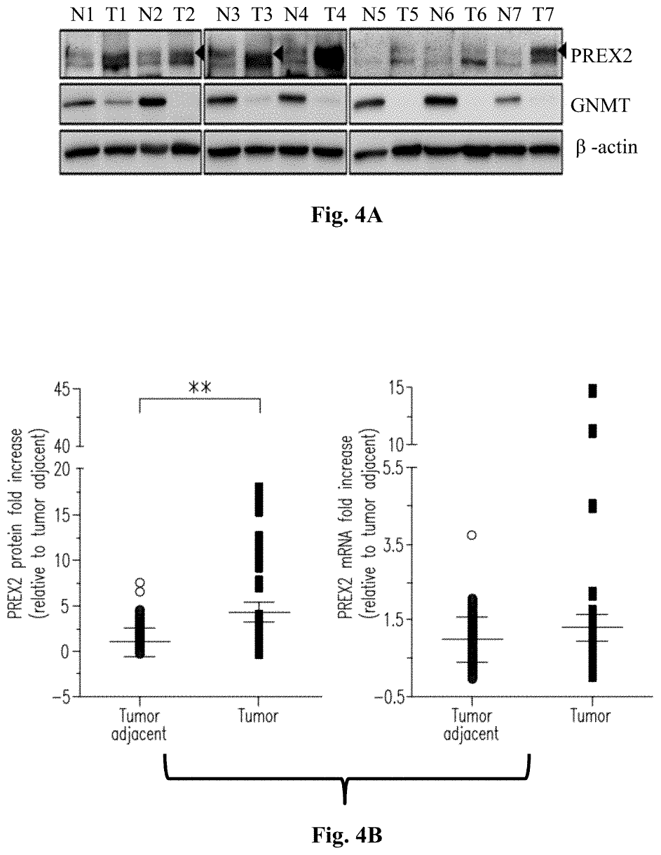

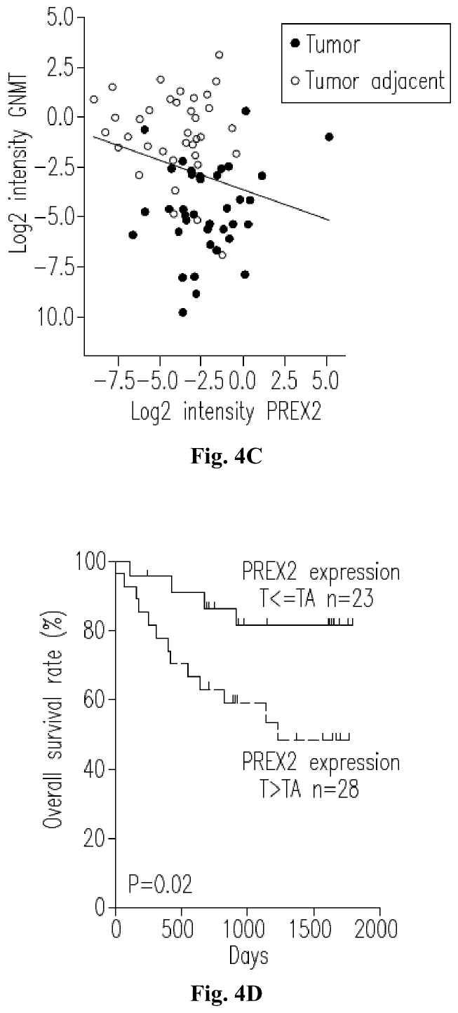

Please refer to FIG. 4A to FIG. 4D, which illustrate the expression profiles of PREX2 in human HCC and the association with survival. As the data of western blot assays illustrated, the levels of PREX2 protein were significantly higher in tumorous tissues (T group) than in the corresponding TA tissues (TA group) in 54.9% (28/51) HCC patients (referring to FIGS. 4A and 4B). By contrast, the levels of PREX2 mRNA were similar in both T and TA groups (referring to FIG. 4B), further supporting the notion that the regulation of PREX2 expression by GNMT is a post-translational regulation. PREX2 mRNA expression in another cohort consisting of 88 HCC patients was further examined, and a similar result was observed. Moreover, a significant negative correlation was found between GNMT and PREX2 protein levels in tumor and TA tissues (r=−0.28; p=0.017, referring to FIG. 4C). Additionally, PREX2 overexpression was significantly correlated with the following clinical characteristics of the patients: viral infection (p=0.004), tumor size (p=0.03) and the levels of AFP (p=0.04). Multivariate logistic regression revealed that the expression of PREX2 significantly correlated with HBV infection (odd ratio 14.07, p=0.01). Furthermore, higher level of PREX2 in the tumor tissues was associated with poorer survival (p=0.02, referring to FIG. 4D). Cox proportional hazards model was used to evaluate the factor associated with prognosis of HCC patients, and the results indicated that the association between death and PREX2 overexpression was statistically significant (hazard ration, 3.36, p=0.03). Collectively, the results suggested that the level of PREX2 protein expression could be used to predict the survival outcome of HCC patients.

2.2 PREX2 Mutations in HCC Tumors

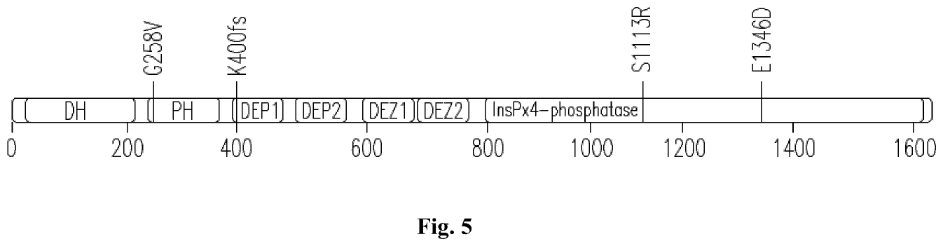

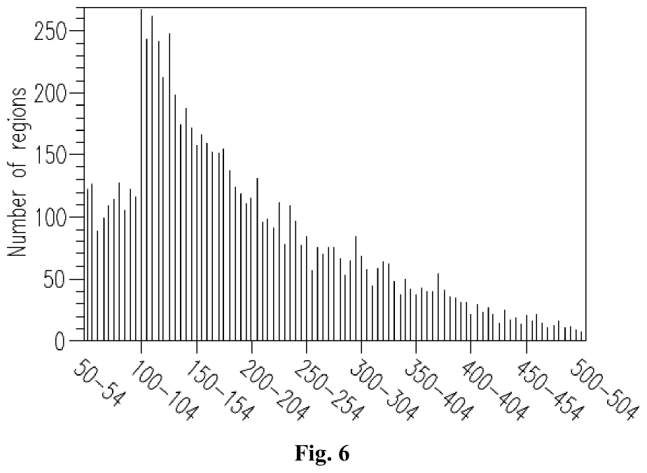

Previously, a 14% frequency of non-synonymous somatic PREX2 mutations in a melanoma cohort was reported. To investigate whether the PREX2 gene in HCC also harbors somatic mutations, the PREX2 genome extracted from the tumor, TA tissues and peripheral blood mononuclear cells of 30 HCC patients was analyzed by HaloPlex target enrichment sequencing. Please refer to FIG. 5, which illustrate non-silencing mutation within PREX2 in HCC tumors, wherein fs means frameshift deletion mutation, DH means DBL homology domain, PH means pleckstrin homology domain, DEP means dishevelled, Egl-10 and pleckstrin domain, and the C-terminal half of PREX2 displays sequence homology to an inositol phosphatase domain. The coverage rate of raw sequence mapped to the human PREX2 genome was 98.22%, and the sequencing depth was illustrated in FIG. 6. In total, 14 (46.7%) HCC tumors with 16 (53.3%) somatic mutations were found, which included 12 synonymous and 4 (13.3%) non-silent mutations (referring to Table. 1). Among these 4 non-silent mutations, there were 3 non-synonymous mutations and 1 frameshift truncation mutation (referring to FIG. 5).

TABLE 1
Summary of somatic mutations within
PREX2 in 30 human HCC patients

| Samples | Number | Samples | Number | Samples | Number |
|---|---|---|---|---|---|
| with | of somatic | with | of | with non- | of |
| somatic | point | synonymous | synonymous | silent | non-silent |
| mutations, | mutations | mutations, | mutations | mutations, | mutations |
| n (%) | (rate, %) | n (%) | (rate, %) | n | (rate, %) |
| 14 (46.7) | 16 (53.3) | 12 (40) | 12 (40) | 6 (20) | 4 (13.3) |

In summary, 20% (6/30) of HCC samples harbored at least 1 non-silent mutation in their PREX2 gene. In addition, the analysis of the mutant allele frequency and genotype revealed that all these three non-synonymous mutations were heterozygous (referring to Table. 2).

TABLE 2
The analysis of the genotype and allele frequency of
PREX2 mutations in 30 human HCC patients

| | | PBMC | | Tumor | | | PBMC | | Tumor | |
|---|---|---|---|---|---|---|---|---|---|---|
| Non-silent | | (%) | | (%) | | | (%) | | (%) | |
| mutation | Genotype | (N = 30) | | (N = 30) | | Allele | (N = 30) | | (N = 30) | |
| G258V G/T | GG | 30 | (100) | 29 | (96.7) | G | 60 | (100) | 59 | (98.3) |
| | GT | 0 | | 1 | (3.3) | T | 0 | | 1 | (1.7) |
| | TT | 0 | | 0 | | | | | | |
| S1113R A/C | AA | 30 | (100) | 29 | (96.7) | A | 60 | (100) | 59 | (98.3) |
| | AC | 0 | | 1 | (3.3) | C | 0 | | 1 | (1.7) |
| | CC | 0 | | 0 | | | | | | |
| E1346D A/T | AA | 30 | (100) | 29 | (96.7) | A | 60 | (100) | 59 | (98.3) |
| | AT | 0 | | 1 | (3.3) | T | 0 | | 1 | (1.7) |
| | TT | 0 | | 0 | | | | | | |
| K400fs G/A/Del | GG | 17 | (56.7) | 17 | (56.7) | G | 46 | (76.7) | 45 | (75) |
| | GA | 12 | (40) | 9 | (30) | A | 13 | (21.7) | 11 | (18.3) |

| | | | | | | | |
|---|---|---|---|---|---|---|---|
| AA | 0 | 0 | Del | 1 | (1.7) | 4 | (6.7) |

| G/del | 0 | | 2 | (6.7) |
|---|---|---|---|---|
| A/del | 1 | (3.3) | 2 | (6.7) |

| | | |
|---|---|---|
| del/del | 0 | 0 |

Thus, it is known that G773T means that glycine (G) at position 773 of the PREX2 gene is replaced by threonine (T), A3337C means that alanine (A) at position 3337 of the PREX2 gene is replaced by cysteine (C), A4038T means that alanine (A) at position 4038 of the PREX2 gene is replaced by threonine (T), and 1200 delG means that glycine (G) at position 1200 of the PREX2 gene is deleted to cause the non-silent mutation for K400fs.

Moreover, it is also known from Table 2 that 13.3% (4/30) HCC samples had a frameshift deletion mutation (K400fs) of PREX2 which resulted in a truncated form of PREX2 protein containing only DH and PH domains. Since DH domain is responsible for the regulation of the GEF activity and the PH domain inhibit PTEN phosphatase activity, further studies are needed to elucidate the role it plays in the oncogenesis of HCC.

Because the presence of the mutation in PREX2 polypeptide (i.e., G258V. S1113R, E1346D or K400fs) indicates that the HCC patient is at risk of developing metastatic and/or recurrent cancers, so the purpose of the present invention is to decrease this risk by providing the PREX2 inhibitor that inhibits the proliferation and/or induces the death of cancer cells. Based on the experimental data above, it is believed that the therapeutic molecule producing GNMT or HectH9 can be administrated to the HCC patients that express PREX2 or mutant PREX2 protein, so as to inhibit development, metastasis and/or recurrence of HCC.

In conclusion, a novel tumorigenic mechanism of dysregulation of PREX2 expression in a tumor environment was identified in this invention where GNMT expression is down-regulated. The present experimental data show that the level of PREX2 protein expression can be used to predict the prognosis of HCC patients, PREX2 and its variant mutants may serve as new therapeutic targets for HCC. It is known that the pharmaceutical compositions and kits of the present invention can be used in the applications, such as diagnosis, prognosis and treatment, of the cancers, e.g. HCC, and have novelty and inventiveness.

Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method of treating a cancer in a subject having or suspected of having the cancer that has a phosphatidylinositol 3,4,5-trisphosphate Rac exchange factor 2 (PREX2) protein expressed thereon, wherein the expressed PREX2 protein is a mutated PREX2 protein comprising at least one mutation selected from the group consisting of G258V, S1113R, E1346D and K400fs when compared to SEQ ID NO: 7, the method comprising:
administering an effective amount of a composition to the subject having or suspected of having the cancer that has the PREX2 protein expressed thereon, wherein the composition comprises a therapeutic molecule for producing a polypeptide that exhibits a binding affinity to the mutated PREX2 protein expressed in the cancer in the subject.

2. The method according to claim 1, wherein the therapeutic molecule is a polynucleotide encoding the polypeptide.

3. The method according to claim 2, wherein the polynucleotide is introduced into the subject via one of the following routes: calcium phosphate co-precipitation, electroporation, microinjection, nucleofection, cell squeezing, sonoporation, optical transfection, impalefection, gene gun, magnetofection, viral transduction, cell implantation and transfection via a dendrimer, a liposome, or a cationic polymer.

4. The method according to claim 1, wherein the polypeptide is one of the amino acid sequence of SEQ ID NO: 1 and the amino acid sequence of SEQ ID NO: 2.

5. The method according to claim 1, wherein the therapeutic molecule comprises a first polynucleotide encoding the amino acid sequence of SEQ ID NO: 1 and a second polynucleotide encoding the amino acid sequence of SEQ ID NO: 2.

6. The method according to claim 1, wherein the cancer is one selected from the group consisting of gastric cancer, lung cancer, bladder cancer, breast cancer, pancreatic cancer, renal cancer, colorectal cancer, cervical cancer, ovarian cancer, brain tumor, prostate cancer, hepatocellular carcinoma, melanoma, esophageal carcinoma, multiple myeloma, and head and neck squamous cell carcinoma.

7. The method according to claim 6, wherein the cancer is hepatocellular carcinoma.

8. The method according to claim 1, wherein the cancer is metastatic or recurrent.

9. The method according to claim 1, wherein the subject is a human.


SEQUENCE LISTING (8 sequences)

SEQ ID NO: 1 - protein, 295 aa, Homo sapiens
MVDSVYRTRSLGVAAEGLPDQYADGEAARVWQLYIGDTRSRTAEYKAWLLGLLRQHGCQRVLDVACGTGVDSIMLVEEGFSVTSVDASDKMLKYALKERWNRRHEPAFDKWVIEEANWMTLDKDVPQSAEGGFDAVICLGNSFAHLPDCKGDQSEHRLALKNIASMVRAGGLLVIDHRNYDHILSTGCAPPGKNIYYKSDLTKDVTTSVLIVNNKAHMVTLDYTVQVPGAGQDGSPGLSKFRLSYYPHCLASFTELLQAAFGGKCQHSVLGDFKPYKPGQTYIPCYFIHVLKRTD

SEQ ID NO: 2 - protein, 4374 aa, Homo sapiens
MKVDRTKLKKTPTEAPADCRALIDKLKVCNDEQLLLELQQIKTWNIGKCELYHWVDLLDRFDGILADAGQTVENMSWMLVCDRPEREQLKMLLLAVLNFTALLIEYSFSRHLYSSIEHLTTLLASSDMQVVLAVLNLLYVFSKRSNYITRLGSDKRTPLLTRLQHLAESWGGKENGFGLAECCRDLHMMKYPPSATTLHFEFYADPGAEVKIEKRTTSNTLHYIHIEQLDKISESPSEIMESLTKMYSIPKDKQMLLFTHIRLAHGFSNHRKRLQAVQARLHAISILVYSNALQESANSILYNGLIEELVDVLQITDKQLMEIKAASLRTLTSIVHLERTPKLSSIIDCTGTASYHGFLPVLVRNCIQAMIDPSMDPYPHQFATALFSFLYHLASYDAGGEALVSCGMMEALLKVIKFLGDEQDQITFVTRAVRVVDLITNLDMAAFQSHSGLSIFIYRLEHEVDLCRKECPFVIKPKIQRPNTTQEGEEMETDMDGVQCIPQRAALLKSMLNFLKKAIQDPAFSDGIRHVMDGSLPTSLKHIISNAEYYGPSLFLLATEVVTVFVFQEPSLLSSLQDNGLTDVMLHALLIKDVPATREVLGSLPNVFSALCLNARGLQSFVQCQPFERLFKVLLSPDYLPAMRRRRSSDPLGDTASNLGSAVDELMRHQPTLKTDATTAIIKLLEEICNLGRDPKYICQKPSIQKADGTATAPPPRSNHAAEEASSEDEEEEEVQAMQSFNSTQQNETEPNQQVVGTEERIPIPLMDYILNVMKFVESILSNNTTDDHCQEFVNQKGLLPLVTILGLPNLPIDFPTSAACQAVAGVCKSILTLSHEPKVLQEGLLQLDSILSSLEPLHRPIESPGGSVLLRELACAGNVADATLSAQATPLLHALTAAHAYIMMFVHTCRVGQSEIRSISVNQWGSQLGLSVLSKLSQLYCSLVWESTVLLSLCTPNSLPSGCEFGQADMQKLVPKDEKAGTTQGGKRSDGEQDGAAGSMDASTQGLLEGIGLDGDTLAPMETDEPTASDSKGKSKITPAMAARIKQIKPLLSASSRLGRALAELFGLLVKLCVGSPVRQRRSHHAASTTTAPTPAARSTASALTKLLTKGLSWQPPPYTPTPRFRLTFFICSVGFTSPMLFDERKYPYHLMLQKFLCSGGHNALFETFNWALSMGGKVPVSEGLEHSDLPDGTGEFLDAWLMLVEKMVNPTTVLESPHSLPAKLPGGVQNFPQFSALRFLVVTQKAAFTCIKNLWNRKPLKVYGGRMAESMLAILCHILRGEPVIRERLSKEKEGSRGEEDTGQEEGGSRREPQVNQQQLQQLMDMGFTREHAMEALLNTSTMEQATEYLLTHPPPIMGGVVRDLSMSEEDQMMRAIAMSLGQDIPMDQRAESPEEVACRKEEEERKAREKQEEEEAKCLEKFQDADPLEQDELHTFTDTMLPGCFHLLDELPDTVYRVCDLIMTAIKRNGADYRDMILKQVVNQVWEAADVLIKAALPLTTSDTKTVSEWISQMATLPQASNLATRILLLTLLFEELKLPCAWVVESSGILNVLIKLLEVVQPCLQAAKEQKEVQTPKWITPVLLLIDFYEKTAISSKRRAQMTKYLQSNSNNWRWFDDRSGRWCSYSASNNSTIDSAWKSGETSVRFTAGRRRYTVQFTTMVQVNEETGNRRPVMLTLLRVPRLNKNSKNSNGQELEKTLEESKEMDIKRKENKGNDTPLALESTNTEKETSLEETKIGEILIQGLTEDMVTVLIRACVSMLGVPVDPDTLHATLRLCLRLTRDHKYAMMFAELKSTRMILNLTQSSGFNGFTPLVTLLLRHIIEDPCTLRHTMEKVVRSAATSGAGSTTSGVVSGSLGSREINYILRVLGPAACRNPDIFTEVANCCIRIALPAPRGSGTASDDEFENLRIKGPNAVQLVKTTPLKPSPLPVIPDTIKEVIYDMLNALAAYHAPEEADKSDPKPGVMTQEVGQLLQDMGDDVYQQYRSLTRQSSDFDTQSGFSINSQVFAADGASTETSASGTSQGEASTPEESRDGKKDKEGDRASEEGKQKGKGSKPLMPTSTILRLLAELVRSYVGIATLIANYSYTVGQSELIKEDCSVLAFVLDHLLPHTQNAEDKDTPALARLFLASLAAAGSGTDAQVALVNEVKAALGRALAMAESTEKHARLQAVMCIISTIMESCPSTSSFYSSATAKTQHNGMNNIIRLFLKKGLVNDLARVPHSLDLSSPNMANTVNAALKPLETLSRIVNQPSSLFGSKSASSKNKSEQDAQGASQDSSSNQQDPGEPGEAEVQEEDHDVTQTEVADGDIMDGEAETDSVVIAGQPEVLSSQEMQVENELEDLIDELLERDGGSGNSTIIVSRSGEDESQEDVLMDEAPSNLSQASTLQANREDSMNILDPEDEEEHTQEEDSSGSNEDEDDSQDEEEEEEEDEEDDQEDDEGEEGDEDDDDDGSEMELDEDYPDMNASPLVRFERFDREDDLIIEFDNMFSSATDIPPSPGNIPTTHPLMVRHADHSSLTLGSGSSTTRLTQGIGRSQRTLRQLTANTGHTIHVHYPGNRQPNPPLILQRLLGPSAAADILQLSSSLPLQSRGRARLLVGNDDVHIIARSDDELLDDFFHDQSTATSQAGTLSSIPTALTRWTEECKVLDAESMHDCVSVVKVSIVNHLEFLRDEELEERREKRRKQLAEEETKITDKGKEDKENRDQSAQCTASKSNDSTEQNLSDGTPMPDSYPTTPSSTDAATSESKETLGTLQSSQQQPTLPTPPALGEVPQELQSPAGEGGSSTQLLMPVEPEELGPTRPSGEAETTQMELSPAPTITSLSPERAEDSDALTAVSSQLEGSPMDTSSLASCTLEEAVGDTSAAGSSEQPRAGSSTPGDAPPAVAEVQGRSDGSGESAQPPEDSSPPASSESSSTRDSAVAISGADSRGILEEPLPSTSSEEEDPLAGISLPEGVDPSFLAALPDDIRREVLQNQLGIRPPTRTAPSTNSSAPAVVGNPGVTEVSPEFLAALPPAIQEEVLAQQRAEQQRRELAQNASSDTPMDPVTFIQTLPSDLRRSVLEDMEDSVLAVMPPDIAAEAQALRREQEARQRQLMHERLFGHSSTSALSAILRSPAFTSRLSGNRGVQYTRLAVQRGGTFQMGGSSSHNRPSGSNVDTLLRLRGRLLLDHEALSCLLVLLFVDEPKLNTSRLHRVLRNLCYHAQTRHWVIRSLLSILQRSSESELCIETPKLTTSEEKGKKSSKSCGSSSHENRPLDLLHKMESKSSNQLSWLSVSMDAALGCRTNIFQIQRSGGRKHTEKHASGGSTVHIHPQAAPVVCRHVLDTLIQLAKVFPSHFTQQRTKETNCESDRERGNKACSPCSSQSSSSGICTDFWDLLVKLDNMNVSRKGKNSVKSVPVSAGGEGETSPYSLEASPLGQLMNMLSHPVIRRSSLLTEKLLRLLSLISIALPENKVSEAQANSGSGASSTTTATSTTSTTTTTAASTTPTPPTAPTPVTSAPALVAATAISTIVVAASTTVTTPTTATTTVSISPTTKGSKSPAKVSDGGSSSTDFKMVSSGLTENQLQLSVEVLTSHSCSEEGLEDAANVLLQLSRGDSGTRDTVLKLLLNGARHLGYTLCKQIGTLLAELREYNLEQQRRAQCETLSPDGLPEEQPQTTKLKGKMQSRFDMAENVVIVASQKRPLGGRELQLPSMSMLTSKTSTQKFFLRVLQVIIQLRDDTRRANKKAKQTGRLGSSGLGSASSIQAAVRQLEAEADAIIQMVREGQRARRQQQAATSESSQSEASVRREESPMDVDQPSPSAQDTQSIASDGTPQGEKEKEERPPELPLLSEQLSLDELWDMLGECLKELEESHDQHAVLVLQPAVEAFFLVHATERESKPPVRDTRESQLAHIKDEPPPLSPAPLTPATPSSLDPFFSREPSSMHISSSLPPDTQKFLRFAETHRTVLNQILRQSTTHLADGPFAVLVDYIRVLDFDVKRKYFRQELERLDEGLRKEDMAVHVRRDHVFEDSYRELHRKSPEEMKNRLYIVFEGEEGQDAGGLLREWYMIISREMFNPMYALFRTSPGDRVTYTINPSSHCNPNHLSYFKFVGRIVAKAVYDNRLLECYFTRSFYKHILGKSVRYTDMESEDYHFYQGLVYLLENDVSTLGYDLTFSTEVQEFGVCEVRDLKPNGANILVTEENKKEYVHLVCQMRMTGAIRKQLAAFLEGFYEIIPKRLISIFTEQELELLISGLPTIDIDDLKSNTEYHKYQSNSIQIQWFWRALRSFDQADRAKFLQFVTGTSKVPLQGFAALEGMNGIQKFQIHRDDRSTDRLPSAHTCFNQLDLPAYESFEKLRHMLLLAIQECSEGFGLA

SEQ ID NO: 3 - DNA, 19 bp, synthetic construct
  source 1..19: mol_type = other DNA
gagattgccgcaccagaga

SEQ ID NO: 4 - DNA, 23 bp, synthetic construct
  source 1..23: mol_type = other DNA
tcaaggacatggtgcataaatcc

SEQ ID NO: 5 - DNA, 23 bp, synthetic construct
  source 1..23: mol_type = other DNA
cagaagttgggttttccagtcaa

SEQ ID NO: 6 - DNA, 20 bp, synthetic construct
  source 1..20: mol_type = other DNA
acatcacagctccccaccat

SEQ ID NO: 7 - protein, 1606 aa, Homo sapiens
MSEDSRGDSRAESAKDLEKQLRLRVCVLSELQKTERDYVGTLEFLVSAFLHRMNQCAASKVDKNVTEETVKMLFSNIEDILAVHKEFLKVVEECLHPEPNAQQEVGTCFLHFKDKFRIYDEYCSNHEKAQKLLLELNKIRTIRTFLLNCMLLGGRKNTDVPLEGYLVTPIQRICKYPLILKELLKRTPRKHSDYAAVMEALQAMKAVCSNINEAKRQMEKLEVLEEWQSHIEGWEGSNITDTCTEMLMCGVLLKISSGNIQERVFFLFDNLLVYCKRKHRRLKNSKASTDGHRYLFRGRINTEVMEVENVDDGTADFHSSGHIVVNGWKIHNTAKNKWFVCMAKTPEEKHEWFEAILKERERRKGLKLGMEQDTWVMISEQGEKLYKMMCRQGNLIKDRKRKLTTFPKCFLGSEFVSWLLEIGEIHRPEEGVHLGQALLENGIIHHVTDKHQFKPEQMLYRFRYDDGTFYPRNEMQDVISKGVRLYCRLHSLFTPVIRDKDYHLRTYKSVVMANKLIDWLIAQGDCRTREEAMIFGVGLCDNGFMHHVLEKSEFKDEPLLFRFFSDEEMEGSNMKHRLMKHDLKVVENVIAKSLLIKSNEGSYGFGLEDKNKVPIIKLVEKGSNAEMAGMEVGKKIFAINGDLVFMRPFNEVDCFLKSCLNSRKPLRVLVSTKPRETVKIPDSADGLGFQIRGFGPSVVHAVGRGTVAAAAGLHPGQCIIKVNGINVSKETHASVIAHVTACRKYRRPTKQDSIQWVYNSIESAQEDLQKSHSKPPGDEAGDAFDCKVEEVIDKFNTMAIIDGKKEHVSLTVDNVHLEYGVVYEYDSTAGIKCNVVEKMIEPKGFFSLTAKILEALAKSDEHFVQNCTSLNSLNEVIPTDLQSKFSALCSERIEHLCQRISSYKKFSRVLKNRAWPTFKQAKSKISPLHSSDFCPTNCHVNVMEVSYPKTSTSLGSAFGVQLDSRKHNSHDKENKSSEQGKLSPMVYIQHTITTMAAPSGLSLGQQDGHGLRYLLKEEDLETQDIYQKLLGKLQTALKEVEMCVCQIDDLLSSITYSPKLERKTSEGIIPTDSDNEKGERNSKRVCFNVAGDEQEDSGHDTISNRDSYSDCNSNRNSIASFTSICSSQCSSYFHSDEMDSGDELPLSVRISHDKQDKIHSCLEHLFSQVDSITNLLKGQAVVRAFDQTKYLTPGRGLQEFQQEMEPKLSCPKRLRLHIKQDPWNLPSSVRTLAQNIRKFVEEVKCRLLLALLEYSDSETQLRRDMVFCQTLVATVCAFSEQLMAALNQMFDNSKENEMETWEASRRWLDQIANAGVLFHFQSLLSPNLTDEQAMLEDTLVALFDLEKVSFYFKPSEEEPLVANVPLTYQAEGSRQALKVYFYIDSYHFEQLPQRLKNGGGFKIHPVLFAQALESMEGYYYRDNVSVEEFQAQINAASLEKVKQYNQKLRAFYLDKSNSPPNSTSKAAYVDKLMRPLNALDELYRLVASFIRSKRTAACANTACSASGVGLLSVSSELCNRLGACHIIMCSSGVHRCTLSVTLEQAIILARSHGLPPRYIMQATDVMRKQGARVQNTAKNLGVRDRTPQSAPRLYKLCEPPPPAGEE

SEQ ID NO: 8 - DNA, 284987 bp, Homo sapiens
  source 1..284987: mol_type = genomic DNA
agcttttgattaatgtccaagtcactgctgtggctgccgaagccgcttcccctccagccccggccgtgtgaggccagagcagcggggccgggcgcgcagggcctggccggagcccccactcccaggagtttcctctgcagagccccgcgccccccgcgccgggatttcagcccgatcccctcctctccctgcgcccagcctctccccagcatgtaaagtcttctgtctctcctcggagttggcggaggcggcaactttccattctcgccgccgggggccgggcagcagcgggcgcgcgggtcagcgctcagcacggcgggcagcgccgcgctgcgcaccgccgccgaccatgagcgaggacagccgcggagacagccgcgccgagagcgccaaggacctggagaagcagcttcgcctgcgcgtgtgcgtgctcagcgagctccagaagaccgagcgggactatgtgggcacgctggagttcctggtgtcggtgagtgtccccggcagacgcagggggacgtccgggcggcgcggggcgcgggtcccggagtggctgcgggaaggacgcgcggcattgtctgtgcggggacccgggacgggtcggggcatcacaggcgcgggaatccacgtgtggactctgcgttcgcgggcgcggtgacccccgcggggatttgttggtgccccgagactcactgagttgcgggggccttggagacctccaggtctgaaaagttagctctagagaagagagctccagtccagggaatcccggctgccctatcttccctcacttttcttttacccgcctccccagtctgctcaagtgcactgtgtaattgattttccttcttgttctttcctggagactttgttttaaagctttggttttgtgattaaaggtccagtcggctacatccaggtacactaagttaaaaaaaatccttgaatgtgctcgagcttgagttcgttactcagataacatggcagtggccgctttagttatggtctttgagacagaggattctggtgtaggggtacagagagagcagcggggtgatgatctctagcagctgttggatattaaagccacatgtggcgctcaccctgtcctagtcttacaattccctcccccccgccccccgccccggcttaaatctgtcttctgctcagtttctggtcccctgtttacatggcccatgagagtgctgtaatcaagcttcgggacttcgtggaagtggggatgtacttatattccacgtttctgcttgactcactgccatctgctttgaaaacgtctcattcattttagggccgaaggggcaaaactgagtgaaagtaactgctgagataacactcaaaagttgaatttttcctattcattgctgaaaagacaaagaatttgagccttggcttctttccacccagattcctgggagagcatcattcaaagaaccaggagctttagtttttgtgatctcatccaaatccttctccttccaacaaccaattccatgcccccttggtctgaaggaaaatatttcaggtgccaaacttcctgcttagtgttctgaaatctgtcaactttctgcttataatcatgacagataaaattcacttcttggctgtggcaagaaggaaagaaaaatgtctcaagtgatgtaaaatgtacaggctgcgttatggttgacttctcaaaatcaatatacttgacttccaggggccacattctctgcaaatatacaggtatttgagaatatcctggtaactggtggtttatagaattattatatacttccttgtaacaaagattgtcaaaaaagtatgtttttaagatttgatttttaatagctttaaatttggctttcatgaaacttttttggtaacaaagttctttgtcttttgaagaatctgagtcatttcttacataattactctttctgaagatctgtgaacaaagtactgtgcagaatgctaatttgcagagacagttaatctaattatattaaaaaccaatttgttgatctttatttaaaatatcagtttttcttctaatcttaaacctgatcattacaacagaaaacttactgttagtatatagagttaggtaagaaagtgtggaaagtataaaataaattttccccttttataaggagaacatcatttcatgatttctttttatctctttcagaatttatgggctcttccacttttcattttctttgtgcatcatttgtgcctgtttatgtttatatgcacaaatgcactaattagtctgaatgccatcctaaataaaagtttactctggcttacaaagcctaagatctatttataatagattattagattttctttttagggtgttatttatagcaacatttactccccatggagttttcttggttacctgaagttgatgtagtcactctggttttgatattagactatgctaatatgtaccaagagcttttaatctatgcaaaccctttggcctggtaattcctcttctaggaacctatcctaagggaatgatcagagatggaaacaagcaatagaagttatttttaaaagttagccatagtattaaataagtcgagtggtaaatttgttgccaaaaattgatagtttactcctaagtcaaattaaatatttgttagattagtcattaatcatttctattattgctttaagccattaacatggatgaaggatgactaatctttattaggaagaggaagaagaggtagaaaacatatttttagttctactaagaacatatttttacttaactgccctataaataagcaaacaaaatggtattactagctttataaaaagatcatatgtcctagcacttttgggggccgaggcgggtggatcacctgaggtcaggagttcaagacaagcctggccaacatggtgaaaccccgtctctactaaaaatacaaaaaattagccgggcgtggtggtggacacctttaatcccagttactcaggaggctgaggcaggagaatcacttgaacctgggaggtggaggttgcagtgagccgagataatgccactgcactccagcctgcctggatgacagagcgagactccatctcaaaaaaaaaaaaaaaaaaaaaaaagaaatcatatggctagagggaaaagcttttctaaaatctttcctatttctgagccctctctctcctcaattcccacactttggcctttaaggaaagagcatcttcccccactgccatccccaagacagcccatgagcattgcctttcttctctaagctaaggtatgggtctatctgcatcttctctgtagttgcccccaggcacacaccgctgtttgaacaaagctctgtgtcttcagctggctactctttccatggtgggctgtgcttcctcaggcctctgcctttgttcttcctggggccttccttttgtacctggtggattcctactgggctttcaaggctcagttagccccttcttcccaggtgctccttgctttgtatccactctaccctgcatttccttcttccatagtacccaaagtagttgatgtgtgcatttgtttactttggtcctcattctgttagacctccttgaggacagggacataccaccttgtgtgcctgtgctttccataattgtgtgcagtgcttgtgacattgtaggtcctcaataaatgcttgctgagtgcatgcatgaatcaatgagttgacccatcttctatcctcccactccatttttaaggatagtgcctcaatcatcctgaacattttgatttcctgttttcttccatttggtcaaaatagatagtagaattttcagttttgctcaggcacatatgaatttatgcagtcaaaaatttcaaattaaacatgtggtacctgcctttaaaacagaaacatgatttaaatcaggtattgaggtattaaagactttgcatgcactattgtttcatatattttaaatttttgtttgagatcatggataggttacactatctagattaattatttttaggtcacagataaaatggtagagagtgacataatttgcttgaatttatgttcttgttttgcttgggaaatgaaattttgtgctttattaaaatattttcaaacatttcttctaaataaatgattacatgaacctgacatccagtatttaacatccaaagtctggacaatagtaagttacaacctgtttcctgtttgcaaacctgtgacagagcctgccgagatgacagactcatgtgagtggacagagaaaaggaagacccagggctggcttcatggatgcacatcctgtgcagtggcccagggttctgagctgagaaggatgcccagcttgttttaatgctctgctattgccatcttgaaattcttgccaattttatctttgaccttgtgttttgcaaatgaagtctgatgggacggtggacatgagcagagcagatacatgaaagaaaaaagctttatatttcagtacctttaatagcattcccccacagccccctgctttttgaacaagaaacctcacatttttattttgcactgggcctggaaaattgtgtaggccatttaactgtgggtcagtggcaatgaggcaggatgaccatataatttactgtctaaactgggacacttctgagggtgaaagggagtgctattaataatgacactgggaaacagatggaaactgggattgttcctggcaaaccagaatgcatggctagtccacattgtgggcatattaaaacaatgacttcaagctatgtgtcttgtcataaaccaaatattttcaagtttgttgcctgtccaggctttgatcttggataccttggtatttgagtcccatctctaatttgctttgtgattttaaacacattcctaacttttcatgcccctttttcttcattcgtagaagggagaacaatagcatttctcaggggtattgtaaggatgatgagatatatgcattgggcttggcccatctggctcataagagggtcctataagtagttactgttattttgctgatgctacagttgttataaagagccatgcctatgatcatcattagatagaatttttgtttctgttatattccttttctaccctttctgtatgtccccacttctcttcgtttcctccttctttgttttccttttgttagtgtagtcttcttaatgatattcttataaatgctcattagttgattttaagacagtgagaaaactttatattccaggtaacaaccacagttagtatggcgccttggagtttaaacaattgcatcctcactctattattcagcttaatatatgcatgtctcttagctctttcaaattgtgctccctggagctccagaggcccttgcaagggctgctggagagctagattttatggcttcaggattgtcaccctggcttctctgtgcatggctcaacctttctcagatttgagctttcgtaaaagcagttagaaaaaacatttgtctacatggcaaattcccagagaaaagggggtttgcatttacttcctatgtatgcttttcttggcctaaacacagggctttgtaaatagcaggtgctcaagaaaacagacaaacaggcaaacaaagcaaaagcaaacaaagcagaatttgaatgcatctgaaatcaaactctttaagtcatgacctgactcttccaggcaaagatgattgattcagcacattatgcttaaggtttatgttatggactgaattgtttccctcctcaggagtcatatgttgaagtcctaatccccaatacttaagaataggactgtatttgtagatagggcctttaaataggtgatagagttaaaatgaggtcatagggtagaccttaattcagtttgactaaagtccttcaaagaagaggagatttggacacacagagagatactgtggtgtacagaggaaaggccttgggaggacatggggaaaaggcagccctctgcaagctaaggagggaagcttcaggagaaaccaaacctactgacaccttgatcttagatttctaagctttcagagttgtgagaaaatcaatttctgcttaagccatcaatctgtggtattttattatggcagcctgtgcagacaaacgcagcttatattaggtgctgggtcatcacaatactctatgggagctgcgacactagagccataagcaaagtcacgtggaggcagtagagattctttcagttggggagtcaaggaaagttttggaaagaggtgatgatagagctggctcctaaaggaggaggaggaatgtgctatactgggagaattatccatccccaaagtgctgcatgtgttatgtgacctcagcgtggcagatcctcacatttaggggtctgaaacagtcttgtaattataccctattataataccaattactttaatataaaaatagtagctattgcgattagatttgtttcttagcaagagaattctgttgacaaggttgaggaacaaagattgaagtctagttacccattaagagtctgtcattgcattagtccaggtaagaaatgtggaaagtctgatctggggtagaagcattggaaaatataaaagaacagtaacagcagaaaacccaaaccaaaccacaaacaaatcaaacgcccaactatgagggcttttttgggtagaagctttaatattttgtatactctcagaagtggaggtttaagtagaatggagaacataggatggggctaagtctctagttttgaagactagatgggtagtgtcattgactgagaattggaatgcaggggacagaacagcactaggaggaaaaggaaaatgaatttgcttttggacatgttgagctgagccacaaatactactatgatttttgaagataagaatgtggaacttgagaaataatttgggccaaaagtgagagatctaaaaattaactaatcatgtattttgaaaccatcaattgagtgagatggcataaggagagcaattagaggaagaaaatacacaaacaaaaggtaggatgttggagagtcaatgtttaaaggatagaggaagagtagagtgtgcaggtgactttgagctatcaaaggctgaaacaaagaactaggagaagttgatactttgaaagccaaggaaggatctagttttcaaagaaaggtctctttaaagtggcaggacttgagggaagtggggtgtgtgaagactgaagaggtcgctggatctggcagttagaatgtcattggggttttagcttcagtagagcgtgtgagcaaaagccagactgtcagaaccagttgagacacgagtggaagctgaggatgtgaaggatgatggataaactattcaaggagtttggtggtaactgcgaggagagaaggggatgattttgtctagggatgggtaaggcagatgagcctagccaagggtagagagacaataaaaagggaactaaagcagtgaaaacagaaagtggcagaaggcaaatggcatctttgggggaagcatagcaagcacccccatggcactgggagataagacaaggcaaaattttctgaaattttctgagccatcttgctgtgtaacatacccatagtaaaacatagtaaaactaagaatgggcaagctgtcacttgtgtattttacaaattctgctgcttctccagtgtacagctagtggaactgcctgggatggtctttgtggccccttgtgaacacataccagaggctctccctcccacactgggacagtgaggtgaactgcatggcacagaatctgatgaactggtttaaaatcctcgcactgaaacttacaaactctctctttgggcaaacaacacagcctctgtatgtctcagtttcatatttggaaaacacatttgataatacctcacagggctggcctgaaattcaaatcagcttctgtaggtaaagtcctgataggtaggaagtgtttcttccttttcttttctttttaattaattaattaattaattaatttattgatctcactctatcgcccaggctggagtgcagtggcgcgatcttggctcactgcaaccttccctcttccgggttcaagcgattctcctgcctcagcctcctgagtagctgggactacaggcacatgccacaatgcccagctaactttttctaatttagtagagacggggtttcaccgtgttgtccaggctggtctcaaactcctgagctacggaagttcccccacctcagcctcccaaagtgctacgattacaggcgtgagccacggtgcccggccggaagtggtttttttttcccctccataaactccaagcttatctccctctcctctgtcttgtctatccacagactgagcttggcacgtgctcagaccagcttgagagtcaccagcctggtctgtcagtcaccctgtcttttccaagcctggaggcataaggacaagtttaatagaagaaggttctgagaactccagcggggcctagggaatgcaggagagggagtgttccgggagccaccagggagggatggctttcctccagaaggaacttatgagtaaggacaagggccactttaggggagaggcaagtatggaggttgcttgctatccgatggtcgttcttcctctatgaagttgaactctggttttctgtctgatgttctgttttgttttgctcttgttttgttgtttgttgttgctgagagggcagtgccctgaagagaaccgtaatgatttggacacacactttcaggaatagaaaagggaattgagcttggacatctgagggtggatgtatgataggctttgttcaattattcttttaaatgtgattttcttaaacacaacatacaaatttaggagctcaagtagagacgtaagcacttctggtttattacatctagtatgaataggttcaaatctcagagataatgacaaccattttttaaaccatggtgcaaatagtataaaacttctattttacatttgatgcacatatcagtcattagccatgtaaaaaaaagaaaatgatttatctgacattttagtgtgattatagtttcctcagcacttgttttaaacagcattttcttaacaattcttttagtatatacctggtattatattttcataccaaataaaattattatgtctgtgttttcagcactattggtttgacctaaaaataattgttttagtttgcattatttctttaaacattttatttatatttacttttcatgagatagattttttgttcttcattaaaagtgttattaaatttattaaaaattttcaaatgtgaatttattagaattatattattagctatgttaaaatcaagatctttgatttaattataattgtaaattgaagaatttacaatttattaaaattatattagctatattaaaatcatctttgatttataacattgtagttgcctcaaacaatcaaaaattcaggaaaagcatcctgtagtaaatgtttcattgattaaaagtgtggctgggtcactttttccctggtattgaacataactattcacatgatgatacatctaataataactgcttaaaaaaatattcccccattttcataaactattgcatttgatccaagagttcaatgattgaaattttaagaaaatgatatgttaatttgaagttatgatttttaagcacttctaattagtatcggtgatttcagactttgatttattgaataaatttgttgtatagccttacatataacggagcatagtgtacaaaaagggatctattttgctcgagagtatcttagccaaggacattgtgttttgttattaatgtgaaatgcatattttccattaaaaggcaagcccttcatttgaaaggttaaattgtatagtacttaagaaatgactccatctgattaaatttagaatactgatataaaaatattctctggtcttacagtgaaaatatcacaatatagaatatagatggataaacatttttattcatctgaaaactttgtttctcggaaagagtcaaatatggattgatatctatgtttgattttggcttgccacttttgttattttaattatctatttaagttgccttcttgccagtaggagaggctttatagatggtgacacattttcagagattgtgcaagaaatgctaatgttttcccttggtgccttgtaatgcctcagcttggccttgctttcatgcttcctaggagttccagccttttcttatttgagactgttgccccaaagaaagaaattttaggattccagaaataacatctctcattccaaagccttatttctatcagtagcactaggagaccatctatggaaaggcattattatattctattaattgacctaaatgttagtcccgcacccacatactccaagattattttaagaattcattattttcccagaggtatgtggatgaagggggctttcattttctatactgtataactatgtcaaatttggctttctataattttatatataaatatatttgtatttatatattttatttttttttttatttttttgagatagagtcttgctctgttgcccaggctagagtgcagtggcatgatctcggctcactgcaacctctgcctcctgggttcaagtgattctcatgcctctgtctcccaaatagctgggattacaggcacctgccaccacgcctggataattttttgtatttttaatggagatggggtttcaccacgttggccaggctgctctcgaactcctgacctcaggtgatccacctgtctcggcctcccaaagtgctgggattacaggtgtgaactaccatgcctggccaattttatatattttatatatttacaaaatgtattttgaatattctagacacaactttatctcaagttttcttgtaccacttcataatgaaacctctggcagagacttctaagtgtccccagtacccaatgttcttttcttctatagtgataaaagttttaggtccagcttaagactacatttctcagcctcccatgcagataagtgtgggcctatgtaaaggttctgggtagtggggtgtgagcagaagtggcctgggtaacttctgcagtgtgcccttattgtgaagagtgtgcattctcttttctcctttccctcttctccctggctggtatgaggatgtgatggcaggtgatggggcagcaattttagaacatgaaatggaaggtacatgttcagggtgacagagcaacaaaatagaaggagactatatctttattatttcttattaagaccatgtcttaaagcaggctaacctatgtcctgtcaaaaatgggcctataagagaaatcaatatttatttaactctgtcctaaaatcatcacattttgataccaaggcctcattgacttatgcaaatcattaatgaattggccaactttagtatcttttttctgtctttgtctaatatttttagtttattttttgttttttctttaataaatctctagttcagccataatttccccagattacgctaaaactgcacttttttgttttcaataattatactttagtttttcctttagaaaaagaatagtattttttttttacctgtacttaacatattctttttttcctgaaggccagaattttgtgtttattcgtaaggatatttgttcagctgccttaactgagaccaaataacaatagtttgaacaatgcagaaggattgtttgtggctaggaaagccatatgtaatcaacatggccttttttgcattctctcttagaactctggttatttaatttttaaattaaatataccccagtggtgttccagtggggttagccgctcagccacatcaggttattcagagactcctgcttcttttatcttattgctctaccagcccctaggaccaatgaaatctgagtatgtgggggtgagagccaggcctcagtatttatgtagattctctaggtgtttccaatgggttagagaaggtgggggaaccagtatagtagggtgttgtccttttccctatggttgaagctgacttaccactacacttttcacattccagccctagggggacagagggtgggagacaaagtggagggcaagcactccctttaaggatatgacccagaaatgacacacatcactttcactctcatccccacgaccagatctttgtaacatggctaatccagctctaagggagactgggaaatacgctgactggctggacagccatgtgcccagctgaaacttagggagttctacactgaaataatgacatgggaataaatattgaggggcaaagggtcatctctgccacattttgaatccattgatactttagtctcctgtcctcagaaaatcatttatattaattattggattcctttactgtaagaacctattgtatttaaaatataatttggattatttccctctaaaataatatattctttcccacatagcaatccatctaacatttatctctctcttcagatatatttgtaagagctccagagctctcaaggttgtttttgtttttttttccattggctttcactacagaaaggctttggggtatttggtgattttatgtaattttaaaattttgtggaagtttccagaaaaaaaattccaagataatatggaaattctgtttttgttatgtgtttcttatgtgttaggcactgcatattcattaactcacgacttagaactatcttcaagcctcggatcattgtaaactcacacctggggacattttggtcagttcctatttgggagagtggggattctgaaaagatccactgcaaaagttatatagctcttccaagatgttctgctgatgaatcacagcccggactcttctggattgtgggaatggtgtgattaatcaggactaggaaaaccaaggcagttgctggagtagggacgcagaacttgttgactccggaagtacacagaagctagaaatcaggaaggaacagtagttcatttcaggcaaaccatcaagcgcaagagagcaacaggagcttatggtcaatccaagacagggttagagccaggcaagcactcaggagccaaagattacccagaagtcaggagtttccctcacagaaataaggaaacacatgaaactttcagaaatagaaaaccttagttctcccattggcatatctgttaatatatgttggtactgatgctattttcagcttaatttaagtcctctgggatcattttggatctctgtgtatttacagtgcttcctaacaggtttgaacccttggctttgtgtttaaatatagctcgtgtttatcctatatatgatcaaatcacaagacatttgaggcaaagatataaaattagtttgtataattaacatatgtaattattatgagtgtatatgtatatatatatgtgtgtgtatatatatatatgtgtatatgtatatatatacacacacactgtccaaaatgtgtgtatgtctgcattcagtggatgttctattgattgcttaggtgaaaagcattaatgtagtttaaacatatattaaatgaatccattgttaaaaatgatcactattcttgagagtgttctgtttgccagacctactttgtttgacaggatagaggagggagagtaaacggccttctggtagtcatagaatgaagtttggtaaatgggtcatgttacatagctagagacattttcctcagtttctagcaaggacaattagcacaaccatatgatttttagggtccaaaaaggaatcatgttattcatggaagagcatagctttcaattaaaataggagaagttgatgtttctcctgtataatttttagtgcgctaataattctcacatgtaagttgggtcttctctgtatcattaaatgttttgagggtagcaaatattctttgtgatttaagatttttaaaaaatttctacttatggtttacttcattatgaggaacatgataagttggattgtcagaaaatttgagagcttagtacttgactggatcttcaattttaaatatatgtatttctatagatgaggatttgggtcagattcacctttgatgatgttgtgaaaatataagcaaaccgtaatgagagatctaggtagtcagctaggatatttgaataaattttttaaaaagaaaatgtctgctaacatttaaagaaaataaattgcctatagttttactatgttgtactatatatccttcctacaaattatttcttttccagtatgtcacaattcaaagttccaactcctctatgatattctgtaatttcccttagtactgaatatgtgttaagtaaaatctacaggtttttttttttccaaataatacagaactaaaaaatctgtaaaagagaaaaaaggaattaccataaaatgcatcaatctgggcagagcttggaaggctgaagtgggagcatcttttaagctcaggagttcaaatctagcctgggcaacatggtgagacctctgtctctaaaaaacaaacaaacaacaaaaaacaacaacaacaacaaaaagaaaatgcaaccagagctatttataaattaatgtgcaaaaccaactttattaagtgaaaaaacttattttcagcaatgaatcaggcaacctaaaaaataacccctaatatgctaagcattgtgattgattctctccaatgattgctttcaaaatccagatatttttattcttcatggtccaaggcatatttcttgagcacctactgtctgccaatcactattcaaggttcagaggatatagcactgacaagcattacaaattcctgctttcatggagctctgactctaacctaggagctacaagataaatgagtaaaatatatgatgtattagctagtctaagtgcctgggagaataagtgaagcagagaagaggaataagtgtgcatgtgttttgacattttatgtaagatgaaaaagcaggcattcactcttttctgtcactcttcttttttgtaaatctctaaaaatatttgaaagaatttttattttttactgttaaccttaacaggcaatatccaggaggaaagtgccaattgatttcttgtttaaatcttcagtgtagtgataattcagaggtgagattaaacccagatgtacattgctaataaataaagttaatagaacagattagaaaaagcatggtgacactaagacttaaggctttaactagtgtcattctttagttgtcatttttcagtctattgactttaatataataggcatatattttttaaaggtgcaatctttaaaatccactgcctcactctgttctgtgtttataccattgaggagcttattttgtggagtgttttgtcaataaggtttcatgcccagcagctccatgtctagtggaactgcagccaacattcaaattcatcatggatgggaacccagcacgctgtgcagttggggcaccataggctgaaaagcttctgctggaaatgaagtatcctttagcagcgaggggatggaagcattgaacaagtggggcaggggtgggaattggagatcttgaatgtgcagcaggctctaaaagccattttatgaccattgccttagataatcccagacgtgagttaaaagctaaggtatgaggtgctttcatacatttttcttatttaaggcctttccctgaaaggggatgggaaggatcaagagtaccttgcattctgagcaaactgttgcaaggacagaaaaccaaacaccacatgttctcactcataggtgggaattgaacaatgagaacacttggacacagggtggggaacatcacacacctgagcctgtcgtggggttgggggagggatagcattaggagatatacctaatgtaaatgacgagttaatggatgcagcacaccaacacggcacaggtatgcatatgtaacaaacctgcatgttgtgcacatgtaccctagaacttaaagtataaaaaaaaaaaaaagagtaccttgcaaaccctccccggctttcttcctgtggtattgtctctctgattgccctatagaaatgactcccttgaggcttgtggacagctggaagaggcccctatgtggcctgtaaagtgctattggaagcatctgccagcgactggggaggggaaaggtcacttgaggactgtggaaaatattccctttctccttagcatcctggaacgtcggctcttgactaaattgttaatagaatctgggagctcggattctggtgctgacatttttttcagggttagttctagaaatgatatttcttaagcagccttttgcggagcttcattctttggttcactgaattcgcagcacaaagaaaaggaaatagctttattaccatcatcattaccaacttcagaataggaatgggtgaataattatattgggcttttaaaaagtataaccaaagggaaggaatttagctgtctgtatttttttgataaggttatgtgctttcttcttcctgtttctctttaaggagacctttctgtggctcatacatcataaaagttaaaaggaagtgcaaacacaattaccacagaaagttgaaatctttcgcatccacttctcttttgtgctatttgtgacctgtctgcctgattgtttcaaagtaaggaaaatcctgggtttcttatctatgcttttcaggggaactggttttgactgtggtttgaatctgacaaaatggagacaatcagccttaacctaatagccgttgtaccaaactaacccacaagaatgcaaaggatacgggtgaactttaaaggcaagacttgcctaaggaaataatgcaagatacagtgcctaggaaggaaatcagatatgtaaaactccttgaactctgtaacagaattttcaatccatcagtagctcttacttggaggctttgagattttctgccgcactctgctcagcgtcttaatgctcatgccagtcatcaggttgtcgtgtggctggggtcaccatgccagagcctgacctggaggccctgagtgctgacagtgctcatttatcaagagcacagtgtttggttggggttatcactggggagtgaggccgcgtttagagtttggtctaagacaatgtctgaatgtggcagggggtcataaaggaaccggggacaagggttaacccagctcagttctctctctgtggtctatgaatgccacagccaaagcagcagcttcagagatctctgctaccttttagagaaatgtcgttgctgttgctgagcccactctcaggtattctggtctcatcaccccctgtatcttttctcatgcaggagcctctggcactgactgtgtagctttgagtgtggcctttaggcttgtgatgactctgagggatggaccatcccataataataaggattaggaccaaaattataacccaaggagaggtagattttgattgcttttaaacttacttcctgagatgattagagtaacatttgagacagttggtttgtagttactccctcaagtccattttccacctaggaaggtttattaatgttttatgattattgtataatttagaagaatatttaccagctgctgggaccttcctcatacatcaagtctggtctcttctggagatggaaccctcaggaaaaacttgaaattcttcctgacagcttgttcgaaggttgccatttattttggtgagtgggttcattttgctaccgctgtgtaaaaatgggaagtctcttccttcagctcacatacattcccaggtgagcacaggtgaaatgtgttgctacaaacaaaaggaagagagttagagaatcttatgcccatcatctgagtctcactccggtctgaaaatttctataatttctaaaatttctatagctcaatttgctctaattcctagtaccttccatattttttcctcttggttgtttatgctgtagttataatctgcttagagattccgtaagtatgggaaatagtaagagggaaatcagctatctccattttgggaagtcaaagtgtgaagttgttttgtttattgacctgatggtgtatatttgtgtaacagttgttgaaggtgagttaatctcgatctgttaatctaaacagatcgaaatctattccttggagtcatagcctctaaatttcatgtaaagcagtgtaaagttcagatacagtttaatcatcaaactaccgccccacactaaaaaatataataaataaaaaggcccaaaccaccaaacctaaaatgagaactattcctattcatatgaagaaatagtgtaatgaccatcagcctttcaacaacatggtattaattatgccagcaaaaataaccatacccctaatctatttttatggtcctatgtctctgatggaactaattttcaaataaagcctaatcacttacttggcagttctcagctccaacaacctcctacagagattctgttgggctgctagtttgggaaaatataccaaacataacttggagttagagttctcacactaatagctacagactcatgttttcttgatcaagaggtaatcctctttcaacttcagagtaagaaatatgaaataaatcaggagaaaatcattttcctcttctgtgattgtgaaacaactgattatcatacaggagcctattccaagtgcatgtcaggaggcttgagggtcaggacagagactggggatgcagggctcacttcatccacctgctaacatagcagcagaaaggtgctatgtgcagctgttttgcaattattattcttatatttatttacccaatgcaaacagattccctttcatctgtcgttctagtaacaaagagtaaatgcctgaggcttttggctagattttacttttcacaggaactgttgaaaaggttgtcccatttcatggtgttgcttttgtggaacagtgattaaagtggtcccattgaagagtaggtgcaggggtgtagagtggtatagggggtgataaaggtagcttggggccagatggtgaataacagtagaggtagtttaggactaattagtagacagtgtgggagctaggggagatttttgagcaaggaagtcaaaacaatgggccttggttcagaaagcgtcgtctgggaggccggcttctagccagagtgcaggaatggctctgttgggcacaggtgagacctgagagagatcaattcattaagctcaacaaaagtcagaaagactaaatcggtgaggaatattgctgatgcttggacctcggtccagaccagcttaattagaatctcagggtgcggggagagagggtggaagtcaagaattgctatttttaaaaagctccccaggtgattctaatgtgggagtagggttcagaaatacaaggaaatgaatacatgacattaatatatatattaatcaaggattgagatacatgctttccaaactgcccctaaaaaataggtaatatcataatagaatgaatgatgagaagagatgcactgacaaaatccacttataaaactaagaagtaactcaacatcgaagaggaaactaaaaagaaatgaaattagctaggaggctgcatttattggagatatagttatccattaatactttgcaatagtaatgagaaatactgttagtggaaaagatatagttaaagttttgatttaacttaggtagaaagcagtcaacaaatcattttccatgtgtctgagcaaggcaataagacaacagtgattagtgggaagtactcctgacgtgggaaaggaagtatgctactttgggaaataaggatgttgtagaaattaatattttaaagtctttggagaattcccatggaggactgatgtattttccaaagttctttgttgtgatgagatatggagaattattttctaagtgccaatttgtgaacataagcatatgcatgccaagggagatttgaagtgcaagaaactgatttctgtaaagccacttgtttgatgtgatgtccttaatggttttgttttgttttcatattttcccttatgtctaaagatataattaaagttcactgccgtgaggggctcaggtgtctttacttttggctgaatgagtaatcgcccttgatttctgattgcagtaatttctgtaataaagcccattggtgatctccataaaacttattacctctacatatgagtaaggaacaaaagtgtaactttgatttcttcctgtttatgccatcagatatagtcatgtattctgtgttgaaagtatgtgaaacctcttctgaatctcattgcctcttgcagctcctgtgtaaattctgtcccaaagctttcaggttccctgactacatcgttgtatttactgctgaatttactgcattctcttcctctttcaactgcttcccaacctgcctttcagaccctttttttaagaagctaggctcctgaaggttccccgtgactatcttttaaccatacctcttagttctgtcaccagttttgagctcccttcacttccccggcattgttcatcagtgttacttgtttgaaattccctccctgtagggcctcccttaggttccctgatgaaactctacagttacccactttttttctaactttcatttcttacctttgcatccctttctacctactttaatcccttccagtaagcttatcaactgaccatggcctctgcaattacctttacataaaccagattaaattatattgctccagtgaagattttgaatcccaaaatcatgtcttctatataaggacattccattgtatgtctcacttttgcctcaaactcaacatgtgcaaaactaaattcatcattttcccttagaggacatgtaactcctctttgtccctcccctatttttctgtaattggtcttctctggtctccctggactggaaggctcagtgtcatctttgtctccatatctttcatcccttgttcagccactctcaatgccgtgttgatttctgtttctaagtgtcactagatatacccctttctccaggcctcccctgcttcttctctgaagtagtctcctttactttaggggtgatacggcaataaaagtatgggcagactgatatccgtccaacaaacattctcagattaatcttcctaaaagaatgttttcatctttttatttcattttatcataaactttgcctccttgatgctcaaacatcctaatttatcaacttgtcattctagagcctttttacaaaaaagggcctttggcatatctgtcaaattaacttgctgtagctactcctgaagaatctctccgatagggttctcaagtgatccatcaaacatcttgctgatctgcctttctgcctctacttatgtatgctcttctttcttcttgaatatccatacttgaatatcttttcttctttcttcttgaatatccatactcaggatccaattcaaacatcacctttttgtaatgacatctcttcctagtctgtttcataccacttttatcttcttgtcacttcctatataggttcttgtcactactaactataggttttattgttggtaacgcatatcttagcacttcagtgttattggtcttgcattactatgtatgtgggttttgtgataatttttttgtgtggtatatcacagtttataaaacattatcatatttgggcctgatagtaaagcaagtcagacattttacagctgggagcagttgtattagtagtagccccagaacacatggataccaagagttaaatgtaaaatgtgaaagtggaatctaagagttaacatcaagaacagtgctagttcaactgttctaactttcaaggtcctttcacttattgcaaaacttaatgcacatttttttccttcatatattctatagaacctagcatatgtctattacatactaggtgttaagtatcttattggctgaagtagattagtatatgctataaataagaaaatattctatgaattataaaaatgtgctatatacctttcttcacttagattgaaaattggagaaagagtgaattattactaaaaattgctttgatgtgttctgataaacagcttttttaagataatgtgaattataggaacctcgtgtttgaaagcttttctttcagtggtgccttggtgtactactttggacttagactaacaagatacaatttgccagtatcttggttgagaagaaacaacatgattgccatgaaaaaagaaaagagagaaaaaaagtaaaagaaaaatctatggagtttttaaaaagcattctcataaacttgattttaaaagtgaaaattgatgaacattcttcatgttctgaaaatatattttaataggtattatatattataacatgacatcaattagctaaaatcctacctagaaatatgctagttcattacattttggagctaacttttccatgtaagggataacaaaggatgcgtgaaagggagttgacatttttattgtttttggtagattaaaataattgggttggccaagtatgagggtgagaacattttcctactatttatcatgcaaattctgagaatttatttggaactcattgtacaaagctaatattaagaaaaagttacttaacttttactggaatgaatagttatgatgatcctcctaacctagagatgatgcagtgatatcattttagaaataattttgtttcaaattcatttattgctttggtctcttgcagttttattaccttcttcatgtaaggatataaacaattggatattaagttagttgtaagaccatgtaggaaaatggacagtttttgtgtgcacatccagtctctgctgcatcctgactgtggtgactcagaattagctccggaatccatccttgctgtgtgttcctcataccacttctcagtgtgggcttgtgcacctctctctggagagctctggcttctgcctttattattcttcttctcaagccttcctgcactgctaagaaactagttttcccaatgaatggatgatttttattttgtccctcgctttctcaaaactgtagctcaccctgacctactgaaaaccaaccaaagttcatggtctcctctagtcggggcctctcagtcttttgaaattataatgaaccaatggatctggctgatgttacctgcacacggcctgtttttttttttttttttttttttttttggtgtgtgtctgtgtgtgtgtgtcctattcattgtgttgctatataagtacctgtaggcaggaactgggcctttttcacatttttattctctataactaatttctaatgcagtgccaggcctatagtgaacctaaagaatgaatgagtgaatacatgcttttaaagtgaaacaaatgttactatgtatctttttttattgcctacctttccaagtatgtagtaatatcttaatacattgaacaaagaagaaaacgagagcaacagaattcagcaaacaaatattactatgaatagctagtgtggcagggaagcagtgcctaagtattctatgttagcatgtctttttcagctagtatacttaatatttatcaggatagtaactgatttctctttctttttttttttttttttttttttttttgagatggagtctggctctgtcgccaagactggagtgcagtggcacaatctcagctcactggaatctccgcttctcgggttcaagtgattctcctgcctcagcctcccgagtagctgggactacaggcgtgtgccaccatgcccggctaatttttttttttttttttgtatttttggtagagacggggtttcaccatgatggccaggctggtctcaaactcctgacctcaggtgatccacctaccttggcctcccaaagtgctgggattacaggcgtgagtcactatgcctggccaggatagtaactgatttttgatttaaaaaaatgcatagattcttctatacttttaggcttttgatgaagttataacacacggttgcaactagtccccaaagttgtcttcgtaatgaaccaaacccccagcagcaccctttctttgattctgagctagctctgaaacttgcaattgaccaatgtctcccaagactaggtcttaagggctcttgaaattttatccctggatctcttggaaaatgtgcttttagaatagctctctcgaagatagctgccatagaagcagtgccactattctgagaccaacttgctgtgagatgcctcagccatgtggagagaccctggaatatgaaacactgagagaaagagggagagagacagagagagagagagagagacaggagagagacagagagagagatgggagagagacagagagagagagagagacagagacagagagagagagaggggagagagacagagagagggacagacagagacagagacagagagagagagacgggagagagacagagagagagagacagagacagagacagagagagagatgggagagagacagagagagagagacagagacagagagagagagacgggagagagacagagagagagagacgggagagagacagagagagagacaggagagagacagagagagagagacgggagagagacagagagagagacagagagagagagagagatgcgagagagacagagagagagagacagagacagagacagagcgagaggggagagagagagagagagtgagacagagagagagaagccaaggtaacaccaaagcactagacatgtgagggatgaagtgatggagccatcttaaaaatggatcctccagccaccccagctggtagcacatgtagcagaatgtctagctgaacacttctcaaattcttaattcacaaaattgtgagcaaaataaatggttattgtaagccactaagtgtggcatagtttgttatgcaatataatggattgaatgtttgtctcccccctaaatgtatatgttgaaattctaaccccgcaatgtgatgggattaggagatgtggccttgggaagccattgggtcatgagggtagggccttatgaataggattagtacgcttataaaaagaactccacagagctctctccccgtctttcatttacagtaggatataaggagaaatcatcagaagagggtcctcaccatagcctgctggcaccccagtcttggacttttcagcttgcataaccgtgagaaataaatttctgttgtttacaaaccactcagttgatggtaatttgttttagcagcctgaactatgacatgcagtgacagataactgaaacaacatacaggaacatgtgtaaattattaataaatttcaccagttaaacacacctgtcgtgatattgtgattttttttcttataataacatagatgtatttggccttcatccccatttcctcacatacaactcctacagtttttggaatcccaggagtgacagagtgacctttgtatgctgttggtttgactgatggctggaatcccctaggtagaatgaggaaaggaaaggccaaagcatgattagagggttgggacttttcagccccacactcaacctctggggaggggagagaaattgaaggttaagctaatcaccagtggccaatgatttaatcaattgtgtctacataatgaagcttccataaaaaccccaaagtactaggtttggggggcttctgaatagctgaacatgtagagtttcctggagggtggcatgcctagaaaaggcatggaagctctgcatcccctcgcacaaacctgccctatgcatctctttctgactgtttacctgtatcttttgtaatattctttataagaaatcggtaagcgtaggtgtttccctgagttctgttagctgctctcacaaactaatcatacccaaggaagggtgaggggaaccccggtttttagctggttgatcagaaacacaggtcacaacccggggcatgtgattggcatctgaagtgggggttagtcttgtgggaccgagccctcaacctgtggaatctgacgctatctgtccacctggtgtccactgcagaattgattgcttacttggtgtgtggggaaatctcccccctcctctccccccgcacatacacctttggctacagaagccttctgtgttgtgagagtatcataggagaaactgagttttttttccccatatccttacacctgtgtaactaacatccagagcaagggacagaatattgccagcacctcaaaagccatccttgtgttccctttcagcaacagccatctccaagggtagcaaccactttgacttccagcaccatagattgttttgtctggttttaaaattttgtatgaatagacttatccattatataatccattatatactcttttcacttactatgtttgtgggattatccgtattattgactggagctgtggttaaaacattagcattgctatgtagtatcccattatatgaatgtaatacgcttcatccattctactcttggtggatatttgggtagtttccagttgtcgactattgtggatagttcagtcttctaaatgtctttggtaaatgtatgcacacttttctcttgggtggtttttcaatggttttatgattgcctttgaacttgttagttttatatctgtaagtagcctataagccactctagagtataatggctaggttttatacatatatatctatatttattcttatgataatgcctagtaaacagagtttatagcttagagtagttattcaagaaatatatgctgattgatcacttagaagttttacagcacatatgcttggttctctaaaaagaagttctaaattaatcatgcactttacactacagtatgtaagtataaaaaataggaaatgtcctttcaattagacattcttgttgaattaataacagcttattggaaaacatttcatctgctcagagctattgttttgcaaatagcgtgagaaagaaaatcactttgtttcttacagtgataaaagacaggatactaataaacagctgaagtttctgtgtactgttatactgaaggggtttaagcttcagtgaatcacttgttaggatagaagtaacaaattatgttggtgtgtacaactgaaaagcatcttagggcactattcttcttttgttgagttatgtccttagagctctatcatatcatttactgtaaggtaaattaatgagtactaaaattacattttcttcacatctaatcaacttgaggaatttccctttttgttgcttttttggtgtatcactggggctatttactttaaaaatcgcaatgaaaaaactgttccaaataagccaggtctgattattttcaatcccagcacacttacttaaatgccaatatcatatggaaaagtttgactccaaaacgccagactctttttttctcttttctggtaaagtgtagctcttggacaaaaataaatgacatggaaattcaaattaatgtcagatggttttatggtttgtggtttgtgtttgtggttgttaactctgaagaatcaggacatataaaagattaaagagtctttcagctactaagcttaaaatatgtatgtttacaaacaaatgccatacactttctatcttgatttatccttacttctttagttactagcatgcttttaaaaaaaatactatgatgtcttttacatcttcccattcatttatttatgcagtaaatatttattgagtgttttccgtggagcagatactactctgagtacttcaagtttcatagacctaactcttccctggaggagatcatggtctactgcaataccagacatgtagacagacacttacaatatgccatctcatagacagacaagcaaactaggttcagagcaactccttctgcttaggggagtccaggaagttttacagcacagataaaacttgaattgagacttgaaagaagtataggaatttgagcaaagaggtgaaaaagtttttctatgcaaaagagtagcgtgatacaaaggcacagagctgagaaaggtttggcatattcagaaatcagtggcaagttccctttgctcagaacttagggcatgttgggtaaagaggagaggggaagagtggggatgaagtaggttaccattttcactgtgagaaactaagagcctagccttcccccagggtctccactctactttttaacttggaatattgcagtaacctgggactggtaaccctttgtggaattgataggagttgaagaaaaggtagacttttgagcagcacaaagaataggattggatgttccaaaaataaacaaaaaggatatgcaaaatggagggggaagggccggcaacatgttctcagaacaaaaagtagttcagttggccattcctaaaggagagaagctacagtttaagtgggaacagtgtttggagggaacctccattagagtactgcagaacattgctgatacggtttggctgtgcctccacccaaatatcatcttgaattgtcataatccccatgtgtcaagggtgggaccaagtggagataattgaatcatgggggtggttcccccatactgttctcatgatagtgagtgagctctcactagatctgatggtttataaggggcttccccctttgtttcagtctgaatttcttgctgctgctatgtgaagaaggacctgtttgctttgccttctgtcatgatcgtaagtttcctgaggcctcctcagccatgctgaactgtgagtcagttaagcctgtttccttttaaattactcagtcttgggtatttcttcatagcagtgtgagaatggactaatacaactgcgcgtggctttcttcatcactttgtccattcaacaacctggtgtaagtaatctagggggcaattggatggaatagcagcaaatgtggtaaataacaggagcaaacaacataattatttttgataggaagtgtaatagcctgaaaataggaaagtacaatttaaagcataaatacagtgtcctatagttcacagttattgggaaaaagatgttgggtgtttccaacaaatgcctaatataagggaacagccttatgacattgataaaagaagcgaatgtcttgtcaggaagacctggataaaggagggtgatagcactgctccacactctgtcaagggattgcaatttgttctggatgccatcttttaaaagagacagttacagagaaaagaatggccagaggaaggaaaccaatgtagtggaagtctgagaacaatattgcaggcaaaacaggtaatgaaacaggctatttaccctctaattgagtaaaggagacatcacagttgttgagagcaagaggggagacaataaccactagatagaagtccagggaggtaggtttagctctgtgatgagatgatccttctaatgcttttccttgtgaacaggaaactaaacaagaccaaataaatatgtctaagtgacaagttcgctgtgtaactgggagtgtttcagctaagggtggtgctaccttgttagggataccgtgagctgtcagagttttacttcaatgaactttaacaccactagaagacatctgagtgtaaaatacagatactcttgatatccttaaaatatatattaatcaggcagcctgagctgcatgcacttaggagctctgaagccagtgagccctgggcatgaggtaagaactatgtgctgtgaggttttcttagtggttttactactgggaagtgacttggttgctcaattgagaggcttccataagaatcgcagctatcagggattgagtgcttgttagtgcctgatgctcagctgtgcactctatatactctatctggtttaatcatcacaacagacttggatagttggattgttttaatcccctgtctccttttaccagtgaacaagtagaacttaaataacttgagtccagctgcaacattttctagctgtgtgacctcaggaaagttatgtaatagctttgtcccaattccctgatttagaagtgtggatagcctctgcatgcggtggctcacacctgtaatcccaacactttgggaggcaggaggatcactgaggccaggagtttgagagcagcttgtgcaacatggtgagaccctgtctctataaaaatttaaaaaaaagctggtatagtggtgtgtgcctatagtcccagctacttgggatgctgaagtgggaggatagcttgagcccaggaggttgagactgcagtgagctatgatccatgccattgcactccagcctgggcaacagagcaagaccctgtctctaaaaaaaaattaaaagtgtggaaaataataatgcctgccatgctgtttgcaattgtaatacctggattccccctcaggtagtaagggatgggagggaatttgatcactaataaaagatacattgacatgtattagaattgtcattgtttatgggtaaaatatttaaaaaatatatgtatttcatttgggaatttggtagagagcctaatgagagctgctgatcacttgcatagatgtatttttatgtataagaacagctgatagtattttgcctttatatttccttctttatgcatacagcctctcaaagccaatctaaaatgctgcctactttataaaatttttctgaccctcaagatggaatgggattacagtttcttttaaactgtcatagaatattctgacttgtctatgacattaatgatttcccgccttaacattatatttacttgttcaattgtctgaccctctccaatagacctcatgctcattctggcatggcctttagcatcttctttgtggtatgtcttggaatacctcaatttcggtatgcaggtcctcaaatctttatttgatttagttgaaccacattgttaagctttagtttttatctctttttcctgattcctttacacaatattgaaagacattggcagaacatttactttatatgggccatagtggtggtggtatttttttaagcagatatgctaatttctaaaattttaagtttgtgtgcttcctcaaaagccaacattactgtgatgaaaacagattttgaacctttgtttggtatctgcatactgatgatgatggatcaatggtgaaagaaacaaactatgttttaccttggccctgccccacaaggcattcatgaaaagagacctgcagatgcaggttgttccctgcattgcccagctgtgctggctgagtgatgttcgaatatgtaagcactttttcatttccttcatctctccatcctcctgttacttagtttttatgctaagattctcacctgagttatcagaatagcctcctaattgaggttcctgcctccattcattcttgactcagataagttcccaactgtgccatgatagtgaacttcctatatagttcagtcacccacaatggccacagggcaagttccaagcagttaagctgcccatgagggcctccaggatcccaaatttccagcagtatcctcttcctgccctcttccttctgccagacttcatgaaatttttgctccagaattgagtgtttagttttcttcatttattgaccactctttgattctccattagctattgttattattgagtgcttccttagtagtgattagggttgtgagctcttcgtgagctctgggtccacatggctgggatcaaagctgtgtgatcctagaagttcttaatctctgtctccttccattttcacatctgtaagatggggatgatattggtcccagcatcatagggttgctgtgaagattgaggtgatacctgtaaagagtgtaggacagtgcctggcacatagtagtgcacatcaatctaatgccgtgcttggtactgctctgggaatttgtattctcatgcctctgatcctgctattctccctatgttgagtgttccccgcatttcctccccaacttgaatttctgtccattctttagaatcgttctaaggtgtcagcttcttagtaaagcttttccacatcaccctttccaccttcagaaagaactgatcactccatcttctcttctacttctgaaccttgaaaataaatatttgtgcagcacactttcatgcaagtattcatatacacatccgtgtcccctactggatcatgaactcattaagaacgaagagtatgatttattcaacattgatttcttagaagctagctcagtgcctggcacatagtattcaataaataattgttgaactgtgctttttgaattgaacaggaaagcactttatcacttaatgttttaggcatgaaatccagagtatgttatcgcagtgtcatggtgatgtgcatacatacatgcatgtgtatgtatgtgtctgtggtttgattagtacatgcctgattaaggaaaataattactgtgagttgttcatttatttaactacttctggtagtttataagaaaggaatttcttcaacttcctaggtagaaatgcaagccaggacttgtggcaattcagtgccaaagatctgttactgttagcctggaagactgcaggtctgccggttccagctgtgctttaataaacaggagctcaaacacttcatgatgggaagtacagctgaggactctcgaatgcacaagctgatcatgctgaatgcttttctgggtcatgttcatttcataaaaacagccaattcccagccttcccagaagaggaaatgctgaaggtcctagctgcgatgtggggtgaggaggaaaccaggacggagagggagagggggcctcaccacctcccctttccttggcaaggaagccagcccaggcacccagcccagtcgcacgggtccctgttaggactttgggctgtgcttaagtacacaagttttttttttttttcttgccttttgtagcgactacgttgttccagtgaccagagcaacatgacatagaagcagagaaaacataccaagttcttgccagttgctaggcagagaaaaacgcttttggatgtttgaagtatgcttcctatgttgatttcctaaccatgacttgttttgaaagccaaacacaatagcatcgtgaatactcatcagcatttggtaggctttgctgccaacctaattaacagatttcagtttagagggatgagaataaaggggatgcttttggtaaagtagtagcttcgtgggaaatttgatttttctctaaggattgtttcttataatttttttgatataatataatatttaatttcaatgaccatatttgtcttaattttaacacaagcagtttttagattatacttaacattgatgctaatatagttgcagtggattttacccaagttttctttttaaaaacctaatgaaatatggaaaaggtagttctctttggtctaaatgtcaatattagagctgtgagtgtgatgatttaccgtgcacttaatttttcttattgtcggagataggaaaatgatcatttcttctgggacaccaagctctcattgttaagtagggcagctggtgaatcctgaactcagaggcagagactccccaggagggccgctgagtacagtaggctgtaagggaactaagaaggatactcttccctgcatggccaaacacagttgcagaggacagtttcagggtgatgttgggcaggccattgggcttggctggtcctaggggcaactgtttccctgtgggcgatggggctgctccatccatgcaagcgacctgtgttccccaccagagctatccagagtggtgtccctttaattctattatcagaccatgtggtttctagcagggaacacagatctaattgaagctttaaggaaactttatgaataggaagccacacatagttgtagaaagatctcaggccagctgtcacttgggagatggaagttcaaggttaatttcacttgactggcaaaagcgttttgagagaaaagttggttttcgtaatctttcttttggttggagacaggaaaaatgggggaaaaattaatatgcctttctactatctcttttgggataaccaggttatattgatgaaaatgattttatgtacaaggcacagtgtgtgtatataaattcttttaatctccaccgtaatcccagagagtaggcactcttatagatggggaactcaggtacagagaaattagagaaattcccagaatcacccagctcggaagtagcagagcttggatttgaactgaagaagtcttgtgatggagttcctgctcttggccagtacactacattgcctgggcatgccgggagcgtagggctgctccatccatgcaggcatcctgtctacaggcatcctgtctgcaggtggcttgactacaggtggcttgtctacgtgcaaatgacgtgcttgcttgctacatgtagacatcttagctggattggcctctgagtcctctgtaatactttcatggagatttatctcattgggtttaagtcatagatttacatgtgtatcacttctatcatatagtgaacttctaatggcaggggcttgattatgagttgctggcacccagcatggtgcctttctttcgagaaccttatggagttgaagaacacagtgccaacaacctggggcagagagtaacagtaaggccaagagcttcaagtgatctctaggactctttttggctcttgtcagtactaatagagataatgtcttgtgttgcaatttggaattcacgaaaaccaaacttaaagatgttttaaaaaagaacatcactggccagggatggtggctcacagctgtaatcctagcacttcgggagactgaggtgggtggatcatgaggtcaagagatggagaccatggtgaaaccctgtctgtactaaaaatacaaaaaattagccgggcacggtgtcaggtgcctgtagtcccagctacttgggaagctgaggcaggagaatggtgtgaacctgggaggcggagcttgcagtgagcagagatcacgccactgcactccagcctgggcgacagagtgagacttcatctcaaaaaaaaaaaaaaaaaaaaaaaaaagaacaccactaactaatttctgatgtatttctttgctcctttttcaagtcattgttttcatttttttggagctatataaggccagtcaactcttttttaaagttaagctagcaaggtatttggctatcaacgctaaataaatcaattcagaaccactattagcaccataaagcttaattctttttctttgtactgtctggtcacctttagaataattcttattcctcttcaatcggctggatttctgcacaggtccaggggttgtctttctgtggggagattgatgtgcactggggcaaaatttcagttaccaagtgtgtccagcatgaagttgcagctggcattgaaaggcatccaccattcctctccaggtacatttcagattcatctcctaccttgttcccccatgtccccatcacgtgcgctggactgatcatctgtcctcacacagatctggtagcttctgcctctggatctggcctcaagtggttccctttgctagaagcgccttttcccctgcaacctcgcctgcctgctgaagccgttctcggtctgtaatgctcacactccatcttctctaagcatctgtctaggataccctccagttccatttaatatcttccttctctgtgctctgtaaattcccatttctagactttcacttgctaagacaagccccacatcaggtatttgtggatgtgtctgtcttcctcaggaattgtggcagccagggagtgtgtgtgtgtgtgtgtgtgtgtgaacacacatgcaatacacgcagtgtctttgcagtccccataggtagaagagcaggtatgcaaagatagtttttcagtgtgaataacccgtttcatttttgttccctcgcaaaatgaatgcctcatccttgtaccatagaaaatatctgaacacctctaccttctagtcatagacttttggattgcctctcttcctattcttttagtttttaaaatatgccagagcccacagttaccttactatctgcctctcctaaattctggtcagcctcttccctcactctggcatggaaccaggcttagaacagtagaattgatagtggtaagaagttgagtgtgactgcacaccacggctgaagctctgctgaaacctttatgaactggactttgaaaccaatttctctaaccaaagtccctattgctccctgagtcaggtgagctctgccatgtgcctggcacaatgcatccatcactttacttagtcctcctcacagtcctcaccagagatgaatgaaagtgccattgttcacagggaagccaggcatccaccgaggtcctacaactgagagtcccatggctgagcccctgatggaagctcaggtctacctgactcttctctttcataatagaacactggctcctgaatgcctgaaatggacacaggagacttttcccatcttctctgtttcccaaggctggtgcccactgctcactttcaccttgcaggcccccaccctacctgtggagtcttgggcgtaagttaggggccttctccttgaccagctcagccttttgtgagaaagttgggaagctcccagaagcttgacaggtcacgcgtacatgacaagaaggtggtggggaaggtgacaaggtggagagagcctgtgccccaccccatggactgtgagactcagctccagccaactatcgccaagaggcagcatggccgtagtgatcctagaacacctgattttccagagtaagttggaattctatattttcatcagagttttctaaatgtttttaaaagcaaaccaagcacgtgtattggccagattcacaggccactgttttgtgacctctgcctggggacatgagtacctgctgtcactttgctcattgtgtctgtctgctctggagccatctgagtatgtgacccccagtttaggagaaggagagaaaaattaatgaggaatggatgtgaaaaaaaagataggaaaagaaagctaatatttaatagaaaaaagtgatgctaaaataagtagaaaaagttccaaatttatatttaaatacaaaagacattataatttagtgactagaaagaataatagatttccttataatagttttttcgaattaaatagttctttttttctacctaaattttgcttaagaatggttttaaatgtatttttaaaacttctaaatcttaactattctaagaatttaaaattttcagatctggttaaatgaaatattaggttactaaataataataccatttatcttctcaacattgaatgaatgacttgtgtttttgttactttgtcattcttgatacattactataatttgataagtatttgaactgtttagccaaattagccgagattacatatatgtagtgttttttaataatgtttctctatacttatacaaaactattgatgattttgccactttaagtagaaagtttatgactttaccttagcgatttaaagttgtctcagtattacgtaaaaatagctttgttattctttaattattcaatgtgttattgctaaattcagtgaccttctgaagactcaaaattatcctctaggctgtggaagtgtatctaaataagcatttaaaataacctgtctagagacacacttgaagaaatgtcacatttctgtaaacaattgtctccatatgtttttaaatatgcagctgatagattcaaaatgcaggcatgtttacaaacaaaatattttctgctagatggaccaataatttctgagttttaaggctttgtaaactgctcatctgtaaaaatgtaaacctctggagtgaatcttttttttttcaagacagtctcgctgtgttgctcaggctggagtgcagtggcaccatcttggctcactgcaccctctccctcctgggttaaagcgagtcttgtgcctcagcctcccaggtagctgggattacaggcatgcaccaccacacccagctaatttttgtacttttagtagaaatggggtttcaccatgtggcccaggctggtcttgaactcctgccctcaagtgatctgcccatcttggcctcccaaagtgctgagattacaagtgtgagccactgccccaggcctggagtgaatcttaagagtcctggtgttgctggatatgggtgatgctgtctgtgccataggtcagtggctctcaagcctgtctgtgtattagaatcaccatgggagattaaaaaaaaaaagaacttaggtcaactccaatggattctgatttacatggtcttggatagggcctgggaatcattttgccttttaaaaacgtgctcccaggtggtttttttttttttaatttttaattttttttgagtctgattcttgctgtgtcactcaggctggaatgcagtggcatgatctcagctcactgcaacctccgcctcccgggttcaagcaattctcctgcctcagcctcccgagtaactgagattacaggtgcccgccaccatgccaggtggtattttatagtatttttagtagagatagggtttcaccaaattagccaggctggtctcaaactcttgacctcaggtgatctgcccacctcagtctcctgaagtgctgattacaggcgtgagccaccgtgcccggcctcccaggtggttttgatgtgccgccagggataagacccataattagccttaaaggagaatgttagacattagtgatacagtgtcagtttcagcactaagctcacctcacccaagatccccagcagcctacctagatcatgctactgggcccttgggccatccacttgatttgagagcttcccagaggtcacagctcaatacacacaatgccatggagggattctcagctaacccgatttgtatgtgtcggtgagttttcattaatcatagtaatatttatggaggcactgggctatatgcttgattttagaatcatgtttaattctccctgaaaccccataaactagttattatacttttattaggagaaaacaaaagagcagtaagaagaaggaaagaatacacccaagctcatacaactcttaagtaagtggtagaatttaaacccaagcactcagactccagagttcatatgcttcatcttcatacctggaggttcaaataaaacactcatggggcaaggtagagagcaaaaatgagtacattagtccatacgcacattgctataaggaaatacccaagactaggtaatttataaaggaaagaggtttaattgactcacagttcagcatagctagggaggcctcaggaaacttacaatcatggcggaaggtaaaggggaagcaaacaccttctttacaaggtgggaggaaggagaagtgcccagcgaagtgggaagagccgcgtataaaatcatcagatctcatcagaattcgctaactatcatgagaatagcatgggggaaaccacccccatgattcaattacctccacctggtctctcccttgacacgtggggagcatgaggattatggggattacaattcaagacgagatttggctggtgacacaaagtctaaccatatcaatgagcagagctgttcaggggtaaacattagggagtggcgatgactgaggaacacagatgaccactgacactcaactttagccaattggtgccagccaggaatgtgggcccagtacagtcaggatatttgatgtttcaaaaaataacctaataaagctatatttttaaacaggaatccttctcgatttttaaatcatagaagtcatttaaccttcattacaaattatactatcttcactgtttttcatccttgttcacgtgagctctagtcaggacagaggtactgaaaagatagcccaagcaatgcattaagtactgaagagggagcgtgtagattccctcagtgaaggactctggtctttcccttggacagtcctgtgttggcctacacttgctctttgtctagtatataccgctcttctgtgaagccaaagctaagatacgagtgctggaatgccatattggtcttaaggttcaatcaaggatgtctgctcacttttcttgctatctctgtggttctgcaaggatatgggtagtatgatgtcatagattgaccatttgttaagacacaaccccctaatcaacttaacattctggtttttctctattttcatggataaatgtctaatttgtatgtattaattagtctcagttgagaagtgtaattaacatttctttagtatttactcattcaacaagcatttaggctgagaaccatttaagaacttagagaaatagagcattttataattctatgggatattacttaaaatctgacttgatcagatattgaaccttttaaaaaaagtgagactaaggaattataagtgatttttaagacacagatttctcctttgatgttctagtacagcatacagtggtttaggcattcttaaaacagtgcagtgtgttgcgagaaacatcattgtcaatggcactatgactgtaggatctgaatattggcactgtgacttagaagcagccaaaagcttaatttctatagcctctgtagcaacggtgcagattcctttagtttgagttattcatctgaacataatgggaactgtcttcttgttagaggaaccaagctttgttctttcagagggcgtagggggtcataactagtacccaacaaaaacagggaaaatgtagttagtaatttttaaggaaaaacttcagctgaacataattcaatgggaaattctaagattattagaaattgcagtttttacttaaaactagatatttgaagattacaaactgcttattttataacttgaaccacttgaaactatccttggcttacgtttgtaagcagatttcaaaatgttcttattttttaaatgaggaagaaatggttattttaaacggctgtagatttagatgtatttataggtggtattctattttctaagaatttgtgtctgtttcttttaaatataaatagttttctcaagttaaaaccttgcttcaattgtatcatctgactatactcacaggatgttttattaaactgtgtctcttttccagataatagcatccatttggaaatcataagatgactgaagaagaaatgtcaaatttgtagttataattctagtttctttgagagaatactatgtcttgcaactacttttaaaaattttacagtattacttcagtctttttttttttcttttttttttttgagacagggtctcactctgtcgcccagactggagtacagtggtgtggtgtcagctcaccacaacctccgcctcccaggctcaagcaattctcctgcctcagcctcctaagtagcagggattacaggtgcctgctgctactgcccagctaatttttgtatttttggtagagatggggtttcaccatgttggccaggctggtatcgaactcctaacctcaggtgatctacctgcctcggcctcccaaagtgctggggttacaggcatgagccacagcccctagccttacaatattacttcagtccattaactatttattgagtattttatccagggccaatcaccatgggaatacccaccacatatacatattaaatgtcaagcattctatccaatacgttctatcggcaatctaattaggaaaaaaatatatgagccaaaggattggaagaaatcttcatagagaaagtgggctttgatctaggattcgaaggataggttgaatgtttgtaaacaggcagatgaagttctcatcattaataacactgataattgaagagagaagtgattggagtcaataattggagcaactataggaagataaatagctgagagataatattttaaaaagtaaattctgcaggaatgatgcataataattatatgtaactttaaaatgattgttacctgtctactgttatctggtcctaggagattccattggttaaagcacacgtctaatggaagccaggctatgttttgttccagtccaaggtcataggctaaacttaattctcagaaacgtgtcccactggtcatcagctggtgggctggctgagatcacagctaacgcttaagaagagacagcttatattagtccagcccaagagtcaccttgtttgcgtttgacaggcatttcacactggcatggacagtataacaaaacctcagttggaatcaccttaatattgtattgttactgtgtattctaactttggggatagagaagagaagacgggggttctttcaatgattgcagccttttgatgtcaactgttttgttaaatcttaaatagattccttgtacttgaagacaatgatatagctgctgatttattctcccataataataaacaattgttggaaaaaaatgaattcataaaagaacagtggagaaaagaaagttatatcctaagaattaaacaggccttgttaagcatctatataatatgtaatgaatctagtcctttgctagattccatgggagaatcagagaaacatatttgacctggtcttcaagaattttatcaacaaccagctttcctgactaaattttcagaatgacttcaccatgaggtccaggcaatgagttaacacatatttattgacctagtgctgtatgccaggaagtgtaaaatccaggggaaatatgaaatccaagaaatatacttggagaggaggcaggggttagatcctataagacattttatgctaaggagttctacatggttttagtataattcaggacgtttatatttctattctaaaaccaaattttaagcatttttaggagagaagttctatttccttttattcttttctttcctattttctttttctctcttccacagtattgttctctactttccttttcttttttaagactacatggtgtacctttatattcactgcccttcatacatactggtaaataataactgttctattaataatgaaagtatacgttttagagttattgtaaagccacataatagaatcacttgagttttcatgattacattttttgtgataatagcattttcttatatctttttattactggttttaattttttattttcactagtatgttatcacaaacaacatatgatatcaatccctgttttgaagataagtgatgggcactttaacttacttttaaattatcatttgtagtcctaaataaagccagatatatgtacttgattatgtaatgctgtaatttgaaaatagttttctttgcccaacagagagacagaattagttcatccttttagtgtacttcattaatcaagataccacaccatgttgaacttatttattttgaaaaaactatatttacgttgaaaactttcatattgtcaacgtgagtgaaaaactattgagttgtttcatcagctatgtaaatatttacttcatcatttttaactgaaaatacatcttaatggatggtatttatgggaagcattgatatgctcgttgtgtttatagctcctaatgagaacgtaatttgtttatccattcactacttgatagtcatttaggttgtttccaatttgtatctattatgaatgaagtggccatgaatgttccaacatgagtctttgggttgacatatgtttccatttttcttggtcaaattccaaggtttgggatagttgggtcatatggcaaatgtatgtttaactttatgagaaactatttttcaaagtagctgtgcttgcaattttacattctcttcagcaatgcataagggttccagctgtttcatagcctcaccaatacttggtattgtcattataaattatgaatttataattttagttcatgatatttagttaatagtattttattctaattttaatttgtattttcctgataactaaagatctaagtacccttttatgtactaatggacatttctttatctctgttatgtccaagtttttacccatttgaaaatattgggtagtttttcttttttcagtattaattttttatttatttttttggagacagggtcttgctctgtcagccatactggagtgcagtggtgtgatcacagctcactgcagcctcaacctcccaggttcaaatgatcttccttcctttgcctgctgagtagctgggaccacagtcatgcaccaccatgcctggctaattttttttttttaaataaatggagtctcactatgttgcccaagttggtctcaaattcctggcctcaagtgatcctcctgccttggcccctcaaagtgctgctattataggcatgagccaccacacctggccttttagtattgttttataggtgttcttcatatattctacataagggccttttgtcaggtgtatgtattgtagatattgcttccctgtctttggctcacctattaatttccttaattgtatttttgatggggacaaaagtcttcaatttcgataaaatctattttacattttttacttttatggttaatatatcattttatcttaaaaaatatttgcctctcccaagatcatgatataattttctatcatttctttctagaagttctctatttctaccttatacatatatgtctattgataaaactaggattaatttttctgtaaggagtgaaataggagttgaggtttgttttattttctccatagggatggatggttctggcaacatagttaaaatgatatttccttctctgttgaattcaactggcatcttattagtttggtgcaaaagtaattatgatttttgccattacttttgatggcaaaaactgcagtcaacgtgagtgaaaaactattgagttgtttcatcagatatgtaaatatttacttcatcatttttaactgaaaatacatcttaatgggtggtatttatgggaagtattgctatgcccattgtgtttatagctcctaatgagctcctaattgtggtttttgccattacttttgatgggaaaaactgcaattacttttgcaccaacctaatagtaataattcagttaaccctcatgtttgagtctgtttctctcttctttttttttagctctacaatagtccccccttaaccatggaggaaacgtcccaagacccccagcagatttctgaaactgcagatagtactgaaccctatatgctgtttccccctgtccctgtgcatacatactgtgattgtttagtttataaattaggtacagtaggagactaataacaataactaagaaaatagaacagttatactagggataaagggggactactgtatttgtccattttatgctcattctatttgttttgattactgtacctttataacaggtcttgaaatcagggaatataagttgtctaactttcctttttttcccccaagattgttttgcccattctaaatcttttgtttttctctataaaatttagaaccagcttgttagatttttcaaaagaatgtaatactgatatttgattgggcttgtggtgaatctatagatcaattttggaaaactcaatatttttaagatatcagttctcagtattttaagatatcttaattgatatcagttctcaatatcttaaaatatatattgtcataaaaaacatttacttctcccaagatcatgatatatttttctatcatttctttctgatatcttaaaaatattgagctcttcaatccatgaacgtggtagctctccatttagatgctattataaatgataatattattattttaagatttttatcgtggtatttttaaaaagttttaccttctattcatttgttcaccactagcttcaaatgtttgaatgagggagcaggacttacccttctgctgggcttgggattggagtactgctgaggatcaggaaatcttttttatattttatagcctagttgtcaaactgtgagagatctcattggctctgcagctgtgttgttaaatttttgcattgctgaggagttttcttagtgccttagtcccatcctggctttctacacttcagaagttctctttctatcctgccaccctgaatgctggaaggtctcagaaggatgcctctaagctcctctgcctggcctccagtttttagtgagtcctgccttatagtggggaaagttctgtgtgccccatgggggacctttcttatttctcctgccctgtccccaaccttcagcgtgctttagcacttggtgaaggcttactggctggtacatgtaagcttggcttatatctaggactctttggaattctaatttcatatgcagcctctataagttcctgaatccaggtatgttgactcatgcctgtaatcccaatactttgggaggccaaggcaggaagatcgcttgaggtgagttcaagaccagcctgagcaacatagtgagacagtgtctctagaaaaaaaacaaaaaattagctaggcacggtggtgcatacctatagtactaagtactcaggtggtgacgaggtgggagggttgcttgagcccaggagtttgaggctgcagtgagctatgaccatgccattgttctccagcctgggtgacagattgagaccctgactctaaaacaaaaacaaacaaacaaaaatttaaaattcccgaaaactttagctgatttctctgtgctctgatctatggtgaattcacactcctactgcagctactgctgcaggtttgagaagagccataggtcctttttcacttatgaagggcttgacactttctggaatttatttcctttaggtttctttgcatcattagctttctgaaagagagcgattttgtaacttatgtagcttgttctgcttgttagagtgggagcaatggtctcttgcaactttctacatctcaattgaaatacataaattctgggtaaagcttttaaaaatattcacttaagatgcatggaggaaaaatcagagctaaactgaaggagattgagatacaaaaaccattcaaaagatcaacaaacccagtagttgggtttttgaaaaaattagtaggatagataggctgctgggtacactaataaagaaggaaagagagaagattcaaagaaacagaattagaaacaacaggatgttacctctgacctagagaaatacaaacaaccatcaaagactactgtgaatgtgaactagaaaaactagaagagatggataaattcttggacacacctattctcccaagactgaaccatgaagaaatcgattccctaaacagaccaataatgagctccaaaattgaatcagtaataaatagccaacaaaccaaaaaaaaaaaaaaaaagcccagaaccagacagattcatagccaaattctactagacatacaaagaagagctggtagcattctactgaaactattccaaaaaaatcgaggaggagagacatttacacaactcattctatgaggccagcatcatcctaacaaaacctggcagagagacaacaaaaaaagaaaacttcaggcgcatattcttgatgaacattgatgcaaaaatcctcaacaaaatacttacaaactgaatccagcagtacatcaaaaagcttatccactatgattaagtaggctttatccctaggatgcaaggttggttcaacatatgcacatcaataaatgtgattcatcacataaaacagaactaaagaccaaaaccacatgattatctcaatagattcagaaaaggcttttgataaaattcaagactgcttcatgttaaaaaactctcaataaactaagtattgaaggaacatgcctcaaaataataaaaccacctgtgacaaacccacagccaacattatactgaatgggcaaaagctggaagcatttgccttgaaaactggcacaagacaaggatgccctctctcaccactcctactcaacacagtattggaagtcctggtcagggcaattaggcaagagaaatagcaagagaggaagtcaaactatccctatttgcagatgacatgatcctatatctataaaagcgtatcgtcttagccctgaagctccttaagcggataaacaattttagcaaagtctctgaatacaaaattaatgtgcaaaaattgttaacatgcccatatatcaacaacagttaggcgagagagagccaaatcaggaatgcaattccacttgcagttgccacaaaacaaacaaacgtaggtatacacctacccagggagttgaaagatctctacaatgggaattacaaaccactgctcaaaaaaatcagagatgacacaaacaaatggaaaaacattccatgctcatggataggaagaatccatatcatttaaacgtccatactgcccaaaacaatttacaaattcaatgttattccttttaaactaccaatgatattgttcacagaactagaaaaaatgattttaaaatttacgtggaaccaaaaaacaagcttgaatagccaaggcaatactaagcaaaaagaacaaagctggacgcatcacagttcccaacctcaaactatgttacagggctacagtaatcaaaacagtatggtactggtacaaaagcagacacatagaccaatggaacagaataaagagcccagaaataaggccacatacgtataaccatgtgatcattgacaaagatgacaaaaacaagcaatgggagaaaaaactccctattcaataaatggtgctaggataactggctagccatctgcagaagattgaaactagatcccttccttacaccatatacaaaaattaactcaagatagataaaaggcttaaatgtaaaatccaaaactgtagaaaccttggaagacaacctaggcaataccattctggacataggaactcacaaatatttcatgataaagatgccaaagcaatcataacaaaagcaaaaattgacaaataggatgtaattaaacttaagagcttcttcacagccaaagaaattatcagcaaacagacaacgtacccaataggagaaaatatttgtaaactctgcatctggcaaaggtctaatatccagcatctataaggaacttaaatttacaataaaaatgaacaaacccattaaaaactgggcaaaggacatgaaaatacacttttcaaaagaagaaagacatgcatgcagtcaaccagcatgtgaaaaaaagctcaacatcaccaatcattacagaaatgcaaatcaaaaccacaatggagttaccatcttactccagtcagaatagctcttattaaaatgtcaaaaaataacagatgctgttgtggttgcagagaaaagggaatatttacacactgttggtgggagtgtgaattagttcaacccttatgaaaagcagtgtggcagttcctcaaagagctaaaaacacagccattgttcaacccagcaatctcattactgggtatgtatccaaaggaatataaagcattctaccataaagatacgtgcacatgtatgttcactgcatcactattcacaatagcaaagacatggaatcaacctaaatgcccatcaagtgtagactggataaagaaaatgtagtacacacataccacagaatactatacagccataaagaatgagattatgtccttttcaggaacatagatagagctgaaggccattgtccttagcaaactaatgcaggaacagaaaaccaaataccgcatgttctcacttacaagtgggagctaaatgatgagaacacttggacacgaagaggggaagaacagacactggagtctacttgagggcggagggtgggaggaggaagaggggcagaaaaaatactaggcttagcacctgggtgatgaaataatctgtaaaacaaatcctgtgacacaagtttccctatgtaacaaacctgcatatgtacccttgaatgtaaaataaacttaaaaaaaaaagaaagtgcatggagaagatattaattttgcctttttttctttttctttctttctttcttttttttttgaggtggagtctcgctgtgccatctaggctggaatgcagtggcatgatcttggttctctgcaacttgcacctcctaggttcaagtgattctcatgcctcagcctcctgagtagctgggattataggtgtgtgccatcacacccggctaatttttgtattttagtagagatagtgttttgccatgttggccaggctggtctttaactcctgacctcaactgatactcctgcctcggtctcccaaagtgctgggattactcccacctccatctcacaaagtgctctcccaaaggcgtgagccactgtgcctggctggaagatactaattttcttaatatgattgtccctgtagttctttgtcctaatagaagcgttcagtgtaccccttgtagtatgtgaacttattggtaattttttgtaatatgggaacttaagaaattcagatgatattcttaggttgatgcttacatcatccaaattaattttatttgcttcttaatcattttttttttcagagcagcatttccttaaaaatgtgagtggttcaagctgcaaaatgtgagtggttcttcctaaatttaagaggtttgggaaacatcattaatgcatttaaaattataatgcctactacatggtggaaaacagagtttgttgacccaggagcttcagaaaagtatagatattgtagtttgcagactggttaaatcaagttatatggctgtttgaatcatgaactttggagctgtggtagtagggaagttgtgctgtttgaggaactcatatttcaatattgtacaggggacagaaagctctctggaggattggtgcttactataataattacagttaatttttatcattaacttcattggacagtttcaagggagatttttatgctactttttattgatttgtacattatttttaaaagatttctgaagactgtttcagggtttgggatttattctaactaaatcctaaacttttataagtccctgtatttctccatgtaatagtttctttgttttttttaaggtggtgatgtatctctttgttgcccaggctggactcaaactcctgggctcaagtgatcctcccacctcagcctcctgagtggctgggattataggtgaatgctgctgagcctggcttgtataatagtttcttagtatagtattttctgattaaaacaatgtgactctttcagtatctaaatatctcagacggcaaaagattattggaggttaagggtctggggccacctttcttgggtggagctagtgagtactatagaccaggaagactttagctgggccctggtaacacctggaacttgttaactgaaagtgggtgctcaattgtatcctgttctgtaatgtggtcatcttaagttacttgctgtaattaggatgagaggaaacgagttctggagagttttcttcaaggagcaatattctgttacagagatataagattactcaaattctgtgatttttattcagttgggaaaaagtcagtaccattagccgagttaacgttaaagagaagagaatttctttgctaaaagatatgaaataatccttttccaaggcaagaatggaaactttggatatcagatttagaaactgactaatttggttcctatatcagcctccacaggaggtcttctggcctgacctttttttttttttttttttttttttgagacagagtctcaccctctcgccaggctggagtgcagtggcgcaatctcggctcactgcaacgtctgcctcccccgttccagcgattctcctgcctcagcctcccaagtagctgggactacaggcgtgtggcacgtgccactacacctggctaatttttgtatttttaatagagacagggcttcaccatgtttggcctgactcttttaactcacctgtggggaagtgtgggcagaatatatgcagcttggcagtgagagagctggagctgagtcaagtgaggattaaatccaggctccttcatttcctgactgttccaccttggagaagctacttaatctccttcagactcagtttctccattaccaaaatgaagctaagattactcccagcctcacaggattactgaagcatgaattgagttaatttgtatggctggtgaaaaaatgaatactttatcatctttgctatgactatttatgatagaaaaagtaagtttgaatggtgaccttcattattctctctctgcctttcttgatttcactgaccttctggcaatgggtagttatttcaagttcattctttcttgtgtgctttcatttactatttagtcaatgggcatcacacctgtgtgcatccttattgaattagactcttgaaaaatcaaaactgtttgtatttcttgccactgataaaaggaccatgtcaaatgatacgaaacattctccttactttagataagcttagtattgatgcaagtaatttcttgcttaaaatactttttaatatttttcttaaacatttgtcatctctaaaaatatataatgcttttaatgtctgaatccatattccgtcaggtatgggcatttgacattgagaggttttcatttatttgtgtcacattggaataaaaaagaattagaaaccaagcatggccacatctcatttattctcaatttattaaatagaacataataaaaaatcaattgacattatttcctgctaatattttttgacggctttcctaattcagcccacagaaggggagattttaatgaagtaacaggaaatggaagaaatagccatgcagctctcccgtaaatggtgttttaaataattttgagaaatggaaaagaattctttggataagaggggattccttattgattgaaattgcttttgtgttgattgaacctgtcctgttgctttatgtattgctttgataaggtcctttgctgtgtttccttcaaccctgtcctccccagggtctctcccataccggtaaatgcactgatattcatgcagttgctcgggcccaaaatgtaggcagcaaccttgatttctctcttccgctcacaattcacagtcaatccatgaacaaaccctgccagcaataacttcataacgtaggcagattctgactgctcctcaccaccttacccacagcaccatcgtctttcacttgtcttatttcagtggaaacctaggtggcccctccatagccagttctccacgaagtagctggagtgagcttcacaccaactctgcttacaaactttcaatggctgcccatcagactaagaataaaatgaactctccactgccatcacccaggccatccatgacctgccctcacctaacaaccacttctcaaatggctcttttcctcccttttgccacacacagtctctccttcctttgggtcgtacatacccctctttcactccaattgttggtgcatttgtccacagatccttcctagatattcccatgcatcgtactctcacttccttaaagtctctcctgaagtgatgtgtcttctaagaagcctttccattcatttgttttatttttcttcgtagtatgtaccaccatctgaacttgctttgtctatttagtggtttattcatctttggcacatccattccctagaatgcacactcattgagggcaaggaactggcctttccattgttattcacctagcgctgagaacagagcctgtcatagagtgggttggttcacagtgactgtctcatgcaataaggatattattcctttttgtctaatgacaagaggtaaaaatatacagataatttcaggacatatgcattagtttcccagtgggacacttgccctcagttgattttgggatatattgttgaatatgtgttttgaaattcaattcctgccattattctttgaccaaaatgtacaattttacagttgacctgcagagagcagctcattagcagtttgctggtcccctaagctcctgtatgttcagcctccccaactgccctggtgcagccctggctgagctggagggtaagaggagcctcctcttccccagcaggcatggtctggaattagttttgctgtcctaaattaggtatcctgggatgaagggaggcaccagtttccccgctgagatgcagtataacctgtctcaacagtgtacatccactctatacaattagattctatcctcagtcttgatttttattccattttttattaggcctggggctgaaacctaaatcccactgcatttggaagaagtcagttatatcctattagatgctaacctttcttcacaaccatttttattcctactccacctcaagcagcatccgtcagggtcacctttagaccagtgatcagctactatatgtcatctccctagagtgaatcctgtcactgttgcctggagtctacttgctgagccacttggggcctccagactacaccccccaatattggacctagttcagatttcaatttaccatgaccttttacatattatgtgcacgttcaagctttaagctctgcaaataattgccctaaaccagattaattatcacacccagcctaggacagtttcatttacatccccaaaatgttggttcttgaggacctaaggttattcagatcccgtgaaacaagaaaactattgatgttaggggatatttgggattttgcagtagttgaacaaatatgcatctttgtggttatgtaacaaaactcatagacaaagctatatagacagacactatatttttttgatggttaattttcataacccaggtcagaaaaaaacttcttaaaatattttttaattatcatagcttttctttgtcctgtggaaggaaagtttccttgtttatttgcagctaatttgcctcatgaatattcagctgtcgatggagctgcaagccaatatccatagaaatgaactcattaggagcattttgttggcagctatttgattcgtgaagtatgtagaagtatttatgatatttcatattttattttttcttgatttttgaaacataaaaggattattgataataattgttatcctaggttttcacagttgagcttatttagtttttctcatatttcttgaatttgtaagaaaatgctttcagatatttgaccacttacacagatttttacaaagtaaattataaataaaaagtcatgaatgacttaaaagcagttcaaaaatgttttacatatttatacatatgcaatacttgttcctcctagttggttattgtcattgatatagcttgctatgtgggtgatgtaaaatagaatggttctcctttttgaaggtcttccttcagaagttttgctgttatttaatgaaaacagcattgaagaagtatatttggttataagaaatagaaaaaatagtaataacagcaaacgtagacattgacactcgatgtaccagttactttttttttatttttgagacggagtctcgctttgtcgcccaggctggagtgcagtggcgccatcttggctcactgcaagctccgcctcctaggttcataccattctcctgcctcagcctcctgagtagctgggactacaggtgcccgccaccacgcctggctcattttttgtatttttagtagagacggggtttcactgtgttagccaagatggtctcgatctcctgacctcatgatccgcccacctcggcctcccaaagtgctgggattacaggcatgagccaccgcgcccggccctcagttacttcacatatattaatttgtttaatgcttatagcaagtctataggagtagataccatattactcttatcttacaaatatggaagctctgcctcagagaggtaaagttacttgtatggtcacacatctagtagttggcagagttgggtttcaaatccaggcagagtggctccagaaagctgttcaccacttcatgttgctgacttctgtcatattttggtaaagactagaaactggactgcctgttgtcccaaaacctggcattctttccccactgatttgaagtgccacctgtttcaaatagtaaattctaaatactcaggatattttctggactgtttctgtgccactgatatattaatacttgtccgtatcagcccagcactaaccgttttagttactttatacgaattccctttaggaatagagtatgagtcaaatggaaaaggaaaggaaacaaattgcagactaaggaaggggtgatatgttgaatgaagagggtaaagacaatatttggtcatgagagaaggagagaatatggtgagtcagaaagggccataggatactgacatgtattttaagtcatgtgtgttttaaggatgatggtggacaattttagaagtcagttttaaagaaaaggtaaagtaagtgcctacgtttctgaggtataaaagttggagacaagttaatatggtttggctgtgtccccacccaaacctcatcttgaattgtagctcacataatccccatgtgttatgggagggacctggtgagaggtgatagaatcatgggggcatgtcttttctgtactgtttttgtgatagtgaataaatctcatgagatctgatggttttataaaggggaattcccctgcacatgctgtcttgcctgctgccatgtaagatgtgtctttgcttctccttcgccttctgccatgattgtgagccctccccagctgtgtggaactgtgaatccattaaacctatttttctttgtaaattaccttttctccggtatgtctttatagcagtatgaaaacggactgacatgcaaaataaatgtgtttaaagaataaaacaagttcatgttaattgaaataaatatgaaaccaatttataaatctttcttttatacctcatggattgcacagtgagttagatcagagtatgagatgaaggaggggagtagccgtcgggcagcgacagatcttcaggttgggatcttcattcatttctctgggttttccagcactaaatacgtttccttctacactgcttacatttccttttcatcggaactgcattcttgaataacgactaaaacaggaatagtgttctgcactttaaattcgggaaatgaccagtttcctgtgccagagaaaaatatgtgcttgagaatattatctatacaatgtactttgtacttcccacttacccttttctttcattgtgctgtgttgttcaagtgatctgatatcaacgtaaattcctgaccagcacaatatgaagtattattatgtgggagacataatagtctactggcatctctatataagtatttgtataatattaaaaataaggcaatttaatagagacattaagaaaaaagttgacaaatatattcatgtaagcttctgaactgcatttagaaatccgttttatttcgtgtttttcaccagctgacaccatgaaacatacttttatatgaaatgataccagaaaaatggtgggatatttgaaatgtctttctttgttgtaattctgaacctcattaaaagtttatttccatctgacattaagtttgtttacaaatataatgacttatgatacataaatacattttaatttaaattttataaactagtcttatgaattgtttttgtttaagtgttccattggtgctgtaaagagttggtagcacctagaagaaggcagcttccaaaggaacagtcttcttttctgaacctggcttttctgtagaggtcttgtcagatcagcctgtcattttacttccagcctgtgtgcttctggtccatgccctgatcttgtgacatctcattgaaattaaagctgtggtctgaccaaacttcacttgcattgttccatttttattaatacctcgtgctctaagttgtttcaaggattgaggacatagacaacaatccctattagcaccttagagtttgatcctcaataaactttttctatcttctactctttgtgaatctccaaatttctgcaatgggtggaagataaatgtatgattagagagattgacatgtataccaaacaactgggagctagcctgggatcctgagcagaaaagatccactacattgtggaagatccaatagttaaaaaataaacagaatttttgcctcgataccttgaattataatacttgttctgatatcaaggacttctccagaaagtagagtctgttggattgatttactgttaatataggagcagagaggaggagaaagggcccttaacttaccctttagttctgttctctctagtgttaataataatcataaccacagtagaagtaatgggactaggctgtggatgtagtaatagtggatagtgataataggcgcagtggcagaaatatcagataacatttactgggccttatttgtgtgctagcactttgcaaagggctttcattacctgcatgtaatcttctccacctctctgtgaggcacatatgattattaacctgatttaatgggtgaggaaactgagacttaaaagcagctggcctctcataaatgctcaccacagagctgaactccaccctgaggagacatcctgtatctgtatgtgtttgagtgcgtatttctaggtgtgtaggggaggaaggtggtgggttaattttgaaggcttccaaacagcccactataaacatcatacatttcctaatcttcgttctttttgaaactgatttctccctagggtttggtgtatccctgaccattcaggaaagagacatcaatgacccgaaacaatacaaggaacacaagatcttcatgaatcaaatgatacttggaatgaatacaccaataagaatttattgccaaaaagttactttattaaaacaaattttaaataaagaacatggagttgtttcaatccttgagaaaactagctgtatacatgcctgccagagctatctctaacaaaagatatcacttctaagagtaaatatctcgcttaattttagattccttggggaactaagagttaagtaaaacatgctccaattttcttaggtttatttttaagtatttttgttttagctttttttttctctgatcatgggagtacattttcgctcaaacacttcaagaacaaatgctattttaaaacttagaactctggaatgatcagtattaaaaagttgtgagtcaaaaggaccttgacaaaaccttaagagtgtggctggaaatggaaatggctacggctacaaagattcccaagtcaaaaaaaatgagtaaaatgggctttgtatttaaaaaaaaacaacattactattagaatacatggcaattaacattcagctttggtgtacatgatgatagggagtagtggtgactgtggcaaaatgaagggaacatgtggcttcttcagctctaacttactgttattcaggtagataggccctgtgttaattttctccaagtgtttgaagagaggctgtaattattgacttttatatgaatactcttattttttttaatgttagcaattgagtcaatgttttaaccacatagtgttgtgttaaaacctacctctactgtttttgacctctgctttagaccaactcattgccttaggccaatggaagctaaatatcctgttaaaataataattttgtaggctgagctgtgagatgagtttgtgggaatcaagactgcagaaacaaactagaagttaaatgcaacaaaaaaattaaacatttaagaaattcacaaaaacacacttaagaaaagatggcatttattatagtatttcactgcagtcatctcaataaattttgtaactttcacctttaattttagtaattttacgcataattgtaattctgtcaataatggtcctgatttctgcttcatcttctgtccagttttactcttgactttcctgtcacccaagtattaaatatcttcaaaactctgtgccttgtatgtaacataccaatcgtatgctgacaaatgatgaattctttcccctgaaaattcctgaagaacgcagttctttcatatttattttttgttattttgaggtcaaatagaatacactataacaatgtggtaattatcgtcacatcttggtcaagttccagccaagggaggtttttatttttattttttgatttggatggtataatgacaaattatcttcacctaagctctgagatgatagtgatgttcattttgaattaataattggtctgaaaaagagtaaaacatcttttgttgggaaattaggcagatattaaaagactctcaataactgacacaaaaaatccttttgtttctgaatcacaaggaaacagcaacaatcaagaggctttcactgaggatttagacatgctgtttgggaatcactgcaatctctttagacatttatttgagtaattcaggtgtgggatattttaatgatttgtagagtgatcaatgcccacgaggctgagtcttagatactactggactactaacaaatgatttattattctgtcagtttctggagttttgtgtggattgaatgttgtcatggaagtttaggaaaaaccacgatctatttcaaagatgaaaagatgctgctcatattctgtaggaacaaagtaacatcattttataattattgttcattgtatccgagtgggaaagagattactaaacatcctgtgagcaccggacagtccttcactacaaaggtttattttaaacaaaggtttaatccagctccaaatgtcaataatgtcaagactgataaaccatgtcatatggtgatagaagaggaaaagagtatgtgctatcctatattccaagttgctaatgaatatttgggaagctaatacactagggaaattaatgcagtgcagtctggtggtcttcataaatgaaaaccattgagttaagaacaaaagtcgttagtctttgcagaagtttattgagcacttaccaagtgccagatattgctgtaggtgctggggatatcccagtgaacaaagcaggtaggctctgcattcatgaagctcctatccaaatgtgggagacggacagtaaacaaatagataaaatgattctgtccttgtgccttttgtcttccccatctctacactgtctccaatggactggggctggtcattttttctctatatatatgctttccctaagttacatctttgtgccagtgacaatcaactttttactgtaagtcctgatccctcccttagaacctcagattggaatggctaactccctgctcatgatttccacactggtgcccaatagaaatctgagacccaacaggtccagaacaaactctgattctcccaccccaaaaccttcttcactttcagtcttctccattccagtgatgccactaccaaccaggtgttcatgcaaacccaaatgacaaacttctttcatttattaggaccaaacgaagcccttatcttccaaagaattgtgatgtgatattattgttttaagttaaagggtaaaaggaaaatatttacccttattgtttttttctcatttattttcatgaaattagcacagtcttttttcctgcaacagtggagtctgtcttaaatttttcctattttgcctttatatatacaatttacctatctgtaaccatattaaatagccaaatctttaaggtgacagggtgctgctaaatagtctactttcagaattttgaatgtagaatatttgttggagtaataaggtttagtgctgtatgatacaaaattcagattggcatcgggtatctagaattaaaagtataaattgttaaaatctgccggacttttgagaacgatctactatgtgtgcctggactgatgaaaagagtgggaagggcaaaaatatgttgaaagagatcattttgtatgttgaaattataaaataagaaaatgcattctgagaatgaagcccatggtgagctttgctttacttttgaactagcaacagcataaatgggcagacacggataaaggagattgctgtcatcttatttcatcttaatgagtttcttccttttgattcttgatagcaaatttgtgacatttttagctttattaaactttaattagtggataatatttatcatatgcatttaccaaaaatacaagataatatggagcaagttaaagagaaaataaagagtactctgggaaaaagcccataactagagtgatatctaggaatattttttgtggagccattgttaggggaatgctggtgagtggagcacatgttcacacacatgcaagctgaaagatcctcggggtgcggggggggcggcatatgtgagagcctcacttcctctgaatgcaacttagcctgtctttgactcacctgtgtagtgtctgcatctccctacccttcagagattattgtgacctgttatatgcagctttgattgtgattttcatgggtgggaggcacgtcctctgcggggtgccagcatccccaagtggttgggtgagagggtgttgtatagaaacaaccactgtttaacactgaattgaaagtcaaagtgaaataagtaaactcgattttcctgttaagactcatttatttctcaaagtctgctttttgttttagtctttgaaagaataaacataaagaattgttcatcagaagatgatctctggaccaactacatcagaatcgcatagaggtcctgctcaaaatgtagatttctgggtcctaaattaatctgctgagtcagacctctggggataggaatctgcatttggaagtaactctccaagtcttaaacacaagaaaaagtttgaaacccactagcataaaaaagctattttccactgcttgatttcaaatatatgtgtgtcaagatgaagattatctgattctgatgaaattttggacaagtgggaatgaatacagagataaaacaatgtgtagaatccttcatgtatttttgtgactaggctactaatgcttccttgattgatataagaaggattaaatatgacaacagagtctgaaaggacactatagatgctggttgttaaaggatgattttgaaattacctatttattcctagctttaggaaggaagatttaaacctctcataaaaaatgtcaaatcgttttcacgattttgcgttctcagcaaacatctaaaatctgccttcactgttgtataaaatgaggaccttgggaaaaagaaatgcttggtcagaaatgctaacaagttccagatttcctttcatatacaattaaacaaaaagttccatattgtcacggaggataagaagataaaaagtcagataaagacatggtttactttcttagtttgtatgttccattctaactcatattttgatgcattgttactacttcctcactttaatttatccatttcagaatgtgactgagttcagtttatgaagaaaaggtttataaatgtagaggtgaagtttaagctttgcttgcacgagaggttacctgtttactatgtggtcaatatgaaatgtaaactatgtaaactaggcaaataaaccaccatgttattttaagagcaaatggaatgagattaaagaacatttttgcgatgagtatgtggaaaatattcaagaaaaacaaaactaaagcaaaaaattaaacagaataatactaataaattgagtacttaaaaagttagaaaattctaatttttttcaccaattatccttaactttagataagagttcaaaaattatatcacttgttcccaatagacaaattagattgatatccaatacatgttcattttatttatttcagctgatgttgaagagttgaggctattccttctgatctgacattgtagttacagaatctgaaacatgaactggttgaatcaggaataaatgtggttctgaaggtcatgctaggagcgagatcttgtggctcattagttttattttaaatagtcttgttcctcttttgatataagttataacatgcttatagctaaatattacaaaaattagaaagtgtacaaaggtcaatttcctattacctaaaaggaactacccattgcgttttggtaaatgccgcctacttttttttcaacattagcacacaccaatattatccatttgtatatctgtatatattcttatgcatctgcatatatgtatgtatgtagcaaatatgccacttaataaattttttcaccagacagtattttatagacatctcgtatacacatgtatgtgtatatatagttttcattacattccatttatggttgcacttttgctatagtagtctataatttgaatatatcataatatgcttagtgattcttttattgatggtcatttagattgtttccaatttttccattttggtaatcaatacaatgaatccccttgtttgaatatttttcttagaataaagtcctagaagtagtatctctgggttcaagaaaatgcccatttatttttatttttatcttttgagataggatcttactctgtcacccagactggagtatagtggtgcgtgtgactatggctccctgcagcctcaaacttctgggctcaagtgatcctcccacctcagcctcttaagtaactaggagtacaggtgtgtgccaccacgcccagcaaactttaaaaatctttgtaaagacaaggtcttgctatgttgccgaggctgacctcaaactcctggccttaagggatcctgcctgggactcccaaagcgctaggagtatagacatgagccaccatactggctggaaatacccatttaaaaatttgagatatattgtcaatttgccctctgaaatgttgtaccattttatactcttattagccatgtcctgagggtgataatggatggtgtcttgaggacataaatgaaatttgctagatggaaattctcttctcctttttctcccctaaactttctttttcaaggctcaatataatgaaattattctactcttttttaatttgtgaaaataatagcctgtttagccattggtcaaaacttaatgaccactgagaagagtccaaatctttgaattactaaagggcttatttgcctaccatagaggtatggagagcccggacctgaacccagaaatttaaattctggcttcttaaaaggctgggggtgggcaatagaggggaaaggaaattctttcattccttcttgaaatgtcttcctgggacatagtggcagggatagctatctatctatctatctatcatctattttatcaatcactcaacctacctacctatcttctatctatttatctatctctaatttcgaagaggatcacttcagaatctctggcccttttgttgttttattagaaatgccattttccattaaattttggtcttgtgctttgcaagtcttagaagtttaggaaacatctgactctaaatgcttacttcacaactttgagatcctggtggataaatttaacttacatatacttgatgatttctggctctaccccaacatggtaatggaagtataattcccagagaggaacagcaatgcacaagggtttctgtgttacaacaaaactattatactcaacaaatggagttttacactttggtttgttgtaaagaaattagtttaattctactcaacacccagtcattttattaacctaatgttaccttcataatgtcttcttgtttcatgcaggcattcttacacagaatgaaccagtgtgcagcatcaaaagttgacaaaaatgtgacagaagaaacagtgaaggtgaggaggtacaactggccttcaacctgagcatgagtttcctatagataaattttgctggtgccatggtacttcacagcccttcagttgatcggcagttccactacaagttgctgttccagtcagttgtattggtaataattttaaaaaggagaaatagggcttaggaaaagagatggaagattaagagaatggcagagaagtcatagagttgttttttttttcaaccaaggccttgggaacatttgaacagaggaagaaaaataaattggaggtgtggtggcttatgcctgtaatcccagcactttgggaggccgaggtgggaggatcacccaaggtcaggagttcgagaccagcctgaccaacatggagaaaccccgtctctactaaaaatacaaaattagccagccatggtagcacatgcgtgtaatcctagctactctcgaggctgaggcaggagaattgcttgaacccaggaagcagaggtgttggtgagctgagatggcgccattgcactccagcctgggcaacaggagtgagactctgtctcaaaataaataaataaataaataaaatgaattggaggctaagaacctcaatgttttggagaaggagggagctgagccgtataagaggattgttagaggtggctcaggaaggcagggtgtgggcaaacgtgacttccagtctctgtgatcactgaagaaccaaatatttcaatggttcactccacataatatccttaattcagaaacagacatataatcgtttcttttaaactgaaaggaaggaaaactgtgaacagtcttgaaaaaaaaggatggtaaatgaaaattaattatactataatgattagtcatttaaattgtatgtactattcctgatatttatgatgctaacttactgttttattttgctggaaatataaaacattctaaggatttttaaaggttcttctgactctatttccccatgagtaaaataactaactttaaaattttcctcccaggagagtattttagttagcttgcttttggttgtaagctaaagaatcctcttactgaaattgaattgaagacactttggctcacatatcagggactccagacagaggagaaggtttagcacctgtgacccagagaggccagcaaggtcgctacatcctctccagctaatcactgccatccaggttatcagctttgtgctcaagctggctcttcctgtagctgcagagctgtcccagcttttacttctacatgtggcaccttccagagggaaaagaggactgtccgttctggtatgcccttcttaggagcatagatgccctttccagaaaagcctgccagcaactctgctggtttccatgcagcccagctttgagaagggagtgggatacctggcttgggttgggcaaccgaggccttgccctgagagccagccatgggcttctggttcccagagttgtgtcggcggcgtgaaggggaagtccagacaaaattgaggctctgtcggcaaggtgggaggaaggcatggatgtatggttttcaggttggagagagaatactatgctatcatagacttaaaaaaattgcttcactactgagcttacttacacatttgtttatttacagatgttgttctcaaacattgaagacatccttgcagtacataaagaattcttaaaagtcgtggaagaatgcttacaccccgaacctaatgctcaacaagaagtgggaacctgctttcttcactttgtaggataaatttctttttattttaaaagttcttttcccctagattttgagatttagctcttggcatagtggtatgtgtgaattcagtattagaagtaaaatctgacaccaaaagtttaaggtcttttaaggccaatgagatcttccacttagagccagaaaaggggagagtgcctcacatcatccattttactgaagaataaacagccttttcttttccccgacaagttagttcttcgcatcctctgtagctttgaattctaatgaatattcaggctgttcataatgaatgaatttttatgtttctctcaaacatagagcatttttcttttgagacagaaaatcctcattcctataatggcacgatgagccttttattagtcagaatgttcccttgttaatcaacttgtgacaaacctggagttgaataaacccagtagcaggaattttccccaaggatggtttctcagaagattgtactgagcagtgcagatgcaataaccagccagtactcagttgtttcaaattacacatcctgtgcctgtatgatcattttcatctctttataaatgatgcccgaaattatttatgcaattaatttgcaaaataaaaagacaactgtgtaatgaaaattgttttcagaactaaagggcattcacaccaaaaaaaaaaaaaaaaaaaagtatgctgctacgtataccgttacgctgatgctaattttgagaataccagtatgaatccatgtatttatcaaaatgctagacaattaatgttttgtttaaaaatgtgtaagcgtatttagtgatagaaataaaattttgtatatttttaattttccatctagtcttttagaaaggttttgatagtagtttcaaaatctgtctttatgtagtgagcagactctgtgttctttttatagtcagaaaatatttcaaaaaataattatattaaaaggagagtctcattgggtataggaagatcaagctgtgtttttcttgggcacagctggaagccacgggtgtttgtgtccactcacttaagcctgctttatttcacgaaagaatcaaaaggatcagaatcttctgccagctggagtagttgctatctcagtccagggctgtgcctgggaatcttgcaccctgacatccatttccttagtaacaccttatgaatctgtcatccctgaatttagctaaactatttctgaagtttattttttttctggctcatatcatttcttgagataagtgattcaatacattttctgctggttatataaagtagcacttaatgtttctaaatgcatgccttcaaaatttcaatgggcgcacttttgttctaacatttgatcagtaaattttttgtgtctttcttttattggtactcttttggtgataaggcactttgagaaaatcttacaaatagaagggagcttagagatcatgatggtacaaatcctcttgttttaaaagaatatctcctatttatctatgaaggctgtttattttgttcctactttagtgacagctctaactgttagaaaatctctgtgttgatcccaaatctgcccatcactgcccttgcgacatccaccactgccttgcgacatcacctaaagatcctagttttatatttggagctacataggactgtcagtcctttacaacataacgctgtttgaaagtattcatgatttcctgacttttctatacctacaaatatgactctctgaaacaacctctcagatgtggtgtcctcagggtctctcattctcttagttgcttgttgctagatattcttcaggttgtcagtgtctctcttaagcacaactgtctagactggtgtatatgaactgcagggtcctgtgtttctctatttctatatgatctggcacttccttttgccttcatctaaatgactttattccgtctccctgcctaggaatcatggattcatatacctggaccttgctcagattgattgatctttcttctacatacattccttattcagttttcataaataacagcaagaatatgtatgtaatttcttcctctttttaagctgtaactgaatcctcatggcaaagctcagccctctcttttctgtgctttgccaccctttggttcatttgtctacttgaattcatcacaatggagtataactgatatacgtctatctccctttttaggctgttcttcatgggccagggaaaataaacaaaatttatatatagctgtatacacgcagtatagctcttaaaacgtagtaaacactcagtgaagtttctttaagcatatcacataggttaaaattgcagaatggtgaataaaatgactaatttattcttacatgaattcacagaaagacaagtttcgtatctatgatgaatattgtagtaaccatgagaaggcacaaaaattacttcttgaactcaacaaaataagaacaatccggacatttcttttggtaagtgtatatttatgtgttactgtatgttgatatgtgttgctagatccagtgagagccaaggaatagcatcaatatggagtaaaaatctgtgggatacctctgttagcatccccaggcgaggaagcaagagtcctttgatgaaaataaaagtgagagctgggatcagcttgtgtttggggaagtactgctaatgccccctcgtggttgattatgaattatctgtgagaccccacaggttcctagcgagagaggagcttctgccagagttcatcactgctttatagccccagttccctagggtgtcctttacttctgctgacagagctgtaagtagggcagtaacaggaaggaagaaaatcgtggatatggtagaaaataagtgtaaaatttaacatttctcctacatacatttcttattttgttttcataaaaacaacaagaatatgtgtttaatttctttaggttagcagaaacaaatgcatattttggctgtcacgggcaaactaatcgctagtgaggagcagattataattgtgcgattcagtgatatttagcagagtgtggagttgcacagccatcccccaaattcatttttagatcatttttgtcacctcagaaagaccttcatgtccctttacagcctatctctgtacccaccgttagcaccaggcatacactgatttgttttcatctctatagattgccttttctggatattttatagaagtggactcatacaatgcaatcttttgtgcctgacttctttaacttagtaaaatgttttcgaagttcattcttgctatagaatgtatccattttctttcattttaattgcaaaatactgttccattgtatggatatgctgtattttgtttatctatttgccagttagtgaaaattgggtctggttccagtttgggctcctataaataatgctgttatcagcatttatgttcaagtctttgaatggacctaatgtttttattcttttgggtagacattaaacgtggaattgatgcctgtgttgtatgataagctcatatttaactaattaagaaactgccaaactatttgtcactgtggctgtgccatatttactggcaatgcatgaggattccagtttctctatatctttgccaacacttggcattttcagtttttctgattatagccatgctagtgggtgtgtagtgctatctcatggtggtttaaatttgtattttgccagtgactaattacattaagcatgctttcatgtgcttattaactcttcatgtctcctttagtgaaatgtttattcaaatcttttacccatttaaaaattgggatgtttattgtatattgagctgtaaatgtttgttatttcagatataagtcctatatcaggtatacaatatgcaaatatttcctctcagtgttgtttgtctgttcatttttaaagtatcatcttttaaaatcagaagttttaaattttgatgaagtctaatttattacatttttctctgttatgaccgtgtttttttggtcatatataagaaatctgtattcaggtcttgtacttactttttattcctttgttttcccacttttgatgctattgtgattgaaattatcttatggcattttaaaattgttcattgctaatatatggaaatttaactggcttttgtttattgatcttgtgtcctgtgatctaaatttatgtatttggtaagtagttcttttgtagcttcttagggtattttgcatataggatcaattatctgataataaagatgattttacttcttcttttccattgtggataacttattttttttcttgccttattgcactggctagaaccttcagtatagtgttgaataaaagtggtaagagtgggcacacctgccgtgtttctgatctcagtaggaaaacatttagtccttccgcattaagtatgatgtttgctattgatttttttagtagatgttctttcttatgtttagtaacttattatatttctagttcatatgatatgatgtgaataatattcatatatattgacaatgctatgaattacatgcattgacaatgctatgaattactattcatattattcatattgtttaatgattcaattattgagaatgagatattgaaatctccatgtataattaaattatctattttaccctttaactctgtccatttttgtttgctgtattttgggactctgttagatatgcacgcattcataattgttagattcatatttctgtttatcatcatgaaatggtcttctttgtccttgtggcatttcttttgttaaagtctatttttcctaataatattatagctatgatagctctcttactatttgtatatctttttctgtcattttacttacaagctatttatgatctttaaaatgtctctagaagatggcatattgttgggtctactttttgtccagtctgacagtctctggggtcctcagtttgtttgtttttaatgtaattattgatatgatggtatttacatgtgccattttattactttttcctacatgtcttttttgtttgtttctctgttcctgttttaccacctacttttgtgtcaatcaagcattttttatgtaccattttgattcctctcaatttttaaactatatcattaggagtgtttttggtgattgctctaggaactacaatatgcatcttaacatatcaaatccatattaggttaatgctgacctaattccagttaaaagtaggggacatttggctataatatttcctgagtagaaatggaaaatgttccatttcatctctcctctactgtgctattattgccatatatattacattatatatgttataaacttcacaacacagtgttacaattattgctttctatatttttctacagtcttttatagttatgtacacatttaccatctctggtgctcttatttcttcctgtgtatttgactctgatattaggtttttcagcccttagtacttcttcttcttgtaagtcagttctactagcaataactttgctcagtctttttttttattttttttaaatctgcgaatgtctttattttgccttcatttttgaaggatagttttacttggacatagaactttttgactggtagatttttttctcagtcagcgctttgaatatgtcattctactaccttatggcttccattgtttctgatgataaggcatctgctataattgtatggttgttatactttatatgatgggtcatttttctcttttttgctgctttctagattttctctttgtgtttttctttcagtagttcaatgatgtgtttatgtgtggggatgattgtacatttcttatttatagttgagctgtttgaatctatagattaatttttcatcaaatctgggaagtttttagccattattttagaaatatttatctgaccctttgttttcttcttcagtgaccccaagttacacacatattggtaagcttaatgtttcctcttggttcctgatgtgctttcatttttctacaatctcctgggctcaagcgatcctcttacctcagcctcccaaagtgctgagactacaggtgtgagccactgtgcctggtcaggaaacatattcttcatgtatgtataaggagctggctctcgtcagccagtctaaagtggtagccaacctgtggtattatgaattgagttcgcaatctatacagtactaatacacaaggcaataaaagtgggctagacagccctgagtgattttaggacacttcagtccagaggtaggtgaggggatggctgctttacagagatgctaacatactggaatggaaatatcactggaaatataatgagatgtagatagttgtgtttcataggattcctctgggggaaaataccatatatagtttgaaagaatattttgataacccgattgcagtagggagtgaaacccccaaagagcatgcttgcgtaagagcatgcatttgctgctatgctttctgaggtgtctgggtgaagttacattcagagtgctgattttgctgactttgctgacttggacacacatattgaggctgatatgcagttgctcaggacagcagaaaattgttctgctactatcagtcttccttttgcatcctttactgatcaacactcagcctctcctctcagtgaacctgaaacagtattttaaaaattgtagtatgaaagttgctaacttagtttgatatttgaggggctctctcttccctctgttagatatttattaaatagtttgtctgttgtaggcactgcagaaagaaggtttgggcaagtttcaagtctgtttcattgcatgttgtttatgaaggtaaaggctttttttttatcagagtcagttttctcttgaccaccactttatcatcaatgaaagctgcttcagtttcaccccactctgattcctgattttgagatttcctcattcatcttgcctctgtggaaatcctatctgacctccttctctgacactggctttccagtgtctggtgcagtgtgatagcaaaaaagcgactcagagatcacttgcttaaaacctgtcctatttatggagaggaaaactagaattctgaaatcaacccaagttgtatcacttgtcagttgcagagcctgagttagaacccaactgtcttgatacttgcccttctacaacataatatacctttctttataagcaaaatcaatctatctatttatgtgtcaaccatctgtttatccaccacttatccatccattaatccatccattcattcatctctccatctctttcccatctctttttcctcagacttcatactcaggagaatataggtgcattaatttttaagatacagattcaaaacacagttctgtaactagtgtttctctttgtctctctgtgagtgtttgtgactgaatatgctcttcttagagaaagaaagtatatgaagacatatgctttcactagtttttagcattttatggtggaagaaagtttcacagcgtgagattattaaagatgtaattaattttgattattttcaccccagaactgcatgctgcttggaggacggaagaacacagatgttcccttggaaggatatttagtaacaccaatacaaagaatatgcaagtaccctcttattttgaaggtattttatgtgctacctcattgtagccattttcttgtatctacatattttgctgatgatctttttaaaaaattattttaatgagtctgatattctaaaggaagcgtagaccataagtattggaaaaaagtggccattttgtcacaaatttatatatagtgtaggaaacagatgttcagtggaaaagccactgaaagagcttttggtgctctttgtataaaacatacttgtttaagtttcatttagaggaaatgcattagcttcaagatagatcccattaagttcattatgactagtttatatagcattttgcagagccttttattttaaataaatccactgctggtatatttttgatttccttaaactttgatgactttgtgatgctaatgaaattaaggtcaaaattatataattgcataactaggatttaaaattattattcaagggaatagtgtgggtcacatgagacaataaatgatagaatgtggaatgagattgctaagaaaacattgcctttagaagatgtaaaacatcctactaatggttgtaggtttgttgcctcattatagtaaccacaatttttattatgaaaaatacactgtaaggtgccaactactgcccttactgcaagggacctatgaataggtcaagatggcaatttttgaaactgcgttttttggaaccataacatcaggatgggcagcctgggatagtggttaagcacatgaactctgagtttaaatcccaagtattgtgcttacagggcatgtaatcgaacctcttaatgccttggtttttcatttatgaaatagagatgatgattatgatgatgatgaatttatagcacagacctatgggaatgtgtgtatgtatgtatgcatgtgtattctgaatacatatatatatatatgcatatatataggattatataatgataatgattaatttaactattttatataaatcaattagcatagtctgtgctttataagtattagccattattattcatatctatttgattccgacttattttaatatataaattactacttggcatctaaacttcacaaaatactcagaaatactagaggcatattaactagtatttttattgatttgctttttaagaaataaatgttgaatttgaactaaaaatatagtttaatacaactcttgtcaattccataaattgggtatctgcgtaaattttatttgaaaatatttgttccttattaaagaaataagagtctagcattgcttgcttaagccataaaaagccacagagaggctgacacaggagtccccagctctgcagagggagtattgttgcaaatgtgtctcctttttttctgagaatccctacagacctctgacttgactcagtttgcatgtgtactcataactgcagttattcctgaccctgattttggaatcccaggggatttaagaagggatagggagattaaaaggttacatcatgacaaagtggttattctaaattactcatttctgattagggatgaactcaggtatttgaactgacctcccaaaagaaagcatcgtggatttatcctttcttagccagaaggtcagtttagagccccttcaagcagtggatggatgacctcctttccgttttctggtagattatctccacaaagcaacatgaaaaaactgttaactcatgtagtagatacattggcattgatatttgtctgtgttgtaccatcagctgaaaaattctgctgggattttctttgatttagggtggttttgtttattcagaaaagatattatatggattaggttgtagtgttaggaaaagatacattgatgttatttgtggagcttgaaattattgagttggtcaatcaacattgggttcttagcaagtaatggccaagggattgtgagtgatggttttctctcactatttcacattatgtaaattcatattcaactccttagataattggactccaaattatggcctgcagactgacttgaccacttgacatagaggcacctgtggtgcctctactctttagagtttctggagaagcctgcatgtctcacttatggatttgtgatccagcataacttttaaaccttgtcttgggagtgatataatatgtattcctctggtgttagctctgcagaacaaggtgtgaacataatgaatttagggtccgagtttatggcagagttttcaacaataacagaactcaaaagaatataacaactgcatattttcccactgtttgttgagaatttagaaaattcttttcaagacttgcctcaggatttctgtaattgtgttagttacgtgatgatggcaaccatagtaaggaaacacatgactaattaaaaagcaatgtggtcccaactgtctagacagtgggtgaagatgtttaattttgtgcttttaaacaactaacaagggctttcaatatgtttaagagattaaggattattctagttttgatgaatgggaaaaatataaataaaaagaccctccaaatcccctaaataccaaaaacaaaaaaggaagctattgaggggtatatggcagttcatttgtcacatagaataagcctgagtaattggggaaaacagcataaatagctcctttaatgatggagtattgtagaagttttaaattaccttgttgtagtataagacattattttaaaattttactttgtttatgacatagttttatagtttctcaaaaatattagaaaataggatttctaaattatttttgaaattatgacatacttaaagatttgttattctgttaaagaccctagaaaaatgaaattaacatataaagatgtgcttctgttagaacaaaagtcaaattattaaaaacataaaaatttatcctcccagtaagatatttgaccattcgaaaggaggaacctaacttaatttttaaaaacaaagtaagtttgaggaaaaattaaagtgactgcaaaaatgtgcatttgggttatacttactcattgattcaaacttttatttttgatcttagaggaatttgtgatgtcacttatggaagtggctctcatgatctttgcagttttcatttcctggtcagtctgaacctgctgtaccagaaagctgaagatcaaagggcgatttgtttttttgtgtataaacaggagttgctgaagcggactccacggaaacacagtgactatgcagcagtgatggaagccctccaagccatgaaagctgtctgttccaacataaacgaggccaagagacagatggagaagttagaagttttagaggaatggcagtctcacattgaaggctgggaggtacattcactttgcttgacaatcgagcttaagatagttttatgttgcaggcctcgtgcagaattactggcgttggatggaaccataaatggtcattttctcagatattacttgaggactgtagtcaattctgaaaagtgctcactttcatcattattttaaagtatctttaaggtggtggttaattgagttaaccttgacccaataagtttgtgatggagccaatagatactatgtgggctataagctttaatattttagcatttatgcatcatgatgatcctgttcctctctcaactttggccataagtacatagcctgaatcctgggagagtgaagaagggaaggaaagttcagtaggtgtgctgtgatattactttcagactctaaattaggtgttcacttaacctctttgttttgatttcttcatttataaactgaaggaactggctatgatcttatttttaaggtttattcaagatgaaaatgttatggcttcctgagtcactaaggaaatttttttatgccaaaaaaagagtatagttcaaaatctgtaaaatgttttcagctaatggtaactttgattcccagttatttcatgcggttggagtcctatgttactttaccactatgcaaagaatgaactaaaattctgtcattacattgccttaatttatatgagtataagctttggactaggttaaattttttctttttataaagtaagcaattaagataatttttaagtgtaggaaagattgaaaatgctcctagccaaatactgtatcacataaaaccaagtggattgcttttatctttaaggctttagatgaatttgtttcttacctttgatatggtatttttcatttggacctaaaattatgttcacccagatatgcaaagaaacctcaaacattgaccttaaagaattcttccgtttgtcccttctctcctgtgaatcttttaaaatgctaactggggttgacctagctgcttctttgtccaactctttccatttcaagtgtccggagtgccctcatattcagcatgaatgctgcctctttcaggcggctaaaatggttgtgtgttttacagttgattcatgatgataatttctttcctggggagctgttgtcttgctgtgatgagaagaatggactcaagtttgcttttggaattctgttgtcactcaaacactggaagaaaaaccgaacttgctttaaattgcagaatttgtcagtgaaatgtgccaggccttttactaaaacaaaaacttcagagatttttttcagcactctagggaaatggaatctgtcataattttgtttcatcctggtgtatatcctaaaaattgagtgtactgcagaaatgagagattccttaaaagttattgatattgtttatacagtatacaccagttactcaaaggaagaatgaatgtaggtggaactatatcatatgtagcccacaaccgagggtgcctgacttcaccagctttcgtggttttacaaaattctgagtgcaagagaaaatggggcattgagacagagaaaactggatggagttaaggaattctgttgtgtattcagagaaattagagaggaattttgccacttgttaggtataagaacttgggtaagttaaatagctgtcgtttttaaattctacagtcagagttccaggaggcacaggggagataatatgaaagttgggttccaagaaagtggttctaagaagaagtggcaacataaattctacttgagtaggaattgacaatgattgcgaaagacagccatacagagattgggagttttgggactttgagggagtgcagcttggagcagggcccagtcagtgggtttggcaatttgtgataagaaccccattgcctgtcccagcccaccaccccatcgaggggggaacttggttttcaggcctggtcccttctggggagttcaggagaaattgctagtctcataggactctctgtaaggacgcagagtcacacaggctgaattcaggccctgggaggaagctgtaatgagttaaagagctgtctttgacttgctgatggtgatccttgctcagcctgcagaagctttgtgtgtctgtggagaggctaccttagcgttaggacagggagaagttcagacgtagggtggcatgacagttacagaatgcagagttccctgacaagtttttgacgttaggagtcttgggcttatgctaggaaacagctccctctccagctccttccacagcctcctgcagaccaccttcagggcctccttagggaggacattgttccttcagtaactaaaattctatttgatggaatccaagtctgatacaccgtgaatttgcattgtttctattatgggtggattattctctcacttaattatctggggacttgcttcactgttaatcaaatgaaagcttttgttttaaacaataaaatctcttaagcacaatgtgcctatctagtattttgttctactaaattatttacattcctgaatgttgaatttaggtaaacaagcaattgaaatttcttgtttgcaaaacatatgacagtgagaaaagggtggcttttgagcagagttggtttttagtctggactctaaccctttgtttggggatgatatataatacctctcctacacagagtgcagattattgcagaagtcaaatgaaataagtaaaagtgctttgaatactgtaaaatgctatgcaaatatgaagtggtattttaaagctttaagaaatttccctcgtttgtctaatatatgcacctgtctactgaaggtagtatagtacttagggttatatggttctaagcaacagaaactaaaagaaaaattagtttaaaagattgggtagcttgcagagtcaatacaaaagctacagtgtcagacctgagaagggtgtctaggcagctgaaactatttgacagtctcatccatgccctagctaaagtgaataagctcaaataattttttccgtctttatatgactacactcaagactgaagatcaaaatccaagattcaactaatcttgcttgggtaaatttgctaagggaagggcagaggatcttgactgccagtcctccagtttctttttggggaaatactgattctccattgtatgcgttataatcacatacaatgagttataatcatctccacagttggctgagatgctgtactctccattgaacagatttaaaaaatgaaggctatttaacctacacgagttcttataaataacaggtgacaacatttagctgaaaacatttagctgaaaacaggcagcttgattctaaagcttatattccttactacgtgctattatatccactcagatacaaatttggcaaattctttggattaagtttgttttcctgcttgtgttttaaaagtaagttgattatcgaaattaggtttttgtcagcatttatgtggtaactgccaccacagaaaatctatagtgtatgttttatttgttcatttttgaagttatgccttttattagtataatttattatatctcggtcatataagttatgcaacattcagaacattctacaagatagtctactgaaggtaattgtggagcactgattaatatgccttggaagagaagtttattagctgtttaatcagagtcactgattcatttcatagaacaggatcatgctctgtctgtgtgttggctgattgattatgttgtaatgataactgtagcctttggagctatactgaagtattttgaaaagggagtatcaataaacattacgttgtcattaattcagctttaagtccttgatacgcttgtttggaattgatctctcaaatatggaaaacactattacgataaaagataagagaaggaattattaatatatgaaggtgttgggataaatgagattcaagataagcattgtgttttagaaagtaaagagacacagaaagaaattggacatttctttactacctttttacatggaaaaatacagtgctacgggaccatttaaaaattacttcttgttggttttggaatctgtttgctgcttccatttgattgagtttgtgcctgacagtgtattgacttcaagtcatgtttcttgttatttcatggatgtgagtcttccagagcttagtgctgggacttgtttctttttgtcctctgtacgaggtgatttggcagatacaaattcatcttctaccttgagatagaattgatttatatatagtcatatagtctttgttatttgaggataagtggttctggaaaaagaccactgaaaatgaatccagtttaaaagaggcgcaaaatttcaatgaagtggaaggggggaaggtttgatgggactttatcttagtaaaatgaaaatttatgttatttgttttgtatgtcacagaaatcagattataattttagttaatagttttaacctcaaaaagagcaaatcaaatcctgagtcttttttggggagggtcaaacattttatgataagacaatataatattctttatcttatgtttgttttcatagtattttttaaagatttatctttgtaagaatgttcaaatgcttcatcaattttttaagagactcctttgatatcaaaactattctctttgaagcacattctagttgtcagtcttgtcaattttcttttcttccactgtgaatgcatctagctcggttagatctttgtctgctgatgcattgtatacgagtggggatgtttcaaggcctcctgtcatctttcatcttttccaagattgtaatttctctctgcagtcagttcatacatatatgtggttaaacagttcttaatgatttttgcctgccattatcctagagataatgcacagatttaaatcatgggaaatcagcagcatggtgggtgttcttcagagtttaaaagataaatgcagtataaaggaaaatgctgtaagctgctgtgttaggataatatttttagacaaatctgcacattttgacatagaatcctttagtacattaacaattcttagggaattatttaattactcaaggaagatatttcatgccatgatggtagattactgtggtagaagatggtgaaaggtgatattgcactaattcttgaagcctgctttaagtgttaaaggtacatcatctcagtgacagaagcatttcagtcagattcagtaaaacacaatctgaccgaagttatatgtatattgtgagatacactttgcggcataaccatgaattctagaagctgtcagtggttgtgcttgtccttgtttactaaaaatttgattaataattgactaaactgatggtttgggttgcttatgaaaagaaatgttgaccttaaagaggtcagtcaatcgtactcttttttgatcaggagtgaggagctgatggggacttataataatgccttagatagaataggtctgtatattagcaaggggcttgagagtttcttgtatcaatttggacaggtgattcaaaaagtcaaacgaacgaacaggatggaggaaaaagacctagcggtttgattgattgtagcagcactttcaatcagttgtaagataacgatgtcataatagttagctcaattttatacaaaatactccttgcatttattaagaaagataattatcaagaagtcaggaaatgttttgttaagaatgcatctactaatatctgtgtatgttaatttgaggtactcgggtgatttatttaatgtgaaaattaagatgatatctgcagtaaccctttcataatgcaacaacttatcatccagggttggcttatatgggtcatgataagaaatggtctaagggagggctattctgtacattttgtgaaattgctataaattaataccactttgcttaatgaacacatgataaagtacatgaaattctgtaattttttttttctccaaatcttcatttgtatatccactttcaggataaaaagaaataataaaggaacagccaggtgtggtggcttatgcctgtaatcccagcactttgggaggctgaggtgggtggatcacctgaggtcgggagtttgagacctgcctgaccgacatggagaaaccccgtctcttctaaaaatataaaattagctgggcgtggtggcatgcctgtaatcctagctactcgggaggctgaggcaggagaatcgctttccaggaggcggaggttgtggcgagccaatatcgtgccattgcactccaggctgggcaagaagagtgaaactccatttcaaaaaaaaagaagaaataataaaggaaaatgtctaaaatttgcaatacaaagataatcaaattaatataaaaaaactttctgaataattccccattttggaagtttgagttgtcgttgtaggtgttagttgtcattttaatttaaatttttacaattgaccagagaaactattatcagtgattattgtggaaatatgaaatttaaaattaatgttctaaattttaaatatgaaacattaaaactagaagcattgagttaaacaaaaattagaaatttactccagatttaaagaatcttgatataattgaaactgaaagtctcttgcagagcacacatatagatctttatggaaaacgtcctttttcatttttttctttttttggtccaactcagtgcattagttatgatagattaggttgtaaaaacaaatagactataatatttataatggctcaaaatagacatttctgtgcatctcccaccacagtccgatactcgtgtccaactcagggggcagctttcctctgttgatgattcagggcccaggctcctcccatcttgtggctcctcttcagttctagtggccagtgcttgtgttaagaattgcctttgtcactagttgtataatcttgatgggcatgctataaacacttatttaattagctagagtgcctttgccaacttctaataaatagggtgaaaggctctgtggaaagttgctccctttaaataatttttctctttttatttcttttcttttctccatcctaggggtggatatataaattgagacctgctaaaaatttagacaatttcatgtactttgtgcctgtggaggctgtttttcccacatactgaggaaaaagagaacaaagaaagagaaattataattgttaacattatgaattaatttgaagtcatctcctttcaacatgacataggtgacatgtattctatacgcttgtgtatgtacacaacacacctggaaggagacacagaatctcttaacagtggttctgtctgctcagactgatcgggggaaactggggggttaggggccctccaatcagtgtataactatctctactgcttgcactttagcctgtgcatagcttgcttcttaaaataaaaacaaccataattgtaagtcgaaaaataattatttacagaagtgtcaaccaagtaagcagacattaattgcatttctcctgacttaggggtccaacatcactgacacctgcactgaaatgctaatgtgtggagtcttactgaaaatttcttctggaaatattcaagaacgggtgttttttcttttcgataatcttttggtgtactgcaaaagaaaacacaggtaagatcctaagcaggacacacttcagaagtggtcacagtttctacctttccctctgtgcttcccagaactcatctatccagctcacagtttctacctttctctctgtgcttcccagagcccatcatccatcagtgcatttcttcacatatattcagtctgctcactggttgttcacttcaccttcttgtaaaacctaaactgaaatgtcactgtcatctgatgatgctgggtcctcagagccctcttgaggagaggtgcggtttgttcctaacccacatatctcaggccctggaggagagggtggtgtgtcttctttgcttcctaatcttgtttcccaatcatttccccctcttccttcaagaatcccagcacatgtgaagttcatgccatgagtctttcctctccactacctttccctcatctcccctttatagaacacgtaatatctcactgtcacacatgaatttagtgatttctctagctcttgcatcattttctttctaccttactcctgttgccattttgggggtgttcaaaactcacatggatgatctatccagaattttggacctgaaagcccactttctcttcacctgcaatgagtctttctgccattacacctgagtctcctgtcctagggccatacttttaccttgtcaataccaacagtcataaaattgaaatctccatttcaggcattctactctgaatatcatcctctatcctcctagctgtattcctctggtattgtcacttcagttattctgactccaaacattgtcgaagatctcttatctcaaattttaaaagtcctttatcttgtgtcacactgtcacccttcatagcagactttcttgcaggagttttatgttgtcactttttctactttctcacttgctattctggcatgagcagcattcaacaagatctttgttcactctcctccactgagctactcctatcagaatctccataagaagcactactgtgcttcttactccagtggtcacttttctgctctttaaggttttgtttgtttttgtagcagcctgtaacttagctgagcaatcctaccttcccaaaacactctcttctctttaatgttattataccccactctcttggctggatattggagtgctgtggggctctgcatttgactcgttttccctcttcatattcatagactcttcccagctgattttatccagtcccatcactcatgtcccacatctgtgtcagttgctctcaaatctctatccttagcccaacattctggactcctacatcctgcctgttgtgatggccatgaggagtctcaaccacagcatggaagaaatgggattctgggttccccttcctaaagtcactcctctcccagctttccctttctattgttttctcaattccaaaaattacgagttagccttggttcctgtttttccctcaacatcccacatccaatccattagaaaatcttcctgcatctgcattgaaaacttacaaacatctctccacatctctccatcatcattactactctgctccaggccaccaggatccctctttggtgacaacagttgtctcctaactgacccaccatatcttttcttatgcctccacaatttatgctctccacagaaaccagagcaccttttataaaacaaatcagatcatgttgtcaccttgctcaaagccctcattggcttcctagaataaagttctttttgtttgtttgttttgttttgagacagggtcttgctctgttgaccaggcaggatagagtgcaatggtatgatcatagctctctacaaccttgaacttctgggctcaagcaattttcctgcctcaggctcccaagtaattggcactataggcgcatgctgccacatgcagttgattttgtttttcagtacagacaaggtctcactgtatttcccaggctggttttgaactctgggccacaagaatcctcccatctcagcctcccaaagtgctgggactataggcgtgagccaccgtgcttggccccacctagaataaaattcacactccttaccctagtttacaaaattctgcctggtccagctcctgcctgcctctcacatcatcacatcccatttctccaaacacaatacctccctccttcctttagccttctctttctcccatcccttgattatgccacatgtgctcccttctcagggcctttgtactggctgtttcccttcccagtgtgccctgtcctttgagatttccatggccagattctctgtggctttcagatctcacttaaaaatcactccctctgtagtgtcttccctgaccatcccatctaatgtggcctcccagtcactatcaatatcgggaaagcacttgtcacttgctatttctttcattatttgtttgtttacttcctgccacctttactagattagactgcaaggttcatgagaacaggggccttactgctccttcaatgcaatttctctggaaattggagcagacctgtgatagacagctaagaaacacagaatcaatgaatgagtgaatggaacacaaactagtcccctgtagagcttagccatcattaatgtaaataatttgtgtttaatgttattgacataatactatttgttttaaagcatgaatatacttttttcttgagaatgttcttactttatgggatgtttccttaaactcttgggaaaatttttttgcatgttgattttaagcaaaatagtagagtgacatttaattgagaagtttcctgagcctttatgtacttcaccctaaccatatataggacacatttgttttagtgaaatggtaccaatgggttcattcaatttaaaggtaaagcagtagttgaaagtacaggattttaaatgactgtttattccaggttcagcatttagaaaactatagtcaaatattttctggtagagtgagaattagagacacaaaaccgattgatttataagacagacttaagggagagttattcttcgtctcttataatgagtcttcttaaatcagttatcaagatgcccagagcaagttagcaaacatttggtagctatgatgatgatatatggtaattctgccatgtggattgtagtattagaattccctagtaagagacactaattcaaaaatgcaattatataagccgagaaaacataggaatacaacatgtatcttcatgagagggaacagattaggattagagccaaaaaataattaaattgttgacatctctattctaatgagttttttaaaattcctttttaccacatgtgaaaatgaatcttttattatgctcttctaggttctctgtgtttgatagcaagattgagattgatagaaattggcttttggaagatgtaggagaggttgttattttacttgtgtgttactcgttaagaaactgtaatgtgaatcaaggtgttttagtgcaattattaaaggtccccatttgagaatgaaatcttttttcattaaaattgatcccatctttatctatgcctgccatcaagacccaaatcttttgcaaaattaacattagtggacaaatcatgatattatatggtcttatgactggaattgcacattaaacacaggaaaagtttaacaaaattaaaatcctgctatttagcaaatgcccttaatgaatgtattatgtggatcaaatacagagtggttagaaatggaaataacaaaatagttcaacatgtccattcccttccaggactggagaagctgcttctccatctgttcccttcacttagaatacttcactcctttatgaacttcacctcagcatatgctttctaagatatagagatctttcagaaaatatgagtaaagggagccttttacaaacttccataatgttgtaattatatgaactagagacttgatgaatttaattctagaattacattctttaaataacagagtgctagattaggcatccttgctctggatatttctaatattattaatctcaggagaaactagttttttaaaaagttatgatgtagtatttgatacgtataacatgatattgcttagtttggcttgtttaggagttttaaaaaatagtatcatagcgtttttagtttcttgcaatttataatttgtattcaacatagtttctaaaaatatatatccattttgtgtgtagcaaaaatttattcattttacctgctgcataatatttcattagttgaatgtatcacaatttaattattcatcctcatcaatgggctttagggttgtcctgaatttttctactattgtatacattcttgtatatgtgtcctggtgaccattgcaacacataattaggaattaaataatagaatcaatacatttaaatttgcaatattataatatgcagcataattattttcaaactataggcttttcagtctttaagcttcagtagagtcattttctgaagagatcaaaatctatctgagctaagaattatagaattgatcaaacacttactggaattttgtgttgaatattaatatctgcagcctaagagaaatatacaaaacccatacagagttcacagtaagatcataaaaaattgatatattgaaaatagaactaagtgatggattcttatatataatccatgattattttgcctgtgactaaaactgttaaggatattaagtcttttcaatatgattggttttaagttagatgtaccaacttacaagaaattctatttttcaaaacaccatgatattattttccagctggaaataatacctgcatattagttacctgcatattagtctgatctgcattcctctctatagtcaacctcctgtgaggccaattttcatagctacctgggatgaattcctaatttatagcattgatttatgcttactaatgaagaacatttagaggttgctttcaaaatgaacatggggagtgcaaggtgatgaaggtctggggctgacaaactgtccagtcagactaatgaatcgatttgcttagttttctttatagagatgaaggactactaagtttggatgaggcttgaggacagctaagtcaggaatatgcaatttttttccaaggaaggagcccagagagtgtgatagcatctgaagatatgtccctggcctaccctatccacaccagggcaggtatctaactgcacgggataggaagagggggcgttgcagtttgtgtacagctacatcttaacttgtgctaggtgtgtctacataacacaacacattaatcattatttgactatataaaattattagccagattttgtcactgagtcccattgaatagtctaggtcaatggaaatctcttgagtgactaggtattttttaatccccttgaatcatttcttagaatttttcctgtttaatttaatgaaagaaaaacattgaaaaaatatttttcctgtgtgcttaatgattcaaatacaaacatatatgacttcctaattcattgttagaaatggtcatctgtaaagctggatttttgactttgtgttacatactttttaagattaaaagctggcactagatgtaatatacacacagttttgaaatgttttgggtattttagaaataaataaggccttcatgaaggattgcaaaggtattaagagacttgttcaacaagtatacagtgattatattataaaaatatgataggagaaatatttttatagctggatatcactgagcttaagaacctccagcatagccaattctttgaagagggatcataagtaataactggaaattaccagttagttactgaaaaatgagtcatgaaggtgaaagaaactcaaatgaaattttaaaatggccagttgttagtgtctgtcagagtctgattgggcaccgctaatgtcttatattgagcaaaagcacatactcactctcccacctgtgtactatccttagcttaaaacttgaatttatttttaaaaattagtatgcaagttgcatacttcattaaagcaaccagtgctgttatgaatgggaggacaaatggaaagatctccaggagtctaatgtttgatattgtcgattgaattggtgtcttttttcaaagaattgcctaaaatatattgttttgacattgttttacattgtttagtaacaaagtctttgtgatttaattccatcttaagacggttgaagaacagcaaggcatctacagatggacatcggtacctttttcgtggccggatcaacacggaggtgatggaagtggagaatgtggatgatggcaccggtaagtgatttgtagattttaaatgggatgccaatagtaaatagaaaactcctttgtaagacagatttctcacttggataattcacctgccatgtattagagcttagaaatactttgttaggtaaagtggttgggtagagaaaagagaaaggtgagataagggaggggaggactactgtgaaagtggaaaattatatatagccaaaacacactaacatgaaacacagatacagagaaatagccgagtagtgtcccatggcattcacgggactgaaacagagagcactgtgcagaaaagagttaatgaatgaattttctttagttatactgttttataagcatatcagtgatgttttgttcctataatatattcactgtaaaatatagcaaactatagatttgtcttagctatgtagttgtttttcatatagacaggttgtcaattttcttgtaagcaaattgtgaatggaggtggtttgtgtaattttaagttcttgttttaaatatcttattactcaaaggtagtaactttttgcttcatagatttgaaagtgtgggatttaataagctaatttatgaaatcatgattagtcacaaacattaagtaaagttaaaataaattgcactttctatttgaaaaaaaaaagttgtctctaaaataaatacttagtaaagtgaattccatttttccccaatgattttagttagaatttaaaaaatatgcttctacgagaaaagacaagatgaaatgcagtatactaactgttgctaaattttagtgatattcgttatacatgtgggattgggacacagatcagctccctccacctctcacataccttcattgtcactctggggtcccagaggggagaagaatatttataatctgtcagaagcacagaatttcaaatgctcacctgtaggcaatttctcaataccattgttagcatccgaataaattgtcaggtacttaaaataatgaactgtgattgctgtaggtaagggaaaaaggaatgcaatgagcaaaactcaggtggtgcctgagactgagcactcagctttggagccatgctattgtgttcatccagtagagaggttcctcagtggctaactgtggggtggtgtggagttgccagacagttggcagaggtagatatctcatttcaaatccctttctatgctacagactgtaatgcagttgcagatactcccgtgctaacatgtgggacctagtcggctatctttccatagcaggatgtacacattattttttacaaggtagcagtaatttattctgtgctttctaagaagactatatagtgacatttagaaatgttatctaacatgctaagagctgtcttgcctctgtaacctgcatgttcaagagactacccaaatgcccaactttgaacctgagttgggaggaagtcagatagtaaaagaagaacactaaaaacttctactctgttctttattggacctgattacccataatgggaccatctctccactgttgcttaggcgcttcctttattttcttccatttttggctttcccatgggcatagagtccctggccttatttcctgagagattgaatatccaccccttatgtcccactttctccctttactccccgtgatgtcagtgccactattagtgccatctgcattggtgatctgactgagttgtgatagtgattactatgattactgtgcttttgggcagctggagaactagataaagagatgtaagagcccaggacctttggaggcatggccataaaatgctgtcttcccgtgtatctgggccagattgtaagggaatgagatcctatgaagggtgacttgaattgcacagatggttcttggattcaattcgacctgaaagttttgttgatgctagtaagattccagcctacaggtccccagttcctgagggcaaaagcattgtggaaggcagggcctaagaccttgttgtgtatgaattagaagaaagacacaaaattgcaggatatttagttgtcataaattagttgtcctatggaacctcttgacacaaattcgcaggcttgattttctttaaagtaagatgaaaacatttattacatcttgtattattcaactggggctttccccctctttactttttcttgaatgtatataagcagcatgtagcaggaatttggctgtacctggtcactacaaggtcacttaaagaattttcagcctgcaactggataccataccctagtttcctgcaagtacaatttagttttctcatggctatatccactaggttcctggggaattgaataatagaaattttgaagaaggataggatcttggtgatcaattaatgtaccccaatagcatttcagatgatcgaaatgaggccaggtttaagaactaacttgtccatgtttaccaagctgaccaggacagagctggagctagagttaagtttgctgggcttccatgtacatgttttgtgacatagagaaggaccaagtggagagcagtgaaggaacaaggcatcagcgtatggaggttgggtttggttgcacccagaaatgatctgggtgctgcatgaactttcttcctttgttgtttccttggtcatttccagcacagcatcttcatttctttttgatgagctcaggttttaagtctgctatattttttactttacacatgaagctctgatgcagatggatctcagcagagctgattaatgtccctatgccaagtactccccatacttggccctggataagttaaatgagggataaacagatactatcaggctcatctgtcattgccaaaagaaaaaacaagcacctataccatatttagttataaataataaaatggatgaatttatatatatatatatatatatatatatatatatatatacacatacatatatatatatgtatttttttaagcatgcttagaactttttcctttttttaaattgagatggggtggtggtgaggagtctcattatcttgcctaggctggacttgaacccatgattctctcaactcagcctcagtaggtgggactacaggcatgcaacactgggcctggcacttagaacttgtgatggaaaatggaatggaaaagtttttcttccatttgaattatgcaaatcctgagtatttttctatactctatgtatgataaaatagttttctaaggaagaagctctatcagatgactaatttcattgatatagctcaaaacttgcagaaaaaataattgcacttgggatgagacaaaagttttcagccaacatgtaaacatcttaaatcttttgataaataatattattgtaattttattcataatacttaatgacttggtttatatcatagttatatcttagtatagaaagaaatttaaatgatggtatgatgtgcaaaagtgcaaaaatttgtatatatatttatttaagtctgtccatgttattaattaattcctatattcaaggcagatgttgacctttgcatttgtggccaaatttccattcaaccattcttcagatttcaactctcagtgaatctaagttattctttgaagattctttctgggtctactagtttgatcatgatgctataaagaaaattcagcttaaaggcaatagttggaaagctattgaacttcctagaattaattttcatggtaatttttatagtgcatgactggaaagccattcggaaagtagtatcagatcattagtattctctcttatgcactagtttatttctatggtaggacagagggcaagaatgttgacctttagaaaatgtggtctgtagttcagtgttgggcataccatagatttcaatatattcgtcaaattcgaactggataagatgaaaaatttttattcttaaggtgttctttaaaagtaagtcaattaaaaaaatttagctctccctttatatataaatatttgaaactattctaatatgttgaatacattttttttataaaattaaattgtgcttattaaagaaactttggacattgcagaaaaatagaaaggcaaaaaacagtagataataatgtcatacaaaggcaaacacataagactatataaaaactatttcctgcaattttccttttaatgcatagatttaaaaatgcttgaaaatgtgaactttataattttgtatcctttttcacttaaatcaagtacctgtattatctcaatgatcgtcatctttatcttcatgccttatcaccaaaaatagcatttattgttgcataccagtcgactgactagataaaccagttactttcacaactgtagtattgttgcataagctggattgagtatgttcttaattaaaacgtaacaatggcttaacaatgttgaaaagaatataagaatgtaatagtttttttttctaatacattttcaatgtttttaatttaagggagtatatttgggagttcagaaaggtcaagggatgaactactttttagatttttaaaaggaaaagtcaattagaatctttttttttttttttttttgccaaaaataatatcatatgctatttgggatgctggaaatttttcttctattatttgataaaaatcaaagatttattcaaaaaatttttctcaatgttaaatatttatgaagaaattataagttttaaagtttcccttagaaatttatgcatccacagtgttgatctccaggacaaggattgaatacattatttagacatcagtttcttgatgttttcagtaatatagtctggcaatttcttctgtttcttagcctgtttataatacttggatattttccatcttattgaaatgcaaatttattgaaagaatactggcacatccactgagatttacagtgtatcagttttggaaaaatcccatctcattatatgaaactgtactgaaataagagcaaggaatttgcaaactgtatagcttcaagtaattagtgacttgttcaggtctgtcccttggatttttctgtttttggaagaaatcagtgactaaagactgttgaatttggatattccacatttaaagaatggcaatttaaaacctattcttcctataaaattaacattttagatccattgaaacttggaaattcctgcgtttgccatttaataaattatttttccatttaaattttttaaacaaaattctgactaagcacaaagcagtgttggactttttaatggctttagtgagggtgttctttagggataagtttaatttttattttgatatatgaagatattgaacaatgtgctatgattaaaattgtgaagctattatatgtcactgtatcttttgaaaaaaatgagaagcaattgcttattttgtataatatttggtaattttgatgctattatgaagtatgtatactacattttgaggcctgtggtatgtatattcaattgtatatgtgtgtttatgtatgtgtgtatatatatatatgtttataattacttattatttttaaattggtgtgatagaaattagctagcatataaactttaaaagggaacattatttcttcatttaaaattatttgacacaagctgttcttttaaaatactagtgcagcaattaagctgttatttgagacagtggaggtattagtccattcttgcactgctatcactacccgagactgggtaatttataaagaaaagaggtttaactggatgacggttctgtgggctgtttagttcatgtggctggggaggccccaggatacttaaaatcatggtggaaggtgaaggagaactggcatgtcttcctggccggagcatgagagaggggtgggggaggttctatacattttaaacaaccagatctcacgataactcactcactatcatgagaacagcaccaaaggggaaattcttccccgttatccaatctcctcccaccaggctccacctccaactttggggattacaatttgacatgagatttgtgcaggaacacagacccaaaccatattagtagatttgacagtcagctaatgatcttggtttctgtacattgaaacaatagccacatgtgatcaatcacatgctcacttatctgattctgagttgaattatgtgctttccaaaagagatttgtatgcacaggctcaattggtcaggccaggcttcatggaggggatgggtccactttcaagaggagtaagaatctgtgaggcagagaggagagtggggtagcttgatgagaaactcagtctggggaatatataagtattcacaggacagccaggagagcagcagacaagaaaagcatggtgggaacatattagaacatagtggaaaatagaacgagtgtttagacagagttgagttattttgggaagaatctttaaagcgaagtctacatgtagttttgacttgattcagtggtgagacacccaatgggtggcttttgagctaagaagtggcctgtttgagggagggccagcagggtgtgaagaggccagtgaccagactcaatccaggaaagaaaggaaggaaaactccctgttatagagatggctggccccggggtacacttatgcggtggcaggagcatgacttgaatgattttggcacatgaacttgtgttccctgtgctctgtgggagggctacactgtgttcattttagagtaagtccagtgtttatgactgtaggctacatttggctactctggttttctttatccagagtaatggttaaaggtataataacatgagtggccatgtgggatcttttaagctgcaggtgcaaggttcacttagctaatcatggcctgacttgaacagagagaagacagcctgaaaaaaagcttgggcttgtatgtgactctggtcttgttaggttacaagtggttagggttgggggaagagttttaaacgtgtagaatttgagaatttctgaattttttcccccagtttattgtaatttctcagttggttatttttatagatattaaggagggattgcactgtaattatgaagtaatattcttattttcctactaatgcccatgaatacttatctgacatgaattatgaagttctcttactttgtgtaggatgggaacatgaattaaacacataaaggtaataaaagtacaaaaaatctttttaaaaattgcttgtaattttttgggagggaagtattggccagtttaaatctctatattatcagaagtcatttttgattattcatggattatggcacctatataaaatacagactaatgtctaatgtcattctattttgactcaaaagtaatctggaagctttaaagactccacaactgaacagcatcatttgataatggatttgtggtattattagtctgacctcatataatgtgtagtgattttggagctaacgaaaaagactatggaaaccagattttgggagaatgagtgccaagcacatctgtttctactcaaaaacttaataaaatagatcatctgagaaaaaaaagaacaaaacaagcaaagtgccacaaagtcattttctaaaccacctttaactttcttgtggcttacctcctcaacactggcctttgctgctgaatgactttcaggagagacacatgaaaagggtcactttaattggtacaggtgctgactcaggcctctgtcgtaggtttttccatcttgggatctttgctttgcagggactgtcatgccagtaaaggtcgtttgcaagttatgtcactattctctacccgctgttaggcagacagctactgtatagggtttgctgttgacattttcaaacccagcatgacagtttacctaaaattatagtgaggtatcaaaaaagacaattaaatgtcaatttatcttcaaaaatttttttataagtttctatttatgtgacttaaatggaatgaagtggctttaaagagtgtatgtcttgacaatctacgatgataatgctttggcatttgtatgtttccatttattgcaattttaggtaattgcactctttgtgtgttccattggtcaatccttagtgtgcagttcattaactaatagaagatttttccatggggcatagactgtctctgaaacctagaatgctatttcaaagaaacattagaaagataaaataaataagacatcttgaatgtttgagaagaaaacaccttgcatgttattcttttattgctttagaaactttgacaatatttttgtgcatagagcatgtaagtgttaattagtttgtagtgtttagtttcatgtaatgtattaacactctgagctagaaatgtccattattgagcactgacagcatccaatatgaaagcattcttaaagtttttaaatgtttatgtttttcaatatacatatgtaaagcaaagagatgttgaacaattcagtggttttggaacttttgtgtattgaatgatagtgtggatataataatattgtgtattacattttgttcatttgcctttacccattaatttacagctgatttccatagcagtggacacattgttgttaatggatggaagatacataacacagcaaaaaataaatggtttgtttgtatggcaaaaacacctgaagagaagcatgaatggtttgaagctattttgaaagaaagagaacggcggaaaggtgggtgtatgaattgggcgctgttacactttgtgaagactctaacttagcataggagcagataatgggaaaacatgagaaaatggaaaggtatatgatggtcacttgatttattaggttggtgcaaaagttattgcagttttgacattgaaagcaattacttctccaccaacctaatactatgtgacgtgagcctcatactaaaagacttatttgatacttacaaaattgttatgtaatacttatatagttagcaaaataattatgttatggctggtagtgtcttttaaataatttttttaaatagaaaatttgttcaattttctttgcattttattcttgctatttctaaagcagcattgccaaatttttctttaagtgacaagcttcaataacataatgcagtgttgtaaagcctccaattgttctcagtatacttaacagaacactaaaatttcttagacccaatatgttagcttcctaattatagattaaagcactttggatttatacaaacttttaaaaattgttaggtctttgaatatagtataatgtaacccaattagttgcttgttcatatgaggaggcctagctccaatttgttttatgcttttagaaaaatttgcaaaaggaatttggcctggctgtattcacttgtgtgaagtttatctgcaaaaaggcatatttctataattcagctcagaaatagaaatgcttgtatttctactaattaacaattgatttcttaattaagaagcatgacattgttgattacttctggagacccaagaataaaacacctcattggtaatgaaatttcagaacacaaagaactctttcttccatcttcagatgttttcccctgaagaatatgcaacattgctaccttgattgaaatattcatggtacatacttgtagaatttccaaaacattttcaaatactcctttggttgatcttggatgaagaaccagatagaaaattttaaaacaaagtttatgaatatatgactgactaagttttgttgaatgtagcagtcaattcgaatagcaaacaaatagttaaaaggtgaatcaaactttcaatcaaattgaacctttttcttaagcacccaagagaaaaaaacaaaacaaaataaaaacccccaaacgagaagaagcacattctctaaaactgcttatgatctttatctttatatttatctttattagactgcagtcttaagggattatttatgaataaagacaaatgataatggctccagcattgtcaaaaataaaaatctggtaggaaattttgcagctagatgactaaaaataatacaatgttgctccttcaacagttcgtcatcagtcctgattaagtgccagtgttcgtaggattttagcttggcactgtttttctctaactagattccttgcctgggttatctattccatcagattaactcccacttttacactgccgatttgttcaactttatgtccagccttgccctctcccctgagcatgctcgtgttttcacctgagtcctggactcccacctgaatgtccacactaggcatacttggtgcccaccggactcctggttacttggatgcctttgcccaatacacctccctgcttccattcagtcactctcatcagaggcccggaaggcatctgatttcttctctttcttccatgcccacatttaaccaatatcttatcaatagtgcctcctaactatttctcaacttcatcctctttgccctatggccctacccttatttgcagtgagttgaacttgtttgaacttttgaggtttctcctaacagtgtcactcttttaaaatcttcccctcttcagtttgttccccgcactgtcctctgagctttgtttccaaagcaacacaactttctttcctgcactaccctacctttctcctcccactggttaaaacacttagctgcatgcccaatacctaaggaatgaaataaattgaaactgcaggttgagcaaggatcttggctatcctaccagctcatctcctatcttttctcttaaaacccatggctccgggcctccaagttgcttcaaatgagttagctctgctttctctagaaatctccattcacacctgtgcattcaagacaggtcccttctgtgaaacctttcttaacatttctcagggagaggtgaggtaggctgagtatgcctctattatattgcattattttttttctaccagaactgtctctagggctactctgtccagttctgcggatctaactctgagcaagggtgcctggtccagggctcaatggaagctgagactcagctgctcttcttactcccctggctaaagctttatgcctttggggcacagtttgcacaaaggtgacatataccctggggatcttgccttgcaggtctccctagcccctgagcttcttgaacaccagggcactattatacagagatctgtatccacaacccccagcacatggtagacctgcaacaaatgcttgcttaatgaagccataactgaatgaatgaatgaagagaattttcagttacctctcctttctttcatttttgataggtttaaaattaggaatggagcaagatacctgggtcatgatctctgaacagggtgagaaactttataaaatgatgtgcagacaaggaaatctgatcaaagaccgaaagagaaaactgactacgttccctaaatgctttcttggaaggtagggttgggagtttgggtgcatgactttcttttacattctcattgtctcacctgttaactttccttcaattagttttcctttaaaaatattcccttaggcacttttgaaaacttctttacagttgccatttcctgggcggtagcaggatgaaggaatggacagagagccaggatcatgaccaggaaacttaaggcatccgtccattcattcatgacttctgagatttctgatatgtcatgatcccgggggaaagtgaggttggggccaggcagggagtggcaggcaggtaggtttgcctgcaggggacaggatgactggttgggcacagacatgtatacacatgggcctgaaagtcagaatcagaaagaaaggaaagataagagacagtctgaactaccagttaatttagaatttaggcagctgggctcaggataaagggtgtaggtaaagagtctgaaaatgtgggtcatgtgaagagacaggcagcaagcaataggcagagaggtcactttggtttttggagtagaacagagtgactgagtgttgtttggttcctttggacaacattaagtgggattccctggctggtttaattagtgcagggccagaaggaaggttagctgccttcaggtagctcttttttaggccagactctgctggcttcttcctatacattatttctaatgctggaatacatacttaggtaggagttaaataccattatacttttttaaagatacagaaactctggatcagggagcttaacaatttcatatggttagtgaataccaatgcccaagtttataaagtagactggagtcatagtttacagtctagactgcaatcaaacttatctattcacttcctagtagtaccaccaatttgttgtgtgatttgggatagctaagtttccatttgctttgaaaacatggagatgatggtagagtttacctacagtgctcactgggatgcctttttaaccagcgatgggaacacacaacataatcataggctacctctgcctccctcatatggtttggctgtgtccccacccaaatctcatcttgagttataatccaaattgtaatccccacgtgtcaggggaggcaactagtgggaagtgatgggatcatgggggtggcttccaccttgctggtctcatgatggtgagtgaattctcatgacatctgatggttttataaggggctcttcctccttcactcgccttctctcacctgctgccgtgtaaggtgtgcctgcatccccttctgccatgattgtaagtttcctggagctttcccagccatgcagaactgtgcatcaattaaacttcttttctttataaattacccagtcgtgggtacttatttatagtagtgtgggaatggactaatacacccagtctcagggaagttctttacagcagtatgaaaaagggcgaatacgctccccaacccccaactcagaaatctataggccaggaaaccctctgcctgcagagcagcatctggctttcttcttcactaagagtgatcctcccaccaggctacacatctgccagtgagctatactgttgcttgtaggtacctcttagctggtagcagctcttgtgcaagggaaagagaagatgtgagtttatcttggcaaagaagccctgtgcagcaaaacgacagtatctcttgtacccaccccatgggcataactttcagggtcctcactgggatatgcccatttgcaagagagtgacttcaagttatggcttcccagtaggttttaataggtatgcaaattagccaccaggggtcatttttaccattgcaatgctgcctagtttagcagttgacaccctgccttcttcaggtgtctaggggagcatagctattatagaaagttagcccagggtcaagtttgttcttattgaagaagaataggttttgctaacccttcacttacactgtgtcatatgggaatagtgcaagttcattgtgatgctatatgaagcttctcccagagcacctgataggtaggagagactcagtaaatgttagattactgcggccttttccataacagcacactactgccgcttacagcgcaagtttgagatcatttctgagtggatccttgtccgttagcagagtgttgatggaatttcctcattccctgagtaggcccaatgataaagccactcattgggttcctttattctcgacctctcactcttaaaaatatttccaatttatagcaatgtttaaattggctctttccctcattgtaatatgacattttcaggaatctagtgccatcagactagagaattatgtcttagttacataacaactatctcaacccacttgtcaaccactgagttagtaaatctcactatgtgccaggccccgtgctgagttcctgacatctgtcatctcatttaatcctttggttgcactatgacattgacattcttttttttttttgagatggagtctcactctgtcgcccaggctggagtgcagtggcacgatctcagctcactgcaaactctgcctcccgggttcaagttattctcctgtctcagcctccctagtggctgggattacaggtgcatgccaccacacccggctgattttttaaatttttagtagagacagagtttcaccatattggccaggctagtctccaactcctgacctcaaatgatccatctgcctccgcctcccaaaatgctgggattataggcatgagccactttgcccggcccgacattcttaatattatctctgtgttaatgatgaagaagctggagtttaaagagctaaattcatgaactcatggtcatgcacgtagtggaaacagaaacaggatttgagtccccgttgtcttatggcaaagtacatgatattaactgctacactttgtttcaccttttcttatatttttcttctgaatttgttatgcagcaatagaaaacgaatggttttgaatggttctcatttaataaaagaataaaaaaaaattaacaactttttctgccttaaagtatttttgtagtctagacaacccatttttagaattttaacctcaattatttgacattagccatttttcttgcttttgtttttgaaattaatgaagaaataatttcctgatgcagtccatagctttgtttctctagccaaaaaaagaaaactagtctacaacaaattcaatagtttatataaaagttcaaatgttagacttttaaaacatgagcaatataatattttttatttgtaaaatgtctctgtatttagtgttctcttaatgataccacgtgacctttttttttaaatcgctacatagctgaggctctcctaagtgatacaaaacatcatccttattatctatgatgcctggggctgccctgcagtggtgacagatgtaacctataatccactgtggccttcacagccacagcagtgtgtggtagacagttctgatggatggttctgtgctggagtattccttatggatgacagatgagatacaataatgaatttaccaagtaatttaacattttcaaaacaatgtagtagaacttttctatgtcttttggtgtacagtagttttctattgctgtataacaaatgaacatatacttagcagcttaaaacaacaccagtcagaagtccaggagagctcaagtgggttctctgcttagggtttcacgaccccaaaatcaaggtgctggccagacggggctcttagattgaggctctggggaaaagttcagtgccaagcttattcaggtggttgtcaggaatcagctcctcattgaggctgcaagaatgaagtccttgtttccttgctgtcagccaagggttgctctcagcttctgcaggctgcatgtcttggtaccaggtcctctccatcttcagagctggcagttgcacattgaatccttctaatgcttttaatttctctgacttcctgtcatgccactagcctgagaaaactctgcttcaaagggttccactccctgaatcaggcccactgggataatttacatattttaaagtctactgacttggagttttaattacatatgcaaaactcccttcagagcaatacctagattagaatttaattgaaaaaccaaggggagggaatcttggtgtggtgggagggcagtgagaagatttctgtagaatcctacctaccacatggtacatgtggttttgttttgttcttccttcctggatctttaaactcactggttctctgtgcctgggaaccacattaaagacagtttagctctttatattaagttattactactctattcagttctgactttagctatctttaggatctgttgcaatcctttagtcccacatttgcctttatgttaatggtaaccttttaattaaagctactctaaaattttcactttgtgtttattagaggcctcaacaagttgaatagtttaaaactaagtatagtttagtaatgtcagaatgtacagacatgggttttctagaatagagttcaacttaaatggctgtaggccttacatatgtgaaaaaataatctgttgaaatgtgttgcaaatattcctttatattttgagattcctttcatcattgcttcatgactggatggtatagaaagacttgctggggaaaaaattaaccgtttagctttttcactgactttctcttgcagcgaatttgtgtcatggctgttggaaattggagagattcacaggcctgaggaaggcgtgcacttgggacaagcattattagaaaatggaatcattcaccatggtaattactttattattaattacttatttaatgcatttattggccatatgtatcttaatcccatttatcagtttagtttacaattccccacattataggatgcttgttgtttttaacaatattttcagtacctaataagtgtagaacagtgtctcagggagtgtgggtttatagggctatatttgttaaccttaaaatgaagagcttcaatacaggcaagtgttagaaaatagagaagtgggattgtggaggaagggatggttagttctatagtaattagagtatttgggagtgcttcatggaggacgggacatgacaagtgatgctgggcattgcagaatgaatagaactctgccagggaccgcagggcagaagggcattccacccggaggaaataacagcactccggcacggtggcgggtgagtttgtggtggcttctttgggcaccggcacagagcctcacgtgggtccatggatgaacatactggcgaggggaggtgtggcaaaaaggcaggcagagcctggattccagaacatatgaagtctcctgctgtggcatgtgggcttattccgaggggggccaggagctgatgaatgcttttaagcagagccatgatcagaactcaggtccacagctgtgaggtgcagcagcacggagcctgaagtggagaggttcaagactgcgagcactaggcccctcagggggccctgctgcacccaggagtggcgggacaaggtcttcagcaggtctgcctccgtgggcatgaaaaaacccatggggagatgctggcaaagctacaataccatattttataactctctggatgtgggtgatgaagaatgaagagagagattaaagtattctgagccttcttctctgaatgactgagatagctaattcaggaggaggatcagggttgaaagagaagatgcaggctttgctgtggaccgtgtaggccagccagtggggagtggccagcaagcggttgagagtttggggtgaagctcaggatagaaataaggtgagaaacacatttggaatcatttcaagggagtactagactcctgggaacactgacaatgaacagacagacaaaaaccaagaaagcagtgttaatgggactcaggacaaaggtttaaagaatttctatggtaaattgtgggggaggagaaaaagccagggaattattaataaatatggatgtttggctatggtattttgatgttcatttcctttcttagatactgtgcaaacaaaccagatatttcccatcttttcttatcacactgggtgttgcctacgtgatggtaccaccatccacccagctaggaatcatctttaattcctttctctccctcatgccgtcaagccctgtgagctctaccaccaaattaagtccctaaacaatgaattctgtccctttcagcatccttaccttttttctagtcgccgtcatctcctcctttgcttccacttttgttctccactcctgtagtacactctccgggtcacagctaaagagaccttttaaaacggggatatgatcaaagcatccttctgtttataaccatctaattgcttacaatcaaacttaaagaaaagttcagacctattacatgccctaaacttcttaccatgacctacaacatccgatgagataaggcccctgacttcctctcagagcctgacacctcgcattgtacctaacttgttgcactgtggccaccctgaccatctgtctctttcttcaacgctccaagatgacttcctatttaggaccttagcttttgctcttctcattcaacagttatttattaatccctaatacatgccaggagcctttctaagtggtagagacagagctgtgaatgaaaagacaaaaatctttgtattcattactcatgtcttccatgaccctttgacttttagaagcccatcctgtcctgtgattccaatgtcctattttttttttttactttgaaatctttcttcatgtatttatcactctgaaattattatgttgttttacttgtgtctttgtttattacctctttatctccctatcccttccctggcctccaataaaaatataagcaccatccaaacaagagcctcacttgttattatatagcaaatgcctggcattcccatcccatacaggtgctcaataaatatttgctgaatgaatgaataattatattttgagtgaacagatgatacttataaattgtatatgattttgcgcttgtttcattagcatcattaaaacatctaaaagcattatacggctgcttttagcatctggtaggctaaaagcatcatttaaaaacttaaaagtaaaaataacattatgactattatatactaacatcataaaatgctaacaagcatatataataattaccctgtctttaataggatcgcatagatcttatattgaccagggaactgtgcctcagtggcagaaattgagtagttgtttaccaaattggtttagcttttgtttttcgtgggtgtgcatacatggcatttcttagttttccttgcagtcaaggggaggctgtgaaactgagttgcagccaaggacatgtacacaaaagtggtgcacaagggaagtgtacaggctattaaaaaaacacaaaacacaaggtgaccctgggagccatgcattaaggatggctgagcctctgtcagaccctagaccctgcatgaggagagcaccatcactcatcctctgccacggattggattttacctcagtgaaaaattagcttctacaacacaagtcactatattagggaggtttgtttgttacagtagctagtgttaactagtggaaaaccagctaggttttcttcccaatatttcctgcaaaaatgacttatcccatcctatcctagcgtatttctgcctttctagaaggaagtatgagagcagctgggtttcttgatggacagcgtaaaaagacagacttaaactaccaagaaataaaggttgctggttcatgttgacatacaaactaggggtgagagaacatattatgcaatttcacattaatagaataattctattagaatgaagaggtatgagccaaatgtgctagtatcattagtatgaggttccagctgcattttggtgaaaatagaattcctaaaataaagttgaatgaaccatctgttttaaacagagcaagcaaaaaagaagttggctattattgctattattgacattgcaaatatgattgagaacttcaccatcagcaaagccttgctatagcaggaactctagcgttgaggaactctaggttgagagaaagcagcgtatgctttctctgtggtatgctgacaatatacatgttgtctagtgctactttttgtagtaggaatgcttatgtgagaattcagagacctcatatcttattaatattatatcccatggtgtgctgaagttggctcatacaagctcctgagagctgataattaaatacattttgtaggctgattagtttttaaacacaattattattaattcaattatataaacatgtaagtgcataaattatgttgcaaaaagggtaaaacactcttttggggggttggggggcagggtcttgctctgttgcccgggctagagtacccagccagtggtgcgatcatggctcactgtagcttcaacctcctgggctcaagcgatcctcctaccttagccttgtgagtagttgggaccacaggtgcacgccaccatacccagctaatttgttttttttttttttaatttgtagaaatgaggtcttgttatgttgaccaggttggtcttgaactcctgggctcaagcagtccttccacctcaacctcccaaagtgctgggattacaggcatgagccaccatgccggacctgtaaaacattcttaaaatgtgtcaccttctcattattttacaatgttttactattatctatgctgtcgaggttatttgtatctattgcttgtgtatagtggaaatactgtgtaatgagtgccaccgcacctttaactccgtgttcagtgactttaggttggaaacttgaaattggccagagtgggaatatttatacaacagaaatgggcaaatgctacaaatcagaacttctcctctacaccccctaccttcaccacctagagagccagttgttaaacatttgcactatgttcctggttctctcacacagttatttaaaagattgcattaagcagctttcttcactagaggacccgaactgtagtttatacacccattttccatgctagataaattgggagagcaaatttgaatgcagaacagttcagcgtttttctaaggcaacagctaagagttgcagattaacccaaggttcattatcaaaggcagaagcatttgccttacagctaggctctccctgtcgaataggttagactaccacttgtttcattgtggcaaaaacatttgggctcaacttcttgagttttggaatatcaagacaatctatttcaatgattattaaggtataaaaatgggaataggtagattgcataggagaaaaaaattgtcatcttaaaacaagttgtagagtttcacgttatttctcttgattttatatttctttcatacaggcttttttacgtatgacttggcttggggaaaagataccaaaaagctgtattgtctatctttttttaaaaagaacaatttttggtcataagccatgtagaacgtttgaataggctggggtaaaagagctgaaacttcacatatgtatgtgtgtgtttatgtgtattaacaagtcttttgactttcttctggatgcatcaaatgtagttaatgagaattctgacaactttctttcctctttcaacatttctagcaaaaaaataaaggtcaaagtctttcatctaatcataaaaatatagctctttgcagcaaattatctcttctacaaatgttgcattccctgtaaagaaaatatttaaatataaactctttgacttttactttgaggtttttactttctaaaacagacattttttattagactgtgtataaaacaacttagcctgaaaaagagaaagcgtagagatttagtaggagattccctccccttccctccagctttatggaggtataattgacaaatatttatggtatacaacatgttttgaaatatgtgtaaattgtgaaatgatcaccacaatcaagctaattagtgtattaatcgcttcacatagttatcacttttttgtggtgagaacatttaagatctactctcttagcaattttcaagtatggaatacattatatcatattttctgtacctatccattcatccatccatggaaacttaggctgatttcttatcttgctattgtgagtaatgctgcagtgaacatgggggtacagatatctctttgacatattgattttatttcctttgagttacacacccagaagtaaaattgctggatcatatggtagttctatttttagctttttgaggaactttcatactgttttccaaaatggctatatgaatttacatctccaccaacagtgtacaagggtttccttttctccacatcctcaccaacacttctgtctttttgataatagccatcctaagaggtgtgagattatatctgattatagtttcgatttgcatttccctgatgattagagatgttgaacaacttttcatatgcctgttggccatctttatttctttttttgagaagtatttatttaggttcttcacccatttttaattgggttatttgtatttttggtattgcattgaattaatttcttatatgttttgaatatttgcctcctactgaatatatggtttgtaaatatttttcctattccatagattgtcttttaattttattgattctttcagttgctgtgcatacactttttagtttgatgcaattgcatttgtctatttttatttgtctatttttgcctttgttgcccgtacttttggggtcctctccaaaaattattgccaagacaaatgtcaagtagctgttccctaatcttttcttccagtagttttatagtgttacatcatatgtttaaatctttagtctacttgagtttatttttatatgtggtgtgagataaagggtcccatttaattattttgcatgtagatatccagttttcccagcatcatttattgaagagactatcttcctccattgtgtgttcttggcacctttgtggaagagcaatgaccataaatctgtgaatttatttctgggatctgatctctattctgttccattggtctatatgtccagtaccatgctgtttttatcagtgtggctttgtagtagagtctgaagtcagtaatgtcatgccttcagctttgttccttttgttcaaggttgctttggctattgtgggtcttttgtgcttctacacagattttaggattgttttttctatttctgtgaaaaatgcctttggaatcttgatagaaatcacattgaatctgtagacagctttgagtagcagggacattttaacaatattaattcttcccttccatgaacatgggatttcttaccatttatttgtgttttctttaatttctttcatagtttgtagtttattatttctttcatggtttatagttttcagtgtacagatcgttcaccttcttgtgttattcctaagtgttacatcttatttttttattctattttaaatgtgatcatttttaacatttcttttttccatagtgcattgttagtatatggagacactactgatttttttaatgttgactttgtatcctgcaagtttactgaattagtttttggtggaagttatatggttttctatatataagttcatgccatctacaaatagagacaattttacttctttccaattgaatgtcttttatttctttttctcccctaattgctctggttaggacttcttgtactatgttgaacagaagtagagaaagtaggcatctttgtcttattctagagaaaaagctttcagcttttgaccattgagtattatgttagctgtgggcttgtcatatatgatctttattatgttgaggtgcattttgtctatgccaaatttgttgagagctttcattatgaaaggatgttgaattttgactaatgctttttctgcttctattgagatcatcgtatgatttttgtcttttattctgttaatgtgatgcatcataatttttcatttgcatgtgttgaaccacccttagttctcaaggataaatcccacttgatcatggcatatgatcattttattgtgatattgaacttgatttggtagtattttgtttaggattttggcatccatgttcatcagtgatattggtctgtaattttcttttcttgtagtattcatatctggccttgtaaaataagttttggaagtgttcctttatcttttatttttggaagagtctgagaaggattggcattaattcttctttaaatatttggtagaattcatcagtgaaggcatcaggtcctgggaataggagtctttattaatatgaaagtcttatcttgaggatggtaataaagaatgaacggtcttgttttccaataagaaaaaaaattaactgacatgtaaaaaagcaattcttaatgtaagaacagttacaattttggcatcagttagatactaaattgatttaagctactctggaggtccaaacacaggataagtggtcaagattggtgtatgtgtggtcttggcctgatcacagtgtgataaaacagatacgtggtaaaattcatttccaagaccgagaatcaaatcacaatgcactagtttgagtatctgctctttgaggggcactttttgctcagctttgtagcattgtgaagaaacagaaaacacaaaatcatatatttatataaatgtacacattgtgtatacctttatacatactatatactcatgtacatatataaatacatatattatgctagctgaatgttgcaaacgtcagtttatttctcaaatcgttatatggaaactgtaatatttttaaataaaatattatcatgaatcttgctgttaaaatactcaattggtattgctgtgttagaaaagcaaatgccttttgtaaaactaaaagttttagtatttgggaataagtatttaataaatttatttgctgtttatctgcccccttttagagtatcgttattttaatatagtatttctatgttcttagttactgataaacatcaattcaaaccagaacagatgttatatagatttcgctatgatgatggaacattttatccaagaaatgagatgcaggacgtgatttcaaaggtaacgacctctcccacaacctctccaatagtagaatgcatgttgtcattcttcagaagataaaaggtagttgagaaacataaaagatttttctagtttagaaaattcttcgactatatatacaagcaagcctctttatttatttttggtatttcaagaattctaacgttcaattgttttctcattctcttacctaattctaaatggtagtagtactgctgcatcactcaccaggttatatccatggaaaacccttttacatgtagcaacatgttgtcaaaatacttgaattaaaaaaaatacaaatgtgtaaaataaatgtaatgtcagattttgtacagttgacaatactttgttgatatatgatgaacaagtattatagatgtattactgtattggtatattttgtttaatttacgttagaaattttatattttaagaaacataatttagaaaaatctttgtatgtgtgtatgtcatatatgaggaatttatagcacaattttaatcctattctttaatgatttaaatcatattatttttatgatatatcatgatatttgaataatttttaaattggtttagattttgtgatgattctaaaatattgacagtgaattaatttgttacttcaaaatttcattgaaatgtcccttgggtaatctcacttgtttttgatatatctctattttacgtagggtgtaagattatattgtcgtcttcatagcctttttactccagtgataaggtgagtctggtttttaagttctgggaaacttaaatggaaatatttgtactatgttttaggtaaatattattcagccagaacttgttggcttattttgttttttttcactttactttaaaatattattttaaggaataatttattgtaaaatatccctacattttcttaagattctaattatatagtgttttccactctttattatgatttctaaaattattttgccattagttcatagtaaattatacacttgaaataatccactttcagttaatatttaataaattatagttgattgattgggagaggatttggaaatactgtttacatatttgccattattattacttgtatattaactttctacattaaattgtataatcaaatattgaatatgtattttattttataatttttcttctcataatcttttttcccaagaaaagattttgtacttggaaattaagaattattttgaagttaataagtataattttataattttattctttatcatgtttttccttctgaagaggcacacttgttttaaaagaggaatcaggattaatttttagtttatctctttagaaaggcaatagaaaactttgaggtttgtattatgattactaacagagtagatcttcaggaaattgtacttttgcaatatacactgatataccttaactgatagaatgttaaactaatttgaaatggtcagatattatttggaatttactgactaaatcatagtccgcaacaaaatatgcctgtaccattttcaaaataaataaaaatgcttttcaatcttggctataaaatccaaatgtttagtgtttctatttttcactttggtttgtaattccaagaaaatgcgaagcttaaaaaaataaagattgcagatattgctaagtatattggaatgcccacttatactgcaaagagaacacactgtttgcaggtcctggaggagaaagcaggatcttacccacaatcctgaaagtgccctatttactttcacaagtagacttcacaaagtggaaaaagatgaaaaagaataaatatttgttgactctgccataattctttcacagatacccataaatattttggataacttgatattaagtacttaatagcaattgggtacttaaatattcttttctgctaaactaagataaaacaataatgtttaatgtaccatgtaacctgtgtatatgtaggttttatatgggcaaaaatgtgttctgaaaatatattttagtcatgtgaggtttcttatcaagaaagctgcatctgtaagagctgataaggaatttttctttggctaaaaatgacaattgtttttgagttgtcaagagagaatttatgggaaaaaaccataaaacagaaggaatgtatgatagtcaggcttttaatatgtgtgaactcagcttataaaaagaaaagttacttagcatgaagagccgtagacttgcatatggtctgtaatagactacgctttttcttcttacgatgcttatggaaaacaagatgaaatgggtgatgaggtgtcagaaacatggagatagatttaaagaacaatgaatatagctagtaattattagtcacttcaagtcttttgcagtttagaatttttgacattttataaagactgccactttttcatttttttatgatccaggtttggaaaataaatgaattataggtttctgcccttgggtattcagttctgatcatgaaatttggttggtatccaacagaactttgaatggtttctcagttctgggaaatctactgaacggagagtctgaggtggagagccagttgatcctcttggttctctggggatgtagtatcactatgtccatgatctaatcaggcaaaaagaataacaggttcttagtgtgtctacttcaggacagacctgcttgttttccactagggcagagctgtgagaataatactgtagtgcaaatccagccttcctggagcattcaggagcaggaataaaaggcatttctttactctacagattttgagatttttagtataatctattgtgatttaaagagaagaaattctattttattattttattaatacgatgtacagtaagttctcacttaatgtcatcaataggttcctggaacctgcaactttaaatgaaatgacatataatgaaaccaattttgccataggctaattgacataaataagagttaagttccaacagcatccagtatttcatttcacttaaagtcgcagtttccaagaacctatccacaatgttaagtgtggacttactatatttccagctaatatagcagtaataatctatttggatataagacaatgtgaaagaagagatgtcctgttttggtaacattgtgagtctcttcccttggtataagctggtggaggatttttcaggtgttttacatgcataataaacttctcttatttgcatgtaatttgggcaactaactcaggttacagtggtaaacttctctgttgcatattgatactaaatgaaaatattttcttgtgcttacctaccctcttgcttaatttttgttttttgtttgtttgtttttattttttcctttatagagataaagattaccatttaaggacctacaaatctgtggtcatggccaacaaactgatagactggttaattgcacaggtaaatagacaaatagaagttgctgagtttcacttgctgctgctttaaactctgagtgtgtgaattaggacctttattctttttcatctgtagcaggcatttaaaaaaagaagtacatacaactaataggaagcagagttaggatttgaacctagatgatcaattttagagtcctcaaacttcacttttgtgttatcctgtttctcaaaaggaagatgttcctatttttaaaatatcagatgatccatcaaaaaacactaatttgagaatctgaagaatagctatgctcatttactaaaatatttttatgttttttacactttgttttaaagtatctgtttattaatgctagcaagttattctaatctccctgtgcctttttcccttttagaaaataattgtgttttgataatgagctttaatgtctgcctgtgtccattttaaaaaagaaaggaatttattgttgttattgttgacctgcttctcaagatttagatgctgactgctagaattgagaaaatgtcattcgcttataagtaaaaaaaattagggcatcctttccatcataagagacattcaaccataaattattccaatattcaaaagtttaagtattggtattcaaatatttaaatgttttatcatactagatcaactagtaaatgataaaatgtgtttcactgattaaagaaaagatgaagaaaactgatacccactgtaactgaacttactattttgggggaggggatattgcttggttgaaatgcaagtcactatgtttagctatgtgagtcaatttcatacacacctttactttttcttaagggtggcttgctttctttgtttcaagtagataatagttgatagtttgtcataaaatgtttattttttgtttcagttgttaatacttaatccacatttggttcatctgtacacacatattttgataatttatattagttaatattatttttaggctgttcaagaacacctatttttatatttcatagcacttacatttattacatttagaagtttttagatgaaactaaacaattacctttagataaaacatcaatttatattttcagtggcttttctagcatcagagcctttcataacactgtgggcaatgtgtgtcattcgagctgatttctatgttcattattggagttggactttcacatagaaattatacagttctagaattcatgtgaacttgtatgatgtgttgattacgtgagaaacttaaagaaaatttatctgaagcaaaaactgatgctgagtaaacctttaaagttctagctcaggaaatcaggtatcaacttttttttttggcacttttactttccaagggttaacattagaagaatatattaaggtgctcaaaaacggccaagttgcctttttttttttagacgagtctcacttggttgcctgggctggagtgcagtggctcaatctcggctcactgcaactttcgcctctggggttcaagtaattctcctgcctcagcttctcaagtacctgggattacaggtgtgcaccgctacacctggctaatttttgtattttcagtagagacagggtttcactgtgttggccaggcagctcgaacctctgacctcaagtgatccacctgcctcagcctcccaaagggctcggattacaggtgtgagctaccacgcccagccgcaatttttttttcttcagtttttcttcgtggtttattatgacaatgtctgcagaaattgtacataataggaattgcattggctgggactcacaacctagtgtctgatgccagctgtgctagtaactagttatataaacttagacaagtcactcaatctctatagattttagtttttacaactgtttactgcttactgcaatagctgtacatgccgtctcttttctatcaccgaaactttatgattctgatattgtaaaacatcctaatgtaagtatcaacataatcaactctgattgcttaacttctgattaggaccacataggctgccttggaaaaggcagaaatagtaatggaaggataagagaacaacaagttaaagttcttaatctaacatgaagaagaggtttttgtttgtttgtttgttttttgtttttcatgtaagggtctctttcttccaagactctgaaatgataatattgaatgtccagtatggactgagacactgaattgatttggacggttttagaacacagagtaaaaaaactggctctactatgattggagtatattctagagcctatagtttttctcttattctgcaatatattgtgcgatgtcatcttggaagacaaaatgagtgttgtgttcttctgagtacatttttcattcactgttgttgtgctgcttctattatagtataatggtttgtaataaaataccctgtttcacaaagttaaattcttatttggcatgtgcagtaatatacctttaaaacatcattcaattcctgtacgttgtgatttgggtatggatcactcatagttcataattaaataatgccttgcttcttcacatccatgcggtcttactgtgctttttctaaatgctcaccaggtgcatatggcacaaggaggattttaaaagagcataggaaacccagaacatgttaggctatgttttttacaccattatgttcaataatattagtctggccagtgtttcatttgaataatcaatcaaataaagcaatcaaatgtcagtgattattaaacaagcttttcttgtaagcttaatgttcaagcaagtgctttgatgatagaatacacaaacaaagcaggaagcaatcatgatttctgccttgaagtgatgttggtcaattgggctaattttcatgtaccctctaaagctctgctgcagcgtcatcacccccaggaagcctcgccagatcctctagcctaggctcagggtcaccttctgtggtcccagatccccaggtatatctgcctcattgggaatatggattaaattggttattggtaaagtacttaccttttaatgaccaaatcctaccctagatttgcattacctcagagcagggaattattttgtgcttgagtggtttaatatattttttctatcacttggcacaatatgtagaaaaaataagtatcttttgaatgaatgcataaaaattacaaaacagctcgatttttgaagagggccagatgcagagtctgaagttacagattatgtgtttagtagttcttaggaggattaatgagggttggatttgtcacagcatgaggtaggacccttattttgctcctttatagacaggtagaatgtgagtgagtggagggtaaagagaagggcattgcagctatgaacaattacatgagtgaaagcaggagaagaacgagcacagacttcaggcaacagcagggaggccaagttgcctacagcaagaggtatggtattgagctttggagagtgaagctggagagatagcataaaaccaagatatggaaagtttcaaaagcccaacagagaattttgatacattatgattctaaaagaacagtctgtttagttttggaaccgagtttctgtgtaagaaagtttaattctggccaggcacagtggttcacgcctgtaatcccagcactttgggaggttgaggcgagtggatcacctgaggttaggagtttgagaccagcctggccaacatgatgaaagcccgtctctactaaaaatacaaaaattagccgggtgtgttggcatgcacctgcagtctcagctactcgggaggctgaggtgggagaatcgcttgaaccagggaggcggacgttgcggtgagctgagatcaagccactgcattccagcctgagcgacagagtgagactccatcttaaaaaaaaaatagcttaattgttagaaagagtccagaaaaaattatggtaatttagaagggagaagaagaaacaccatatgaaggtattgacagtagaaatggaatagaagccagagaggagagagtagatttggtaacatacctaagtgctaaaagataggactggggaaaaatgagataatatgtgagaaaaaagctgcttgaactgtaacaatacaactctaaggtcacacacacacacacacacacacacacacacacacacacatgcatgttcatatacatggatccatacttaaaggcataaaataggtgttttctaaatgaaaaccaaaagaaaaaggttgctttgggcgcttttgttggattcagttaaatgatctgatttttttttttggcaaaataatgaaagagaagcttgtatttgctagctcagccaaacgatttacttgtaaggtcttacaagtcatcgtagattaataggtttcttcaggggtcctcacagggcaaggccatttggtggaagggaatagttagtgactgaggtcatacataatgattgtttaattagagtgttgtcaatttacaggatatttccctgttgcataattggaatataaaatctaagaaatctaatctaagaaataacaggcatcatttatggtagcaataaatttttgttacatagttttgtgtacaataaagtatgtttgtttctcaagaaatattaaatctttgttttacttaatgccaaaagaaaatgttttcataatcaactgaatgttttgtaacaattagatgcattctgtcttataaagcaatgcatgctttataagcgaagctttgtctgaatttggtgctgggaatgttaaatggagcaaagctgccacaaagcctttagttgcctattaactcccacagtgcttttttggttaacagggagattgccgcaccagagaagaggcaatgatatttggcgttggactctgtgacaatggatttatgcaccatggtagggattttttaccctgggagcttacagatgtatttcatcacgttggttcttaggatactgcaacacccagtgctgcagtgaggtcatttagggatgagccagcaagactgtctgggtcttgccatgggttcaggttgggtcaccacaactgcattcagttttactgctggtgccatccctatgtctaattgctgttacctaccaattgccatgactactatcctatttttctagactatgctgattctagaagaacaatggcttgagctagaagtttttctgttttgtcttgtttttaagctcataaacctctttgagactatttcagattagacttatggttttattttgaatgccagtatatattagaatgaataaatgacagcatatgtggttgactactgttatttttaagaaaaatgtttttaaaattacctcagagaacaaaattatagatctctagaaaaaaattatttttaatcattaaatgttaattttttttcttgtctgatgatttcaagtaagtcaccacaaaacctctttctgctgtgccctgggatgacttcaagtaagtcatcaaacaataatttttaaataaagttcttagatttatcagggtaatgatagcaagcttttcctgttcgtaacatcatatagaatccataaacccaaacagtagtctctctctttttatttatttatttattttttgagacagggtctcactctgtcacccaggctagagtgcagtggcatgatctcagctcaccacaacctttgcctcgtgggttgaagcgattctcccacctcagcctccggagtagctgggactacaagtgcatgccaccacgtctaactaacttttgcttttcttggtagagatggagtttcaccaagctaggctgatctcgaactcctgacctcaagtaatttgcctgccccagcctcccaaagtgctgggattataggcgtgagccactatgcctggcctctttttaattattaatataatatgcttataaacatatatttgatagcacttatacctgtttgtcagttactttttcagaatatctttacatttttatgaagaaggaagtacagcaaaggttacagtgattttgagaaggtccttggttatcatttttttgggggaaattccatcctgtttccctatctccagatcactctgtgagttgtcgtggagaaatattggtccttgaagaacaaactatgaacggaagaaattacatcatgttaggggattaaacctgtaatataaagctgaaaaccttcaaaagtttcaattttttttctttaaagtaaagcaccaacacagtaattttatctaagcgtttattataaaatattgagttacatcaaaagtttttcataccaaagtctgacagtaaagcatgaacagtgtagctcatgcctggctttattgatgccctatcatgctccttttaaaagacaatttggtggagggaaacagctgtagcagaaatattgtgtgaactttgggaaactgcttagatcattaagtcaatctaaaataggttctgagctaggcgcagtggctcatgcctgtaatcccagcacttcgagaggccaaggtgggaggtattgcttgagcctaggagttcgagaccagcctggacaacatagcaagaccccgtctctacagaaatttaaaatagtaacaataaaatagattctcacttctgttgctaattatggcttccgttgtttttcaaatttgtggttgatagtttcgtttgttaactcaagttgccatccagtctttcaaccaaagatcaataaagccattcatccagtcaacaattatttgttggagctctgtaaaaataatgaaaccaaatagctatcccttttgactgcttactatatgccaagttatttatatttattacctcattttggttttaagcaacaatatgtagaaggttttattgcccccactttacagatgaggaaactgaaactcagagggattaagtaactttcttatctcatttccagacctggaagttaaattcaggcctgtctcatcacaaaacctaactttttctgctgtgccctgggatcactttcttttagtctctggcttaatgaatcaaccttcaaggtattaaggacagagaaataagaaatagataacttttttttgtaaagtattttgacagctgtcaggatctcactgaatacaaattcttcatatcagtatttagccttatttttaaaaatactaacaaaacataaactttatttcatcagtgcattccaagaatcttaggggatttggggggaaatgctaatgttcagataagggggaagaaacagacctttctagaaataaggttatttgagaatattgcttttaaacagaaaagaaacaggaattcgtggaaactgaaatcacatttaacccagaaggcatatttctggtttgaagtattggaacaaaagtgaacacaatgagagttgttcacacaagaaggcaacacttaacccatttgaattttcccataattgtgtgggatattaaaaacatgtttaaagtgtttactacaaattgaaaacaccataaatttctatgttttggaggcacgcatttttggattcattataatcttttatttaaatttggattttaatctggaaagcgaaggacttggttaaattttaaaaagttagccttagaagaaatactatttgttttaaaaaattcaaaatcctgtgggtaaaagtaattgaaaggagacattggctcgaaattatagaaagagaaaaaaaacaaaacccggggctacttgtgaatgtaactagtcctaggagtataaagttgaggttatgggtgcaggtattaatttgtaaatgtcactgctattgaaaatgagtgcgatttttgaattagctgaaataatttgcatctgtctttttctgtagtccttgaaaaaagcgaattcaaagatgaacccctacttttccgttttttttcggatgaggaaatggagggatcaaatatgaaacatcgacttatgaaacatgacttaaaagttgtggaaaatgttatagctaagtcattattggtaagtttattgatatgttatataagttttcttcttgattagacactagcagaagactgcgttaaacatgtttgacggtgatgcacattacttcgttctgtttgacttgtaatttttttcatagttgctttatcaataatgttaatattttgcatttcagattaaatccaatgaaggcagctatggctttggattagaagacaaaaataaagttccaataataaagttggtagaaaagggatctaatgctgaggtaatgtaaattagcgttttaatttaaatttgttatcatgacataaaaatagagaaattctaaacaaactgaaatctgcctttaaagataatcagccttgaaattatctttatcaaacataatgtctctggataatcttactcacaggttctactgaatacctttgggattagcagcagctgagtcctgtctggccactcagggataataggggccgggaggtggtgggtattctatagcagccgtccccaaccttcttggtaccagggactggttttgtggaagacaatttttccacaggcggagggtgggggatggttttgggatgaaactgtaccacctcagatcatcaggcattagagtgtcataaggtatggtcaacatagatcccccgcatgctcggttcacaattgggttcgcgctcctatgagaatgtaatgccacggctgacttgacaggaggtggagctcagggtttgtgctcactcgcccgccgctcacctccttttgtgcgtctggttcctgtcaggccttggacccatactggtccgcagcctgggattggggacccctgtcctagagtccttcaactgtgaacttcttgaccttctgggaggccaagtggctcattctttgtccctggagcttcactaatcacagactgcaacctggagccccgtggagactgcatagggtagcactgtattatgcacggcagatggttcttcctttctgtcaagagctgtgtcttctgttttcccatcaccgttgatagtgttttatatgagtataactttcatcaccaaaggacatgctttaactattttctcattggattaatgctcctgatgttctctcacttcatcagcctttttgagacaaagtctccctctgttgcccaggctagagtccagtggcatgatctcggctcactgcaacctctgccttctgggttcaagtgattctcctgcctcagcctccagagtagctgggattacaggcccatgccaccaggcccagctgattttttttttttttttaatagagacaggttttaccatgttggccaggctggtctcaaactcctgaccttaagtgatctgcccacctcagcctcccaaagttctgggattacaggcatgagacactgcacccagcaactttaccagcctttcaaacaaaaaaaacttctgatcattcattcattcattcattcattcatttagaagtatttatttagtgcctgttatatactcagcagtgttccaggctctgggaatacagcaacaaacaagacacaaaatcccccaccctcataggccctacttttcagtgctgtagcccaggtatatctagtttaaagtgtcctcaccttggtgcccctggttggaagatacttaatcctcaagcaaacttctcaatatttagatctttaatgaatatgaaaattaacaaaatatggtatttacctttgaacctgtttaaagttagtggtaaactgtcacccatcctcctatctgctaatgcccaaggtctctgttttatcaaaacacaggttcatgcaagacaaagctttctgtacagcacactaatttcaaacaaaataactctgatcaacgtatgcactgtttagtggacctggccaacgaatgaccatttttagtaattgtcaatgagaaggaaatagtttctcccaggcattttcagaggatggtcttagagcacagctgcatctgactatgccactaataaaatgccctctctcaggctttcatcatttagctgacagtcaaaacagcggtagtttataggcagtaaggctggttgagaagtgcatgctggttggcgtggagggataatatcttccttgtcctcttttcagcagcttgcactgttgacaactctatgctggttggagctctcttctttggattccaggctccctctgtctcctgcactggagccgcttgagctatgtcaggctagagatacatatttgggtgtcattgatgtagatacagaagttgacactttctgagttaattggctataggtggaaaacacagtaaaaaaaattaactttggatttgattatagttaaaaggatgaaataagctaaagaacagtaaaatataacagaagaggggagtaggatgttaagtcagggagggaaatggggataattttggtaaagttctgtgttcaaatttaaaactatgccaaagagaatgataactgtgttatctacagttctgctttgtgaatttgtataaaaacacggcaggtgtgttaaaatgtcaatatctataagctctgaaacttttcatattccaattttgtgaaaaactcaaaatttcttattaaaatatactttgacttattttttgtaatttctctcttagatggctggcatggaagtcgggaaaaagatttttgctattaatggtgacctagtttttatgagacctttcaatgaagtggattgcttcctgaaatcgtgtttaaacagcagaaaacctctaagagttcttgtgagcacaaagccaagagagtaagttgtatgatttatgtgtgattttttaaaagttatacatagataagtaacacagaaaagataactaagtaaatgcttagaattaagcatctcattgcttggaacgcttgaacctcaccggtatcattaatactggtttacagatgatgggagaagctacatcgcttgtgatgctgacatacttgttacttgaatcatgtagttttagtggaaacaatgcttttcttttgctgcccacattttcctggtctgggttgaattgggttttactatgagtgaagaaaactagtagaacatttgagaataaggaactgtgtttctccatgttcggctataagaatttctggtgctatcagatgattagataccagataccatttataatttaacactcataagagatgcaaaaaagactgaaagataatccatacttgcatgtgcttagtaatttgtccatttctttaaaaaattctattaactatctacctatgattagaaaaaaagttatgtttattatagaaaatgtagagaatacaaatgtttctaaagggagactttttttcttgcaatatcacccataaatagccattgttattcctttgaaattaagtattaactaaagtttttaaagtattggtacacatatacacaatcaggtacccacaaaatgagagcatgctctgtatgctactttttaataacataccatgcatatacttgcaagacatgggcttttcttgtgcaaatcatttccatcaatggtttagtatttccttatcatatttaccttgaattgtttaaccacttaattaattgagataatttgtacaaactatttagcataatatatcaaaattattgtttaaattttacttgtatcatttgtcaatcatgataagcaacttcattagttttaaagcaaatgaaatttagcacttcaaaaataagattttttaaattttaactacctattaaaagtacatgcagtattcttccctgccttaaatatattttaatatgtaaaaatacatatttatatgtaatatgtttatatgttatatatattcaggaatagctggatgtacttctgcagtgttaagtatcttaagagtcaaagtgcagtgcagtgggctgggttctccctagtgagtctcttcaaggcagtgcgaattcattaaattagatgctttagcttctaccaatagtttaccaagaggtctgaaagagtgagcctgacactcactttttactaatgtaggaagacggggctagagggcaggaaagaagcacagcaatgtcttccctctaaacctaattatggtctgttcctctccctttattcaactctgtctaggagaagactgtcctgcttgcccacctcctgacactttgagggcatttaaaaggaagaagacagactggaaaggaataagggagtagtattgaaagcaatcattacgtcctccctagggccccctgatttaaccctacaaatactgatctactgatcagagacattccaactgttagagactttgaggtcatcaacttcaaaactgaataaataggagcaaatcctcagtcatagacttagttgctagatgagcagaatagcaatctgtgtcttctgactcttcttagtgttcgttgcgctgtgtctagaactctagattacaggttcaagcctctcttccactacttgctagctaagtgtataacttcacgtcctcatccataaaatggggataagagtacctgtgccatggaactgcaaagaatgttgaataaaactaggtttgtaaagaacttagtgaagtatccagtacatagtaagtacccagtgatgttatatatgatgctgttgtcttctcattttattatgtttcattgctactttattatatcaagagcatattttacattgtatagcatactcatttttttgaagatggcatactcatttttaaaaaacttgtttgaaataaatgacttttcctttatgtccttattctaatgtatgagttctataaatcactgtcatattttcttacaatagcttcttggaatttaagtagtatcccattttgaggaaaagggaaaaatatgagagataaatgcaaaagaattttcttttgattctagctatatttcatacttgatagcatcaagggatttttcaaaagactttgtagcagataatttagtacacttattttctgtaggaaatagaacatccgtcttgtataaacgtaattcttttttaatagggaatataatgttctcaccagggaaacatgttttctaaagatcattttactaaggggcttccaacatttggaccaaaatgcatgtcatggtgttttgtacaatttcgattaaggaaaaaataattctcttaaagattagagtcaaccttccatagcagtgttagaaggatagaaaagccttgctcagtaagattgagaggccactgtgaattactcaaaatccaaaagcaacaataattcaccatggcctgaaagttctatgttgtttcagcagaagtgaactctcattcgtaatatatagaagcatgaacttcttctgattttgtttcaggctcaccaaaatatgtattcattccctttttctttctttggtcagttatctttcattttcaagtgaaactttgacaaacactgataactatgtgttattcaaaggaaatgaatacagccctgtacaccaaaaactgtttaattaaggataaaggatacattaattctgtgacctctatcccacatacatacagataacttcatagtgtataacctaatttataataatactttcaagttttccgagagcagaaaatcttcatttctatccctgaatgcctgcctaggttatatttgagattgcagtgccagcctctctgccttaggcctaaatgtagcctatggctttctgaagtgtttgagcccttagttttgctcaaactctcccacagcaatttcataaactgcagataagtcctgctgtatctaccttcagactctgtttaatatgcccttgaagtgctgctgacatttgctttgtgattaactcatcttttgggaaacacttttgtttcttgggcgtcaagggctatctatcacaatttattgcgttgtattcatttgtttttgttcccggctgcgatatcatacttgtattgttatgatgttccctcgcatcttgcactctctgcttttcattgttgctatctgatttctggctttcttcagtggagtatcttatttatgagtgccaaattggacacccctgactggtttgccgtgacagcttgctcaagtttcccatctcttcataatcttttttctttttttaatgtagattcattggaatgatttcttctcttattcaggtttgccttctgatgtattattcttttaaaccttcattgcataaatactaaagtgaatcattttcttaaaatattttgataccacgtttgacagatctgttctttataactagctctttgttgttctttgataaagctcccaataggtagaaatgtattaactctgtatttctgttacattaatttttataaagaatttcataggtccctcattgttttctttgtaacagtaaactaaggagaaaagtttctgttttattaagtacctgtatagctcatagcacagtacagggcaacttaccaaggttatttctttttaattgccacagataccctgcaaaataacttttaacaatgttatctttctgtgtcagagtagttaaggagcttatctaagttatccacctacaaactgactacttgaagattcaaggctacgtttaagcaggtatggtggctcacacctgtaatcccagcactttgggagactgaggtaggaggatcacttgagttcagtaatttaagatgagcttgagcaatgtaggaaaccctgtgtttatgaaaaaaataaggtaaaataaccaagcatggtgtcaccttgtgcctgtagtcccagccacttgggaggctgagatgtgaggatcactgggacagagagattgaggctgcagtgagcagtaatcaagccactacactccaatgttcctttttcaggctttactgtgtaacgactgtgttcagtttagaaacactgtattgcatgaggttggggaccatgttcgtctccaaggatttctagcccaacatctttcacactgtagatcctcaatatgtatttattgagaggtgtaaagctgtacacgacgattatttcaaccagttttgattcttacgcttcatcttaccttattttctgattgatttaggacagtgaaaattccagattcagctgatggacttggcttccagatccggggatttggcccttctgttgtgcatgctgtaggaagaggtaggaaattcgtcttgcagggaaacagctttccagcaggtttgggatgtgcccgatggaacaggaatgtgtttaaaataggcagaaccatgctgtgatgattctcaattatattttcactgtattaactcaatggtagcacattccagaaacattaaaaaaacgaaaaaattctaagccatatttatcattgttaaaaaattgatataagaattatgcctaaatctcacttttctgcatccttcagtttatccaaagaatataataaatttaagattttctatttgtatttttcttgatttcaagtaattgtatttctttgtcagtaatccaaagttgtgagtttttaggtttaaagttgatctgtggactttataacctattgctatatacttccttcgggtacttttataaaccgtggttaaatcccatcataagatgcattttaaattcaataattttagtgactaaaaagttatgtttacacttattcttttttgtttgattattgacatctcttcttatcatatgtattttttaaattttaaattgacactagcaacaattgaaggaaataactgtcagatcccacattactaaacatagtaagtaacttgcgcctatagcaagaagtgtcactattagcataagaagtcatgtgtttatctaacaagagacatacatgctttttattactaaagaatgtaaagtatcttgatgtgatcacccaaaaatttggcaagaaaattttaagagggcatttttatcagaatcaaatttatcaaggacatactagctagacctttattataacattactttcattattgtatgtatgataaaaacatctgatttcagaaagctgtgtttctctcagatttctaaaacatatagatggatagacagttaaaatcttcccaataactcattaaatattgtcaaactaaaatgggatattgaggcaccaaatatcttctgcattttcatgaagacaaaaaggaataattcacattaatagtgctataaaatagcttaaagcctgagtgaatcaagctgtacctcttcccttattacaatgtcatttaaagagcaaggttctatctttggagcttagtggtggggttggcctacgtctggtttctgtgctttttgcttctgaagagtaaaagacagaaatgctcaagtgctttctctctcagatgtgtatgaaatggaaagatatttggttattttttggcactgtaattttgccacaagaaaaagagatttctcgtgtgggtcactgcagtcacgcttgccagcatccccaggctcccctgcctcccaccctcagctgcattctagctactgcacttcagccttggatcaacctaaccacatcttctctctttttcccttgtagctggaaaagactgtcatggttattgaattagtgtctcccaattgactgtcttctactgggcccgtattactactcagcatttttaaaaatgtgtccttgatcagttttttttgctcctcaaaattgcttcctatatcccaagatgaacttgttcctacgttacagacaaattcagcctttcagacaggaagccctcggagtcttccatttatagcaaggttgagactcttagtcaaggcaaagacttcacagttggctctattcaactctccaatcctgtctcttccatccactcccattctaagctcacacaaacagaacagcttggatgcttccaagtggccaggtgatttcttgaagcaaggccatttttcaggctgttctttctgctgataattttccattttcccctaagactcaattcaatgcagaaagccacttaacatttctaggagtgaattttatggggattaagaagaaatgcaaagatatattcttgaatgtaaattattttcttaaagatgtttaataatttctttatgagagacattggtcacaaaagaagtctcctctaatatatttacaagtttataatcatttgtattaattatttgcattgtattcattacatgtatttattaatggtattttgttaatattgatttgatcacactatcaaataacaataattggaaggttgtgagaaatcttagctcactggttaggatatgttactttcatatttaaatctatatcaatgcaaacaaaatgacaggatgagtgatgctatctcattaattctgttctctacaaattcccatctttctgactttttgtatatatgtcaaactcttatttttcttggaaaaaccagtccaatcatagttgctcattgacttgatttttctcttaaggattttcttttttatgtgcatgttaaattgttgtcattttaaaaaacagaagtgaatactatttgaatatttagaaatgttgtagaataaaggaaggtaagtaaggcatatgtttctggtcttctcattttgccattatatatgcatatttagaaagactgactagacatttggaaggaaaaagaactgattagtattataaaggtcactttgcaacatcccttcaaactaattcttagggaggtgagaaatattctattgatcacagtttgaaagttaagttaaaaagcagaaaagtgttgagtgggcattgctctcagaaccaagtttatcaagattatactagctagactttgatcatattttaaaattgcttccataattgtatatatcatacaaatgaatcttattcctgaaatttgtttttggttattttctcatttgtatttataggaactgtggctgcagcagctggtcttcaccctggacagtgcattatcaaggtgaatggaatcaatgtcagcaaagagacacatgccagtgtcattgcacacgttacagcctgcaggaagtacaggcggccaacgaaggtaagtggcccttcagataatctggatctgagtgactacttgcataaagacctaaggggttgaggtgggatagtgccagagatatttgtggtttccaattttaagtttaaaattaaacacagcattataaatctcataatgattgctttatctgcataattaattataattgattgtgtttaaatggttacattttagtattacagtaccgccatctgtttaaaaagtgatctggggtattttcattttcattgagatacaagttctatcaaattgaaatccaaaggtttgaactgttttcttggcccttgtgattgctttagaatatgtaatgatcattgtgtctgtgtctgtatgtatgaactataaatacctatattctttattagaaaacttctgtgaaaaagcaaaaattgtgggttttataaggtccatgattgattttgaggtatatttatttaggtaactggatttagctcttaaaggaaaataccaaaatgtatttaggtaactgcatttaggtcttaaaagaaaataccaaaatgtactcggcgttatctaaaattgtagaacactatcctttgatctacaagtgccacaaaaaactaaaattagaatcataattcataagcaaagctattatgaagaaaatgtgagtattggcatataaaagaaaactcctgcaatgtaattagctagatgcaggcagaactttacaggagaagccttgtattttcctgtcatggataatgaagtactcatttctggattggatttgaggatgcaactatgtccaaacctggaagatgaatggccattttctcataagccagggctgatggcctgcaaaatgctgaatctacatgaaaataactgatcagttttacttaatgagaaggtcagacctagcatagcaaattggaagttatatataggctcataggttactctggttgatagaagcttgataaatatttgttgaactgtattattgaatgaatgtttaaagtctcatcggaaatgatcaaatcttttctcttctaccagaatgttaataagcagagaaatcgatttgagtttcatttctttttattttccctgcctaccctcttatagaggtgttgatcagtgaaataattaaagaaaagcaataaatgggcagaaaaatccactaacatgctctaccagatatccattgccacaaaatagaccactgcaaaacttagtggcttaaaacaatgataattgactatttgtcttgattctatggggcggccgtggggtggttcctatgaggttactgacgtggctgcattctgctgggacccgacttttgcttcccctgttcgtggcctctgtttccatgtggcaggtcaccctccagagcacctttgcatggcctctctttccagtctctaacggggttttcttttcagtgtgtgtctgaattttaagagggaacattacaagtgataaatgttctacttagattgtacttgctagtatccctttggacaaaccaaatcacgtggccaagcccagggtcaatgtgggaaactaagcggtgcggctcattggtggccacaacagggcaccaagatgtaatggaatgatcaaatgttaattagcattttaatgatttgtagcaatatattaattagacacacttgagaatcgaatacaaaaaatagataatacatattgtatcatacttcaataatgcagtttaatttgatttaacatcttatctagaaaatctataacccttaaagtaatgcaacccttgaagcatttgtttggtagtcatctcatctctctcatttctctaaataagactggaactgctgttaaaatataggataatttttaaggaaatcgcccttaaaaataggatgttttttatttggagggattaaaagatagttacattacttgggagaccaatgagatgaatgatgtccttccaattttcatatttgtttgattctaaaatagcagtaaatgtattagaatatatctgactattaggggaacatgtaaatgatttgtacaattatagataattgattcatcagacaagtgtccctctcacaattgttttattttgttgtcttattaaaaagagatggggtctcactacgttgctcaggttggtcttgaacttctgggctcaagcgatcctcccacctcagcctcccaaagtgctgagattataagtgtgagtcaccatgcccagccacatttgattattttaaaatgtgatttataacttacttacgatatacttgaagcatttaagtaagatattttggaatatatcctatttaaaaacatacatctttggaacatatcttagaagataaaacttgaagctttaaaaagtcagtttgttaggccaggcgcagtagctcctgcctgtaatcccagcactttgggaggccgaggcaggcagatcacctgaggtcgggagttcgagaccagcctgaccaacatggagaaaccctgtctttactaaaaatacaaaattagccaggtatggtagtgcatgcctgtaatgccagctactcagttggttgaggcaggagaatcgcttgaatcagggaggcggaggttttggtgagtcgagactgcaccattgcactccagcctgggcaacaagagggaaactccatctcaaaaaaaaaaaaaaaaaaaaagaaagaaagagtgtttgttaaacaatataattgtccttgagtttttattagtttctgatgttaattgtatctctctttcaaattttactaattttaaagaatctagaggaataaataaggccaagtctccaaatgggcatgtattttaggaagcactaatacctctgaggtttctttcccccctcatttccagcaagattccatacaatgggtttataatagcattgagagtgctcaagaagaccttcaaaaatctcactccaagccccctggagatgaagcaggggatgcttttgactgtaaagtagaaggtaggtttcttattttcttcttcatgtgcttatagtattcttttccactgtgcatgtgcctactcccaaatattgaaggtcctgtacatgttgaagccacattcttaagtcgttatcaccaaacagatgtctgtcaaatgcataagtaataatagctagtagctggcaaattctatttgttaggcattttatgaagtgtttttgtggactatctcacttagtcatcaaaaaagaaaatctcctaaaatggaaatgaatatcatcattcttttttgagtggagaaaacactaaagcctatataggttaagggacttgctcaaactagttaagtagtagagtgaggattcaaacccaggttataatgtgtttccttgtttggagtttcagtgttgtttagacctggcctccagtgtgcctcacagagcagcagtatcaacatcacctgagagctagactgaaatacagaatctcagctgtaccccagatctactgagtcagaattctcattttaactagatttgatttgtatgcatattaaaattcgagaagccctgctctagaagattcttttttacttttactgtattctaacattgacagtaaggaaaggagcaaactagtagtgtgaaaatgagagagaaacagagagagagggagagagagatcttttgataaggacggttatcatttctgtttcctcaatggagtaaaccagcaaatggctctgttcagtggcttctttgccaagggcgacagaggcaattacatttatgtctgcaattgacaaataccttaaattattgtttcaaaacgttttcacagaagcccatgaatcacctcttatattttcttggtgtctttccactcatattattggtggatgtctctggatatctttgttacactattaggttcctcccaaccagcacagctggagaataattccctattctgaggtgttttcattcaaaacccttctgacatcctgtatgtgtggggttttcccccataccaaccaattccccaaatctcctaacaccaactgggtgtcctacaattcaatttagttgtaaagccaggtgttagcacagacacacaggttaggggatcagccccacaaggttgcccccatttcaggcgcctgtcacatgtcccaggctttcacctgtatttctgaccaactggctatatattcagcgggttcccacatcccttccttgggttcagttatttgctagaacagctcagggaacccagaaacttacaatcactggtttattataaaggataagaagtgaaaagcctgatgaagagataaatagggcgatgtctggaagagtgctgagcaccgagctgtgtagtgggaagctctgtgaggggttggggtgtgccaccctccagacacgtgggtgtgttcactcactcagaagctctccaattcctgtcatttagggatttttatggaggttccattccttagacgtgattcattaaatcatcagccattggtgactaaacttgaactctagcctctctcccctccccagaggtccaggggtgggtctgaaagctcctaccccctaatcgcatggttggttcccctgacaaccagcccccatcctgacactagctgtgggctcaccaagtgccacctcattcacataaactccgttatgttctaaaggggcttgttataaataacaaaagacattcctgtcacctctatcactcagtaaatcacaaggatttaaaagctttttttcaggaacggggccaaatacatacatacatacatacatacatacatacatatatgtgtgtgtgtgtgtgtgtgtatctatgtgtgtgtgtgtgtgtgtatatgtgtgtgtatatatatatctttctttcttcccttcctttcctttcctttccattttttttttttcagagtctttctttgtcacccaggctagagtgcggtagtgtagtggtactattatagtttactgcaacctctaatttctgggctgaagtgatccgccgtcagttccctaccccttccagtagctgggactacatgcacatgccaccatgcccagctaattttttaaatttttgatagagacaaggtcttgccatgttgcccaggctggtttcaaactcttgagcttaagtgatcctcccatttggcctccctgactgttgggattacaggcatgagtcatggcgcctagcctgtatttcttactgtatattccctatacacactgatcgtatggggggtacttaggaaaagctgttttctctgcactcctggaaggccacttggttggatgctacccaaatcatgggtaatatcagaaacatctcaactcattggagcaataaggttgttatggtaatacataatatttatccaaaacaggacaaatctcaggaccttattctgaggtttgtattcctaatttggaataaatcaacgttggtgccacattgaaaggtagattggttcctataatgctctggaaattgatttgtagctattctaggacttagttactttaagataaatcaatattctacttttgcctatttaatatttactgtttatgctatttttgggctttgttgttctaagtaaactaatattctagttttgtttatttaattactattaggtttcccaggagtttcttacttgagattttaggtatttttcaccagctgacgtaccatatgtaggctaaaaaactataggcagacattttagatggaaattttattatagtgaacaatgagaaattttctcaatgagccctctctgtaagttgctgtttgggaaactgcaggcaccctgtctgccaaaataaaatgattcttgcatctcctttctgcttcttcaatatggccttctttatggatttttttcctgctttaagaaaatattcatacaacattcgaggcagttttagaaatcctgactttaagtcatacatgtctttaataaaaaaaatttaggtatagtatttatactgatataaataggtagacatatggtccacagattgaagtagatatgcaaagataagactagtcatgttagagtgctgaggtttggatcactcaagcatatgcttttctcgaaatgttattttttaaaatgttaacaagaatagatgcttcatttaacacatcagatttaaccatcccttaaagaactctttaaaaattatttaaagagacacaaaattatttgaggagattaacaaatccttcatgaatttactgagttacagttgtctttgttccagaggttattgacaaattcaacactatggccatcattgatgggaagaaggagcatgtgagtctgacagtggacaatgtccacctggaatatggtgtcgtgtatgagtacgacagcacagctggcatcaagtgcaatgtggtggaaaagatgattgagcccaaaggtttcttcagcttaactgccaaggtaggagcgatgctgaagaaataagattttttgttctaaataaaggaactgaaatcctccctctccttcacttaaaaaaataaaaatagctatacatgttgcagctgtcagctcattcaaacttctttttttttttttcttgagacagaatatcactctgtcacccaggtgagagtgcagtggcgtgatctcggctcactgcaacatctgcctcccgggttcaagcaattctcatgcctcagtctcccaagtagctgggattacaggcatgcaccaccatgcccagctaatttttgtatttttagtagagatgaggtttcaccatgttgcccaggctggtctcaaactcctgacctcaagtgatccagctactttggcattccaaagtgctgggattgcaggcgtgagccgctgtgcccagcctcattcaatttcttatttttctccccctcctccactatttggtatgcatacaacttccttttattgatccttttggagcacagtgttaagttagatgaaacccttgcttaggcaggtgttcattgatatggcaaagacttcacagcatttaagtttgctgtgtctgtgaattcattattgtgaatccaattcagatggttttcatgacagatgctactttttcttaaatgtacataatcctctgaagagtattcaaagtcgtgaaatacattataagaaatttcaaatttggcaagtgccatgtttatttcataaaatttcgggaactgtagagatcagtttatgaatagatcctctgatatttatgtaattctcatttggaaaggacatcacctttgcagaggtctttaggctgaaacaagatggagaggaaggaagggagcctttatccaaattaataaaatacatggtaaagaggcaactgctggcttttcatggcctgcctacaaggtaaatccatcagactcctactgtatcctccacaggtgttggatttagtgtttcatactgctctatattctatgagaattcagatatcacaagtaagaaaaatgagatgtagctttcctacctttatgtctctgtttgaattgttgtttaaaatatgaatagttgagatagatgtagcatttctaacatgtatttttaaaattttagtgggaggaagtaatatagacatctaaatgaactattttaaaagtaattattttatcttttggttttatattctgaaagttagctttttaaaaatctcatcattctgatgatgttgtacctctctatatatctactgatatctaaaatatatatttttaaaatgcagtctcttcccgtctacattatagtattcatgggatccatgtaaaagtttggaaccaggttattataattattttctcattttccagctatttcaagtttgcagaattacatggattattctatatatgacagcatatgaactagtattacaaatatcacttggattttacacatgaagaaaaaacagtcaagaaagaaatgtagccaaggtcaccctgctaatgataaagttaactctaagttctagactagtgattaaataaaaactcatactccatagtctgcataaaagggaagtggaggtggtgagggagctcagataaccagaacatatatacattttatgtttcacagaatattataaaatattgattatgggtataatcaattcttgcaatgtctgattaatcagaaatgctacatatatatgtgtatatatatatatatatatatatatatatatatatatatctcacataattaattctcaggtcattggaaattcttagtgattgatggtacaccttccccatcaacacatattattacaattgattcaaatagtcaaaagaacttactaattgaaaacatcacataaatacttttatctaatgcttttatcagcaagagctcagatgctgcaagtaacattaatttctaccttactgaaataacttttcatttattcatttcttcagcaagcagttgtcaaatatattctttttgcacttgccagactctaagaaacaagtaagaatgatattggattcctttatgtttttttgttctaccaaattgagaaaaaaaaatgttctgagtgactctgactaaaatatagaagtgtcccacagcagaatgtactgatgtatccagcagggagtctttgaagtccagttacctcaccaagcaggctcctatagttagcagaaggaaaacctgaaatttcaaacagacaagtcagatcacttcactgacagcgcctcctgactgaaggtagaaattccctggcagctacccagagaacaaaataggaaagttgccctgtattgttaacttaatgaaaatttgatatggctacttttgaagtttttcttcttgtttcgttttgtttttctatgtgtgtgtctgtatgtgtgtttgtgtgtgtgggtgtgcgtgtgtgtttgtcattgcagattcttgaagccctggctaaaagtgatgagcattttgtacaaaactgtaccagcctaaattctctaaatgaagtgattcctactgaccttcagagtaaattcagtgccctttgcagtgaaagaattgaacacctatgtcagagaatatccagttataaaaaggtaagtgttctattgattttatacacaattattgttgaaattattatttgaatgtcaaatttaaatcaatttttgatattttctttacatatttgttaggtaccattttactgagatgataacattatataacattttagagtttttgaaatggcttaacatccattacctcatttgatctttgcaaacttgtaagttatgttactgtcccacatcttcaaaatatagtaactgaggcctaaagaattaaattattttctagtaaaagatagagctgggcattgagtgcagaccacctaattctgaagtcaaaatttaaaattatactatagttcttcttttcttttctcccaactaagagaatgaaaactctcaatcagaaagtacatagactgtgggcttttaaaaattaattgattaagtcattcatttgacatatatttattaggcaccctctatgtgccaagtactgttctaggttccaggaatatagcagggaaggagacaatttctctcctctcatggtgctcacattcaagtgggagagacagcaagtaagtaaataaatccctgcaaagtgaggggaatcaaatattgagaagaattatgaggaaatcaaaatagggaagtatggtttaagtgccaggttgatccttttgactctgtggctagagaaagcccctttgagaaggcttgacaagtagaactctccctttcaattggagagagaagcctccagggagaaggaacagctggtactgaagccccaaagcagaaagaaggccagggtggccagaggttggtggccgggcttaaggtcagaggcttaggtggggccaactgtgaagagctgtggcagctcctgtacattttgggacttgggaaggagtttggattttattcttatattaatgaaaaccagttggaaggttttaagcagggggatgacttcatctgatttaggcttttgaaagatcatgcttttgtaggaatggattgcaagggggcagaaatggagaaatagagactggtaggaagctgttgcaatggcccagttgaatgatgttgatggcttggactacattgaagatgaaggagacaaaaaacattagatgaatttggggacgtattttaaaggtagaattaatgggaattcatctccaaaacacatgctaaaacaagtttaaaaattgtttctatttataccaatagaagactaattgattaaattatttccatccaaatgaagttggttgcttaaaatttggtgcagccgtactagataatattctttatctcaatatatatgaatatgtgttaagtatatgtgttttatgttcttgatatggtaattaaaataagcccatatatgtctatatttggctgcatgagaatagggatgtgtgtaaattaatacccaagggactcacaacattggttacctaaactaaatgagtcaagggaaaagagatcatgattttttcctttaaacaattcctgtaatgtttgggttgttgagttgagtttgcattactattgtatcttttaaaaagttactcacatgaagaaaactaatattcagggttgctggttacctgccacatttgttaatctgcttttcatgactgctgagttcagttcacctacttgagtttggggaagattccatgaatgtaccttgttggagttgaaggacagaagattcttttcttacttctcctttctctataaaacatgaccgtgctttgaagtaaaattgaagtaaaaggtctaaacaaatgcaaacgaaaatgctaaacaaatgcaaatcagtattctcctttcgaagaagttttgaaaaagagagttgttctactttagaaatggatctgtacaagaaggtgtgatacaggttcaggcagattctgggtgtcaaactggtcaccgtgcctgggacatttctcttttgctttcttcctgctttcctctcttcattctttcacacaacaaaaaagtggaagtagcttaaagaagtatacatggaaaatggtaaactatataaacaaacataatcttaggatcagggtatattaggtagcaaattgatgcagctgctttaagaataaggaccaagagaacacaggcttaaataaatgtcggagatcctcaatagtcaccttggaaggtgagaagtatgttccatttactcataggtaactgagatcctagaagcttttaaggcgcagatgggcaggctaatgggcaatgatgttagatgataaaaataatgtcacccaattatggaaagctcttttatgtacattattgttagttgagtctcaaaacagccatgtgtatggactcttctctcatcctgcctctttcttcgggagattaagaatttgcttaaggtcacgtaggtggcaactaacagactaggttcctatggaatgtagtctcacttgtgtttaatttaatttagaaataaaaaagagacatgtagtcacctagggcctatgtgctttaaaagctctccttgtcatcataccatttggccataaacatatttaaaataactggttttctcattagatagagaagcaaggttagcaagaaaacactgcaaggatcttttcaatttgaataagatttaggtgtctaagatggtagccctaataggtcatgtaagattagctccatttatgtatcaaatgcatgtcaacagaacccatagcatttgcgaatatagatttttaattttcacacatatactttaaaactattattagcaacatatttttcttcctgatgaaggctttaacactcttctttttgtgaaataaaatgtatccatagaaactaaaatctcaaagatgctggctttaccttattttgactctgaaagttttaggttttaaaaatagaaaccagttgattgctatagtttctgtagtaagactgctgcaattataatagatttcctggctggttgtacaggccacagatgttttttggcacatgtgtgaaacctggtcagaccttactgatgacctggatgaatcttaagcgagatgtttttctctgtggagcaggatttggggacaattcggaagtaaaattatgccctgttgggaggaacactttctccttaaattgtcctggaaacgcttctttgttttacatttgttcttctgctttagaataattccataattgggcttattttgggtattgggagtacttcctagatagaaatatttaacatatattcaagagatagttcctgtttacacaaaaatattacctgcaggaactggaagtcttcctttgaatgtctgaagtccctccttacttattaatgccatgtaattctcacactgatatatggactgggaaaattgcaggaaaaaatcttccccgtgtccccttccctcaaaagggtacaatacaaaacttctataaccccatgccaaaaagattttatgcttaattgaaataggaagctatctgcaaaatagtagcttaccattgtattatttcatttagttaagtgactatagttggattaaaatgtgctgctatagtcaataattcacgaattagctaccaatgcattaaaaacctgaagttgcatcttcagaaattgtttattcgtgtcagcgtacaaatatgttttatatcacctacattaaaaagaaatagccctcccctaccccacactgttccctttctttgatctttctcctggaaaaggctgtctatacctgctttctcaccttcattaccctcaacacactccagtgggcctcttcttccctctgtgtaactgaaatggctgtattttgccaaagtctattgactatcctgatagcattccttgtcttagtgggatctgatgcagttgaccgtaaacctctacttggtggttctccgtgaccccacatatgcctggtttttgtcctttcttgtgggggctcctctgtggtatcctatgctgactccgccttttctccccttccctttccttggtaaagctccaggactcaggactccatcttctccttttctctctccattttcaatcctctggcgatctcattcagggtcctggcctgtttgacctctgcaactgggtttaatccatatctcacgcttcacatgttccaaatcagctcttgatccttcccctgccccaccccagtgctcctgcagtttgcttttctgctgtcttctctatctcgggcttatgcccatcctccccattgctcagcctaaaacctcgaatacactcttcacttccctttctctcacaccattcttgtggctttaccttcaaacatgcagcctttcctccctacctctactgctggtggccttgggtgagccactgccatctctcctgtaagtggtatctttgctgctaccgaccaacccacagttgtcctgagtggctgtacacttcactcatggtcaaagccaaatcctttcaatggccttcaggatctgtgccctccctgcctgcattgcttctccaatcctgcctcctctgcctccctctcactgccgctctggtcacactggttttcttgatgttccttggacacaccagccctgtggctttgcactggctcttccctctgccaaaaatgacccctttccagatgctcacatttctaactttccactccttccagactgcttaaatgccagcttcagtgaggcctgttctgatcatcctggttacaactggtaatccattcttcagttcacctctgaaacttccaaccctcttacactgctcaattttgttttcagttacatttaccatctatcaacattacataattgacttactgtattcactgttgacctctcctctcactggaatgtaacctcccggaagacaggaatctttgttttgtttactgatgttccccaacatgctgagggcagtacctgatgcacagagagccttccacaagtcattgttgggtaaatgaataaaggtggcagtaagtttgggcttgtggtatccatagtagatctaaagtcctgtccctcccaatacacctctctccacacccaccctgtgcatttctttattgcaaaattgcagtttttatttggggataagacctttttgttttgttaacctttccctgggagtttctccaaagcagattaatgagttcagtgtttcctttgatgttgctaaaaagataggcagattgatgtttacagggccatttgcacaatcaggatgttctcatgcatgaaccaccatcaattcaagaactgtaccttccatattttaggttcaagcttcagaaaggttttacaatttcacagcacggcatgctgtctgggagcacagttttgatcttcacagtgtaagcagcacatttccagtacctgtgactatggagttcctcctgctgcctcctcctctcttaggaatatcccaggatggaagacagcactgcattcctgaggaccttccttctcaagaaatgctcttagcagagagggcccctgtctgattcttaccctttggaacagttcaggcacccttagacacagtacacagcccttcctagcatgtgggcagggggacatcctgtattcacaggcatttgtattgagctacaagttggaaagccagtccccctagcaagagaatcttctgccagctctccccgattactccattaacacttcttgacatcaccctattcatttccttttcagcttgtaccaaaatttctaactatctggcttttacgtgcattatataattgtatatatacagtcatgcactgcataacatttcagtaaatgacagaccatatatatatggattatatatatatatatatatggatatatatacacatatatatacacacacatatatatatatatacatatatatgtatatatatataaaatggtggtcccataagattatactactacattttggctctactttttctatgttcagatagatgcacctattggtgtatttttcagaatacatccccgttgttaagcaatgcattactgtattatatatatggcacatgtattatatatatggcacattgccgttacttttaatggcaaaaaccgcaatcacttttgctccaacctagtaattgcaataaacaattgttgtatatagcaatactattgcattgtttttcctgtggtgcctactagaccatgaactccaggagggcaagggctatatctccagcattgagcacagagtctggaatagatgcctctcttcttgcttttgaatggatggataattgtttaaaatggaagaaggcaagagaagcataacttcctattatggtggatgctgttacctaataacattgatttcagtgcctttatttgtgccatgggtgccttgtgagacttttatcctgcaataacagtgggaaagaacttttgactgaaagttttttgcatctagccttaaactctatgtcccatataagcctccaagtcaccgtttctttattagtaaagtgatgggataggtcaggatttctctaaattcctttcttcaaaaaatgtttaggataaattttcactatccattattattaaatttgctcatcattttgttaataatttcctgcatttcaattcagttccatttagattcctactgtgcgctggcacatggctgaactacatagcttctgacatgtgaattgaagacatagtttaatacacacattatacatgcgtatgtatatatatcacatatatatataatctacacgtatacatataagagagtccctgttctaatggtgagcttatatttatgcaatgtcaattttgatatttattcttgaatttaatctagaaaagttagataatgaccagtcatagtcttaatttttattcttattcatgccaaaattattactctactagtagagtaaatagataggatcatggttatagttttcaagggagaagagaatatagacattatttaatccatgtccattgtgttacgaagaatgcaaaaaagacactgacttgcctaacatcatatcagttattgtgacagctgtaactaaaacatggtagctagtgactctgaatcctttgcttttgagactacaattcagtctaactctatgagactcttactgatggtacacagaacagacctatgtattagcctcatggggtttaccatctgccgggggagctagaaataagacaattatttatacctatataataggaaggttacagcgtgctggaacttagcagtagtcagggaaattgaacctagactatgagtcagggaaggcctcctgatgatttgatagaaattgaagttgcaaaggaaacagttcggcaatgaaggaagaagaacattctaggcccactgaacagcctgtagaaagggtcagagtgagtagatagtgtgtgagacgaactaagtgagggccagaaggagcttagggagcaaggggaagaaagcttgagatagagaagttaggtgaggatctgacttgggctgctttggagaccttggcgaggagtagatgcatttgaatggaaacccatggcaaggtttgaagcagtggagtggctaccttggaagtcatataaatcatgtgagtatcactttattctcaaaccataaaggcagagtgcacactagtcttcagtttgatcctgactcaatgattagcaggaggcatgggaaacagacttgtcctgccattctgggcctcagcctccacaccctactgtctgacctgtacacagactagtgctttggattagctttatgcttgtgtttgggacatcccatcttcctccacccttgacctggcagctaccctgttttgccattttgccttgtttggaagatctccattctctcctggtgtcctgggatgccacttgccctgtcaggctctttcagctgctgggagcctgtggatttgagtccaagtatattgtgctggccacagaaattcatttcttttaacacctaagttgcaatcacagtttcaaaggaatcatatgcatttgctgcttatattgcttggctttattaagaatgcttcagttttgaaatcacaatggaatttctaacaacatataatttatttcactcattccagttgacatgatttaatttgcctttgattttgttgtgaatgatgcaagtgaaaaaaagatgcctgagttattactgtgtgttcaactgctttttatgttttctttaatttgcagttttctcgtgtactgaagaatagggcctggcctacttttaaacaggccaaatctaaaatctccccactgcacagcagtgatttctgccctaccaactgccatgtcaatgtgatggaagtttcttatcccaaaacatcaacctctttgggaagtgcatttggtgttcagttggatagcaggaagcataattctcatgataaagaaaacaaatcttcggagcaaggtatgctagcttttgcaactttatcaaatcatgaaaagcaatttaggcaatcatagctattcatgcatccctgaaattcaatagatacctggtgtgcaaacaagcatgaacttttataaaggaaggatttcttgctgtaatcatttggataattatgcctaagtgtatactgaatgaaaatggagttaggcatttttgttatttttcttctaagaaggatgattgctcttgctttccttatgcatatgataggcacagctcatcttgctgtgaaagctgctgatctgaaatggccctgaaactttgaggacaatacctgaggaacagctgcagcaatgcacagtgccattctgtaccatcacagtaggacaagggatcaaactatcctctagtttttaattacagctccccacgggctctaatttctgaactaaattcagcattggatcagcattttattttatggaatctcaaatttaatttaactggataaagaaaatggggtatataaacacaatggaataccactcagccataaaaagaagtaaatcctgtcatttgtgacaacataaatgaacctggaagatattatgttaaagtgaaataagccagacacagaaagacaaatactatatgatctcatttatatgagaaatataaaaaagttgatctcatagaagtagagagtagaatggtgagtagcagggcctggggtggttgggtgggggattgagaagatgaaggtcaaaggatataaaatttcaatcagataagaggaataagttcaagagatatattgtataacatgctgagtatagttaacatattgtattcttgataaatcctaagagagtgggtataaagtgctctcagcaccaacatgataactatgtgaagtaatacatatgttaattacctagatttagtcattgcataatgcatatatacgtcaaaacatcaggttgtacatgtaatacatataattttatctgtcaattaaaaaataaatgaaattaattggactgggttgttatcgcaagttaaaggtgatacatattactaaggaatgactttaattcctcagggaaactgagccctatggtgtacattcagcacaccatcacaaccatggcggccccttcaggtctgtctctgggacagcaggatggccatggtctcaggtatctgctaaaagaagaagacttagaaacccaagacatctatcagaaactgctgggcaaacttcagactgcactgaaagaggtggagatgtgtgtttgtcaaatagatgagtaagtgttttgtactacacttttaagtttgccaaataaaatagccataaaaatgtggcttagaaaaattcaatcattctctctttagttattggaaatttaggagatttgtgctgattatatagatcctcttcattccaaaaccttaaaaatgttttttaatggaaaaaatacatataggtatatattttaagtaagcatttaatactagggaagagaatgacaaaattctgtcttatacaaggatactaaatgagtctcagtgtaaaaatattaccttgaaagatatatttgaaagaataccagttttgtatgaggatttccttgtattctttgtagttcagggtgcaaattttggtgaatatgcactcaatagatatttataaaagacataaagtataataaatgcttaacatacttgttaaatgagtaaatgcaaaattatgtatctctaggacgaatttttttttctgttctgattgaattgacagcctgctaagcaataacccaaatacctgtgccagtatttttaaccaagagaaggtggaggcagttaatgagcacttttccaaataaatcagttctaattcttatgtaagaatatgatggttggagaattgcctgagcttggtaggatggtttgtcatatcatggtataggacgatgcttcacatctagtccagacagctctgtatagtcagaacagcatggggtttgaggtagctcagtttctcatctacagaagtgactgagtaataataatgaattacaagtttgtggtcatgaaaattatacaaattaatatagaaagtaatcagcacactgcctaatatagtgcttgctcatccattcaagggatggaaattattgaatatatcccaaattggcagattctgaaaataatagaaatactgttatcttctttttattgtactctcttcgtgaagttgttgatttaaatggctacggatgtgaactcttttaacttcacacacacacctatcatcctgtacttcatggataattcagttttgacacaaaatatttagaacttattccaagcctttgttgtaattagacattatacggatgaagtcataattgacacgatctctcattttacacatttttgaaatttagagatcaaaatcacattagatgcatcatattttaaaaattatcctcaatatttagtatttactttgctaattttctgaagttttatgagtgatttagaattaagattttgtttttctatttctaatatgctagttttgtatattatactgtgacttttcccatttacagatgaattaaaccgttcaagattgctaagagaaaaggagagtatttttttaagcaattgcatgacatctttgaattgtgttggaatccttgtgacagcagtttatattttaggatatgatattttacttcatttaataaatgagattttattgcatgatatattttagaaaccagttattgtctcccagaggtatttatgaagtatatagaatttttattaataaggcaataaatagtggcattgaatcatagctcctaggcttcaaagggattttaagaaaacatctggaccaatcccatacttccagttccaagagttgttattgctattttgggatgagtcttgtcagtggcaagattattgatgtgcctcagctgacactgctcagggactaatctttactgcattgacgatctagcactgcctcacaatcctccagactttgtctttgttaatttattgacttttttttttaaggttcaggtaatgtagaaatatttttaggaaaaatatgtaaacccaaggagctccttttacttggtttaaaatatttatatttaagacactagtttcagactagttattttttattaactaaaactacttaaaatgattgtttttcactgaagatttccccaccctgttattctaaaaacctctacccaactgagtgtgtttattagaaaagtggcacacagtgagcaaatgattggagaaaccaccttggtcttctttgggtacaccacatcatgtaattctaagtataaataggttccacacaacttaatgtgcaatataatccctgtaaaacagtacaattattttaagtgcctggattcccccttgcctttaatattcagagatgctgccatgcagtcgaggttaacacattatttaaaaatctagtatctgaacaattttctcaccctatagtaattgttcctggtcagagatgtctttgcaagtgaagaagcagatctcggatgtgttgccaacttttaagagaaactgcctgataacacctcagtgttagcttttctagaagttattcatccctgtgaaaggtcctggggttgagcttgtcttatcataaagtctggttgttctgtcacctgggaatacctctcttagaaatgaaatttcctttaacacataggcatttcactaaatcctcctacttagcttcagagccaaagatggaattagagatgaatttgctgtctagtaacatgaggtaagatgatgtaatgaatttaatcaaggatttgtgattccaatttcactttggaaagttaaagacttttgccctgcaatgggctatttactgggtaatgacacttaggtacaagaaataagtaaaaggaaaaataattaatgtccaaaacctttgacagggtttttaaatagaggatttgtgtattctcacagggaggaaaggggactggtagacataggggatttgagaacagacttcattttgcaggtgccagacgcctcaggattgagtggtgaacaaaccacactttaagacagttagtaacccgggggttacagataggaatggtggtggtggtggtggtggtggtagagtaatcactgaggtttccccaagcattttgctactaaatgtacaacaagaggggcacaccataaaagccaacttttttatagatgcttatttgccaggcaagattacattattatatacattggcttatttcattctcacagtacttctgtgagttgagttatgaaacaccgggatgcaccctactccctccagagtgtgcaagttagatgcagggagaggaaatcctgcttaatttgcacaggatatgtatgtaacaatatgtcttgcctaccccacctctaaaatggggaaaataataatacctacttcataaggtggttttagagattaaatgaattcatacctgttaaggacttagcatcgtgactgtcatgaaagctgaacagatgttagcaatgatgatgatgtttaaaaacacacaggtcattgcaacatactgtatacaaaaatctttcttatcaatggcacaaggagaagattcacctggaattgcatgctgctgaaggttttaattaatggacctctagtaggtaacttaatattttataccaatgaaagtgaagggatttctgcaagcacaaatattatggtttgatgccttgttgcttttcagaatctagaaattttatgcaaaacatggtgttaaaaatacagtttttctatcatttgtttgtaagcctaacacttgtcccacggggtcaatgcaagcatgattgcagctgttttaaataaaaagcaaatatctgaatacaattcatataactttgaaaaaaattatgcttagccccatgacttctaatttaaaagatacataagcacagaggcctcttggcaaaatgagaactcctgcattttttccctctttggtattcagagttcatctgtctgtgcagcatggagcttggataattggagttagtctaatgttttaatcttttgggtggagagggtgaataaatagacaatctgtcttacaggacataaaagtatctttccaaagagaaaaactgtgtttaaaatcatccatagcttttggttattaaaaatgttcacagggcctcaagcaatgtaacatagctccttcaatttttcactggtgtctaaaacctataaaatgtcaaaatattgttcaaatctcacctcgctgtgagacctgacctgactgcaccactgtttgaagtggcagcttcacgcttccttccctgctttctttttctccttaccatttgctgccatttgatatactagaaagtttacttgtttttacttgtcttctctcagtagaatttgagctccaagaaggcaggggcttttgtattttgtttttgttgttgttgttgcttgtttggttggtttttgagacagtctcactctgttgcccaggctggagtgcagtggtgtgatctatgctcactgcaacctccatctccagggttcaagcgattctcctgcctcagtctcctgagtagctgggactacaggcttccaccaccacacccagctaatttttgtatttttagtagagacgggatttcaccatgttggccaggctggtctcgaactcctgacctcacgtgatccacccacctcggcctcccaaagcgctgggattacaggcatgagccaccgcgcccggcccttttgtgtgttttataaactactatatccacagcaccaagaacagtgcatggtataataaatgtgtttagtgaaatcacttaataggtatttattcaatgcacagcattgggtttgacattatggaatatgcgtaagaggtataagatatcgtcaccgtcttgaaataacaaacattacattgctatggaaaaatgagaatcaagatatgtgctactgattggaactgaaaatcacaggtatttagtaaatgcgaagatggctgctggcttgagaagccctatatgggtcctttgtttatacatggtttaattcattttgcaagttttttagcatgctctttgataataaagttcaattccatttttgcctaattttgagttagttgtaacagaaacatgccacagccaaatgttcaagcctagagtgttggagtcagcagctactcaaagtcctcagcaggtttcacacagttcagtctgactgcggcattttagcctggagccaggtggaggagctctttctgtgggcagatttgggacatttcatcctggctggcttgcagatttaccccatcctcacattgtatgtaactctgtttagtgtcttgtaatccccaaggccaagactcctgtttgtacgagcagggagacggatagacaatccaatattctccctgccaaatagggaatgcctgggcaaaacccaaggcatagatagagggtaggtgggtttttaatactgaatatgagccagtgaggacatgaaggcccatttatagtttgggtttaatatttggctagcactttaaagattactgagtattttctgtttttcttttctttcttttcttttctttctttttttttttttttttttttttgagttggagtttcgctcttgttgcccaggctggagtacaatggcacgatctcggctcaccacaacctccgcctcctgggttcaagcaattctcctgccttagcctcctgagtagttgggattacaagcatgcgccaccacgcccggctaattttgtattttttagtagagacggggtttctccaagttggtcaggctggtctcgaacttccgaccttaggtgatccgcctgccttggcctcccaaagtgctgggattacaggcatgagccacaacacccggctgattaccgagtattttcataaggtggtgatggttgtgctaatggccaggggctagaggtgacacccattatgcaccaggaatattcattctcacagcagtcatggaagccagttattctcattatctccatttcacaagtgtcaaacaaaactatcacagctttgttttagctatagtcatacatggcacaacctgtatttcaatgaagctttctggtctaaattgtatgttgtttgtatgacttattttgtgaagaacaagagaactttgaatgacatgctttttatgcaatagaatatttacttgatatgaaacattgaagcactctagaaatatttgtaagcctattgtattcagcaaataaggtagttatatatagcagacagtttgaaaatgtgcatttttttttaatattacattgcagccttctgtcttcaataacatattctcctaaattagaacgtaagacatcagagggcataataccaacagacagtgacaatgagaagggagaaagaaacagcaaacgggtatgttttaatgtagcaggagatgaacaggaagattctggtcatgacaccatcagcaacagagactcttacaggtaattcactaattcctcaaactcattttcttaacaaagtcaaatatgatgctggattttccacaaagatcttgaaaaaagtttttgattgtttttcttttaattggtctttcacaataatcacctgttactaccgacctcagacaaagcatagtgggacaatgtaatatttcaaatccttcccttttctaatatgagtagcttggaaaaagcattttttcgctggttgtgtgagcctaatttgggagagttgttctgtatatggattgtgtaacacagatagctggtaagaaaattgtgcctatacaggattaacattgttttcaactatctgtttctgtcactggtttattgggtagtgaataggagttcttttggctcttttttctctcttttcttttcattgtctatgttccaacctaattggactaatgttccttgcctacctcggagacaaggcttgtggtttttactctcttggataccttctgtattagttttgtagggctgctgtaacagattactacaaactgggcagcttaaaaaacagaaatttattatcacacagttctggaggccaaaaatctgaaaccaaggtgtgggctgtgttggctccttctgaaggctgtggaggaaggatccgtgccaggcgtggctccttgacgcgtaaacggccatcttctccctgtgtctctgcacgtcatcttccctccatatgtgtcttgtgtccaaagtttcccttcttatgaggacattttatagggtggcttaaggtcacgccctaatgacctcattttaacttgattacctttgtaaagaccctatctccaaataaagtcacattctgaggtactagggatcacttttcaaacatatgaattttattgggagacctagttcaacccaaaatactttccctccaaactctgttattgtcttcctactagacttccaaatgatgtctcctcacgtgaacttccttaattcaaactactttttgttccttgaatccatgtatcacctgatacctcactgaggactgtcataaccagccacataagctgtgtatacaacttcaacatatttttcatttatattaattgaaagcagaaactatcttcttcactcatttattatgcagtgttttgcaatgaaaagatattgattaaatagtaatcaaagttctcatcagagtataactagctacaaactaggagctaacattaaaaatatacttttatcaaacacatgggggaaagaggaagattttacataatcctcaccgcaagcctcggtggcaggttctgataggaattccattttacatatcaggaagctgaagcccagagggctgatatcatttacctatgagaccaggtggtctggcttcccagtttgtctcttaaccactctactattccaatttctatgtttccaaaaaggatcttaatgaacacataagaattattctagagatttaataatagatcagacagattaaccacttttatcacttcatttacttttcacttctaagcatcccaacctgctctttaataggaatctgtagagtttttcccagtgggaaattacatgtctaaaaatactttcttacctcaagttctagttatcagataattaaaattaccctgacagtttccctctaggctaagatagaaattttaaatgtaaatttaaagttaaggacgtcctttttacttattctgagaggaaagaaaatactttatctcctgcactatttctgaaatttgtccctgttcacacacaaacatattgaacctttctttccttgggcattcatcattatcaagcacttccctgtgctaaaagctctgaggtggagacagacaagtttcctgtcctccctaaggaagctcagagtgtaaccaataagttatgatttaagccaccagaaagggacagaaatcacagaacagtattttgtctctattagcaagttatctaattactggcatgtatcatgaaacagattataaagaattttaagtttggcatgactcctagttttatagctaactgcccttaaaaaatcatttaggattagattatatgatacaagaaaatttgaaatataattgaatcaaaaatatgccatctttggccgggcgtggtggctcacgcctgtaatcccagcactttgggaggccgaggtgggcggatcacaaggtcaggagatggagaccatcctggctaacatggtgaaaccctgtctctactaaaaatacaaaaaattagcctggtgtggtggcgggcgcctgtagtcccagctactcgggaggctgaggcaggagaatggcgtgaaccctgcaggcggagcttgcagtgagccgagatcgcaccactgcactccagtctgggagacagagcgaaactccgtctccaaaaaaaaaaaaaaagacagaaaatatgccgtctttaaaaagtaagatagactgagttgaacaagtttatctttctcatagttttctctaaaacatactaataaatataactgatataagtttatgtcataagaaattgcagggacacctgggcagatgcactcttacagtaacatgaaacctgttgttttcagtgactgcaacagcaataggaattccatcgcctccttcaccagcatctgcagcagccagtgcagctcgtatttccacagtgatgaaatggactcaggtgtgttcgttggtgaaggcctgtgggctttttgatattagtcctataggagataactttaaggttttaatttcgtagatccatatgaatttttatttttaccttttatcagtcactctctgtggccacacatactgcatttcagttttatgagcttgatagtctatttcccaatcccattttaaagggagtgagttaaaaagcaatgaaataagcaaactaattgtaatcttcagatatgctccattatctctgtatggttctatgaaggattataggtacacaggaaatatactggaagtattaggcatggttgatggggatgggcaggtgtggagtttgaagcatttaagacagcaggttcacctgagctagctttatatgtggatgttgaggattgagggtaatgagagaaacagattgatataattatcttataaatatattatcttgtgtgtgttaagaacaaagacctttcattccaattagggaaaatcttaatttttgattatgcagtttatgtcaactcttcaaccactggaggaacatcatttgaaagagtctttaacaagtatcttttctgggaggcactccgccttcagaagaaatagtaacatcccaggagctatgaaatcttttggaactctttctctgaacatgagaactgtgcatggaataacagcttgtgtttaatttttacattttattgaatattacaatattttggtacgttaaacataagatgagtgattttggtttttggttttgtttttgttttgacatctaggtgatgaacttcccttaagtgttcgcatatctcatgataaacaggacaagatacatagttgccttgagcatcttttcagccaggtacaatcgggcatttgtggggcttttgttccacaactaaactgtgaggagtccctttttcatatgcagtaaaaggagctcagagttagaacagaaaggcctcaatctgtccttccacctcctttacctcaatttctgacatttaaactaggaatcaacattccttcagccaactaacctaaaggatttttgtgaatatcaagtattttacagtcctgggaaaattactgttatacacataggagttaccctttttagtatttttttttgtttttttgttttcagaaagctttatgatatttcttgctcaatattatttctaattttaaaaagcacaagatattgccttggatgctaattttgttgttttacattaacaaaatttatagccataaccaaaatttgtagtagaagattttaccactttgaaaaggattccaataacttatacagtaaatttatattttgtgttaatatatggtaagatagtaagaaatgtcattgcaaatgttatcaggtgcatttatttcaatattttatgtaaagatcgatttcacaagcatcatggggtttcaaaaataattgttgctttaagaaaaaatattttataagatttacttttagaaatacgtatcatgtactatgagaaatatgtaactcggaaatctctactattgataggtggattcaattaccaatctcctaaaagggcaggctgttgtgagggcctttgaccaaaccaagtatctcactccaggccgaggattgcaaggtaagccttgaaaaattaactagtagaagcaattgagaaaaacaaggtggtagacaatatagtcatgaagcaagcagattgcataagaggaacaattccagttactcatgtttcataattcaccaaacactacttctattctgataatttttgaatatgtaaaaaagtcagcataggaatttttacatgtctgtttctttgaaagtttaaaatgtttaatgcctataggggccaggctgagcaaaaataagttaattgaagatacataaacatgaagttgattttgctgaaaccaagcgtaatgtcttcttggctgtttcccagtggtgaggaaagggtgtggggaatgagagttgatgaatggaaaggatggagctgccactctgacaagtaactagtttgggataatgaaagtagagttttcatctaaatttgcttcgtttaaacatttataagaaaataacatgaaggtttgactttttacatttaacggattttctgatggtgtggcaaaagaaatgaccaccatgtcaatagtctaggacaacagtgacagaagaaggttttgaagcctaaaggcttcattgtttaggaattatttgagacttaagagagattttctgtgtttatttagaatttcaacaggaaatggaaccaaagctgagttgtccaaaaaggctacggcttcatatcaagcaagatccttggaatcttcccagcagcgtccggactcttgctcagaacatcaggaaatttgttgaaggtcagcatgaaaagcaaagaacattgcatgatattagcaataatcaatttaaaacccactaaaaccaaaagtttggtaatgcctgattattttttgcagtgtttccttttgtgaagaaaataaaagtactgaaaatggtgagggaggaagagggcaagaaggaataagattatagggtcttgtgtcccaaggttataaacatcatggagaatattctcagtgaataaaataagggctcaaagttatgaagacaaatagaacgtgaagagaatttaagactctataaaaggagacaatatcataaacaaattctgaaggatttacaaaaaggcatgaagcaaaagtttatccacaaaaaaaaaatcagttgtgaagctgtatgagaatcaaaataatcacataggttgcatcattgatttttaagtttttatatcagaagtgtttgtctaagttgattgctattaactaccataacattatttacatgacattctataattggtaaaacctgtaaattggagatatatgactttgagacgacttcattgagatagattgcatgttgtttagaatcagtgtgattaagtgaaaattctgagagtttcaaaaatgattatggtggaaaagataaaatgagaattcacctagttgtaaaaatatataactcagaaattttcttcccagtgattagaacagtggttggcaacatctggaggtattggttgtcacaattgtgggtgtgctactgacctctagatagaggccagggatgctgctgaacattctagaatgtatgagacagttccttacaaggaagaattctctgatacaaaatgttaatagtgctaaggtcgagaaacccacacaataaatactattcctgtgtttacatttttattcttcagtcatttgtttgtcagttggttgtggtcaatagtattcaatggtatttaacagaattacagtgtaactgcagttattaccttaagtggggtaaattaaagagagctgtaaattttgctgacaaaagacaaccccttatatcaaatggagtctgagttagtcacttaactcttttaggcttcaacttccacatttattaaatgacagagatggcttaaatgttctgtgattactaagtcttctgcatagacttatactcaaatataacaacttatgtttagagattagtagaaaggaaatttgtacagatatggaatacttccccaaataaatcatagtgattctatcttgcgataaaatcgtgtatttgacagctggttttcttggctatatttttattttatggcctaatgtaatgtttatctgtgatactttataacataatatttaatgaaaagtctgaatttaaaattccccaaaatatgaatagcataaatgttaaatgcatttttgaaaaactattatgttttagaaatttaagtatatactttactgagtaaaagacatttctgctgaatagatgccagtattatgacactactagaaactataattctgcagttgttctcatcatctttgagattttatatttgttttaaggtaaatgcatattggctaggaatggagaaatccttaaatcatctcccccacttttttgttttgaacaattttatctttcagatagaaatttctaatgctctcattagattcaaagaacttcttggcttctgcgttaattttactatttatccaaaagttattcaagggcaggttattcaatatccatgtaattgtatggttttgagtgaatttcttaatctttatttctaatttgattgtgttattgtctgagagagtggttgttatgatttcacttcttttgcatttgctgagaagcgttttatgtctgattatgtcatcgattttagtgtatgtgccatgtggtgatgggaagaatgtatattctgttgtattgggatagagatatctgtagatgtctatcaggaccatttgatcagtgctgagttcaggtcctgaatatctttgttaatcttctgccttgatgatctgtctaatactgacagtgggttgttgaagtctccaactattattgtgtgggagtctaagactctctgaagttctctaagaacttgctttataaatctgggaaattaatgagagcaaaggtaaagcataccagactctctgggacacagctaaggcaatgttaagaggaaaatttgtcgcactaaatgcccatatccaaaagttagaaagatcttgatttataaacctaacaccacaaccaaaagaactatagaagcaagagaaaatcaaacccaaaggtgccagaggacaagaaataactaaaatcagagctgaactgaaagagattgagatacaaaaaaccatttaaaagatcaatgaatctaggagttgatttttgaaaaaattaataaagtagatagaccactagctgggctagtaaagaagaaaagagagaagatccaaacaaacacattagaaatgacaaaggagatattaccactgaccccacagaaatacacataactatcatgttatgtgcatttacacataaataccataactatggacacctctatgcacataaactacaaaatctaaaagaaatggataaattcctggacacatgcaccctcccaagactgaaccagaaagaaattgaatccctgaacagacctccataacaagctccaaagttgaatcagtaataaatagcctgccaaccaaaaaaaaaagcctggaaccagagagattaacagccaaattctaccagatgtacacagaagagctggtacagtttctatcaaaactatttcaaagaattgaggaggagggactcctccctaacttattctaggaggccagcactatcctgataccaaaaactggcagagatacaacaacaaaaagaaaacttcaggccagtattctttatgaacatcaatgcagaaatcttcaacaaaatcctggcaaaccaaatccagcatcacatcaaaaagcttacctaccatgatcaagtagtctttatccttgggacgcaaagttggttcaacatactcaaatcaacaaatgtggttcatcatataaacaaaggaatataaatcattctatcataaagatatgtgcacacgtatgttcgttgcaggactgctcacaataccaaagacatgaaatcagctcaaatgcccatcagtggtagactggataaagaaaatgtggtacctgtattccagggaatactatgcagccataaaaaagaatgagctaatgtcctttgcagaaacatgaatggagctggagtccattatccttagcaaactaacacaggaagagaaaaccaaataccacatgttctcatttataagtggaagctaaatgatgagaacacatggacacaaagagaggaacaacacatactggggcctatcagaaggtgaagggtaggaggagggagaggatcagaaaaaatacttaatgggtattaggcttaatacctgggtgatgaaataatctgtacaacaaacccccatgacacaagtgtacctatataagaaacctgcacatgtacccttgaacttaaaagtttaaaaaaatatatttctaatgcaggataaaaagtttaatttttcaaaaagtactcattttaagaatcttacattttcatttcaagacctgcttttgtattctctgtaactcatgtcacaataagcagctataggaatgataactatatacatactgattgatggattgattgatatgtgttgtgaatttgggtgccactcttggtcatgggagtccaatggctagttgagtgtggttggatgggctgagagtcttatagtcaatggtgtggggagggctctctgtagaatttagttgcatctctggttgcaaatttcagtgagtttcagtgagctgtttgggtaacagctgtgattgtcaccatgttaagtgagtctttaaaaatgattacattttgcttcatctcaatggactatgcaagcgagagggcatgctaccatttcctggtaatctgtgtgatgtccggtcctgttaggagttaaaaaagcattaggatcattgaaatcagttgcttgttatgtggaggcactctgagtaactttgctagttcagcatgaatttatcctattcaacttaagtcattttcattctacttcctagactgtctttatatagtgaatgacttaatgaaaggttagatcttggaacttaatttaacctggtgctaccaggccagaatttcttggtttatataagcagctgggatgaccccatttctaatgtttttcctagtctccgatttcaaaataccccatctgttatataccacaggtgatatcaaggtgtcataattctatgatctttatttagaactgttgcagtgctattcaaaaattgtatcattatatactcacccattcatagcagattaaactgtgtgtagaagagagcagagaagatagtgaattaagtgaggacagattaattatatggtgtccctaagacgatgcctatctcctgcttaacctctgaggtaagttatggcctcatattatcagctgaatgaagagatttttatttgagtagcccaccatcaccttaaattcagatgtttattagtgaacacattttcaactttctgttccctataaagaaaaatgctttattctaaatctcatattttttttagcaacctatattttagaaagttttttccctgcatttgtcccaatattctaaagagtgtctcttcagtttttccctttttcatcacacttcagtaattacaatgatttttctggctaacgtccctctccctatctttttatgattcttaaaaccattacgagattcatcttcctaaaatatcgtgttcataatttcactactgtgtcacagagtcttgcggtgattttctattaggtaagactaaacacattgatctgttagaagccatttttcagtggggctttctaatcatatcccaatctcagtctcttaatgatataataaaatctctgtaggctagattgttagataattgttattcattctctactatattcctttgctagcaaaatctctcaatcttagaatatgtatctcttttcttgacagtgataatttaaagttttatgaaatattttctagtctcatttagaattctataccaactttggccagattcagattacaataataacagtcttcacctgtatataggattttaaacaaatttcaaagacttttcacacatattctcacttatattttacacagacctgtaaaatagataaggcagatgttatcttcatttatgaatgagttaattgagactcaaagtaatttagcaattttctaaagccacaaagctagtatataacagcagtcttctgactcttagcttattgtactttgctgcctctgaaatccttttctgctttcagtctcacttgtaaaatgtggtgccctttgaagtatttcctgcatttcttttcagtcaattctgtctttctgactctactgtgaatctgtctagaataggatataacttaatcttttttttcccataatcttgggcatttagcaggaactttgtaagtttttaaatttacctattgcacataaactataattatttgttagttggaaacaaatggacatgtaactcattggacacctagcaattcgtatgaaattcatacacatttatcaaacatctgtgattgaccctgtgtgggttggagttggaagaagagtatatatatacacacacgtatgtgtgtgtgtatatatatgtgtgtaatatatatatacatatatacgtgtgtgtgtatatatatatacacacatgtgtaaactttgaaagtttaaataacagtagcaaaaatattgtttgacagtttattgctagtgattacaacagttgtagcttgcagaacaccaagcaggctaaaacagctaaggatgacctaaaaagctgccaggatttgaatggtgtttcattgactgatataattcagagacatttcaggatcttgaacaatggatagaatttgtgtaggcaaacaggcaagcgtagcatgtcaagtgaattgatttttattatctaatattaggttggtgcaaaagtaattgctgtttttgccatccattgaaagtaatggcaaaaccacaactacttttgcatgaacctagtccaataagatcatactaattttgactacacagttaaaatatggtatttgtgacagaaagagtgaacaattaactgatgtgttttctttctctgcagaggtaaagtgtaggctactcctggctcttcttgaatattcaggtaacattttgcattttatttttttttactatttaagtgattgtggcccaggatcataaaggccttgaaatttgaggacaacgccttcaaccatttacaaaatagaaatatgatccctaccttctccaaagctttggaaagattagaaaaagtactcacatcattttaataaaaatgcaaatatatatatatacatatatatatatacacacatacttgtattctcttataagcaaaaccatgaaaacatatatgaatatagatttaaacaaaatattattaaacaggagaaagggagtaggttttgctaaagttaattgtgtctaaattttgtttatgagcaagaatgaatgtatgtaaacattttacaaatcttacattaaaacacttttaccatgcacttagtgggagctcaaaaaatattggtaatggaaaatgacactaaaggacaattaaatatttgatgagattctaaattgcagatgatagactgtaaatgtcataaaggcaggcactatgtctattttgttcatcagcaaatgcctaaaatagtggctggcatatagatattcaacaaatatttatggcaaaaatgaatgaaaaattatatgaatgaaaaaaggaaaaatgtatgaaatatttcaacaaggttttcattcaaaatgcatttacaaggaaatagatcgtaaagattgggaaaactcaactacatgccatttaattacagtgaacagtttgatgttagcctgctgtgcgtctctttctgtagattcagagatatatagatatgttgaatgagttacttttattattttaatctatttcccataaagtatgtgtatgtgtatacacagaaattctattacattaactagatgttgatagggatttgttcctaaaaaatataacctgaatgattacttgtgatagcagttagcttttgttgtgtttaaaaaaattccccaaactttctggcataaaacaatgaccatttcttattgcttacctgtctagtgtcatctggccatttctggtggtctgtgccaggttttattgatcttggctgggtttgcacatgtgtctgcagtcaggtggtgggttggctaggcacaggtgatctagggtggccttgctcacatatgtaacatttggctggctgttagctgaggtaacagggctgcctggactgcctgtctctcatcatctatgaggctagcctgttcttgttcacatggtggatttgcagggttccaggaagacagtgggaatgtcgaagagcttgccaccttctactggccccagcatgtaactggcaaggccagacttaaggggtagagacatagattctacttttgaaggaaagggtctcaaaattacattacaagagtgtggatacaggcagggaaagaatttgtgaccacttttccaaagtaccacacttggaaattttaaattatgaatggaccgagttaaccaacaaggcttttttatttgtccattggttttttgccctaatgaaaaaaggttttgccttgctttttgactgagtaataataatgacaaaaatatctgagagtttctgagctttactaataacacaattcatggacctcattttcttggatttcactggatccccacaacccactatgtgagaatattattgtttccacagttttaaaggtgaagaaattgacattctggcagattaataataaccagcacatagtatacagtcaacactgacgttggagcatctagaaggttaggcatattgatgccttattgaataaatgaataaatagtaggtggcagttttgaaattcaaacttaggtttgccaattccggaggtgattctgggatgcttcgttttattcattaatgctcaaatggatgctctggtagatttttattttgatttcaattttatttttctattcctcttatgattctttattttcctgctttatttgctcaccctctctcccctcttctatataggatgacaccaaaacaggagccaaaatactttatatttttggctgcgaaattatgtttatttgaaaagggtagactgtgtcttttaaatattcttaacctccttttatttctacctttctctttttctaatctccttttttgttgtttattttgctttattttccctgcaatctctgtgctagaataatttctgagattgcttttgtgtgtctagaaaccatctcattctttttagaccagctagctaattacagatcttaaacttctaaacagccaagattaaatagtgtaatttaaaacatgtgttagaagatgagtttaaataggtgcttaaatactgaaatgaaaccgaagcctttgataggtacaaaaaccatttcttttgagtgtactctcactataagaaaaatattactctatgataaaaataaataataaaaatcttactctacaacaatagtaagcaaaaaaaaaaaaaccctcaaattctctgtttttcttcttttgctagtagctgaaagcagatgtgatttttgctgtagttctttcttattcataaaaaataggaaatgaaggcagtgtgccctggtccatgtttataatcccagcactttgggaggctgaggatggaggattatttgaggttaggagtttgagaccagcctggatatcatagtgaggccctgcctctgcaaaaaaaaaaaaaaaaaaattaaaaattagccagatgtgatggcacacacctgcagtcccagctatttgggaggccaaggtaggaggatctcttgagcctgggagtttgaacctgcagtgagctatgatcgtgccactgtactccagcctgggtgacagagcaagatgctctcttaaaaaacaaaacaaaacgaaacaaaaacacacaaaaaacccaaaaatacctttggtaaaggatggatacttgtaaattttgaatggttgcataattgaaagaaaaagacctggatatttttgctgagagttagtagattttgttgtatttatgtaacaaattgctccttctccatgagcaataaagctgagcacctactcagtgccagacacggtgctagatgctaaacaaaaagaagaatcacaatgataatgctacaccactttactttgttaagtgctttaaatactttttatctcatttatctttactgtagacctaagaaggaggaatgaaccccattttccacatggggatctgatacctaaaatatggcttagaatggttggttgaaagcagatgatatgcctctaaggagggtgagcctaagtcacaggccacttctctggccttgatgtactgatggttgcttgggaacacagactcataaatgaatgcccactgtgctatataattagccctccagtagaaggatacccccaggtctatggtgaccagaagaagagccccagtacttccttggggagaggggaatttcacaaggggagataatctttcaatgagtttcaatagtttgatgagttgactcttaaagattgatttaaagtttgctaggcagaggagaaggacaagggcatgcaatgcatctggaagaaaggtataggcccagtgtacgccatagggcatttggtatttgaggactgctgagcggtactgtgtggctcaaagaggacaggtctgtgtaccccacacttctgactgctttcagatatgttgttttaatgtgctctcccaataattctgtgaacattgttgctgtagtgtggaatgaatggagggaagtggtatgttgagaagctgtaaatataaataggggataggtcacaaggtcccgcttatgccactgttttctaactttgttattcactgtgagatgatggtttgtaaaaagttaacttgaaaagctcttgatataatatgtacattttgaatctgtgcgaacaattcatctactctctgctgaaaataggaaggtggcaacagaaaattatctcatgaaatatgaatttttattcaatatggctgaggccccagcttggatcctctagtagttgttgcagctgtcaattcctgtagggtattcattcagtgttaaacatttgaattggtgaatgaaaataactacaagttatcaacaggctttggttttgcacactgaaaagttgacgttaattgcaatcagttctgatttgataaagaggttgaaggaaaaatcttattttggcttaaatgcaaatagtatatagtagcagagtatagaataaacatctaagtattctttttaggataaaaatagaaaagaactttttcacaaaagaaacttggaaataattatttctaccagtggcaaagttattaatcctggtgatttttgtgaaagcttacagtgcagatccttctttctctagatttttgcttttcactcaatatccataatttctttccaattagatttttattaaatcattttcaaggtatgggtgggagtatttctgccaaatctactgaagtatttttaaaaattcacccagattttctgcttcactatagaacatatgaaaaacaaagtaataattttcaacaaagatgtagggcaatttattttacttatttagtttctgtcgtgaacctcagtaccctaggtgcacactggagctaacaagctatctgaattagcctcttaagtaaaattaaatccagatatttcaagaaaccagagaatgaactcatgtcttttcatttcacttttttatttatttttagtgccactgactctttattttaatcattgacttagtaaacatttgttcaatgtctactgtgatagaaattgtgcacaacaaaataggtgtttagttttttttcttgaggattggacaaagttcatttccagtttagagattttatagttatctataaatgccctttcctatctagtagcatggggcacatgttgttggttaagttagtaaattgagccatatggaatggccacattccaccattttaacctaccaaagcagcatgtttctatagttgagcctaataagtgaatattcagatacaaaattaaatattttaaatatttaattatctttctaataaaattttcaagagtaggttttacagtgaaaccgagtatcatagaaactaaattatgtgtacacatgcctttgatggttattaaaaacagatgttcagtgttcttggtaaatcttaaaaattatgccttttttgtcaataaaacaggagctcaccactgaataaagtcagcatttatctctgtgttatacactaatattgacaatgcttctttattccagaagagtctatgaacccttgaaatatttagtacacatttgagaggaggttatttttccctttgttgtttccacttctagtggaagtgattttccaatatgtggaatgtgtggctttgtgtttctgtagtttgtatatatccaaccacctgaagaaatgaactctgtgtgttagcttctttccgtttgaaaaatctctagttcagaatttcacagtgtaaacatatcttcaaagtgggtgtattggtctattttctcactgctataaagaaatacctgagattgtgggtactttataaaggaaagaagtttaattgactcacagttatgcatagccaggaaggcctcagaaaacttagaatgggagaaggcagaaggggaaggaggcatgtcttacatggcggcaggcaagagagagcagagagcgtgtgtgtcagaacacaggaaaaactgccatgtataaaaccatcagatctcgtgaaaattcactcactatcacaagaacagcatgggagaaaccacccccataatccaatcactttccgcccggtctcttcctcaacacctggggattacaattcaagatgagatttgggtggggacataaagcctaaccatatcagtgggttaaccaaaactccagtcaattgcaggataagaatcctggtaaacatatacaaatcaattcatactaaaactataaatatattacttatttgaaagaattataggaattcatattcttatatattagaacatacttatagctcatgaaatttaataactccattcaggagagacagatttgcttataattgcaaacaattgaacatcgtatactctttttccccatgtgtatttcacattagtatttgagtaggtttagttgttaatttttaaaattgtattaataatattgtatctaatttgccaacaaaaactgacgaattggactttgagttttgtttaggaaaatgtcattgcagcaaatgcaaattgtgtcccatcaggtatagaaaatataaataaatgaagccttatttttctccctcagattttatcaaacaatttaatgattctagaatagagtcacaatattgattaggaaaaaatattttaatatttctaacgttttttggtgtttagtgttttctgtaaagcatatttgctagaatgtgtgtggtcatttatagagctcaaattcacatttttcacatgcgtgagtttagtgaaaagatgaaatgctagtgaatgtagatgctgaacgcattggttttcaccatctgcaacatttttcgaagcattctctatgttctctttgatcacagatagtgagacacagctccgtagagacatggttttctgccagactcttgtggccactgtctgtgccttctctgagcagctcatggcggccttgaaccagatgtttgacaacagcaaggaaaatgagatggaaacttgggaagccagcaggaggtggctggaccagatagcgaatgcaggtgttctttttcactttcagtcacttctgtcaccaaacttggtaaggaaatcacatgactcccactgtctgtgtcaactgtaacctgcactgtaacatgttttgaaataagaagtagttatttctttcccgctggttatctctatgaatgtgcgtgcattcagctactttaaaaatatacatttttctgtggtccttcaaattttaataagtatgtagaatgtattccttctttcctgatttgtatctcttctctgtggctgctaaaagctgagaaagcctccatctgttactaattcttgtatccctacacgctgactggagaaggtcatcagaagaaaaagggtctttctgctgctgttccttattcccagtcgaaggacacaggctgctgtgaaaccaaagctcctattgagatggatgtcctggagcaggcttgagagccaagcttggagagctcaaataccatactgcctgacaatctgtttaaaatttgcatatgttaaataattttagttcttttctttgtatgcatgtttgtcctgttaaatcgaagagcattgccttaattactgtagaatattctatgtttatgcattgggtttccacagtggatatgatgaaagcatcttgaaatatttgaaaaataaaactgtggtgaacttaagagttttttcacgtatacctcctgtggtttggatagttatcatcatgtttaaaagcaaatgtgttcactttaatatataaatgttaggtgatcactgaatagaactgtgattttaaacatgagagcctatttcatctgttaaagtacacatagaaaatgtagctaccaagtaacatgtttgagcttaaaaacaaattttgtgtgtgtgtgtgtgtgtgtgtgtgaattacatatgataggaagcctactttttaacacatggtaggcatcaattatctgttgaatgaatgtgtaaatgagtaaggctttattttatttctattctattttccttacttttaaactgccattgtctaatcaagtgccggtgcttttactttaattttattggaaatctcaaagaatatgagaaaatatttacaaatcatatatttgatgagtgacatatgcagaatatataaagaacccttaaaattcaacagtaaaatgacaaataaccaattaaaaagtagacaaaggatttgaatagatatttctccaaagatataaaaatgtccaaaaagcacatgaaaagatgatcaacatcattagccattagagaaatgcaactcaaaaccacagtgaaataccacaatgcatgtgatatggatggctgtaatacaaaagacagatagtaataagttttggggaggatgtggagaaattttgtcaccctcctaagttgcttgtgggaagtggcacagccattttggaaactggtttggcagttcttcaaaatatgaaccacagagtttccatattacctagaaataggatttctagataatatctaaaataaatgaaaacttacattcacacaaaatcttgtatgcaaacattcctagcaagattattcatagtagctaaaaaatggaaacaacttatatgtccatcaactgatgaatggataaacaaaatgtgatgtgtccatataatggaatattattcagcaataaaatggaatggagtactgatacatgctacaacatggataaacactgaaagcattatactaagtgaaataagccagtcacaaaagacctcatatagtttgattccattatggtgaaatgtactaactaggtacattttagaggcagaaagtagactagtggttgcctacagctgatggttgggggcaggaaaatgaggagtgaatactattgggtattggttatctttgggggttgataaaaatattctaaagttagacaatggtgctagctggacagctctgtgaatatagtaaaacccctgagctatacattttaaatgggtgaattttacagtatgtgagttatatctcaatgaagcggtgtggttttttttcaactggtcttgtgaaacccttaaatttatttaaggaattgattttaaaaagaaaatagagaggttgctaaggaaagaaaaattcaagaaacccatagtctttctgcttctgtcacaaaggcagaaagaaaacatccacccaggaatgtgaatggctctaggagcagcgctctggtggtctgtgtgcccctgagtgcgaggcacagccagtggtcacttgggcctggctttggcaatgtttgctggaacaccatggcttgtgctgtagctctcaaaggccacactgtctggaacatttcttgctttgtgaatttctgtctaccggaaaatgcactgattcttcagtcctgtcctttctttctagggtcatgacatttttttgctgcttcaaaaggatttttttaagataatggtttaataaacatttttccagtacttactgtgtactgtgcactatattaaacatcacaccaaggtacaaaggtgacagttcttccaacctctttgtgtctccacgctgccctcctttggggtctggggctacctccactgattctttttcacagaatggaaattctttaccacctccttcatcttctactgatgctgcccccagatttggctacgaggttacagcaacttttttttttttttgagacagagtctcactctgtcacccaggctggagtgcagtggcgcaatctcagctcactgcgacctctggctcccaggttcaagtgattctcctgtctcagcctcccaagtagctgggattataggtgtgcaccacgatgctcagctaatttttgtatttttagtagagacagggtttcaccatttttgtcaggctggtcttgaactcctgacctcaggtgatccacccatctcagccccgcaaactgttgggcttacaggcgtgagccaccatgcctggcctacagcaacttttacaaatgctgtatacaacatgttaaactaactgtttaaaaggatcattgacattttattttttttaagagatggggtcttgctctgtcgcccaggctggagtgtagcagtgcaatcttggctcactgcagcctcaacctcctgcgctcaagtgatcttcccacttcagcctcccaagtaactgggaggttacataggtgcatgccaccatacccagataattttttgattttttgagagatagtgtctcactatgttgctcaagctggtcttgaactcctgggctcaagtgatcctcctgcctcggcctcccaaagtgctggggttacaggtgtgagccaccgtgcctgaccattgacattcttaaggaaacaaatattacctttagaagataagctatgtatgacaaagttaattatattttcatgataaaactattaaaagaccctgtaaataatgcattaaactcctaataggaaggccagaagctagtaccagaaagatgacatcactgagacacacactaacttttttaatgtctgggccttacttgccttagctataaaactatatgctataagaatgagacgaatgattaaaacaaaccagatctgttactcacaagattctagactaagacgccataaagccaagtgaggtgaaaatgattcttgctcccaagctcgttggaagccaaataatcacacattaaaattgattttaatcacagttactaaatatagcaaatgaatatgaataagttatctcattataaaataggatagctattatgtagtttgtcttttaaataaaacaacatttttgcttttcagatgttataagattttagaagtttagatgcaaatagcatgctttttgtgttctgtaaataataaatgaaatcaatatgattagaatccagctacataatagactgaaaaatgtgtgtataacataaccatttaaacaatagagaaaagggaaaatataaaaccataaaagaaacttgtaactttaagagatttgttcatttcattttgggcagtaaattattacagaagtctttataattttgttttgtacctgtatataaataaaacacagcaaagttgttttggtctgaatttacattatgtcatgttacgttatattgctttatatcatcttgtattatatattgtttttctttctttgtagacagatgaacaagccatgttagaagatacactggttgcactatttgatttggaaaaggtttccttttactttaaaccatcagaagaggaacctctggttgcaagtaagtaattaggtatttacaaaatttatttggcatcaaataattatatatgatataactttggtattaatatatttgataagtattttatgaactgaatgattgaaatgtatccttttgatttagaaattttccatcactaatctgacaaaggcagtattttaaaagttatatattcagataagtagcaatatgtatggtttaatgtccatatcttttactttctgcaagttatcattatctactatttccacattcccataaatatagttaaaatatatctatcagtctaagaaatcttcagtagtttgttattatttttatatgtctcttgttttttcaaaagtatgagtatcgtgtgtattaggctagaaggtgtaagcttcttcaggttttatttctcacattcctaatgtccagtacaataaataaggactccatacattttcatttgatccatgatctgacttaagatgataatcactcatttattcatgtatcaaaagctccttaaatgcctacttcttgccaagactattcttggtgttggattcagtagagaactaaacagatagatatctcttatggggcttaagttaaggtgaggagagataaataataggcaaaataattttataataaattagaaggcaattatgtgaaagatatatagggaaacaaaagagagcaaaggataccaatgaaggggcagatggaaggttgcagataatgaatggggtagcaaggaatctttacctaaaaggagagcaggccctgaaaaggttgagggagtaatggcgatatcggaggagcagtgtcccaggctcactggactctgaggctgggagcagacatggcatgctctcaggataggaagttggcctgcataggtggagtggagcaagtgaggggaacagaaaaagagatggaaacagcaaggaggccagactatgaagggcatgccagctgttgaaagagctttgcttcctaaccatgacattgaactgatgaattacgggatgtgtgttccatattaaaaggctgtctctgactgctgtgttaaatagactattctaggaccaagggcagaagcccagtgatcagttagaaagctacctctcccaggtgggagaggatggtgacttggcctggagggtagctgtggagattatttaaaaataaagcaatgtcaatgctgtttaatttatgaaaatagaacagaatattattaaagtttcatttcctctgatgaatgaggaagagaacaagaagcacaaagttagaaatttaggttatatttattcaagaatttttctggcagagggtatttgataaggataatattatttgaacaaattgtgtggttttctgtggatatgtatagaataggatatattttcatttacttgaataatgtgggcatagttttgctgaaggcagagagaactctatttttgtgatataacctgatatgttgtttaaagttttgcctgaggaactgtaagttcataggagtactgttttccatggttctcctatgacattattggcaaatggtctgttgaacaagtaggagaagaaccaatatcataagcttgataaaatacttccttactgcatatctactgtgaaagacttaactttggaacacacactaaatataaaaaataattgggaccatgtaagcaataatagtatgtaaatgtagatgtcactataacagatacaggcattctgaatatatttggcaatacattttaatgtgtctcttcaaagcgtaacctcaacttctgaaaatagttctctagtgaggggctggttccaatgctgcactattgaacactaaaaacagcgaagataggaatataattcttattttaagttgatgcaggaaataagagaattattctgtattcagaggtcatcacatcttatttaagtggtgtaagaaaatgaagctttattatgcattgcatgtagtttgatctttctgttacttagatgtggagaagtcagattcactaatcctgtatcactgggatggagactagttggctgcatgcctttgagggaggactaagcacagagatggggaagcatgaaggtgtaaagacaccattggctttgaagtcaagaaatccaggtccgaatcttggcttcattcttatctttaagcatgagaactatagctttccctaactcacagggttgagggacttaaataagataatgtatttgaaagactctagcattacatatggctatactagtattcagtaagtatttgaattcatccttcttactttattactttgcattcttctcaaaagctatatttatagcatcgcctataatctcacttttgcatgaaaagaataatgagattctgaagaattatggcatgccaagcaagtagaagtcaaatatcttgattaaagacagttcattttcattgaagaaataattattaaatatatataacatgctaagtactgaaccaagttctagggatatcatggtgtgcaaattaaacaaggcttttttggcatggaacttagacactaatgagagatacaaggaagtaaatgggcaatttgaccacttcgtaataagggaaatacgaggtgcttcacacaatgtgaaggagtaataaactagctaaatggtaagtttgtctataattacatttattttgtggaaaatctgcatcagtttaggagaataatgtaatgacagcaggtcttctccttttcaatggtcatatgggtcattttatagtagttcattattaggagaatgaatatgtttgagtccccactatccttatatgggccaggcattgtgcctggaggtgtgggtgccatcatggatgagacatgcatggttcctgtcttcatggagctgacagcctagttgggaataaggaattaggaatcaataatgaatgaggaatgaataatcaacaatcaataagaaatcaatataaaaataaaactgtgataaatcctatgaaggaaataagcagatgcataaagaaaaaaggcgaggcagcccttcaaggggtgactggtggaggattctctgcagaagtagcttttaagctgagatctgaagcataagaaagacccaaccctgcagaaagtcagggaagagcaaccaaggcagagggaactaacagcttcttagtcaaagacttattttgaaactcacgggcccattcaatgattagaggctatgtttacaaggatgggatggcactgttgaaactcctctgtgggatgaatccctctataggatgctaccagagaccttttcctcttgcaattttttgaaacttttaaatattatatagtcctaattatcaagtttgttttttatagctaataaatttccacaaataaaaggcaacaattttcaactccacattagtattatgcactttttaaaaagcctttatatttttagagcagttttaggttcacagcaaaattgagaggaaggtacagagattttccatacccctgtccctacacatgtacagcctccccagctatcaatagccctcacgatagaggcacatttcttaaaatcagtgaacctatattagcacatcaaaatcactcaaagttcatagtttacattaggattcactcttggagctctacattccctggatctggacaaatgtgtaatgacatgtatccaccattatagtatcatacagagtatttttactgccctaaaatcccatatggattgcctgttcattcctcctccctcccactctgcacccaacccctggcatccactgattttctctactttctccatagttttgcctttttcagaacatcatatagttgggatcgtacagtaggtagcttttcagattgtcttctctcacttaggaatatgcatttaagcttccttcatgtcttttcatggcttgatagttcatttctttttagtacagaagaaaattccattggctggatgtaccacagtttatccattcaaaggacatcttggttgcttttaagttttggcaattatgaagagtggctataaacacacatgtgcaggtttttctgtggatataagttttcagctcttttgggtagataccaaggaccatgattcctggatcgtatgttaagagcttcttagtttggcaagaaagtgccaaactgttttccagagtggctatgtgcatggagactggtgagaaatacaaaggcataaatgggcaatttaaccactttagtgataaggaaactacaaggtactcccatatcatattatttaattagtgagagaggactaaactagctaaaggattaaattttatatcatgaaatttattttgtggaaaatctgcatttttccctccagtgatgaatgagtgctcctatcactccacatccttgtcagcatttggtgttgtcagtgttctggattttggcccctctatgagtgtatagtggtagctcagtacggttttaatttgcatttcccttataacaaatgataagagcatcatttcatatgattatttgccatctctatatcttcttgaaagagttgtctgttgaggtctttagcctatttttaaaattggtgatatagtttggatgtgtgtccttacccaaatctcatgcttaattgtaatccccagtgttggagatggggcctggtggaggtgattggatcatgggggtggatttctcatgaatgatgttgtgccttccccctggtactgtcttcatgataatgagttctcatgagatctggttgtttaaaagtgtgtagcatgtcccagcttgctctcttgctcttgccatatgatgtgcctgctacccctttcgtcttctgccatggttataagtttcctgaggcctcctcagaagctgagcagatgtgagcatcttgcttcctgtacagcctgcagaaccatgaaccaattaaacctcttttctttaaaaattactcactcagacatttttatggattaacacactttgattgtttgttttcttattgttgagtttgaagagttatctgtgtattttggaagacagtttttatcagatgtgtcttttgcaaatattttctcccagtctgttgcttgttttctcattcttttgacaatgtcttttgcagaacagaagttttacatttgatgggatatagcttattatttctttcatggattgtgcctttggtgttctttctgaaagtcatcagtatttccaaggtcatctaagttttctcttacataatctggcaggaattgtatggtttttcattttatatttaagtctataattcattttgggttaatttttgtggaggatataatgtgagtatctagatttatgtttttgcatgtagatgcccagttcttccagaaacatttgttgaaaagacttatctttgctccattgtattatctttgctcctttgtcagagatcagttgactatatatatgttggactgcttctgagctctctgttctatttcattgatctatttgtctgctctttggccaataccatgctgtcttcattactgtagttttatagtaagtcttaaattaaaggagtatcagtcttccaactttgttcttctttttctatgttatgttggctattttgggtcttttgccttttcatataaactttagaattagtttgtcactatccaagaaataacctgttgggattttacttggtgtttcactgacattgttcatcaagctgggaagaaatgacaccttgacaatagtgagtcttcctatccatgaacataaaatgtctttccatttactgagttcttaaaaatttttttcatcagtttttcagttttccttatatagatcttgtaatattttattaaagttatacctaagtattttattttggggaggtgctaatataaatggtattatgttgttaatttcaaattccacttgttcactgctggtatataagaaaggaattgacttttttatattaacctgtatcctgaaacctagctataattgcttattagttccaggagcagttttttgttgatattttcctttcccaatcattatagtttcttttctcttgtcttgttttattgcaatagctaggacttccagtatgatgttgagaggagtggtaatgggctgggtgtggtggcttatgcctgtaatctcaacactctgggaggccaaggctggaggatcacttgatcccaggagttttagtgctcttagtttgaacttcactttaaattaatatactttataattttcattaagttttagttccttgattcctaatataacatctttatttgttctatgttagttctataatatgaaactccaactgttgtatcattatcactatttttctttcaataacattatccatttgttagcatagaggttgcctatattgatgccaaagatgaagggtatggaatttaacacaaattaaaatatatatgtgagacagtgtgtgaaaaggggaactagaggatacggatatttagtaggtataaatctgtattttctttagtgtccatagttcttttagtctttattttagaaatcagaaaaaaaagattttcccaattattcaaatgctattaattttcaaccctttgggtgagaaatctgttcattctcttttctaaatcgaatgcttcatgtcttcatttaaaaaatagtaacaaaaataaaataagtactttatatggcataaaggagataaatagatgttaatgtcatgtttcccatattggtatgcagaattaaatagatagatttaataaggagcctggaatctgtgatcataaatgccatttaaccttgaactctacttttatatacagttttatttcaagaagcaggtcagaagttataaatactgagtgaggaacatgtattaaatgccgattacatttgtttgtccttaatttttaactggttattatgactattcagccttttaggatgattttacattcttttctagtgagacagaaatatttgtcatggataagtatcatcaaatggagaggatttatggtagcattgtgaaatactccgactgaaatttagttttcaagttgattggtttttttttaagtaattaaatttaggatctttgaaaatagctacaagacttgggacaatttttaatgactgtggggcatcaacaaaggctaatatatttccgatctggtgctcagggaatagagcttgcagaacccatgctaattttgatagtggttgctatgtactcaatttcttgcacactacttaagaagtataccccaaggactcattctgactaaaatgtgaatgaagcagctgccctgggagattttgattccaaactatgtgtagctaaacacctcatttagttaagtgagacatctgatatgtcaaagtgtaacaaagtttcctattatggaatttttataatatagtaacacatccattgctttaactattgagaactgatttaatactttcactgaatagcactgtatgtcgaatcccaacttgataaacatgatcacgtttaatatattggtgtcgagtaattctcaggattctttctgaaagttaacaaaagtaccatctagcatatccttattttaggaagtaatatactattaaatatcattttagatgttcctcttacatatcaagcagaaggaagtcggcaagctctgaaagtttacttctacattgatagttatcattttgaacaacttcctcaacggctgaaaaatggaggagggtttaaaattcatcctgttctttttgcacaaggtaaactgtttctcttttgatggctgctatactttaattattttatctttattaatgcttttataaatgtgattttaacagttgggatttttaaattaatttgaaaatattatttagccaatatatttgcttctgggaatgtttattaaggaaataatgcatgatttaaatatttaccaaatacagttgcagcattatttatagtggagaaataagacatgagtggtgtagataaagataaaataatttgggttaaataaactgcttaagtaagttaaattatggtaaaatcattacagtttcatggttaaaatattttatgagatagaaaaagttaatatatgcatgcagattattaaacagaatttatactttagtctcaattatatcacacatgtatgtctatacctacatatatgtatgttaaatgttttcagtatcaatgggtggttaaattgatgcttttaaaattctttatgtcaacttttctttttggtcagaaaatatatattcctttgtcattttgttttttccaagagtgactatcaacatctgcagtagaataaattaaggttagcatgagcaagaaaattcttgtagtgagctctgtttctgagggaaggtcttgaatctttcttattttggggtcataaaatgctacctaaaaaagatcagaagtcttgattattgtggcgtgtcctctagtctattatttccctccacattctaaaaaaaatgtgtgtcatgtcacaagattgtatcagtttctctgaatgataccatagggaattttatttttttcttcttttgagttttcaagaactacattggttttgctgtggaatttcaatgctctaataaaaaatgagaagagtagagggaataaaatgtatctatctatataactaaagaatggacaggcatatgaaatctaaattcactactaaatgaaaattttgcattataccatgatatggtttggctgtgtccccacccaaatctcatcttgaattcccgtgtgttgtgggagggacctggtgggaggtaattgaataatgggggtgggtctttcccatgctcttcttgtgatagtgaataagtctcacgagatctgatggctttaaaaatggaagtttccatgcacaagccctctcctcttatctgccaccatgtgagacttgcccttcaccttccactatgattgtgaggcctccccagccatggggaactgtaagtccattagacctctttcttttgtaaattgctcagtcttggatatgtctttatcagcagcatgaaaacagactaatacataccacgtgcatattattttaaaaccctgaattcaattaccatagtttaaaaactgtgagtacctcaaatatacttggcaggaatacattttaacagatgaagattgcagtgaagagatgattttaggagttactagaaggtattttcatttctctttggaggactgtgatttatagacagggtcagtgtaggtaatccagattctataatgaaaaggaagagaacaaaaaaagaagcagtgggcaaaaatggacctttggagacaagttagaagaataatttaatctaaaatatgttgggctagttgcggtggctcacgcatgtaatcctagcactttgggaggccgaggcaggtgaatcacctgaggtcaggagttagagacaagcccaggcaacatggtggaaacctgtgtctactaaaaatacaaaaattagccaggtgtgttggtatgcgcctgtaatcccagctactcgggaggctgaggcacaagaattgcttgaacctgggaggcagaagttgcagtgagccgagatcatgccactgcactccagcctgggcaatagggtgagactctgcttcaataaataaataaataaataaatagccactacagggatagatagtttgagaagatagggatggacgttggtctggaatgttaacttttacagggtttagccaggacttacaattgttaaagactcttttcctttatttctctcttttcctttatttctctcttcttaaaaattttgactttgaaattattactgagatgacagatggcactaagcatctttaaaaaaattattgcatctatgacacatcagccataaatagctgaataatcatatccaacagcaaatgtacacataggcaaaaaattattttgcaagcaacctctattttataactttggcaaaagccaatttaaaaataatccctaaatatgtcttatttcagtgaatatactatagcttttaaaatgtaatttaaagaaaagctatagcaaacattaggttttggtatgctcacagcagcaaaatatgaaaaatgatttgaataagtaacagtagaaatacttctacagttgaggtcacagataggtaaacaagatggaaaacattctgtaccattaaaaatctgtcagtgataaagttgtttatttaacgggggtgcagaagttcttctgcctttataaaactcccatgaggaatcattagtagcttagtattcatagattgagagaagtacaggaaaatcagacctagtttgagtagagattaccatctggtaattgaaggcagtggtaaatgaaagagattccaaatgtcatctcttcctggaatgaaagtttatttaaagcattttagttttgtatatacatgactctttgctagcaaaaaaatagaagaaatggatgaatttgaaattttcctaggaaatttactaaagtcaaccctcctaaagttgtctgtggtaccatatactcagagtagaacaggaagcaaatgcgtcacccctttctcattttccttgcctccatggatcactgggaaaaggagggcacagtagttgatggggcatccttcagagatgggaccagctattagatcaggaatttcagggttggaagtgagcagggttggcggccaatgtgtattgctgaaggcaagaactgcggaagaacaggaacctaggcagacaaaagtgatacagattttcaaggtaaggaacacagaaacagctggtcaggaaagtaagtgcaaagcataaccaaccaaactgctagagtgtgccaatcacactaaaacaatcttctggctggtattcctaagtgcttcctacaggacagggaatgctgttgggatccctcagcccttggtgagataaagcaagcaactataagttggcagaaaatgagagtacaaagaggataaatattcagggccacttctctacacgctccccactgactaaatttcctgatctgtcagtggttttcagtacttagctgcacattaatgatcttggaaaggttttagagaatacccaggctctgccccatgcaaaatcaattaaggcagaatctctgagaagtggagcccaaataccaggatattttagaagctttccaggtgattctggagtccaccaaaagtgagaaaccactctaggtctgtgtggattatgaaacgctgtgcaattatccagctaatctttatttaattagactaattcatcttacaaatgttcagcatgtctagattctggggagctgaagcaaagggtccaggaacaatataaaatatttctgcatgttaggttgttaggatgcaggctgtccttaggaggaggtgtgatcttagtgggatagttttctccagcaaaggctggagagtgaacagctgagagatgtctgctgcggctatcacagcagccgggcaatgagcccatcatttctgagagaggatctgagccagtggctctttgagcatcctctgtagtccatgctttgcacctgtcagacccatgtctttgtgtagggactgggagaggcttttccaggactcccctgggcctcctttctggatgggggaggggataatttaggagagaaaaattaaaaggattcgctatagcccttgcagccccagcttgtctacagctgccacagagtctcactttccctcttctgctacccattgtagattagcctcaccctctgtctgaacctctgctggtctctgtggctaagctgtggcatgaccaataccttcatcccggaggggcttgatcctctgattactctacccttctcagaccaggattattgcagttgtccatttatcatcaaatttgtgcaaggaagtactaagagggaccccagtagatacctgggtgccaaacgtattccttctggctcccattgtgtaacaatagccctacctgcttctgatgattagggtggagcagctttgccaaggtggcgattgatcttcttggctgtgggtgccttggaaacccacagaccaagtgtttcagggattggaagctcacattacccaagtgagtcattggaaatgatgcgttcatttcccagtgctgccctagcaagttaccacaagctcggtggcttaaaacaacagaaatgtattctgccacagttctgaaggtcagaagttggaaatcaggatgttgtcaggccatgcctcctccttcctcttttggcctttggtgtcttccagcagtccttggcattccttggtttgtggcagcataactacaatctctgcccctatcctcacacggtcttcttccctctgtgtgtgtctgtctctgtgtttctacatacaaattgccctcttcttataggggcaccaatcattggatttgggcctatcataattcagtaggacctcattttaatttggctacatctgcaaaaaccctatttcaaaataaggtcagggctgaatgtgatggctcacacttgtaatcccagaactttctgaggccaagatggaaggatcacttgagcgcaggaacttgagaccagcctgggcaacatagtgagacctcatctcttaaaaaaaaaaattagctggttctggtggcacacacctgtagtcctacgtattcgggagactgaggtggaaggatcacttgagtctaagaggccgaggctgtagtgaattatgatagcactactgcacaccaacctgggcaacagagtgggaccctatctcaaaacaaacaaaaccaaaaataaggccacatttgtaggccccaaggggttaggactttgtcatctctttttgtgggcacatacaaacctcgacagatgttaagcacggtcatttctgctttcacccttggttcctggacctgggtattctacctctcaggaatgtggcatcatatacaggtctttgaatatatatagtgaatataaagtgtcctgaagtatagcatcccttccttgtaaagtgttgttaaatacctatttcctgggtctcctgatggtagtttgccttaaaccaaataggatatgtgatgctggtcttttctctgccagcatgctcttgagtattctcttttttttaacctcctatgaaaaaaaaaaaagctgccacccaaaccaaccacccctaagtcttacctcttgacaaggtatctttctttgctctgagaataataaaaatcacatagaaacttgagtgctttctcttttaaatgaaaggggacgttttgttataaacattgtcattttctaagtgccctttattcaattttttttctcctaaactcttcttaaggctctttaaaagataaatcttggccaggcgtggtggctcacgcctgtaatcccagcactttgggaggctgaggtgggtggatcacgaggtcaggaaatcgtagccatcctggctaacacgatgaaaccccgtctctactaaaaatacaaaaaattagccaggcctggtggcgggcatctgtagtcccagctactggggaggctgaggcaggagaatggtgtcccgggaggcagagcttgcagtgagtcgagatcacgccactgcactccagcctgggcgacagagcgagactccctctcagagaaaaaaaaaaaaaaaaaaaaagataaatcttttagttttccatttgggttttacacaatatttataattgaaacaatatagcttttttacaaaagaagaaatcgtttccaggatggagatttccaaaatgttaggagtctgtgggaaggtgtaaacccacctctcttcagacagcacggttccaatcagcctagttgtgccagctcctattggaggaggatgggaggtttcacgacgactcaatggccaattaaatgctttcaaattcatagtttattgcaaggatttcacacaccaacaatcatgcaaatatactcaatgcacacaggcagacataaaagagagggggaacaaaccatggtgagtagctcaggaggcccgcaggctgactgaggcacaaggaaggcctggatttccctggaaacactgaccagcaaggggatgggggtccctgctcatctcctggcagagcttctagcagccactaccaatggcgaggaggcctcagagttctcctggcaaagcttctgcaggcatctcaactattgtcagtttcttgctgtttttatggatctccacaggagtgtgtccgtagtcatcatctcagcccctttagcttcactttcctgtggtgtctctgaggtgcctggccatgggaggtgtcatcttatcacctcagtccaccatacatgtgcttatcactttgaagggtcagcagtttcactgtgggctgagtgacttgtcataaatgactccatttcaagacatttagaatcacaaaacattattatgtatcacacggccattctcacatgtcataatgggatcaacaaatcttgcttctctcccaataactgggaccaattctgaggaattatgacccatggtgaccatttctggggaattctagcagtgcgtgtgctgtgtcttatttaaccaaacaagtgtgctatcaaaagagaatgttagcagactgacactgttcttaaaattattcttcagagattctatgtttttcctgattaatctctttcttgggaaagccttgaaaggtattttgagtaagtggaaatgtagcccgcttcagtctaagaataatttaatctttaattttctctaacattcattaggttttccttttttgctgactacctaaatttaatggaatgtttttcttcctctacttgaaaagtagtagaagtaaataaactaaatattcaaccaagtatgttgatactgcttttgagaaatatattattttatggatgagaactaaggaaattagacaaggaaatgccaaatttaacatgtttgaaattaacatgttatttaatttggtagaatgtcattctgggttttttataacaataaataaacaagttgtttagctaaaatagggaatgttgtcatctttcctcttctctgtcatacaaattttagtttttaaatataaaactcatctttttttgttcattccccacaaccctatacatgtgacacagaattcttactaagactcttggcaaaattgttaggcacttttccaatttttaaaaaatgttatgtgattgtgaattttaactgccttgttaatttggaataatattgtcaaaaatgtaatgaaaatggaaatttatgagattgtagaagtattcattaaaattatattaaattatgtttacatacttgttcttatataaaatttgggacaggactggccattctggccaaagttgaaccatcaagatctttacaaaaattagttcccaggtggtttcccccaagccctctcccctcacagttccactgctgtgccatcccactgttctccttcctgtctttctggttttaagtcacaaatcaaagtgtttttgcatggacccttctcttagtcctacaaactttgtgtttggctaagcacactctgcataatcagtatctctttggaattaaattaggtaatacctgtacagcatttggcatgttgcctagtaaataaaactcattcaataagtaagaatactcatgagctgtcttaatactagtatttaattaaattctttctcattaaccataattagtaggtacttttatgttgcaagtcaaaaatgcagaacctgagaatctcagatattaattaccttgaccaaagtcacacagcagaaactggaattttactctaatacatcagctttccagctgttaagagtaatttatcttgaagtatgactattgacagactgaatcctcctgggatatttacctttcctaaatagcagcttgtgaacatggcatgtatttaaggattattttatcagggagagtttcttttatttcctgtgtgtattcaggaccagaatttcgttgagccagttacaactattctaattaacgaagatgtaggctctaagagagaaagcctgtccttttccctttgaagggttgctgaaaatgaacttgcaataggcagattaataaggagacaaagcatacaaaatttattgaatgtgcacggggcaaaatcatgggagagtgattacccaataattcagtgaggtccagatacttatatacccttcttcataggcgatgggagaggtggggaatgtaggaggcagtaaaatgtttattagggagaatgaatgggcccaagagacagaaattaacttgtaaatgattgtcttggaatttgaatgagctggagaaatagatattatcttgtgggaaaagtccttctaggtgggttacattccacagtcttcttttctaggatagataatgagatttccagggagggtagggaaagcaattgtgttttcttttgcaggtctagtcttaaggtagatagggcatatcagagaaaagcctcatccaatatctgctgacttgcagaagcctttaatttaaagtaatcagtataccaaagtgtcatattttaagggtaaagtttcctgggctccttcccagaagccataagaaataaacaaattaaaaatctgtgaaacaagcacacaaatttcattttctgtactgaagtaaatgtagtatgttttctccctaatcttccacaattgcttttctcaagtataaactggttttttaaatttccatttaaaaaaaatctgcaaatttgacatggatctttaaaattttattgtttttcttcagtgttctggaattctgactagtttaataaaactatagcacagctaaaatgagcagtcagtttcctatttccccccctcaaaatggtgatagtttctttcactttaaagaaatctcgtgatttagtttagaaactagtgcatttagcagtcttgaaagaagattgttttgaattttgaaattgtttattaaaaattgaggctcataagtagtgatgcatttcaagggaaaataatttacatttccaacttggatccatagccattcctgttactggcaacctttccacaggaaatcttgtatatctgggcccttgtcctgtgatttatttcctctccaaaattttttaaatgatacctcataagggttaaaggaaaaatactgaatcaaaaatgttattaaaaaggaaatttatatgcttttcaaaatacatttaaaattagtgagatattataactaaattgacaatttgagtagaggtgattttctaagatttcaatttatactttttattacattaatatttcttaatgaaggttatacaagaatgtgaatatgcatggggagtgaaaatttccatttttttactctgacatgtttttgttgttgttactgttgttttagaagacagagtcttactctgtcccccagactggagtccagtggcacgatcctagcttactgcagcttcaaaatcctgggctcaagttatcttcccacttcagcctcctgagtaactaggactacgggtgcatgccaccatgccaggctaattttttaactttgtttttttagagacagggtctcgctatgttgcctaggctagttttgaacttctgacctcaaatgatccaccagccttggctctcaaagtgctggaattataggcatgagccactgtgccaggcctctgacatacgttaatgcacagttctgcctctcatacatatgttgcgaatatgtacatatatgtgtcatatgtaatagacgtgtgtgtataaatatcaaatacattggaagtgtgagtttagcttcacaaagaatgttgtaggggaggaaaaataattttctctctacctttcatagttcttcgttgggatggtccccatagcaaaagacaagttaacaagagaaaaacaaacagaagtttaataacaggtatacctcatgtatacatgggagatacctaaggaatagtgactaaatctgaaagaggtagctttgaacttaggcttaaatataaccattttctactgagaagaagggtctagggaaggcccagttatggggaggtgaccaggaaagcaccataaaaaaaagggtaagatttgttgtgcagataaaactatctgtgatcatcttccttctcttcctagtacagagggggagacactgttacaagtagaggtttcctttatggatgtaaattttccttatcaaagggaaacttctactcggttttcagagtttctcctgtgtctgcgtgcagtttctcaaaataaccagatcaaaataatccttatgccaatgaggcatattttggggtgatatattctggtctcctacagttatattttggagcggcatattttgggctcccacaacatatacaaaatgttttaaaaatagaggacagttaaaccagaaattttctttgcccattggacagaaaaatcaccaaatgtttgatatttattttgagagcagaacagttttcattcatttttggacctaacatagtttttttgcatatatgttgataattggtcctatttgctaaatttactaataatcaataaaattatactacacgtggagaaattgagttataaagttgcttgtaaaatatgtttacttgtttacacagcactagagagcatggaaggatattattacagagacaatgtttctgtggaagaatttcaagctcagataaatgcagcctcactggaaaaggtcaaacagtacaaccagaagctcaggtatgtaacaatccaactgaaaaataatgagtgtctatattttgatagaaatgtattaataggacttttgatgctatttatcatattagattattaatgacatttcttttgatataaattcctcggatttagggtttaatattttaaatattctaatttgttatactttttcccagtaaaaataattaagtaaaagtgtgttggcatttttatgcaaatttaaaaatttctgaagagataaagagaaggggttaaatatgtagtagaccgtttccaagaaagaaaatcactgacatatgttatagtggtgtcccctataagatttattggattaagggtaaaacaggtttctctatgtaccaccaaatagctggcacccccacacagagagaagaatatgtgttcagcatggaagatatgattgatcatgtgcttggtattggaaaaatatgtactttcaaatgtgtcccttaatgcttgcattttactgtctttttccccccaaaaaatagtgttactggtacaatgataaatgacagggaaaattaaccacagtaagtgaaaagaaatgtgttataattcaaaagctttatttgtcaacaaattattagtgtatgtgtgtgtgaatatatatatgtatatatatattcacatatatatgtgtatatatatgtgtgtatatatatgtatatatatatacacacatatatgaatatatatgtgtatatatattcacacacacatatctccttcttatcttatgaattaccctgttgacagatcacttttaaaaacttaacaaattaaggcttttatttttaaaagaaaaaatatttttgttctatcttgagccttaataaatcaaaaatactattttgacatttttaattttttgtcctgtcaacttaaaaggaagaacctgagacaaaattagtataagtagagagtttatttgggccaagcttgagaattgcaagccaggagcatagattcaaattgctctgaatgttcactcaaattagcagcaattacaaatagatttttaaaggaaaaaatgggagacaaggagtgagctgatacaaagttgtttgtcaggcattctcactgatttacagaaataacattgacaagtggttggctatgcattgttaagctctagggtgtgggttgtagcatcaagtgtggcatctttatgttaagttacagctacttatggtgatagccagcagtttcaagagataaatacatagcgcaaagggactaggaggatgtaattgctgtttcattttaatgtctctctgggcctgttaattaaaatagcttgcattcctcagatgaaagttattttcttctctcatttcccccttttgatcaaaaatctttcttcttaacagcattgatgatcaaaatttgagtttcaaggtgtccatcatcaggaaggctcattcccaagtagttctgtcaaagtcattgatttatggctgctgttgcttgttgaatcgtagctagacttcagagtatcgtcaatttagtttcatcagaagaaggaaaacaacaagagatagtggaaatataatccatgtaaggattataactaaaaataaaatttgaatgccagaacagcaacaaagacagaactattccattaggcagtcaactaaaaatatcatgaagaaaattaaatccaggttcttctttagagactcatagctgggaagtaattcaagatttaatccaaattgtaggcaaataataaaactcaaaaacaatggacaagactagaatctaataaccggcatactatagtttttttctgaaacattttttctttatccagttctccatttttaccaaagacaaatcttagttgaaccaatttatttgcaaaataagttttagtgttatgatgctttgcctacttatttgcataaagtgcaagaagaatagtgaatggccatatagtctcttttaagttggctttaccgggacttccacagtagctttagattcagcttttaaaagccccaaggccagccaatccaaggatttgccctcagactgtctgcagtacttgtaaaaaatgggtgaactctgctcttcttgagatcccaaaataacctgagttcctggtcctgtcagaaagtgacattctttagttaccatgaggacagaaaccttataaaggaattgtgcagacaaagtaccagaccagtctttccaagggtttttttaattggctctataaagtccacatcaattcctcaaagcagtctgattatatctgaaactatgccattccagtcaaagtttcagtaaaagaaccagtatatctaattatgtatcttaaaaaagaatacagatttttgttgaatttatataaacaactacattgctagaaattaagagtactcacaaatagttcccaaattttggagaaatgtgatacagagaaagtcaattgttttggctttactcacaaaagtatacttcacccaattgctataagctgtaaatggctcacaaagaaaaaaaatagttttcttgactctagaaaataaaacataaaaagcatcagcaatatctcaaacaaaaaaatcattaaaaattattttagtcctctattagtttagtctcatggaattaattcttgttccacttgatgttgggttagcaattctcatgaacatatcagctcttcagttagaatcctggaagttttttcttagtccaatggtatgatctccaagtaatcagaaacctatattcacatcagtcttttccatccttgcatgaacttctttgaagacacaacactttagaattttacttgcttgtagagatcttttagaagatgcatcagaattaagtaattaactgtgcaaatgacttaaaatcatcatagttatatatacaattgaaaagtaaatttgtttatgtctgtggcctataataatttaacataataagcataattatcactgataacatatatcaaggcaaaccagaattttagaaagctcatataattttaggacacatattactaacacatttatataaatatacccaaagaatgctaaacaccatttcttatttgatagtacttcctgtatgaatgtaatataccaaataagcctaatatgtctgtcttggacttacaggggttactttgggaaacacagttttagaatttgaaatgtgatttgaggaagtatgtcaaatattaaaggtttaaaacatttgttcaaaataggatcataagtaactacaaaatgagattcatttagccaaaatgataattccaagatttcaaaaggcaaaaatctttattctttgatagagagaagacttagtttttcaaaaaatcaaaatacctaataaagacagtatgagaccaacagaatctttctcttttttgacagtttactcaaaaggtaaacaaaagatttttattatctcattaatattgtgtaaaaatcctattcaaaagaaaataccaagttctacctttgcatcagtgtattattaatggtaaagttattttaataaaaccttataaacaaatccatccaatcttaatcagctttgaccacacaagataagattttcataaaccttttataaccttttaaaattttcttttcttttctttctttctttctttttttttgggggggggacagagtctccctttgttgcccaggctagagtgcaatggcatagtcttggctcactgcaacctctgcctcctggattcaaatgattctcctgcctcagcctaccaagtagctgggactacaggcacttgccaccacgcccggctaatttttgtatttttagtagagatggggtttcactatgttggccaggctggtctcgaactcctgacctcgtgatctgcccacctcagcctcctaaagtgttgggattacaagcgtgagccaccatgcctggcctaaaattttctattagagagcagaacaatgctctaagaaaaccctgttattctaatagcacgccccagatcctgaccctgcatcagtgtgcttttaatattaatgttaagtttttagaaaaactaaataatccccttttaatttcagccaacttgatcacacacaaaatttctttcacaagatccatcttccgcaaaccttctacagcttgcttaaaacatcagttgtatcctacattttaccataggacaaaaatttactttcccttctcccttattgtgaccatataaagttctctctcaagtaaaaaaaaaaaattactctatcttttcaactttctttacatcgtattttctttatatgttctgtatatagacatgtttctcttatatctggtagttttagttacacatactgattacagtttgcattagtttgttctcatactgctaataaagacatacccaggactgggtaattataaaggaatgaggtttaattgactcacagttccatatggctggggaagcctcacaatcctggtggaaggcaagaaggagcaagtcacatcttacatggcagcaggcaagtgagagagagcatgtgcaggggaacccccatttagaaaaccatcagatctcttgagatttattcaatatcatgaggacagcatgggaaagactcacccccataattcaattacctcccacttggtccctcccacaacatgtggggattatgggagctagaattccagatttaggttgggacacagccaaaccatatcacagttttaacctagtcactaattttcagtgaaaaacctaaaaagtaagtaattttgaacgattttatactagtattttttgatgaaaaccattttataattccttagaaagaggttttctcaatttctgtttactaacagatctaaatatatttagttttactatgccatatagaaatatgatattaaagtatataaatgtgaatttatgtttaacaattaatgtttcagtattttaacttacttagaaatgactcagacattttatgattagctattacttaatttaacataacacaatcttaagacttttaaattactgaaaagaattaggaaattatgaaacaggtaccctctataatgtcttccccagttgtcctaggtccctaatatttacatggcatccaaagatggcaatgaagagaatggctcacctggttcctgaatttccacaccaagtacagagctcagaacagaggagagagctgtgaagagtatgattggaggatcggacccctcccagcatggccaggaggaaaagctgggccagggagaaagaggccatgttaagctttattttgcattttagttggtggtctaggctctgaggacttgtccccagacctcactgtggccatctgtccagatcccagatctacatgctcaaatccaaagacataagctcacagacaaattaagcaaatattttaaaatatcatacaagctacagttttatgaccttaaaatatctagcagagatagtataaacttgtctgaccagcaaacacagacaaaaatgtccaaattatgaagctcttcctattttattttaccaacaatttttaaattatctttatttaccaaagattactaaaatcgtgtgaatttgaaaagcatttggtctagttatttaatttatgggtacacttatgtacaagccagtttggtgttatagacaacatacaaaaaaaagatgtgtacacatgtatacacaaaaatacaggcagtcataaagatcttataggtttagttttaaaactttagccatgagtcagataaaactctctagtttaaaaggacagttgtattcaaactgtgtctttgtaaatggaaaaggttaaagtttatctgtttcatgtggctgaagcccttactgaattttagagaaacagggtagcaaatttgcatctcaaatcaaagatagaaaatgtaatatttttaaagaatgaatttgggtatgttagaggaaggttaaaaatgaatttcaaggtaacacaaaatcataggaatttaccatagaatttcataaggaggccaatttcatttagataggtagcttttaatttagtgtctgttttccaaatgggcttaaggtggagcccattaaggaacagggtcaacaaagtatttgcagtttttaaagctgaataatttaaatacgtgaaaagcagtcacagttggaaggcagagcaattagatttttttaaatcaagcattctaattttgcactgaatcctggattctcccctcacaaagaaagggaaacaccatgggaccaggccaagcaatgcttctacagtgcactgtgctacaaagacatttctccaaggtttaaacctcacctgtctcatctaaactcccaaggaaatgagtagtgtcctgtagcaataaccatttattgtaaacaatagccctcagccacctccaaaactgcagttctgccagtgactctccagtcatcacatacaccaaggtcaagttctctcttagtacaaagtaatctctggtagtcctgaaagccaacgagatcaggtaacacagtacaaaagacggcggtagggtatgagacctcagacgaatctgtccatgactctctagactttacaggaagacagatgacccaaaaaaggggtcagtggcacctttttctgtgtttcttaaacagtctgagtcattagatatctctcccagatcattttatgtggtacctaagatggcaaagaggaaaaaggagtcaggaaagaatagaagtaaacaatagaacaatttttaagaaagaaagtaagcagagggaaccagcagataattaatgaaggatttccatccacaggaaaatagtagtccccaaaacaggatccaaagagaaaaagcagaaaggccttaatatgtattatatatatatatatacacacacacacatatatatacacacacactatatatattaatatatgtatataatgtgtttgtgtgtgtgtgtacttgaatatcagcttttagtcaagctgacttctgaccatagcatccttttaaaaaatacattttaataaatcttttattatcagattatagccaagaaaaacagctggtgatctggcttttttttctttctttttttcttttttcttttttttttttttgagacagagtttcactcttgttgcccaggcgggagtgcagtggcacgatcttggctcacctcaacctccacctcctgggttcaaatgattctcctgcctcagcctcccgagtagctgggattacaggcatgtgccaccacgccaggctaattttgtatttttagtagaggcagggtttctccatgttggtcaggctggtctcgaactcccaacctcaggtgatctgcccgccttggcctcccaaaattctgggattacaggtgtgagccactgagcccggccaaacctttttttttttttttttaccaaaggcactctcctgagtagcaaagtcaaaagtcacaaaaatattaaaccaaaaggagctaattccctaatcaggagtaaaacccaggccctggtggtaaaagcacaaaatcatagccactagaccacagagtacaaggctttcttacttttgccttttttttttttggtttttaaatcttgctgggtattcaaagcagcctgtttgagcatttagaggattttaacttgattcagatgtaatctcagcaggagtgctgcctcagctcattccctggatgttagcatttcaacatcagcatcaataattcgtcttggtttaaagaaaatggtcggatctacatgtttataatctcagtacttttattttcattcatttccccattttagactattagtcttttaattacctgttatatcgacctaagcaattgtcagctaggcaacaaaggtgtattttggagacccatcttgttcattgggtggtgtttttattcatttatttatttacttaagtttctctttttttatcccccatcccctcggtgttttcacggacgggttttttagttgagcaaagaatagtttgagaaccaggcattccccagaaccagaagttgctcacagcgactccaggactggagtcactatgtcacaagaggagtttcagggagggagtcaaacacctagggaattccctgtggtggtggtgatgggtatgtactgtttacctcctgaatggaggcaattgtctcaggatttcttttagaagtttttttttcagtaccattggaagtaggtctttaattatctggttccaaaaccagctttttcttattgtgcacaaaaataaattattttaggtatttcaaaagatccccagttttggcaaggtttattgtttgactattctatgttaggtgggtccactttcctagatatttacaagaagatgtccatagtgttatatataaattcagctgatgtttaataggagttggtcacagatgaaaaatgaagcccaaatttcaaaaataggaaagtatctgcaatgttttaaagcatgctgtgtaaaaccagaccactggtgcctaactggcaatccttacccaaacctctgctatagacaggggcaggaaaactttgacctgaagaccctggtcctgagacaggctgaaggaggcagttctctgcagagctctttgcctgaatcatctgtctaaagacagagactgaagccctcattcgtaaaggaaagagagagggtttgaaaaacagcccaaataaagtatagacctttgaccaaagactgggaggtctgaattcaggtgaactcacctgaaacacccaataggacttctgaagatggaatgtttgtgctggtaccaagccctgctttcagagaaaaacaccaggtggtccgggggagtcactctgaaatctgccaagttatgccaaatatgtcgacctaaaaggaagaaacgaaagcaaaattaatataagtagagagtttatttgggccaagcttgaggattgcaacctaggagcatggattcaagttgccctgaataaacactttgattagcagcagttaggagtggatttttaaagggaagaaagggggatggggagttaatcttgacttcttaagaagtatgtctactgactaagaaacattgatttaagaagtttaagtccaatcttgcaaaaatctttcaattgtcaattgtcatcacaagagaaattttgccttgatatctgacaaaatatattcttctgtggctggatgcagtgatttgtgcctataatcccagcactttgggaggctgaggtagaaggatcacttcagcccaggagttcaagaccagcttgggcaaatagagagaccctgtctctacattttttttttaatagccaggtgtgatagcccagttattccagaggctgaggtgggtggatgccttgagctgtggaggtcaaggctgcagtgaactgtgatcatacagtgcagcactccagcctgggtgacagagaaaaaaaatcaaagttaaaaatttattcttgcgttagttttaaacaaaatgcaaaattaaagcatttgtagagaacttgtagctctgagaatataactcttgactccagtttaagaaaatgggtctacatcattgctgtagcaatttaatctccttgttgggaagaaccagagttgtttgaatttactgagaattttgcttccaaataataattttaagccacgattcacactgtgtcactgtcctgctcaaagcgctttagtgatgtcttattcccacctcctaaaaaagtcacaaactcctcagcatggcagttgggatctttcatgacttggcccaagattgcttttgtctactgtctatggatgcatcagcaatgatgtgttctcttctttttttttttttttggagacagagtctcactctgtcacccaggctggagtgcagtgcagtggtgcaatcttggctcgctataacctctgcctcccaggttcaagctattctcctgtcccagcctccccagtagctgggactataggcatgccaccatgcccagctaatttttgtattttttggtagagacggagtttcaccatgttggccagatgggtcctgaactcctgacctcaggtgatctgcccaccttggcctcccgaagtgctgggattacaggcatgagacaccttgtctgaccctcatttctaatcatgtgctgtacttgccttcttccatgacttttcacctctcacttagcatctgcttccaactgtttataaaactactcttggtgtaaatcaatgtccagttctagtatagttccctttgtaaaatatttttcttttagtattggactggaataaaagctgagcactagaattttagaaagcttcttaggagattctactgtgcaccccaaattggaaacaaatcccctaaagtggttcttaaaatatgatcccgagacaagcatcatctgtatcatctgcagacttgctaaaagctcttggatgggcccagcagcctttgttttaataaacttggtgggtgattcttatgcacattcaaatttcagaaccattgcccttggccaaaatgaatatcagatcattctactagtctttaaatgtgctttatttatggttattacagaattcagtgttcctcgaaaatgatacagctacgtgtacggatttctcatctttttattctagtataagctctttgatacatgtactacgtttatcttggtatcacccattgtgtttatcaaagtattttgcctaagaatctctccacaaatatatgtgtagtgcattaaagcttttaggtataactagcagggagaaatgggaatgtgacataccagtcacactcctgtggatcttttattaaaaaacagaaatgtaatatatactcatatcaaacagacagctgacaagatgtttttaataattatactttattaagaaaatagcatgagcattaattacatgataccctgttctcctaagaaaacattttaatgtctctttcttggagaaagcatttttaaatgtacaatctccagtacaaattaggtgcctttgtcagaattacctgctgtatatcctccttgttgtgtattagatttcctttgcacaggaagacaattatcttccttttgactttcaataatgccttctcattttgcttgcttcttgggtaaggtgttgatttcttcctttatttttcctgttaatctgtaaccacaaatactcctaaattgcttttgggcaatctgctcttggttttacgggccacaagcccacactgaccaattctttataggggctcttactgcctgtgttaaaaagtcaccctgaggtgtcagtctttccttaataaccgagtgcctttttcacacttttacttcaaaccatttcattctggaaagtaatttgtttacttactagttggacctcagaagatgacttttttttttttaacttacaacaaactctttatcattgtcttagaaagcattgtttagattataagtttcagaaaccaaactcaaactggcataagtaaaatactgacttggctcgtctaacagagatatccaggctgtggccacttcaggcacagctctatccagggggattacaggtcctgaagataccatttctctctccatctgctgaagtttctttcctctgtgtggccttccttttggggtaaagatggttcctacaaacgagtgttctattctcagaagaagaagagaaggtgaaaccaccattcctttctgagagccccagtgaaggacctggggaggcaatcagccagacccaggccacgcacccattcctgggtgggcttggcactagataacaccaagggactgtcaccaagagtgagggtcagaatgctggacaagcaaagtcaatgaatttcactacaattatggagtttgttccagagatgtggaacaaactccataattggagatggaacagacgtggaacaggcacgctgttccagaacagcaaagaggtagaagtcattttatacttaaaagaaaaagataaccacaatacctaccaaattttaagaacatattaagaatcagtcacaatgttaggtgagttatatggattgtttgaaaacccagtgaattccatattattatttccatttttaaggggaatttaaggttacccagataatcagtgggaaaatcagaatttaaatcaaagacattttctctttattatccatattgtttacactgtattaggccatctgagtcaatggagtgaacagtagacggtgagaacaaattctgtgttactaacctttggtgtcagatattatcagttgcccccaactgccctgattccgttccatcaggtatttatttatccagtcgatgttcaggagatcttcattcttgtgagaatgagaaagacagaaaatgaacaagtaaacaagtccataaacaaagtcttttagatggtggaaaatgcagtgaagaaaatataaaacaagctgatctactaggaggttagtaattcagaggaggtggttaaggatgacttaaatactaggtgatatttgaacattagcctgaatcataaggagacagcttttctaaaatcttgggtaagatcttcccagagccagtgaatggatgcactcaagtcataggaataagcttgtcatgtttgaggaagacagagaaggccagtgggagtccagcacattgagtagaagggtgtgtgttgagagggatggacagagccccagtcatgtagggctgtatgtaggccattgtaggaagctgctattttattgtaaacagaatggagaggccactgaagggttttattgagttattgctgtccctttgtatttcatgcatcgattatcagtagtcatgaaaatagttaaagtttgcacaaggcagtagtgaagccggccaaggccacatggctggcctgaccattttcccccggtctcctgtggaaacacgaacagagacaggcacctggagagcctttgccacatgcattatagccttcatcctagttgcctggagaaaacctggtttactttttgaccccagtgaataaactgagctagcagaaatgcttaacagctgtttccacaatataacctcctcctgggccaagtcagttgattgatttctttctatttattgaagagtaacagcatgtaaagtgtttaactctggaattctgacgaagaaattcaggggatggggcatttttatcccctccattcaccattctcaccctgttttgttttctcttttctctcttacctaagtctttcctttttagtctctctggccatgagctatcatccctatcagcctcatttttccctgccccacttaaaggtaatatatctttaccaaacccattcattttaatgtattctgcttattggcaggaagcatcaatgcatggtaaaagcacaactgcatttacatttacagataaaatgtaaatgtgaacagtgttcttcgtgtcaaagtattcctttcagatttgaagggtttagattattatgtttagaatattattttcagtataaaatagtaaaatgagataaaaatcaaaaagagaaacaagttatatgttagctactgagattatttttgcatgaagtgacaactttataatgaattttgggggcttatgatttggcatatctgaatgtaaattcatcatctttactgaaaggaaagaagaaactttttcaaagcaagtattctctgatggtgtgccttcatcaatgaagaatggcacattttctttaaagaaagttttaaaagccaaatatattttatgtaataagtagaaggaagatagatattttattcatgttaacaagaagtgctacacaaatactaatctgagaaccatgtattttggacaattttttcaactgagaaatgtctggccagaagtgtgttttcactgttcgtatgatagtgtatctatgaaatgtgtcacttctcactacttctgcaggaaaaaaaaaaatcggtagctatgctcccctgaaatgcgatgaatatgaagaatgccatggacttactgccatttcatgtctttaagtacagctgaggaacagcatgagaaagtgaagggacagtgggacttcagcttccatgccctgtctctgagtgtcggcacatctccccatccctttctctttcttccctgagccctcagctgtgtgtcacatcccttgtctgacaacaaacagtatatagaaatagaagattttgtgttctattcataagaatcttggaaactacctgaaaccacaagagtatagcttaaaatctgagagcaagaacaagagagagagaaagagagagggagaaagagaatattaattagcatgtaaagaatgcctttgtccttaaattttttttatttatagagctacagctattagagatattacgtgatgtgatttctttttaaattaacgagaaacattccagatgcatatttatctcctctatagcagttttatttttgttctctcttttagcagaatttcattttttaatatcttaagatgcattcatattttgctttctttaggactagctgtaaatcaaatgtgtatattcatattcaattcatttctgtgtgcagacttggaaatcagagttgtcatagttgtatgttttgttttatttgagaagtgattctcccctggcaggagaaaacaactaaatctacaaagtatttgaattagtttataatgcttgaaagttttgtggatgtctttcctacacatgaaatttatgaaagatctaccatttacaaaatatgttatactcaaatgccaaattcttgcacaaagtaaaactaaagaactgcgggtacttgcatgtattcaacaaatactgtgctcctgtcaagtaccaaggacagcacctcgtgcccgagagcctcacaagagttaaattgctgcttgccaagaacattttgtctgtaggttatgataaatattaaacaaaattttaactaataatttatagcaatatgtgaaacaaattatgtaggggaagtgtaaagtgctatgcatgagtacaagaagataatcaatcttaagccattggggatgtggtgatgtttgaaggtttcaggaatgaattagaggaaccaatctttaatctgagatctgaaagaggagttaaggttatagtatttgggggagaagggatattttctagcagagggaacagcatggagggaaaatcctaaaggggaaaggagaaattgtagttatggaagctgggattcaagaggaagagggacctggaacatacttgtgaccagaatcaacatattttcttttaccttctttgcatccagctcttcctcaactccttttaacccagccttcacccaggacatgcttcagctccatttctatttgctcaaatagtgtgattatttacgactgatttcaaatgtatcttcttgcagaatgcctttagttatagcttcagctggaattaatcaatcactctcttctttaaaacctaataacctctttgatttcatgtgttttaattggacttgttggttatttgtctgacttcctcaatacatattgaaggcaaccatgtcctagttcctcactgtgcttggtacaaagttagtaccataaaaatagttgaattgaattggaaaccagattagtcataacacattggccatcttttctgtggtattatcattttattgaatttattccagtatccagcctactttgttttatatccaaaaaattccaggccatttgaaaggtgcagatgctgactctgttggagacatctcaagaaaattcatcagcatagccaaggttcataaatttcacatgaggcaagatcagataaagttgaaattgtagtgacactttgaaaacaatatatatcgtcttaatttgaaaaatagtataattctatgaaaccttgccaccattgctttactgagaatgtgatataactgcacgtacatttatgcatgtagtattatgtaacttacctggactggacttattcttattttaattcagactaattcatttgaccttaagaagttgtaattataactaatatctattttactttcatgttagaaatttttaatgcaatttttaggtaagggcaacaaatgagattagaattgtttacctatgggttttttatttttgtgataagtccttcgttgtgataatatttttaaggatgaatagatattctggctgacaagtaggagcagatgaaatcatcttcaaaaagaattctttgaagagaacctcctctgtctcaaagctaatacaagctattgttttcaccaaagcaaagggaagatcaaagtatttgtgctctgaatttcctttggaaataaatgaaagttggaagtgtcaaaggaaaaataagcataccccaaaaagatagttagggaaatatactcagtgaggaaaaattgtcagtgcttatggaagcaagattgatttttatttttaactctgttgacttttctatctgatcacaaggacaatccatcttaaaaatatattcaaggcatcttagtcaagctccttcagtgatttttttcctattgcttttgctcagctcctttttgctgacatttcagacttcttttgttttgtggatgtaccatgcaggtgaatgagtgattaaaaattatactttcaaagctttatccctgcacctgtcaggaaatttagcttgcagccagaacttttgatagaaaagccatgattttaacttagaagaaattaatatggatttactgaaactcatgatttgtgtaaagaatagatctttagatttataaaattagctgcctggttaaatgctgccctacctccccccaacaccaaacttcaagtaggggcacagttctctgttatgtagattcaagtttctgaaattctgtcagtcatttaggaaaagaatcaacacattttggcttggcttggcttttcataattttagttagaatgacatccatttcactttccttggccaattcctacccatcttttctcacatgcccctaaatttgccaccactttccccactgaggtcttcctactcccctatatcagtcttttaaaaataatcctattccatgattctatggtaacacttaacccattatgctttatatactttttgtgtctgtttctcctttcaccttgtcttttttgttgttgttgtttgttgcttgttgacaaagcagctactaatagatgattattaataaatgtttgtatgtacgtgaatgctcaaaagccagagcaaccaaaccaggacacaagatataaacagaggcaggttcttgactcacatcatgatgacttagcagacgggtcttcctagctccttccattctgttttgtgaactattctttatggcttaacaatattgtgcacctgtatgcaagcgcaggaatgtgcacagcactgaggatacagagatgaaccggacaatatgctcttgagaattttaaaaggaagagtcaatgaaaccaacaggttattttgaaatactctaccgaacgccatggctggtgtaacaggatgcctgagatgcccgaggagcacagaacaggagcatttaggaaacactgaggagtccagggactgggggcagttctgacagatgactaccggtgggccaggcggagagcagtggagaagctgtttcagagagagaaaacaatacaatccaaggcattgtgggactataatatgtgggaaggggctgaaagtgaagttgggggattgaggggcaggagctggccacagaggacctcagatgccagctgagacacttcagcagaagcctgtaagtgaggcaactggcttgtgacttctaagtggggcaggccggggtcggagaggatgcatattctactgtgcaccctgtggtaccattgaggacaaattaggggtgaggagaagatgagccagagatactggttaggaaacataaactatttcaggcaagcgataatgagaaccctaagtagatacaaatggagatggagtggaagaaaaaggcagaaggaactgaaatagttggagtcttactgttacttgaaaacaaaagaaagtatttctggtgtgagactgtgaatcaggcccactaaagtgttcctgctctcataatgaatgttcctcatgcaatatttcacatggtcagggaagaaggattcatctgtatcaattttaaatgccatcaaaatgcattagcagccatattctttgtatcaaattccaaaaaagtatcaaaatatatagtacataatattatatatggtaacatataattttaactgtataatgataaatatatatctttaaatataaaatgttttaatagaataaagcagagcttagtatattttcttttgtctatttaaatattaagacatttcagtcaagcagaattgaaagcctatagttctattccattcataatatatttcatatgattgtaaaggaaatcccagaactttatgagattgtatacacattaattcataatcccagaacttgggagattgtgtacacattaatatttgaaaaaagtctgggatgtgcttcagtttctgtaaactttgaacttcatgtacttgagataccagtgaactcagatgtaaaattagatgcattcagaaaacacaatgagagatgacagaggaacaaatccctttctagtcttaaggagaagtattttcagtagatctaatatgacattcattgcatatcttctgagccaggtatgaaaaaatatagaaatatgcttaatagaccaaatgacatttaggttatggggagaaagttttcagtttgttttgggtaaatatgaaatttctgcagtggggtgagtgagaagacacagaaagttaatgatgtggaatttgctgcattaatatactggaagatggagagttggccaaacttggattccaatccccacccttccattgactagcttgaaaatattaagaaattgcccccaacaggcttgatattaatattttcaagcctgttttctaatctgtccattcaggctactgatgcttcacagactgtcatgagcattagctgagaagaggtatgtattatcctcagtatttgcctaacctatagaatggctaaccacagagatttgtatctgaagatgggtttcatggaaggtagctttcgtgagtttttcggcagagcaacttattcagagaacaaaattaacatcaagagtttctccagggtgcttggtgttgaggcaacaggccccactgagtagaaaagacgtagaagtaacttgcatgaaatagctatgcaaaatgcaactggaagagccttgtttgcacgcagacatgttcctgagaataatgtagatactgcatttgatttcacactagtaaacattataaagatagagacaattgttaaaaaaaaaaaaactatccagaagaatgtacagcttcagctgtgcttccagtcctggatttagcccaaagacagaaaagataattgcccagatctgctgagaacaatgatacaaaaggcctacaagtacctccaatgggagtatttgaagtaggaactttttatgcaatgtgtaattgaaagctatttgaaaagtatcacagtcaggtctgttttactatgccttgaatgctaccagattctgtaggcagttaataaagaatggctattaagtcatttctgttattttgcaatcaaaaataaaagttgtggccaggtgcggtggctcacacctgtaatcccagagctttgggagactaaggcaataggatcacttgagggcaggagtttgagacctgcctgggcaacatagagagaccctcacccctcatctctacaaaatgttttaaaaattagccccatgggggcacatgcctatagtcccaactactcaggtaggtgaggtgggaggatcacctgagcccaggagtttgaggttatagcgagctatgattgcatcattgcactccagcctgaatgacacagtaaggccctgtctcttaaagaagttgtaaatgaaatagtaattccccaaactcaacatctttccatgtacttttttttccttcaacttttattttaaattctgggatacatgtgcaggatatgcaggtttgttacataggtaaatgtgtgccatggtggtttactgtacagatcatcccatcacctaggtattaagcccaggatccattagctattcttcctgctgttctccctcccacaacaccatacaacaggctccaatgtgtgttgttccccccacccccttgctctatgtgttctcagcgtttagcttccacttacaagtgagaacatgcagtgtttggttttctgttctggcattagtttgctgaggctaatggcttccaccgtcatccatgtccctgcaaaggacatgatctcattcctctttatggctgcatagtattccatggtttatatataccacattttctttatccagtctatcactgatggacatttgagttgattccatgtctttgctattgtgaatagtgctgcaatgaacatatatgtgcatgcatctttataatacctttgggtgtatacccagtaatgggattgctgggtcaaatggtatttctgcctctaggtctatgaggaattgccatactgtcttccgcaatggttgaactaatttacactctcaccaacagtgtaaaagtgtttcctttttctctgcaacctcgtcagcgtctgttattttttgactttttaattgccattttgactggtatgagatggtatctcattgtggttttgatttgcatttatctaatgatcagtgatgttgagctttttttcataggcttgttgtccatatgtatgtcttcttttgagaactatctgttcatgtcctttgcccatttttaaatggggttgcttgtttttttttcttttaaatttgtagactctggatattagacctttggcagttggatagattgcaaaatttttcttccattctgcaggttgtctgctctgatgatcatttcttttgctgtgtggaagctctttccatgtattttaataaacactgcatatgccaatatttaaagtttttaatatgagaaactaatagctagtttcttgcttggtaatatatctaatccaggttggaatgacaacatagtttacagggaaaagttgacatttaaacttttgtgtttgctttaataacgctcttggtagaagaaaacaaatctcatagagatatgcttacagtaaggaaggaagagatttcaaagcaatttctccataaaatcccagtgaagttgtattgcaatcaataaattacatataaaaacatcaattttatttctctaatttagttaataatgttaaactatcatttttaaaagtctgattaaaaattaaaatattgcagagatgacagattttaaataaagatggctacttaagttttttttcagtttttatacacttttggtttataattaacaaacattacttcttgtgatgtggccctactgtgcatgatgatggtgcaaccgcatgtgacttgttgctctagtctctcctctggtgaataagacaaaggcttttgaatgtttgtgtgtcagggcagtataaattgttacacagtttgcattgaccaaatctagtttttaaattcaattttagacttttttagaaatctaatttctaaattctcacttacaatttctttactgaaggctgcaagctgtattttgagtgacaatgagctaggggacataggttgggattttttttttaaagctgctgagagatgtagctttggcctattatatatctgtgtgtgttctttaaacagggaaggaaattggaaattgagctgagagcagattctgaaagagggagagcaagtttgctttacgcccaggtgaaaaaatgtcattgaaaacaacactggggaaggggtcaccctatagtagaaaagcaggttcagaaagagccagtgttagcagtttctgttttcccataaaggaaaggctgcacaagttgggttcatccaacacttgccaggcaaagaaggacacgcccctgactcctgagtactagatgcatatttcctgcaggaaggagctttgtctcttgtctgctgctctacagaatattgctttgtaccttaatagatgctcaaaatatatttgttgaaataattgactgaaagtattaagaacacatccttatgtccagaattcataatgtgtgattagtattttttgttattctcatgtttctattgttgaatgccacagggtttcaggaaggttagacagattttacatagtttatcaacaataagaacaagtgaaaaaaaatctcttaagtctcatatcatttacctcctcactgctggcagaatcaatgataaataagattgtctctatttctcctcttcccaccttttgtctattttctcctcctctgttttcttctttcatcaattgtttccttcattcaattactattcatatctgctgagtaatctcccaactgctatttctattgttcatacctccccctcttttcagggaattaaatacctttccagacttaaattctatgatcaaaatgggacattccttttctcacccttaagattctccttatgtcttaaagactcttcattcactttgcctctctaatacatccttgtcctagtgtgtttttccagcactctgctaaacctttgtagatcactcactgcactggattataattatatctttattttcctcttttctaaaccacaccattggttctttacagatgaatattgtcttatttttacctttttatccctagcacttggtgctgcctgatttatgaattatttgtttatgaacaaataagtgaatacacaaatctggagttcagagttatgagtttacagatttgggtccctaagaggaaaacaagatggcctgggaaaagtttcaaagtgtgaatagtgaggcaagcagaacctaagaaaacagatgcttatgggataggcagtgaagaggacacagagtaggtaaagcagggaaagtaggtgagcaccaacaaggtgccagggcaaagtcaaaagggcaagggaatagagaatttgaaggagtgggtgctacatttaagaagagattcaggacttgatatctagaaagtatccactggattggaccaatgcagtcattggcgtccttagagggaatggttctcgtaggatgacagtccgctgaggagagtgggttgaagatttggaagtgggattagcagattggacttcttcaggagaaatttgaatggaaaaggagaggagttattggactatgtctagaaggggcagggagtgaatgaatatttatttcttcactagaatgaacttggtcaagcttacatttctttgcagggaagattcaatattgcatgagattgtcccttgttattcattattaattttcattttgtctttaaattatttacttttgattcattaacaatatttactgagcaactgctctctgtcggttagttgcctaggtattgagcttaaggcgataaacatgacaaagtccatgtcctcatgcagagtatgttcaattccagctatttccttaagattcacctttgatcggtctgttcatctgaggagtgaagttaccaaacgggtatcaaaaggaagcttcaattatttgatatatttctcttacggtattcacaatgtctctaaatactgctagtgacatctttctaatgtctgtatatctataattctagccatattccatttttcctggccttccctctgtagcagatgtctacagctatttataagatgagactcattcctgaattgtctgccttaatatcttagaagatggtgacaattccatactgcttcagttgatttgaattaagcactttggaagtttaaaatagggtagagacctggtttgtctttctcagtcagctatctttttcctattataactacaatttataagataaatagtgaaaagggatgtatgttttgtgatggagaaaatttattatgcttggcaggtttttttcttgagcaacagctaccatccatgatggataattttcagtattttttgatatgaaatcttacttgaaagaagatacattgtccttggaaattgtttggagctattattcatcaataaattttggaaattaattatacatttatttattcaaccaaaatatgttgaccactcactgtgtacctggtgctgtgtgttgggataaaagagaaactgtacattgtctctgttttcaaggtacttatggtttagtggggaaataaaaatgtcaacaaatccttacgtcacagcagatactgaagcagcagatactgaagttaagggatgtgccaggtgctggggtatagggaaaaactactgacttgaaagatacaggcaaagtttgagtgaggaggtgatgtggaagatgagtctggaagaatgaataataataataagcaatgttgagtgacttactgtacttaaggatcttgaattttgccaaataaaggaaagctcagcctcttattgattctagtgcaaaagtatataagctaagagttaagcacttaaaatctcagtataatagcatgcaaccatgtttagtcttttcttagaactgaacaagaattttgctgtgtcatttcttgctagagtaatttataggtaaaacggtgctattgtcatcacacttattgtgtgacttgcaagggagaccctgagaatatttttctcttgactatagttgtctctcactgaaggagagattggtaagcatactcactctctaattttaagttctgatgattgttaaatactttcctttaagtattatgtttcaatgttaatggagttgtagctaatatttataagcttaagagtaattcctcctgcatataaagaatcctcttaaaaatgtacataatgatttaatctgtctttaaaattaaactctgtaatacattttcctctgttagattttatgtatatatatagtttcatagtgtaagtatagtaacttcaaccctttctagctctcatgaaataaagttcgattcagttcaatactacattaagaagtattaggtacatgccatgtttcaggcactcattgccttgcattatgggattgcaaatttaaaaaatgggtctctgcccactgagagtgcataatttagcacaaggaacaggatgagtgcaactcagtataatagagctggataaatttgtttcagtggagaagttctcaagatgtcacatttgagctaagttttgaagaatcagtagagttttgccaagcagaaggaggtgagagggaattccaggcaggagagaaccatatgcaaaaaggtgggaaaacttgcatgtggatggtgtgttgggggagatcaagtaaggacatggcaaggcacggtgttgagagatggaatgggaagtttgttggcaaataattaggaaggcccttgaatgcttggctaaaatgtgaggcttttggcaccggagaaatttaaatgatattaaagcaaggtaatgaaatgattgtatttatgctttacaaaggtatttttagagatagattggagttaaatagggtatacagagaaaggacatggtggaaaaagatccaacccatgaacttgctattcatagtgtcattgtaagaattttattattttgaacctaaaaagttattctttggtatgaaaatgcagaattatcttgaaacacaattatcaaatgaaatagaattattttgaaataattggcttgactttaatgtttttgtatatgacttgcctttaatgtttttcctaggtataatgagatgttttgaaaggatttgcttcttttcctgtccatttccatggagataatattccctaatggcaggacattgtgttgccattcgaaaatatgtgtatatgcagtattttaacagtaggtggcatccacagtgatatgtacatatcgcaggagcaggagaaggaatgtcaccttagcagaatcataaaaaagagagcaaaaatcaaagtggagtgacggagcatttggaaagcagagaataggatttcaacctagtcaggtcactaatggaggcttctcaaaaaggagaagagtgagagtattgacagcagtgaagagtagagaggtgtgtctccactacctctcacaacatcattacattttcaggggataaaagaagtcagggaaatgcttttttaaaaaatatggtaaagtcttactttctatagtcacatttattgtcttgtcttgttaatattctagcaggttttttaaacactcaagtagaaatgaagctgttaattggtaataggcagttcaatgacaactgtgaccctaggtgctccatttttgtcaggccaaaagacatccaggcaaatagaagattatctcattattcagaatttctcttcacgtaactttaagtaaaatgcataccagagaagtcaatgctgatgcttatgggcatggggaaggtaggtgggatagaggtgcaaattaatattctcccacctttccactcatcgtcgtgtattctttaaccacatgtggccgcaggttgaacctgtctggtgattttcatttatacaagagatcagtgtctcttcatgagaattttttatagatttatattttcctaatttaggtgctgtagtagtaaagttcagtttaacagtgaaactcatagagatacccagtcacacctgtagtcaactgctgtaccattataatgacatatctaatcatgtctataaatgatatctgtaattgattaaacactgttaccattcttttctgaaatgcagaacacttgatcaaatccaaaatacttgtaaaatgcataaaagcaacctaataccgtttagaatattttgggataggaagcaatatcaggagaagtatatatctgtctgaccaagtgagaactttctgtaaatgattataaatatggagaatatctcataagcaatggtctggagaagaagcaagagtatagcagaaatctgggctgaatagtatagttccttcttatttacaaagcacgaggctcttaggccttaatcatctctcttaagaattggatatcctccatcatgccaagaaacggggggaaaaagtttgccagatgagaagaatgccatttttgtcactgatttaacctctgtatatttcctatgacaaaataaattggtttaaatattttaaataaggcaattgcattatctttccttgtgtgaaaaagtgagcatccctggagcaccagcttaaccctattgagctaggattctcattacttacccatttgataaagcactttcctgattcttaaattagtatgttaacttacataactgtctaaaccaatctcagcgttcctcagactcctttactctgtctcacggcaaacatcagttccctatttatattaccacagtcttgttttgaatgctgaatgattctttcctttttagggtctctcgcagcattcagttctctcaattttcatatttgatgagtcatgtatttcctctcgttcatccttttgtttttcctttttctgctgatagctaagtgttaattttgtagtgcttcttcactgcttaccaagttaattacaaatctttggtttgatatttaatttccttataatgtggattcagcctacttagccagccttctctctctctctaccttacatgaacccagtcttcagtcacattggctccactcattctgtacctgctctgttgtttagggcattgcttccacttgaaatacagctttcccagtttgacctgtcagaattctacccatgcgtcaaagtctaactcctatgccatattcttatgtggccttccttctgtagacccaatgactctttatacctttaatttcacctatcacttccctgtcttgcattataaatacctgtgtatttgtcttcttgattagatggtatgcaataaatgccaagatttctttcattgtagttttttgactggatatatttataccactgcttactataacttacattttaccacctctctcattggtattttatcctatagggactaaatgaaagaaatagtgtcataatatatttattttacatggcagagttaactgacgaagttatgacagtgaggcctgctgtatgccagcatgaaaggaaacatactctgcagcccatataaaatagtagccatactttcaaaatgtcttaataagattttctatactcagacaaaggaccatgatgtcattcttactaacctcagaagcataataaaatatggttagttttggcattcatcaaattttgccattaacttttcccataaaatttgattaaggagcattgcaagacttttttaattaaggaaagtgctttggatatttatgtcctgtccacagtttgtcaaattctgaaagatactgaagttcttcagatacctgtaaacattatgattggtaaaatatctgaacaattttttctgatgagaaatacctgaaattagttgtagcctcaagtataaattcttattttaaaaaataattgaattaaaaaattattggctatttatattacataggagtatcaaagtggctttattttctggttatttctctggacttcatccatgcatagatatgctttataatattttattgctttccattaccatggattaacaaatcttaactgctagcacaagtctggatgagccaatatatctaattctaaattttcagtaaacgtttgtcttttaagagtattttaaatgttttaaaaaatgtgtaaaaaaagcttaagaaacactggatgtttttctatcttagttctgctatatagcagggtttttttgttttgttttgtttttttagatggagtcttgctctgttgcccaggctggagtgcaatggtgtgatctcagctcactgcaacctctgcctcctgggttcaagcaattctcctgcctcagcctcctgagtagctgggattacaggcactcgccaccacgcctagctaattttcgtatttttagtagagacagggtttcaccatgttggtcaggttggtttcaaactcctgacctcgtgatccacccacctcagcctcccaaagtgctgggattacaggtgtgagccaccgtgcccagcctatagcagttttaaaaaggcagattgttgctaaataaagtttcacttgatgtgggctacattttagacctttaaaagttatttctgtaacttatttttttcctttaaacattcgcaccattgaatagttgaagacttgtatcaagtcaatttaagtgaatgtcgcagaatcagatcaacatgatctaaacagctcttaactggctgaatcaatgacaacttttttttttttttaaggaacatctttttcagaaagaacagagtgctccatgcaaccagctgtttctgtagcaatgcttcccttaggtggtactgatgagctatgggctgcttttacagaatagttttcttcccagataattcaaggtttagaagagctgcacctagttaagccgtttaaatgacagtcttctttcctttaccctgaaaaacattcttttctttttaggcctaaattctacttgcacactgatcatgaaattacatagattccaaatatctacttaatttttatttcttaacatgtgtcataaaatattttagattacctccacggaaagcaggaaggctttggccttaatattatgccttatcatggctaaaaattgtccaatgatgcttcccactgtgtgtacttgagttatagttattattgtctattgaatgagtcctatatgcttatcacactaatgatcatttcatatgcattaatttaatcttcaacatcatgctatgtcaggcaattctaatatccattttgaaaataagaaaactgaccctatgacaggctaaataatttgactgtgacttgtatactattttttaccacattgctttttctaccaagtttacttgagaaaaggcatgaagacatctgatcgaggtacttctgtgcgttccagctttgagatgccgcctctgttccaagcatgtaacaaatgctagataaaatatgaggataggaaatgcaaaaatcacagctgggctcaaaaacagtttgttatctctgtggcccaagaatggagatgaaatgcaagtgatgagttgtgggctacactgaggcctctggcctgctggactctggaggtggacagagggggctgggacagcgtttgcagagcaataggacatgaatgttattgtacactaggtggaaatccagagctagcctcaccatttgaaaccaggtagtagaggactccacttctgaatgaaatatgaaagtatgaaaaatctgctgatcactgttgcttgagactagcttttgtgatgcttggtgtttaagatgggaagaggaagttgttttaggcatggccctgaactactgaaccaaactcaaccaaatcactcactagtttggggacatgactggtgataatccagatgtcaaaacaatgtcatagatcagatatgcaactgcttacaaatgtgtgatcctgggcacaaaaccatttttaagaggatgatgagagagagagaggatggagctatatcaaaaaaactaaataaaaataatttaaaaaccatcccacttcaaatgaacttacaaaacataattccaaagcacatgaggaaacctagcagtaagaaagacagccaacaaaatggaccattaaaatgaattttccccagaagatttaatagatcgaaacaggcttgcaaagtctattttggatgcccaaaggaatgccattgtattagtctgttctcatgttgctaataaagacatacttgagactgggtaatttataaaggaaaaaggtttaatggactcatagttccacgtggctggggaggcctcacaatcatggctgaaggcaaatgaggagcaaggtcacatcttacatggaggcaggcaagagagcttgtgcaggggaactctcatatacaaaaccatcagatcttgtgagacttgctcactaccaggagaacagtatgggggaaacaacccccatgattcaattatctccatctggccccacccttgacacgtgaggattataacaattcagtgagacttggatggggacacagccaaaccatatcagccatccataaagaaacaaaaatgtcaagttattatttatttacaaatatttattaagatcatactatgttccacatactattacaggtgctagagatgcagaaatgaataaaaataaataaaatccttagctactagaggatttgcattctagtggggagtttacattttagtggcagaaatgaaataagaataggtgaacaggaggcttccactgtatttgtaatattttatttatttaaaaggtgaagcaaaagaacaaaatgctcagacttgttaaagcatggtgcataagtgggttttcacagtttaatcctacataagtacctgtacaattgacgtgaaaggacacacagacctttcagtagaacatgaattgttggacatagcaataatatttaaaacgtccagtcttctggcaatgaggcaagttccatggccatttacctctgtggctcccaacttgcttagggtaaaaccttatcagggcctgtaaggtcctggggtctacctctctggcctaacttgctctttcactctgccccagctcctgccccattgcatgttcctcacacaattcaagctcatttccttttatgatgacagcactcctctcagattatatctctttcagatactggggaaaatcttgaaaattttaaaatgatgtctcaaatgagacctttgattttcatcatggaaataatccctcagtcttttattttgctcttgatctagtgcttttatagacactaattcactcagctagttagaactgtatttcatggcacttgatggatgttccactttgcagtggctcatgtcaccatgacccagtctggtgggttgctgcctggggttgctgtagcccccacactcccatcagtgttatgtgcttaggctgagggaactgcagatggtacctggaaatctcttaggaatgcaaagagagttataatgagtaacagccaatcttggatgctgagttgtttacaaaactttttgaggaggattcaatatgtattttagacaagaaggaactgcttctcaataaagatgagcataattgtcattttggaaattagtctttagagctaaagcattcataatttaaagtgtaaagcttgaataatccatgttactttattttaaaaagacaaaacactattgctagtaatgggaatatcactgtgtgtgggtgtgaacacatatgttcagtgggactcaaaaatcccacacagggtatagcgttgtcgctcactgattttgcccaattatactgtgaatttgataataatttaccaacactttgaagtcctacagatgctctgaaacaacaatataatcagcttttatttgaatatctaatatgacaactcactctaatatcatgggacttattttgttccaccgacattaaaatgtcacattctagaaatgtgaagtttgtcacctcatcaaaaatagatactagatggtgaatttgaatgtatttaatgccattgaactgtatgcttgagaatgctcaaaacggcataatatgcagctgtaaaaaacaatgagattatgtgccttgtagcaacatggatggagctggaggcattatcctaagtgaaataactcagaaacagaaaatttaataccatatgttcccacttacacagagatagaagcaatagatactgagggttgaaaagcaatacacacttttcacaacccccttcccacctcttcccacttgtggagaggtaggaaggggatgagggttgaaaacttacctattgggtacaatggtcactatttgagtgatgggtacagtagaagcccaaatcccaccattatgtaatatatccatgtaaaaaacatgtatgtataccctctgaatctaaaataaattaaaagaatggttaaaatgctaaattttgtattatactgtatacattttaccatcactaaaaaaaatagatactagatcaaataaaatgtaactattttatctccatagatgattcccttaaaatttctgtttagtatcgagccacaagcaaagcaaacacagcgataattatttggaaattgctgtaattattgcaggccactaggattccactttgcatattaaaatggctattttaatacacaaaatggcatgaaatactatacagccataaaatagaatgagatcatatcctttgcagtgtgtcctttgttttttgcacaaagtgtattaggccactaggattccactttgtgtgttgaaatagtcattaccatattgtatcctggtaataaaaaagagccattgaacccttagagagagtttcacttctaatcggagcataacttggtgctggaaagaatcgcctcagtgttgtttaaagttaggtttttatgagccttcgttccataatctttctcacaataaatgaaaattggataacgttcttcaatagtcacaaaaatgtatttcttcaactttttaataatattgatcattataaatattaataagcacttattctgtgctgtgcactgttctacatattttatctgcattaactcatttaatcttggcagtcttatgaagtagaagctaatgctttcctcaaattataaatgagaaaactaagcatagacaagtgatttctccaagaccacacagtttctaatagacccggtggtaattttatccatctttttaaaatggaaaccttttgacatttgtgcaaccccaaccttctaatgttcaatgcttttgtttattcacttgtttttaaaaaaaagtcagtgattcttgaacatcttcatgcatattatgtcttttcctaacaacaaatgataatttatttcttggattcttacagggcattctacttggacaagtcaaattcaccaccaaactccacatccaaagctgcctatgtagataaggtaaaaacagatgattatatttattggtgctttttaatttaaatatctccctttttaattttctgctgttgttctgcagaaaaattaatctaagtagttttaatctaagtagggagctagtctgtcatgagacagtctcttctttattcatttatttatttttatcatactttaagttctggggtacatgcacagaacatgcagttttgttacataggtatacatgtgccatggtggtttgctgcacccatcaacccatcacctacattaggtcagtttgattttgaggatggtgtttacttatttattcaaatttacttggcactgaatccatgcaaaggattgcagttagtgctattggctgtataatatcataaagtgttcatgtcagagacatatgagtaattataaatattagtagtctctaattccattaccaccaaaatgaaataaaattttagactattctttaacagctataccaacctatctaaaagaattttactaaacatgttgaacttgacgttcatcactttttttctgcagctaatgaggcctctcaacgctttggatgaactttaccgactggtagcctcgtttatcagatccaagcgcacagctgcctgtgcaaacacagcttgcagtgcttctggggttggactgctgtcagtttcctcggagctgtgcaacaggctgggcgcctgccacatcatcatgtgcagcagcggtgtgcatcggtatgtgaccctcccgccttgcttgcctctttgttagatcacactttatttgatggtttttgtttacattttggcttcacaaaattaaaactggaaacattcaaagactcaggtgtatttggaataagattttggacgttttctggttttaattcttgtattttcagtgactatttagtcatacacatgaaggcaatcccacgtttatatcattgtggcccaaaatcttatcatagttgcatttatattaacatacttacattttaggaaaaatgtgcattcattttattcactagagaaattagtaggagctgactcaactagctcaacaaaactggttttctcactgcctttgatagacattccactattcagtggttccaatccttacaacccaagtgttgtgagctagatgctgcttggagttgcaactgctttctaactactggtgaaattatggtccaggagctcacacaatgtgctattccagaaaatgtaaggggaaatatttgatgggttcttttcaggtggtttatcttaggcaactgtggtctgtctgaggcagccaagatgacaggcataaaaattcctgtagatacatcagacttagactcaaaagtgcgagtgggaatccctgagtcaagtccctcttcataatcatatgctttcgcctctccattgccacaaggaaatagaatcaagtaaatggaaataatagaagagaattttagtccttgttttaacctaagtagggagctagtctgtcatgagacagcctcttcttctatttatttatttttatcatactttaagttctggggtacatgcacagaacatgcagttttgttacataggtatacatgtgccatggtggtttgctgcacccatcaacccgtcacctacattaggtcggtttgattttgaggatggtgtttacttatttattcaaatttacttggcactgaatccatgcaaaggattgcagtcagtgctgttggctgtgtaatatcataaagtgttcatgtcagagacataaaggatgaagagcaagcaaaaaaaatttcaaaaatattaacaaaaataacataatgataaaagtacacacacatacaacccacacaactcaagaacaagaagacataaatggtctaggaaagttacagctaaagagtaggattcagagggagaatagattttgagatggttagatatgggaactcttcatggaggaagttatgtttgaattgggttttcatggatcaggttatagaggcagaaaagaaagaaaggtcattgaaggcagcagcctctccatctgggaaactccattttgccttgagatggccacattctagttttcaggccctatgcagcatgaatgtgcttatgaaaaatggcaagagtatttctcttaaaagaacattaattttttgtggttggcaaaatatgcagaatttttcactttaattttcattgctgtgtactttgctctctattgcaagcttattaaaagcttaattgaaaatatcatctgtatctgagtggatcaaagctttgaaagtttttttgctagcaaagaaataatatcttccttctgataagcttatgttcatatggtctgttcctattcattcattcattattcattcattcaataaatatgcattgagcaccaatcatgtgccagcactgttctagttgctggggatacagcattgagaaaataaagtccctactttcatatagtacgtactgcattggggggaaacggttgataaagtaaacaaataaatatgtaatgtggcaagtggaataaatccaaaaaagaacagaattcaaaggaataaaggaagatagggtagagaaaagataggcagattgaattagcattaatatctagatattttaatgccaacctgaatcagatggggagatttgtgatgggggatttcataagatactggtataaaatgaaggagtggccgggcgcagtggctcatgcctataatcctggcacttccggaagctcaggtgggtggatcacttgaggaggtcaggagttcgagagcaaactcatcaacatggtgaaacctcatctctactaaaaaaaaaaaaaaatacaaaaattagccgggcatggtggcaaatgcctgtagtcccagctactcgggaggctgaggcaggagaatcgcttgaatctgggaggcggaggtcgcagtgagcggagatcatgtcactgcactccagcctgggcaacagagcgagactgtgtctcaaaaaaaaaaaaaatatgaaggagtgggtcatgcagatatctcagagaaagagctttgagaccatagggaccagcagcttgagtgtcctcaagtgggaaagtgcccagtgtgttcaagcaacaacaaggagtccattgtggcctcaacaaagcatcattgccttgagtatttgtgctcttctcttaccctggtacaagatcatagttatcagaggtctggatacactggaaccaagggggtccttcagactttgaaactataagcacctactcttaactccacattaatttactgaaaggtaattcagttaaatctgccatttaaccctagagaccatgactaagttgtccaaattattctctcagatgtttcacgagcaattttagaattagactggtattgtataagagaaatagtttgatgttccacagctattacttactatatgcattgattagtgtgttagaattacaagggacattgacattttaaaatactttaactttcttgatttctacaattcaaaataaatatagctttagttaattcacttcacgagacatttaccaagctgtagacaccaatgccccataacgggctgaatttaaggtcaaaactatttgaaaattgtatagttttatttttccattatgcagaataaaatggggaatgtaaaatactctttaatttttcagtgaaggctatgcttagatttttcatgatgataagatatgtgttaatttatattaggatccaaaaccccacagaaggctgttcctaaaatagtcctttagtaattctaatttagacagctagagaggaacccaagccagatccacacctgcaagcaacattaaacacggaggtttaccctcaggttctgacagaggggagaaattcacagcatgaatcagaataaatattacacaagccatttttatcatttacttttaaaatcaagatgaaaaaagtcaacaatgtggtatctacattagatagtcaatttcctgccttttctgatttcactggacagaaataggatctaaaatgttttttaaaactattgaattatattgtatgtgtttgatattaaagggaagaatgtggctttttggaacgaaaaggcagatttaattaacattaatgtctagatgttttaatgccaatctgaatcagatggcagagctacatacaacagtgaatatgagattagtaggaatagaattcaggatgaattattaaaatcttcccaaagcatttagcctggttttgtttttagtaaattatcttttctggagtactcttttgtgtgaatgacaagtctcctgagggacttccttgtctgtactctgttcaacttcgcttaagtttaatgtcatcattgctattaaatagtggcagaggagtaggctggactcttttgagttgatcagatttttgccttttaattaatataatataatttaatttttgctttttaatcgtataaatacgattaaaaggtagttaaatatatagaaaaacattctattctgacatcctttttcattttcattttgcattatctctagtaacttctatcaacaacacacatctgaacatagctttatatattatatatatgaattatatatttttttatatatttaataaaatggagacactatatatatgtacatatatatatatgtacatatatatagtgtcttcgtttacttgtgctgctttaagaaaatatgtgatactgagtgatagggtttggatctatgtccccacccaaatctcatgttgaaatgtaaaccgcaatgttgaaggtggggcctggtgggaggtgattggatcgtgggggtggatcctccatgaatgatttagcaccatccccagttttcatgagatctgcttctttaaagatgtatggcacctcccccccccaactctctcttcctcctgctccagccgtgtaagaggcgcctgcttccccttcaccttccaccatgattgtaagtttcctgaggcctccccagaggcagaaaccacgtttcccatgcagcctacagaaccatgtgccaattaaagctcttttctttataaattacctagtctcaggtatttctttatagcagtgcaagaacggactaacacactgagtaatttataaagagcagaaatgtattttctcatagttctggagatagggaagttcaagatcaaagtactggcagattcggtgtctggtgagagtcctggtttctgcttccaagatagtaccttgaaccctatattctcacattacagaagagcaaaagggagggaagagctccccaaacctattttataagggtgttaatccattcacaagacccccactctcatgactaaatcacctctgatacggtttggctctgagtccccacccaaatctcacctcaaattataatccccataaaccccacatgtcaagggcaggaccaggtggaggtaattggatcatgggggcagtttcccccatgctgttctcatgatagtgagtgagttctcatgagatctgatgcttttataagtatctggcatttcccctgcttgcactgactttgttctgcttccctatgcaaaaggtgcctgcttctcttttgcctcctgccatgattgtaagttttctaagccctccccagcaatgtggaactgtgagtcaattaaacctctgtcttttataaatcacccagtctcaggtatttcctcatagcagtgtgagaagggactaatacaacctcctaaaggccccatctcttaatattaccacattggtggtcaagtttcaatgtatgtatttggaaggacacatttggaccagagcatatagtttaataatctgccttatacttgtggcagattatatatatacaatatatatataatctatgctatatatacataatatatatgctatatatatatgctatattatatatatgctatatatatgctatattatatatatgctatatatatgctacatatatacatataaaatgagcacatagactttcttattctcttttttttgctgcataatatgtcaaccagttttataccacagttaactgaactattagtttggtgatttatcttctttctcaattgtatataagaaactacagagcggcatctttctatatgttactgttcaaatttcagttttattttccattaatctaaaagaaaaaagccaaacgcttagataaaatcactgtgtcaaagagtttaaaccttcttggttaatatattgttcaagttcccaaaagagttgtccttatgtacagtacaccagtggaatagatagttgtgaggcactttcacccttgccatgtctgcagaaggtattagaatccatttatatattgtacttctttactaggtttacaattatatttcctttgttctttaatttgtccttttttgatgaacaaataaaagtatttaatatcttttgaatttgtttctctactaagacttgtgctatttatatttatatacctgctttgtaggcagagttactttttgccaaattgaatctttttaagatattgagtcagcactgtggcttttcaaatttgcttttgtacccatgccagttattgtggcaggcaatcaaaaagcttgctaccgggatttctaaatttcttttatttcttattgcagcatccttccattcaatgtggtgttttaatttgttttggttctttattcccaccttgcttaatagctgactgtttttctttaaccaatataaatcagtatacagcaaacgttataaagcccgaagcatgagtctgactgagtccaaaaacaaaaatgccattttcctccctccacattaatttcggcctccttagccagtaggaacaagtgagtagaaatttgcagagtctacctgaaagaataaaggatatgtgtaagaaatttctagtgaaggtgagaaatctggaatcacagttttcccctgtaatgacttgttccagagagtttttgtcttcatattctcttcactctatatttttaaataataggttatcagtggtgtcagagtcttggtctggccatgtgggaaggtgggtgacttgagtgcaggcctcatgctctgaatagtgtgtgatgtcttcaaatcaaaacactggagatctgtatatatgagttaccttttatgttcgtgctttgttacgagggtgaatctattccaggaaacagggtcctggatttttaactagcataccctgtggtccatcatctggtgggggttgttagtggcaccatcaagaaggccttgctgcattggcatgggggggttcaccagcacccagttccaggtggcatcataaatatcctgtgtgcagagccccacacaggtgtgagagaaagtctggcaagaccagcccattaccattggtaaaacacataatagtgatgaccatcaagctagctactgtccatgtaatccagtgtcagctacttgaatatgtagtagaagttagctttaacatatgtaaagttttttccccaaaatgcacttattagttgatgatttggaaattcacatttaacaatgctgggtatattttcacatttaacaatgctgcagactggggttgaattaccaaggctctaccgagccttctcccactaacagaaactaatgtaaactgtcttctaactagaacacaggacctaaaatatgattataagcctttgttaaaaaagtgaaatttataagggaaaatataaaaacaactaacaagaaatttgtattaccaatatggttaatggaacagtttttatttcaaatgaagatgaacagtataatgactctcacttcatataagcaatgtaaaaaatcttattcaggtaactaccaattaacaatttaaaaacccacttttgttggaacttttaaaggactctttctttgtaatactttactctgtcattaccctgacatcaaaggacaaattgacaaataggcccagattaggtaatgcagaatctttttaaagtagagcattcaaggtaacctgtcttaggtaatcaaagagttcccatagggcagacgatgaagctgcatcttttctctactgcacaatgaagacgtattaagtaaaaggaataggtttactctggggatttctatatgtaggttagattctatgtatgggacagaaaaaataacttcaataagccaataaagctttcaaatgtatttatttactttttgtgcaaaaaagttttgaaagacccagtaacaagattagaggagacacctgaattttatatcactagagagatggctaaatagatcagaagaaatattttttctagcttatgacccaaggatattatatagtccccaaaagaagttataaaatatatgctttaatttccctttggaatggaaaaaagaaaatactacaataacaggggcctggcagaaagacaaaatatatcctactcatgataaattggtaccactatcttttcataaagataaatcttaattactctcagatgttcccttcattataaataagtacagaatttgaaattttactgaattgtaaaataggcaaataaacattaaaatttattctaatttagtcaactagataaataatacaggtaaattgttaaaattataggaagaaatcaaatattgtaaatatgaaaaggtttcattgtattacttttttccattatctgtaataaaagttctttatccttaaaaaggtggtcatatttgcatgtgatatcattgtctcttttttttttgcttgattaatatgtaccctaataaattagtatcttgaatcattttattttaaaatgtacataattttcttaaaattagatgtatggtgagcaaggattaataaatcagtatatgaaagggtgacatcttactgcagtgttttctagttctgacatctaggaatcaggtgatattggccaaacaggccttgcttttggtgcatataaaaggagaggatgtgactgcctttcattattagaataggaaaaacctaggtcctaatatgtcaaacatatgaatacataaatattagttatatttcatctgtatctgccaaacctaattttactatttgagcatattaataaaatgtatttacattactacattttatttgcatagatatagggataaatacagtatctactttaaaaacgattaaacagagcatagttctttgtatttgtttcaaataaactgaagttattgtcttttctaagtctaccaaattatattataaataaaaattccagtcattttctggaatatattactgagatctctgtggttaggggttttttaactactctaataagtgacgtaagataaattttccatcttccagaggagggaagaaatatgagtatctcaccgatgtaaatgattctcttttttttgcatgttaagagagcaggagagcaggtagtatggagggtgtttgtattagggttctctagagggacagaactaatagggtagatgtatatataaaggggagtttattaaggagtattgactcacacgactaggtgaggtcccacaataggccgtctgcaagctgaggagcaaggaagccagttcaagtcccaaagctgaagaacttggagtccaatgtttgaaggtaggaagcatccagcatgagagaaagatgtagtctgggaggctaagccagtctaatctctccacgttcttctgcctgcttttatttattctggctgtgctggcagctgattggatggggcccacccatattgagggtagatctgcctttcccagtccactgactcaaatgttaatctcttttggcaacaccctcacaaacacacccaggaataatactttgcatccttcagtccaatcaagttgacattcagtattaaccatcatagggctgaaggaaggaagcagcattcctgcctttgtgatgttgatcgtaaactggcaccaggtcttctaacatattcaggaagagtatagaggggtgagatttaaatacccaggaagggagtaaggtaacacctattttttttttttttttttttttttgagatggagtctcgctctgtcacccaggctgaagtgcagtggcacgatctcagctcactgcaacctccacctcctgggttcaagcagttctcttcctcagcctcccgagtagctgggattacaggcgcccaccaccctgcctattttttgtatttttcgtagagacagggtttcaccatcgtggccagacgggtcttgaactcctgaactcgtgatccacccacctcggcctcccaaagtgctgggattacaggggtgagccactgcccccagccaggtagcacatatttttaaagcataaccagccctcaccaggcaatgaaagtggagaagtaattacatggctggaggacacaggaaatcctgtgctcttgctgggtggtcaagtgagagcgaagcagcgctgagaggaaagggtagcgactggacaggattggagagtggaaggctctggctgctgggtgaaggatttgtgactctctcctggaggacttaggaagccaatgaagggttttcagctaaagaataacatgattagatattttttaaaatatcaacaaaaataaagattactatcatggtctgtaatgggacaaggacggaggcagggagacagcaggattttacagtattacagcaagagaagatgagacctagcaccgaagcactggcacagaggttagaagggaaaggctagttgtgaaaggtattaaatggcagtcaacaggacttggtgattggtgagaaataaagaataggggagaggtagaatgttcagtcacagttctgccaagggacagaactgataggacagagagagacagagagacgaagaagggacttactaggggatttggttcactgcactgtggaggctgagaagtcccaggtagggtgtctgcaagctggagaaccagggaagccagtagtgtggctcagtgcaagtcagaaggtctgagaaccaaggaagccaatgctgtgaaactcctggtccaactaccggtcacactccttggagataaagcataaaatttacccttcctcttcctttttgttccacgtgggtccccagccaacagcacagtgcccactcacattgagggtgaatcttccccactcagtccatggacccatatgccagtctccttcagaaacaccctcacagacacccctgggacagctcaatcattctaatcaaatgccacactacatggttttccctctcagcagaacgggctcagtgcctactgaagcactgaaaagaattagtgctttaccagctgtctgggtatctctgaatccagccaaattgacaccctgagtcaaccatcacagaggagtatcgatgattctgactcaccagcttggaaataaatcaggatggagggctgctacaggaagaaaatatgcattcatctttgaggggattgtggagagggtgttgaattaatctgttacactgtcccatgttgaattttagatgtcctttgatgggttcacagttgacaaggacagccatcaggaggctatggaaaaacaaatgtggaggtgattagtacagagagagaaagggggatcatggcagagaaggctttacagagatatatttgagctgggtttaaaacattactaagaatttttagggaacacactttgggttttaggagcaggtattccaggagcaggcatggaaatgtgagaaaagaataatcagctgccaactatagaagttgagaaaagaagaaagatccccaggcagcacatatagagtattaatattccctggtgcctcacatccccgtcctctgtgcaccctgatgaggagcctcagggtaaaacagaaggatccactcaaaggtgtcaaatgcacaaagttctgttcagtgtctcagataaacaggccaaagagaaaacaatgatggtccagtgtcaaagaactagagatatggatgaaaaccacatactagacatctctttaaagtatttatgtcatcagagaacctatgaggagtaaggacaaaagtcaaatttgagatctagagaacgaataagaatgttaaggaacttcgggtagctgggataggtacccaacctattcagttaaattccccccgccccacccaccaaaatagaaatgaaataaattggtgagttaaatccaggctgaagaatctaaaatgtggagaagcagattcaaacctggtggctgcagtatttaaggtcctaacacactgctgtttcatagtcatatacattcctggcatggcaaatacttctccaagtgcctaaatgctcattacccaaccccagaaggtccctaatcacctctatgctaacctccacaatgaaactgtgtcaaagaaaaacataccttactgggcttgcatagaaagtaacgagggatgtggaatgtttttgcttggcagttgatttttaagcttagtttaaagataaatgctgagaactttcagagaatgtactgaaagtggtaacagaaagtcccattatctttaatgagaatttcatggctaattccctgtgcagcgctgaaaatgcatcccttcaattcgattgcttttttgccctaagatagatggagtgagtggagtattcaatcaagttctctgtctgcggccaggatcagatcttacccagctgttacccatctctgctgcattctgttcagttgttttccacagggctactttctgcctgctttccctctgctttcttttcttttttctttcttctttcttttttttttttttttttttttttgagacacagtctagctctgttgcccaggctagagtgcaatggcgtgatctcggctcactgcaagctccgtctcccgggtttacaccattctgcctcagcctcccgagtagctgggactacaggcgcctgccaccacacccggctaattttttgtatttttagtagagacggggtttcaccgtgttagccaggatggtctcgatctcctgacctcgtgatccgcccgcctcggcctcccaaagtgctgggattacaggcatgagccaccacgcccggccccctctgctttcttttaagctactcggaaacccttgtcttccagctctcgggctatgactcatagaaacccagtatggtcataggaagatttcaccagggctgtccttttaacaagagtcaaatgatcaatcatattgaaagtatcaataaatattgggaggttatttctataaattatactaaaaacttgcctgagtaactactggcttttgaattgcagttgttagcctgtcattcaaagtttcctaaaaagagagtatcaatatttaataatttagataagagaacttgttgctgaaacaaatagtctgtttgtcctttttttctccacataaaagtatttatgtgtttcatttaaaagctgcttctgtcctacaggaaaataataacttaagtgtttgggtttttcacattctcactgacttgtagcaattttctcaagactttcgcttgtcaccaaagccttgctcagggtgaataaggtgggaaagtcaataggtcagctgcaggcttttccgtatttattttgcagtctctgcctatgtagaaccgtaccagcaacgtggggattgatcactatttaacagactgcattagtcctcagatgtacaattcttcctccatggaaggtaatgaaagggatcagggaaaatataactgcaaaaccttagagaattagtttcttcaatcaggtgcttcattctagctgtcaatagacttttcacagtcgtgctctgtaaaagtaattagaagccagaataaatgtgatcattacaggaatgaacacactttacacagaacggtgcattttatgcagaaaaacgcaaatctttagtcaagaaggaacacattctcccttcaatttatttttactacttgatcactttacaatcaatatacttacttagcatgcttcaaacctagtttctttttaatcatgaactcattttagtatgatgaagtctacattcacagtgaagtaatttggaccgcctgaattgatgtctacctaaacaatgcatttaaaatataaatgctgagaccaagcttgtgattgttgaattatggatgttgggagctggaattctctaaatatattaatctacgtgattcgaatttggtagagtagaaagagtcctagattttgagtccaaaaatgggttttgaatcctgttgtttactcgctgtgcaagggagtgagtgattatatcacctcgtctctcagggacttatcggtgaaatgggaataatgatttctttctcacaatgatggcttgaggattaaaagagcagcgtatgtgaaaacagctagtacagttctagcattcctgactactcagaatgttcagttgaaatctgtgcttttgatagcgaatccatcttctcgttgcagagctagaaatatgaagcacatctgttttaaatgagattgtagctactaaaaataggataacccctgtaagagctattaaaaatcacagtaatgggtcaccagagagaatgatagggaggactatgaaaaatacaaaagttacctcttctgatctgtaagaaatggagtacacaaagtgctgggtcctattagagctttgaaaaaggcattcaagagcagcagaagagctacccttgaattcaagactagggttttatttcctagtatcaaaacccaagagcgtatggcagaagacaaatgtttaggatttaaaaagaattagacgtttattcatctatttgctcatccatttatttgttgagcaagaaaaaactgtggagcactgctatgtgtcaagcactttgctaccccagcctgggagaccccaggatgcctgcctcattgctcttagagtctgatgtcttgacagtggagccgcattgggaagccagacagccgtagtatgtagcgtcagttctttttaataatgaaaatgaaaacaagaaaatatagtccaaggattgggttttaagtgacccaaaggagagcatacatacttttcaaactacaatggagaggaagaatatatacttgtcagtttgacaattctcttgaggtttcaaaaatgttcttctggcttaggaaaatatcgataaataaatggacaattttgatgatataacaattaataatataattactataagactaatcagtatgctctaatatatatatatttcaagtattaccataattcaaacagagttgttaaccagcagatgtggtaggagcaattgttaaaatccagagaaagggaaatgcaaggcaatggttgtgaaagaagaatcatatcccatttagaagccataagagaatctgtgatttctttttgctaatatttgttatctccataatgcattgatcacttcccagaatatatttaattagtgaacacttcttgagcaaatacttggaaagcacagagtataaataaataaatctagtcttttaggggcatggatataatatcattggcacacattaggaattcatgtaatttttaaaataagtatgtattgaatgcttactgagtcctaggaagtatgctagggtctagaaaagctacagtcaatatagacaccagtacagcctggtggtggtggtggtaatagtaatgaaaattttgaacgtttattgagaataaatgacacgctatacttcatactaagtattaggcatgaattatttcatacaatcctgaaaacaacactgtgtgttgtatattattattaaattcaatttacagaggaggaaacagagtttgaggagcttcagtaacttgcccaaggtcacacagttattagcaaagccagggtccaactcaggaaattctctctcttgcccatgttcttaatccctacaccttccagatgagtatgaaacagtggggtaagagatactgtgtgtgacttcaaccagggcatatgagatcatacaaatatgggaactataatagttgatgattgcagaaaactacaaaatcatttaagtatggagtagaagtgaggaaaacagggaccatattgtgaagatcttgtatttcatgtgaagaaatgggaagccattcctgtgtttgtatttatatctgcattttaaaaaggtcaccttcactatggaggaagaccaatggaaagaggccagacaggaaaggttagagtgaggttgcaccaatctaggcaggagatgatattagtgcagctagagaggagatatttgagagatagtataaaatatgaaattaataggcatgaaatgtgctgtagtgaatacattaacacactgaactgtacacatgcattaatctcttgtaatggatgagtttaatttgataacttggaggtttataacatttcttactctagacatgaaataagtcataaatcacgaaacaaagatttttggaaagcaataattttaatcctgaccacacccactctggcaaggttttaaaaaatgggaaggttgaggccactgaaaaaaaaaatcatggtacagaaatttgtaagaaattaaataacttctatgtctcatttcttttaacttctctcttatcaggttcagtgaatttctaacacatctgtttcatgcagatgcttattttttaatagctgaataaaatgggaccacgagaaaagcagaggaagaggtaaaaagcatttggaaactgagcagaatcatttacattcttccgacatgtagaaatgtatctttagttcaaagattgctgaaactaatctgcctcaggctttccaggatgggtgttcagatgttgctttggagtgcataaaatcacatgatcagcagtgtcttgcatctgtcaaaagaaacttctgagctgggcatagtggctcacacctgtaatcccagcactttgcgaggctaaggtaggaggatcacttgagcccaggagtttgagatcagcctggacaacatagcgagaccctatctccacaaaaaatttaaaaaattaatggcaggcacctgtagtcaggagcccaaggcaggaggatcacttaagcgcaagaatttgctgcagtgagccatgatcctgccactgcactccagccttagcgacagagcaagacctggactcatttaaaaaaaaaaaaaaaaaaaagaaagaaaaaagaaaaaaaggaagcttctgcttataaaaagagtggtgtacaaataggaggagtagctgctctgtgctatgttccagcttattgcatttacttagttctcttggggttgaaatagatagtcattattcctttctaaaatttgaatgctgcccaggtaacttcagcttagtaaagtatcatagacaaccacatctgattagattactaataacttgtttgctatagtcatttgaagaaagataatgacctggcttttaattaaagaaggaaaaaggagaaccctcataaagtaggggaaatgattttatgttctaagaactaaatctgtaagattctaaatataaatctatagctatataaagacataattcatgtgaagaatattatcacactagtaggtttctcagactgtccttatatacataatctctgaagcatgcattttccccaggggaattgaatccttcttgtttccatgactaaaagtagcctcatagttataaagaacagtgaaaatgaggtaatgtacgtggaagtccaaactaaaaaacaaaatcagattgtgatctgatttccccaattctacattggggaaaaacttactgatttcaaattcgttaacaaatattgatttccctttatatttagtacaaaatcaaactaacctcgataagaatgcaagtgaatcaaaatgagtttcaaggggttgtggctagtacacgctttctacagctgatcaaactattgtttggaccatgatcccttcaactcagaaaaccctgacatatgcctgtagtgtgtttgccaaatatcatagtttactaacaggaaattcttctgctaattataaaactgcactaacacaaaacaagatcatatttatccaagtgattcagttatgttattactgtacaaaagagttaaaaattgccgctcaagtaactttcaaagtctaaacgtgtatccctaaattcagtgatttcagtgtatttttctttcagggataatttttagtggtacctttgtaggtagcattattgagaactatagaagttttcaaattaatgtttaatcccaccctcgattttaaaaatgtggggaaataaaaagcatcccctactaaagtttacatttcccatttgtaattactttaaaaaatttttttactgcaacatctacataatgtcaatttatggcaaattgtcatgactacaaattcagagatcatcaccctagattttcatgtgttttatttaatgtgttagggtctgagtaaccctcctctactagtaatacaaacgtttggggttgccctcaggaaatagcaatataatgattggctaccaagcaaaatatcagtgttcctgacagtgggcatgctgagagctagcaaataaatccacagtaggaaaacagagctcagaaccataaacctcgaattgttctggtttcccatgtcatttcattgtgagttactgaatcacccttgtggcccaaaaggggtgttacatctccccaagtgctcaataaaatgcatttcagtccttgagagattggcttccttgctcagtacagtttgaatcaatattaaatgaggcagtagtaggcttctggaggttttgaaatatagaagtgattttagaataaaatggttgtttttgtctgcaatcatggtaaatgaccaactgtaggaggcccgtagttttcgggagactcttccatctaatttattatagaaatgttgagtcatcttaaaggggagggttaacatgtaaacaatatccactttaggtaagaactgatatcttaaaaagtgaactaaatatagtgtaatgaacctaagttagaaatggttgaaatacaatattttgcatgtaagccaatttaggaaacgaagctttgtctctcttctgagacttagttgaggaggggacgagagacacacctctggcatctagaaatacagttgggaaagaggagaccagttcacatgcttttcctgaccttcctttagttctacagtttgctgtgtaaaataaactcaagatctgatatctaagatcaactgggacaaacacctgaaaaaacaatcacaaagaatgttatcagcaaatgcccaaattatagtcaggtggactctactttaactgaactcagtttataaaacaaatattggcatacatttgtctttagcttcatgtaattattataatcttataacatcataatttactccaggtttactttaatgaacatctctttagaataaaaaggactataaaagtataaaccttttaaatagttgaaatatgatgagatctgagaaactgggatttacttgtgttccataagtttttccaaataatgtactgcccataaagaggaggctcctctacacagacttaccatggccaggtatcacactcatcacttcaggtcattcttgctgcactttatgaggtcagtacacctgctttcctcaattcacagaggagatgcagcctgagagtcgaagtaacctgggtacagctccataagattgtcttcaaacccttctgttgtcagctgccattttgcattcagaaagaggttccaagatagtttgaaagtagtgaaagacagagtaaaaatggacacgggattgggggaggggtttgtacaatggtactaagatgctgactgaaagcgtaaagaagttgtgggttgtggggagtaagtaggatactgggacaaggaagtggaaagtgggagaggatgaagagtttgcctttgaaaatgttgccactggaaagcaactgaagatgctttgaatacagtgttgataaactgttatggttgggtatttagtaaattactttggtaatttacgtaacaaattgttctttgatatcatgtaatgtaaatatagaaattgtttcatgtcaaccagagaagtaactaaacccactgactcctaaactctgatgcccaaggctctgcatgtttgagcccaggctgtttctccaccctcatctccttctccctgtgtgcaccagcatattgagctccacacttgttcatgagcagactgaaccagcattaacctcagggcctttgcacttaactctccttgaaatggtcttccccaaaacagtacctccttctgccattacacccttcgctagtaccattccactttctaatctgccacaacatttatttttatgggtaattcattcccacctgatattattcatctgcttctttattttctttgtcctctcactaaaatttgatcatcaaaagagatgggacttggctttgttcactgttgcatctcctacacactggagtaatggagacttgggggtctgaagttaagtaaaataaagtcagaaagaatttagaaaattacaaaacaaaataggaatgaaagttaatattcagaatgtaagaaaaataaattgcaatgtttaaaatctgataagtaacacaagcattataaaatccacaaaacaataatttttgttactttatgaactgccccacttctgtacattttttctccacaatttttgtaacatactctttgcctcttcaaattataacaactttgcaatataattttctatagagagaatagaacaattattttgtctcttctctagcatggatgatcaaatttaaattattaagagtttatcaaagctttccaacatcaaaatgagttattactaatgtcttatacatttttaaattgctgtcaaatgcaaaaaaagaaaaaacttctctaagtttctttagcatatgaaccataaatttgagagaatatgccacagagtagtttctggctgtatacagttccaacctttttgtcctccaaaacatatatatttcccatcctgggggcctcaggatatggacgtattacatcatgacctctggccctgcacctcttttccagcattggatgactgtgtacaatgggcagtaggaatactctggatgccatttctacacaggatactacaataacccagccataaatgaaagtaattattgtccaataaatacatcccagcaaacctagcctgactgcatcccaactcaactttctttagctcagtttcaaaaataaccacagcgaattgaacgctacccaacatgacagaaggaagctgggatgtaaagaagacagcagccatatcatttgcagttaaaacactttgcttttgcacatttgtaaaggtatgtgactatgtgaacacagtgctaaggcccctcccagaacctggaaagggattcgtacaagtaaggggccttgaaactcaagcttttgtagctccacagtaaatccactcctgccagcaacttgtggtatttgtaggtacaaagtagacaggcaaaaaatgtttggcaaaagaataaatgaaagaaagaatcatgcaataaaatatattttaatacatcttccttagcattgccaaatcaaagtgtccttcaaattaaatgggacccattagctgcaggttacatttatcatattaaaactgtgctaaatatctgagtgatttgggatctattctcagtacaaaatatgtcaaggagatttattaattataagcacagatatattgaccttttcctttttcttcctatggggaaattaaacatttctgtataactttgctaccttatcaagtgtgcttcctcacatgtatactctaagcagagccattacttggaatgtcttattactaaaattaaggacaaattttcttcattgcctatgttacaattgcctttttatgaaggacaaatactactctgtgacatttattttgttattttgtggcttctagggaaagaattgtgtgaatattttttaagtgatgttgtagtagaaggagctgaaaaattgaaggcctatattttccaggtggggtaaaaatacagatttattcacattttaatttaattgtaaaacagagttcatgttagaaagaagattggggcatcaggtaagaaactccatattgggctgggcatggtggctcacacctgtaattccagtactttgggaggctgaagcaggtgaatcgcttgagacaaggagttcaagacaacctgggcaaaatagtgagaacccatctccacaaaaaatacaaaagttggctgcatgtggtggtgtattcctgtagtcccagctacttggaaggctgaggagggaggatcacctgagcccaggaagtcgaagctgcagcgagttgggatggcgccactgcattccagcctgggcaacagagtgagaccctatctccaaaaaaagagaaacctcctattgaatgagggttcctggcctttaatatgctcctttctttcttttgcccagggctagcatatcaatctctctggtcatcagaaagtgcctgtaaaatcaaatgagctctcgtaaacctgactctgagtatatatttatgtaccactgaaaaactgtgtgccaccttcctggacctggagtccatcagagaatgccagcatcagaatgtttcttgcagatgctgattttttccacttgacattcaaattgagatagttgggagaagatgaaaagctatggggcaaataaaagtgacagaaaagtggcagcattcctgagaggctttctttcgcatgcttagtttaggaaaatagaattctgtgatgctgtgagtggaaatacaaggccttggttggacccatcaaccatgaatcaaattacaggcaatatttcccacctttatgtgattgcctctttttaacactttttacttttttaaaatttcaaattaattttgattcctaattggaagatacataatggttagcatgaaaagacaaataaaggtgcagccatctgttttggaaatgctggggaaagaaaatacacatatccatttgaaggtagatttttctcttaggtattgtgtgattggttgcaaatcacacttggaggtatcctttcagagagcatgttcctgggcacattcgcttcctgattgcagtaaaatcttttaaaatctctggcgggtaatttgctgctgtactgctgcttactacaacttgaggtagggagaaagagatcagtttaaaaggattaagaaaatgagtagtaattgtaggctgtctgttaaaaacccttcacagtgtgtaagtcttgtttataagtgagtacacgagagataaagcaagttctgctttatcagtaaagcacagacatatttcagcatgattagaaacagataaagattttttttttcctccccttggcttcattcagctggtgtaatgaacattattgattaaaatgttaggatcaataaagatttaaaatcgcagtggaggagaccttaatgaggagaaaaaggttatggtctgttgagtaagctggcagttaaatgctttgtagaaaactgattttttttttcttttttttgagacagagtctcgctgtcgcccaggcttgaatgcagtggtgcgatctctgctcactgcaagctccgcctcctgagttcacgcccttctcctgcctcagcctcccaagtagctgggactacaggcacccaccaccaccacctggctgattttttttttatttttagtagagacgaggtttcaccgtgttaaccaggatggtctcaatcttctgaccttgtgatccgcccgcctcggcctcccaaaatgctgggattacaggcgtgagccaccgtgcccggctgaaaactgattttttaaacatgaactattagttattactgcttggccttcattggcataggctgttctgcagcttgcatgtacatgtaagacaatatgcattgttatcggactcacacactaaaagtttgtgaacatcatgtacaactagaacttaatgatgaaacttgaacatgtgttaataataataatgaattctctgtaatgagaaagctgcctctcaagtcatttaacagccagtggaacttacatatttctccttagaatgcagttatatgtaatcacacagttgcactttgtgaggttctaatattacaacaagaatgttttaaatccctctctcctctttttcttaattatcagtatcaaaacctaccaaatgcattggacttctgtgttagagcctcataaccaagaacttctgtgttacagccacatagctaatgagttagatttttctcccataggaacaattgcattgtagaacttacttttatttgcagggaaaaatactagacagatctaggaaaatacacaaatcaacaacaaactacagggaggtaaatgcaaaataaggaaaaggctaattaaaatacaaatatgttggaaaaaacaaaacaaaatccattaatacaattttaggagaagcatctaaaattgacaatttttatgaaatgtatgcaaaagaacatttttttaatgttctttttagtgcatgcaaaagaacttttaaaagaacaattcagagaccagcacctgaaaatgttaacaaataaacataggctttcaggggtactgtagagatcaatgtggaagaacataaaagaatgccttaaagaaaaggaaaagaacagccgggcacagtgtaatcccagcacttgggaggccaagactggtggatcacgaggtcaggagattgagaccatcctggccaacatggtgaaaccccatatctactaaaaatacaaaaattagctgggtgtggtggcatgcacctgtaatcccagctactgctacttgggagactgaggcaggagaattgcttgaacctgggaggcagaggttgcagtgagctgaggatgcgccactgcactccagcctggcaacagagcaagactccatctcaaaaaaacaaaaaaaaaaaaaaaaatgagagagagagagagaaggaaaagaacaaaggctcaccagtcactaaatgctggttagaacacttttctcagggctactctgggtatcacatttctatataaccagttatgagttccttttggaacttttacaaatactcttttagattgagtccaataaacacattttaaaaaatcaattctgacctcagtaaatagtaatttcaccatttattcttctgctatatttgtgcacaaatcagtataaatctattaactatcaatatgtagaagttaatgataaatattctttagtcatttcttctaaattacatgaaatatcattaaagaataatatttagtaataaaacttgttctaattctggttatttaattttctgtagataagtgctcttataaacagttcatcatgggattgttttaaatttaaatacttaatttctgaccccagtaatgtacctttataatctatttttaaactggtattttatgtagaaaaatcagcggttcattgtctagctgcttttattttggctggtgctttctgattttgtgattagtaatccacatcatctcaaagggtgtatcaggaaccatggggtctgacttgcccttgccttggcccacaggtgcaccctgagcgtgacgctggagcaagccatcattctggccagaagccacggactgccacctcgttacatcatgcaggctacagatgtgatgcggaagcaggtaggtctcatgcagacttgggaatagttgttttgtaggccctggacttgaagattcctttgttcatttatcaggggtttgacaggtgcctggatgtgatggacagtttactaggctcagaggtaactcatgaatgagaaatgctcccagggagaagatgctgcaggcaccagtctgaggttctgcaagaatcttagacaactctggaaaaagggaagtctacggagtaatctgcaggcagaaagggacttgattaagtggaataaggcagcagtgaatttggcgtttatgtaaggaacaagtaaaaggacatatggcattttttggcaattgttagtggtaaagcttgggacggagaggcacagcagcaaggaagaagaggaaagaattgagggtcctgctggttcctggtgactgccaagggacattcatgccatactcagtgtgtacatttttctgaggataaagcacttacctcaaattttcctttaggggataaatagcagtctttggactgtcttcgggtcactggagtacttataggcgagaatatactctcttataaatagaatgaactttattctatacagtagcccaatagtattctaagtgtggcggtttagtttttaccccttaaagttattttaaaaaatgcactaagctccaatttactaattacaaaaatgtgcttaatctagcagaaaagatacttaacccacagaaagacaaaagaaatcaaaagacaaattttatattttattctaccaccttttctttgggctaccacaagaaattgatttaacaaatttctctacttgctgacttggtttgagttgcccagtgacagtgagtgatttaatttttaagatatgctttgtaattatgactactttgtttattggaagcttagtatctaactcgcaggcaaacgtataaatccctctgggcttgctcccaagactgcattgcagaatgccaacaaagcccatgaaaaagtcaaatgaagaggccaacatcaagctggtgtctgcaatgcgaagatcaggcacagagagagcatatacattggtacttctggttagggctcaggagctgaagattgaaaaccaagatcttagaaggtccaagagagatttgttaagtaaagtttagagctaagaagcatgtgacatactgtgggccagctggaaagggtaaacacagatgtgatttgccagcaggtttaggaaggactttttggatatatatacaaattatgttttaaaaagtagatccaggtaaacttatgattatccatatcagagttgagatttaggttgctaagaaatacagaaagaaattaattgcctttctgtttgattatgttgcttattagagtaggtttgtaggaagacttgggaagcgccatctcaaaattatagcttataacaagcaaaagagactggaatttactttcttttagaatattccactaaggaggtagttatgagttggtgacatatccatttctactgtatagatttgaatagtcataattatatcaaatacgttaacttcttaaaactaaaagtggaaatagagaaaatagagaaaaagtaatattgatattatgcaggccccgatgactcacttaactggtgtaaagctctttaaaatacaatgtaaattatatttatatagtattactatattgatgtgtattatgtaagtatagtataattttaaaactcaaaagtcatgggttccttataaaattcctgttggtatataaactgcactattttgtcagtatatatcacttgttcttcccccgaatttttttaaaaaatgaatacatttggaataggtagctttcttcctttgttatttgttcctctgccccaagttgtgacgtcccaatggctatgagaggcaaagaaataaggtttaacttctttgtgaaagatcatagagcctgggaccctctttaaggaaactgggcagactggtggccccagagatttcttcaacagtcccctgtcgcggattctccttccagtattcagcagtagcagacagtgagatttgtggctgagggccgatccacaagccaactcttacctacctatgatttgagcagttagaatgcaagtctgaatcaaaggtctagcctgggttttctctcctttttacctttcagcctcagggacaattacttaaccatttagactcagctttttagagaccaagtggttagatatttgtcatcttcagtatatattgaagtatttgcattattgcattatcgaatttgtattatgatatattagagaataatatttactattaaagttttataatatataaatgaataatataatgaaaaaactaagattttttaatttttttgggaataagaaactttttggcatgatttattgttcttatttataatttcaatattgttttcctctgcagattttgtgtatatacttttcatacagttgttattttaattcactgcttgagtttgaaggaaagataaaagacagaagagaagggtactatgttttcatgatgttttgctaatgattgtaacaaccttgggcagcactttatgtaaccagctgatttgattaataaaaggcttgcagtagaaactgtcatttacattataaatttaaagttttctatgccataattatatattcctagtgttctttgttggcagcctagaaagaaatgtcttctcaacctttttaataaaaactgggattccaaagtaccctgaccccttaccttcagtatcaaagcatcacactggacacaaagaacaaagacatgaggttgacatgctccttgccttatctcatggtggacacatccaaaggaaaatcaccatgccacgggcacaagcaagcaagtttctccagggtaaccaacctcagtgtatcaagactcagaacagggagcaaatgagtagactgagcccctccctggctgggataagtcttactcatttttgtatcacagtttcccaaaatagtacctagtacaaagtaaatgccaaaagatacttgttcaatgatgtcattctaactaggatagctggcgaagggcttcatgcagaagatgaatttatgatagcgattaaaagcacagaacaagtttttgttttgttttagatttaaggggtacgtgtgcaggtttgttacatggatacgttgcataatgctggggtttgggcttccattgaacccatcacccaaacagtaaacataatacccaataggtaattttttaatcctatcccccttccttcctctccccttttggagtccgcagtatctgttgtttccatctttatgtccatgtgtactcattgtttcgtccccacttaaaagtgagagcatgcaatatttgattttctattcaagtgttaattcacttaggataacggcctccagctgcattcaggctgctgcaaagaacatgattttatcctttttatggcatataatattccatggtgtatatgtaccacatgttttttatccagcccactgttgatgggcacttaggttgattccatgactttgctattgtgaataaaagagaacagactcttgagccaagcagccacagtttgagtcccaacttcagtgttggctatctgggtgattcagggaaatattcttagcctctttggtacttagtttcctcagtattaaaacaaggataataatagtacttgtttcatagtgttgttgtgaggattaagtaagtaaatatacttaatgcacttaaaacagtacctgatatggtgagtgaccaatcaattattaactaagatgatatttgatttggaacttggaagataaatatgatgcagacaggaagggaagggggcaggaagagtatttaagtagagggagcaatggcaatataagccagagtgtaaaaatgcaagtagctggagtgttgagtttatgaacaatatagatgaatttaaagtgaaatgtgccaatagatgtcttcagcatgacaggcatcagttttatttgaaaccttttaagaaagcaaacatttttcttaatggaattacttaatggaaatattataagactatagacaaaaagcattgtacacaaatacgtcacactagttagtaaatctatttttcatagggtatgatttgggaattctaaaactaatttatgtgttactaagaatgaacaaataagatattgtaaataacgagaggctgatttctcactgtctgagaaaaaagttacaaataaggaaaggaagaaggctggaatctggaatgaaccctatgctactggattgaaatcacatgtatcagtatgaactgataaacacacacacagacacatacaaatatggatagatgtagaaatgaatatagatgtatgagtatgtttgtatttgtaaattagccctgcctgctggaaaggcctagttgcagttattctcccagtagtaaagagcacactcagtgtccaaatcctggcttctaaataccattctccaataaaaatagccagggccccttggagaattggtaaattaccagacgagaaaaggaagacaagtctggaacatcttgaggtgccagaaaataaaggagtactaaacaatgatggcagcatattaaaaaggcacaagaaacaaccagaaagagctcccagtggccaaaactgaaacaattttagcaaaaaaaaaaaaaaaatgtatataagtattagattatagctcatggaataaaataattatgagtccacactgatttaaatatatacttgaataaataaataatttggggagaagagattactctttttaacagaattccagtccataaacaaaacaatattattgttgtagacaagatacaccaacagaggctaaaatcagtgagtgaaagtttgaagagaaacaggatattgcatagtctcatagtatgtccctaaatatatttattaactaccaaaaaaaaaaaaaaagcaatgagtaactttacagttccaaaatctggcagacaccagagtgatcaaagttaatgtcagcagtgataagatatgccaatatcctgtatcccctgataggatgcactgagaagggcatgacatcacttctgtgattttcttgcccaaaatgcatttcctgaatctaatcgtgagcaaacatcagacacacccaaactgagacattctgcaaagtaatgaccaatacttatcaagagtgttaaggtcatgaatggcaggaaggacctcaggaactgtcacagattggaggggagtgaggagacaggtaagcgaaaggcaatatgggatcctaggttggatcttggaacagaaaagaaaccttagtgggagaaaatggagaaatttgaacaagatgcgcagtttagttaataatattgtaccaatgttaattcttggttttagtaatcacactgtggttaagtgaagggttaacgttaggggaagccacatggagtatgtgaagtctcagttctgtctgtattttcctctaactgtaaaattacttcaaaataaaaagttttctttgtttgttttgtttttgtttttgtttttaaagactggttaagtgcagtggtgaaaagtggggaaagagcagaataatgagttcaattgatctgtaactgcctatgaacagtcaattgagataactgcttttggattagccaaaatagtttttcttaagtccttaattgcaattcctaaatttcttcccacctttctttttgatgccacacccccttccctttttgttttcctattttacgtcctttttttccttctttttatcttgtttctttccttagttcagaatgtagtagctggagagagagtttcagagtttttgaacaactctccttaaccttttccaaaccccaaactctggtctttggtcctatgtagtaagtaattttcatgaggaactaaagtgattataacttcattcatgaacttgacagactgtgaagaatttttcattgccaaagtagattttattataaactctgaggcagaattactagatttcatagatccacatggacatagcttgtccaaaaattgctttagaattagttaagaaaatatttttttgctaaatactagtaaacttaatgattcacgtaagagaaaaacaagtgcacaggtgtgataaaactgtgatttctgttccagaaaagtggtaacacagagcaggtatttatagtctgctcatttactattttcaacatctttttaaaatttttatttatttttattatttttctttttttgtgagagagggtctcattctgtctcccaggctggagtgcagtggcacaaatatggctcactgcagcctcaacctcctagcctcaaacaatcctcccactttagcctttcgagtagctgggaccacaattgcgtgccaccatgccaggctaatttttctattttttgtagagacagtctcactatgtttcccaggctggtcttgaactcccaggctttagtaacccttccacgttggcccctcaaagtactggaattacaggcatgagccactgtgcctggcctgttttcaacatctgaagtattgatcgcttctaggaataaagacaaagcaacataacagcacatttttattaaaagcattgattttgaaaatgctcagacagcgatgtctgtctttctcttagctaatctcccttgatgttaaagacattgtttgaataagtatcctacacacaaatgttaatggtatgttaaattattactaacacactgtagggataaaagtgattttcctgactttcagggagcaagagttcagaacacagcgaagaatttgggagtcagagaccggactccacagtctgcaccaaggtaagtgcatcccctgctctgcccttgcccgaaagatttgccagcattttccccggcagtgtccctccgctaatgcaactgatctaggatgtgccccgtgtcgtactgattgtaattacggagattgcccttgaaacacccaatggctaagtgtgattacacataaatgctctcagaaataaatggatattattctattttgcaagttctatcatgaattcaaaaagaaaagctcacacaggcactactaaaatttgaaatccagctgtgacaaacaaccaaaatcccattttcatttatacttctgtgactgtacatgctgttccttctagcatgattgcctttcccatatgtgtttgtatgggaaactcctatttcactgcaaaacctagcttagatgtcattttctccttggagtcttgcttgagtttccttcctctctgtactcccttagcatcttgtcatattgacctatgataaactttatcatatcttgttgcaatatattgtcacatgttcctttgcccaacctgaatatgagttttctggaggcagacatatttctttggattttatttattactcatcatagtaaacatcaaatatctgaacagatttcaagaatgtctgaatgaagaaatgaatgatttttttgtttgttatctcaaatatttacagttcacacagcatcataagaaaggactcccattgactttaaaataaatgaacataaataatagccatcagagaaatgctctggttttgatttccatggtgatgagggtaggcaaccatctatcttgaaggggggaaaagctaaataatttcttttacatttgattgcttttacacatttgtattttaacaaaaaataggttcttccaaacactgccacagacaaaacactatttctcttcacagagcagttaccagagtgggaatgatggctgcagcatttgtgaagtaatatttcattctgaaaaaccatgtgggtagtgaaagtgctttcaaagggaaaacaaactttccccctgaaaatggttggagcactcatgttctaattgcacttatttttagtgctgactcctaactctatttaatcctgaagttttgttaatgtcgtctttcacagttgattaggttagaatctaaggctttatattgctaaaggattatttttacatacgcattctccatccagcaccttgggaagcatggtatatttggataatagacacagtgaagtgttaaaattccaataagaaaacaagaccatggctcagcaaggtgttcttagctagacctataaacactaataaacagtggggtggggatcagacttcagcaccctgatcaataaaccagtgtgcaagctgaacacaagaaaagaaaccaatgaatttgctttttttttctttattgggtgatgttaaaaacactcgaaacgaaacgcaataaaatgagccagccataaagttgacattatctacaagggagtaataggcctgagagagaaagcaagagtaatttttaaaatttattttatttattctttcattcattctgcaaatatttattgaggacctcagaaaggcaaagcactctgctatgccccaaggagagtataaacctgaattagaaatagccctggccttcacacagttttcaattaaaaagatgaggtgaagaaggcataattacaaggaaaggtagaaaacaggaatcaccctgtggaggtgcagagagaatgccaaggagcacagaggggctgggatctctcccagctgagaacctggactccagccccaaagacagtgagttgaagaacacatttggcagtcaaggtaaggccatccatgagcaaagctgggataatatttctggaaacatcttcatggagctgaagtttgctatctctgtctgagatactatttgattcaaatcctcaacccatccttatcagagttttcctcttaccatcctcttgatgttttaaaaaatttactcagagcaatatatattgaatttctgctgtgtgtctataggtgtcaggccccttgctaaaagctagaggactaaagacatagtgtcctccaccagttgttgaagtgtagtagagtaacaatggaatagaatgaaaatagtacacagtgtgtgatggaaataggcatagattgtcatgagaacacagagaagggacataatgcagcctaaggacagagagaagaggtcacagaatactcctgggcctgtcagcattgtaatgcatgtcccatactggggatgcaatagtaccaaggaccacagaagaagtgatgttttgagaactatgcctagacctcaatgactggtggttgaatgtgattcaagtatgatgtgaagtgagactggagatataggcagagccagatcagagagacttgtaggagtacagaagagttttgattttaacttgaagacaatagggaggcatgcataagcaatcttcttatctggatgatttgaattttagaatttatctctggcaacatcaggatggcccatttgaggacagctaagactgaagcaggagcttaaaaaaaattatggtagtattgaggccaagatgagaggatgggttagagagatgtttagaagacagaatcaatagtctttggacacgaattggataaaggagtttagggaaaaggaagattctaggatgaccaccaaatctttggcttggataatgaggtgctggtggttctttcctctgagacagtgggtgcagaagggaagcacacgcacagggaggagatggggagctcagcttggtagtactgagattgagcatcaatcctcatccctggagaggacattgtggacagtggaatacactggtcaaaagccaaggagaaaagaataggtggcactatagtcctatgagtcttcgatatttaggaggtaggtgaagacacagaaatcaatttcgttaccacaatgtatgggtaagagagatgagaggaggtcaggacaccctcaggggatgtcagcactgaagagaaagcagaggaagcggggctacagaatctaaagtgagctatggactggagatggttgttctggcattcaaaaaggagcaatcagccatagtcacactactgggagagtgaatataataatttaggaaatcagaaagctagaaagcagagtatcagagtttaataattctgaggtaggtaaagcctgctctggggagggatgtttcttggacacccttggctccagtgcattttgcattctcccctttagcaatctgtgtctaaaagatgtgtccagaactgcttttggcattggggtattaagatggatgagaaatgcttcatgcacacaaagagcttgcagcctagtagaatacaaagacaaaataaatttagagaatcaagtgaatgggtattgtgataggatagaatgggtatgtgaggagggagtacaaaggaccttccccctcccagtttcatacattggctcttcagggcaataagcccctcttcctcaggaaccaaatattacattccagatgtcggattgcacatccagcacactggctttattcacaaaataaagtttttgttatgtcacaaaatctggaactcttgtgctatgaaaacctgattttctttagttgcttaaccaggagggggcattctatggtggttaaaagtgaaaaaaattggccgagcatggtggctcatatgtgtaatccctgcactttgggaggctgaggtgagaggattgcttgatactgggagttcaagaccagcctgggcaacaaagcaagaccctgtctttacataaaataaaaaataaaaaatggccgggtgcataggctcacgcctgcaatcccagcactttgggaggccgaggcaggcggatcatgcggtcaggagatcaagaccatcctggctaacatggtgaaaccccatctctactaaaaatataaaaaaaaaattagctgggcatggtggcatgctcctgtagtcccagctactcgggaggctgagacaggagaatctcctgaacccaggaggtggaggttgcagtgagccgagactgcgccactgcactccagcctggacgacagagcgagactccgtctctaaataaataaataaataaataaataaataaataaataagccgggctgtcagcctgtggtcccacctactcaggagacagatggaaggatagcttgagcctaggaggttgaggctgcagtgagctatggttgtaccactgcactccagcctgggagacagagtgagaccttgtctctttaaaaaaaaaaaaaaaaaaaaaaagaaagaaaaaaatggctatgtgaccttatttagtcaggagttttttcctgtctgagactcagttttctcatttgtgagatggggagaattgccttgggacagagtatgattgatgctttaaccttgaaagaggcgtttactttgtggtctcctttcacataacttttattgttgctgtgtagaaaggtgaaaggcaccatctatagcatgaagttggaaagttctgctcttggcagatttgcatttggtcaggaaatttgggctacctaagctcaaacaagaataggagaaagaaggccaaaaccaaacctttttcgtgcagggccactgatgtgagtggaatgagtatacaaaagggccccaaatgatgggcagaccaaggcctcactctgcccaacaatttatctgccaaaaaataaatgcttcagtgcaggatgagagtccaactcttatctattacatttcacttagaaatttcatctgcctgttctgaattcccaaagctctgcaatttccattacaaatgtgtttgaactcaaataaccctgagtcacttccaagaagcctcacagaagctgaatctgcccagttccacaatacccagagctactatttcattaggtgatcacagggagtagccttttcttcaatttgcaaggcagacatttcaaagactcgtctacccagtgctattagttacattatgtggaaacgtgctgagcaaggtaatggaatgtgatggctcagcccagatctcatggggcaggacattcgccacccatcttcccattcctgtcctacgtgactaagcccattgtgccttttcaatcattaggcagcactgtcagggaaataagaggaaggaaattaagaagaaaaagagcctccttattctttcattatcccagccctgggaatgcaatttgaagtaacaccatattcttcagctgcaaaagaacctttgaagtacatcgggtcaattgaatgtattcttttctctgggaaggatgaaataagcttcttctgatgtgagatgtctttatttcagtattcgcctcttgcatggagataacatgtagtgcatcaaccaccttccggacttgaaatacgttttgcaaacctccaaagtcaatagcttcttctacgtgttctcatactaggaaagctcattttgttaaccaggagttataactcaatattaaatcttgctcattcttctcctttgatctataattagcatgggattaaatgtcaaagggagaggaaatttataatttgctaaattgatcaacaacattgaagctgttttcaatttagtcggtaaaagggattgaatcaccacagctagtcctgggttgtagtgcttttatgaaaagatgccatgtggaaatctgcagcttatattttgacatcattagtccaagctctgcagtgggaccagtgtggccatgggtgctgtttagagaacaggcatagtccaatccagcattttagttcagccaatcaatctctcaggatatgctgtaagtaatgcatatggccttgagatcactagggctatcaagcagctattatgttcccttttactttggttctgctgttatcatttaaccactgaggagaatgtggagcaccagaacttgtgtgactgtagttaaaccccctgagttttccttgaatgctgtatagtcttgaacaactagacctgggctgcaatgggaagcatttccaggcatctccatggtttaagtggatccttgacccacttggagagcccacagtcgggtgagtgggccctgggcagagctgggtaatttatccttaggaaactctagccaattgtgcctttgagatttaggtctcaggaatcttgccatgtcagcctatccacagccaactgcctgcactctggggatagtacaaaatgaagcacatttctaaaacaagtcagaaaatcagtagacccctatgagataggtgccatcttgctacaaaataatcatgatagacatgatggtaattatcgtaataatatcatttcctggaatccagagagtaggttttaccacaagtgtggtgaacagcctgggcggttggcactggagaaaaagcagcaggtttaaaaaattcaggattgcttcatccacagccatcaagctatgcagaggggcactggtgttttctaaatcatagaatctaatttttggagtttgccaattcatatgttatttttagtgaaatagacaatccgatcatgactcacgaaacaagaaattatcatcaatgtatttgttctaccaataaagacacctcatgtaatggtaatacactaagtcactgtcaaactttaccatgccttttctaggctgtacaagctgtgcgagccacctcccccagctggagaagaatgaaaagaactcccaagaaaccaggcaggcagaagctcctgaatgctggactagacaaactacatgctggctaaacattctccactgaagatacatcaatgcttttttttttttttttttctgtaaatctttcttcccattgcaaacaagtagtgatcagtttatactttgatatttatgctggaaatggatagaagttcaatcagacagttcagcttcacgaactgatgtctctctgtattgacattaagtattcacttttagagtaaaatccatccctgggacataaagaaaaaaatattagagatttaaaaattaaccaccatgcctcctctcccattcattaaaatggttttattatgcttgttttcatctttcccaagtggacacatgtaataatggagagttcctcttgaagaaggccattattgaagaaacctggcgacatcttgcaaatcatcatgagaaatgttaaggacaatatagactgtgtgctatacattttcagtcccccatttgcaagagagaaagtcattaatgtgtaaaaggtggaaaagcatgctttactctttctcctaagggcttatttcttattagaggagaagaaagctgactaaggcctccagcaggactcactttcttctagctgcctttgggcatcctttattcttgtggacagctctagcaaatcaatgctatttttggtgtctgcaatgggcatggcttccttgggccatgaatactgggtgctgtgccataatggccctggggatcagggaagtgccctggggatcagggaggggaaccaggatgaagaaggggactgtaagcctattttaaaactaaacctttaagttagatacctaaaactcttaattttatgaaggctctcatatgagaggtcagactcttcaaagaatacaccatcatgagaatttttctgttaagatatatgtataagatatgaaaataaggctttatttgtactttaaaaggaccattaggggacttgtaatttatggatgcacaaggtgtattctatccttattctaaagtatatgtaatgtttatcatttctacattgatgcaataacattgtttttagcatagtaaatacagtaatatttattttaaatccataattttcaaacaaaacactcatgattgtgataattatatgggtttacactaatttagtatgtgaaatgcttttcatatctaaattatgtgcaaactattattaattttttttttattttttgaaactgagtctcactctgttacccagactggagtgcggtggtgcaatcttggctcactgcaacctccacctcctgggttcaagccattctcttgcctcagcctcccaggagctgggactacaggtgtgcaccaccatgctcagctaatttttgtatttttagtagagacgtgttttcaccatgttggccaggctggtcttgaactcctgacatcaagtgatccacccacctcagcctcccaaagtgctaggattacaggcgtgagccgctgctcccggcccattattaaattttaatttatgaaatttgatcatggttttcaaactattattaaaacctttatcattaaccattatgtattgaataacaaaaataccatgttacatgtgtaggcaatatgtgtatactactaggtaaggtaataataattgttatttggcacagaagctctagagacttttgtgagaagtccatatacaatgaaaaacaaggaacctaacatcagagttttcaagaaaaagttgaacattttgaatttaataaagatatctttacaaaaataaccttcaaggatttaacatttgtaggactaataaaggaacacagctgattagctagcaagtcctttcatattgtcatgactgttttctcatcaccatcctggaagcagtaatcctcattagaggcatgcaagggtcattaaaatgatttcagttgtactttgccatatttcatggggcaaattatcagctatattgtggtatttataagctaacatattttatatttttaaggatctactttcttcataatagaatgtgcttcatattatttttagggttaaacataagtgtgtcgtctatcatgcttggtacttactaaatgctcaaaaattagtaatggtttatattaacatcagtcactctaacttctgatcgtaataaaaattgtagctaacacttattaagggattactacatgcccagtaatacctccaaatgctttgcctgtattctgttgttattgcttcacttaatcctcataataatgacgtgacatggtactctttttatcgcgcccatttttagagttgaggaaccaaagcaaagaataacccagtgacttgcacaaagtcacatgattgacaaggggtagaaccaaaatttgaacccaggaagtctgccttcttaatgcattatgctacttttccttatcaatataagaactattgtaatattacgatgatttgtaagtaaatttggagttatgcggcatgtgttgtcattaaattatacatcttgtattaaaaaacggtgaatggaaatatgtaaatgctttaattatatgaaaattatagtgtttcctggacattttacaactctggatgcacattttgaggtaactcaacagtgttgcaatctgaaaaactattttgtatcatgttcctcctcatcaaaaataacagttttagatagtacatctcatacaactttattataattccaacacagtggtaagagtaaaaaacatacctaatggatgaatgaataatgacaattttttcaatgccatatgtacctccatgtgacattgggcattaaatggccttgaggttttaggatgtgtttaccttaatgctacatgctcctctataaccagtgcaccttcaaatactgctaaaacctgctaacttcttccaggttctgagcatggacaactcccaccgccccccaacctaaaagggaaggtcatgcatagaaagtttggattttaacttagagcctgactcaaaaataattgtgaactaaaaattcatagtcaatactttttaagcattctattatctcctataattttttctaatattaattccaaatattacaaaatttaatgtgatattaaagtatgaaaaatttgagaagattagaattttacatgtatttccattcctatttaaagagagaaaatggggagataatttctgtgctagttttacttatattggatatataatattgaatttatagtataagctttattttatcagttcgcaaatctgaagtctcatactgttttctgtctctttaaaacacattcttctggtggcttgggttcattgtgcattcattggccactcttcttgcagcaaattttaaatcaatttaaaattaggtaaactttttatgatactataatatatatgacgttgaaatgacattggttctgagtctattaatttaaattttgggatcattacattcagaatgaaatgaaattgaccttgatactaactttgagagccaaaaaaatgaatgggtaaacaaagcctaagggtcatgttaattaatttaacaaaacacactattttcagtcctttgggcttgtacattttatagacaaaattagcatttattgatttggaagtgttcaattgagagtaaatgactctgtgttttgaaactaatacctttgcatttaaaatactgaagtattattttgttagttgaagtagatcttttcctgtgtagcatgatttaatgcagagcagttattcacttatacttctgaaaagccataatattgaaatttaaaatgaatagtcttactttaaggtcacttttactttacattttactttaaaggtcgtttttagactttatgcactagtggtattgaaactaacctttaatatttgactaaattgcttgtgaatttttggaaactatgtttaatttttaaagacattgttatgtgaattatcttactaaattatcacttaagaactctctcatagcagtaatttcattaacacatatatatactaaccaatttgctaaatgaattgtcggttgtcctgcttaagtactagggccaagctttgaaatgacttaaagtgatctgaattttgattgagaaaaataatctgtcttctatcgaatgtttgaacttttagtgatgagcctcagcaaagtttcaccctgcatgaagcagaacaggtgggggaaaattacaagaggggtctgtaccaaaaatggccccaggatattccattgaacaaatattgaccattatcttcctaggtgctacaactccatactagtcattacagttagtctttggtttcacaaaaatgttactgcttcaaaggcttggcattaacagtgatgtcagcattcatactgccaacagtctgctggtcatacatggaagactgttaaagtgtggtctgtgcatcattgagcaattgttatgtagacacaaggcctgcttcactcaggatagcttgctcctctcgctgagaagagatacctgaagtctactaagtctactaaatgagatatatatgtgtatgtatatgcctatatatattattatatttactagtgtttgtgataatgttataaaaattaataagattgttttgttttgtatgaatgtacgacaaatcgagttgttcaatgtattgaatgaatgtcttactgtaagcatcagagctgtccaaccatgtgtaaatgtacctggtcttttgatcactgacactactttgctgttcaatctggaatcaactgacctggctctgccattgagaaataactaatttagaacaaaattgttaaattttaaaattgcatttctactagaaatagctttggagagttaattttaggtgcgaataacaggataaatgctatctgatcaagcattcatccgaaaaatgtttttgagaattcactttgtgcaaggcacagctgttggggctggtatatgtgtggacatgtttgatttgaaaaccttcgtggtgtctctgtgtgttattgatcatctgtgtcttttgcttaaaatggcattatttgtaacacaaacctaatatcatatatacaaatatactgtatgtagccaagaacactaaattggtaagacgctttagaaaatatgcatttgaatttgaatatctaatgcctcaagcaaatttattagagtttcagatgaatatatatttttgtaactggcaagtaaatgagtagaacagagctttttgcctaaaaatatttttataagtactcatttacatagtattcattattttaaatgacaaaatatcaaattggttctataaatagtaattgttttgaatatatccatacttaacatcatatgaaatccaaaatttaatgataaatttcatctggatgtgttattataaaagtcatctttttttcctttgtaaaatatccatgtaacatagcttccattactccatattttatgtatggttaaaattatcaaagtgcattatgtattttatattgtttatttttgtctgcatgaaagattggaatagtaaccgtgtatgataaatgttaataataataaagtgaaatgtagtatgaccaa